(19) United States
(12) Patent Application Publication
(10) Pub. No.: US 2012/0015942 A1
(43) Pub. Date: Jan. 19, 2012

(54) SUBSTITUTED HYDROXAMIC ACIDS AND USES THEREOF

(75) Inventors: Emily F. Calderwood, Framingham, MA (US); Dylan B. England, Milford, MA (US); Alexandra E. Gould, Cambridge, MA (US); Sean J. Harrison, Belmont, MA (US); Liting Ma, Needham, MA (US)

(73) Assignee: Millennium Pharmaceuticals, Inc., Cambridge, MA (US)

(21) Appl. No.: 13/184,595

(22) Filed: Jul. 18, 2011

Related U.S. Application Data
(60) Provisional application No. 61/365,524, filed on Jul. 19, 2010.
(60) Provisional application No. 61/426,243, filed on Dec. 22, 2010.

(51) Int. Cl. A61K 31/166; A61P 35/00; C07D 211/34; A61K 31/4465; C07D 401/12; A61K 31/4545; C07D 207/34; A61K 31/40; C07D 401/04; A61K 31/496; C07D 471/04; A61K 31/4375; C07D 309/14; A61K 31/351; C07D 295/26; A61K 31/5375; C07D 413/04; A61K 31/5377; C07C 259/10

(52) U.S. Cl. 514/235.5; 562/622; 514/575; 546/205; 514/319; 546/194; 514/318; 548/537; 514/423; 548/558; 514/426; 544/360; 514/253.01; 546/122; 514/300; 549/424; 514/459; 544/159; 514/238.8; 544/131

(57) ABSTRACT

This invention provides compounds of formula (I):

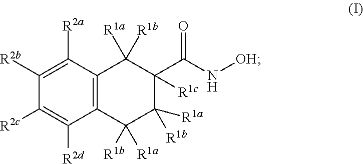

wherein R^{1a}, R^{1b}, R^{1c}, R^{2a}, R^{2b}, R^{2c}, and R^{2d} have values as described in the specification, useful as inhibitors of HDAC6. The invention also provides pharmaceutical compositions comprising the compounds of the invention and methods of using the compositions in the treatment of proliferative, inflammatory, infectious, neurological or cardiovascular diseases or disorders.

### PRIORITY CLAIM

This application claims the benefit under 35 U.S.C. §119(e) of U.S. Provisional Patent Application Ser. No. 61/365,524, filed Jul. 19, 2010, incorporated by reference in its entirety, and U.S. Provisional Patent Application Ser. No. 61/426,243, filed Dec. 22, 2010, incorporated by reference in its entirety.

### FIELD OF THE INVENTION

The invention relates to compounds and methods for the selective inhibition of HDAC6. The present invention relates to compounds useful as HDAC6 inhibitors. The invention also provides pharmaceutical compositions comprising the compounds of the invention and methods of using the compositions in the treatment of various diseases.

### BACKGROUND OF THE INVENTION

Histone deacetylase 6 (HDAC6) is a member of a family of amidohydrolases commonly referred as histone or lysine deacetylases (HDACs or KDACs) as they catalyze the removal of acetyl groups from the ε-amino group of lysine residues from proteins. The family includes 18 enzymes which can be divided in 3 main classes based on their sequence homology to yeast enzymes Rpd3 (Class I), Hda1 (Class II) and Sir2 (Class III). A fourth class was defined with the finding of a distinct mammalian enzyme—HDAC11 (reviewed in Yang, et al., Nature Rev. Mol. Cell Biol. 2008, 9:206-218 and in Saunders and Verdin, Oncogene 2007, 26(37):5489-5504). Biochemically, Class I (HDAC1, 2, 3, 8) and Class II (HDAC4, 5, 6, 7, 9, 10) and Class IV (HDAC11) are Zn²⁺-dependent enzymes, while Class III (SIRT1-7) are dependent on nicotinamide adenine dinucleotide (NAD⁺) for activity. Unlike all other HDACs, HDAC6 resides primarily in the cytosol, it has 2 functional catalytic domains and a carboxy-terminal Zn²⁺-finger ubiquitin binding domain. HDAC6 has been shown to bind ubiquitinated misfolded proteins (Kawaguchi et al., Cell 2003, 115(6):727-738), ubiquitin (Boyaullt et al., EMBO J. 2006, 25(14): 3357-3366), as well as the ubiquitin-like modifier, FAT10 (Kalveram et al., J. Cell Sci. 2008, 121(24):4079-4088). Known substrates of HDAC6 include cytoskeletal proteins α-tubulin and cortactin; β-catenin which forms part of adherens junctions and anchors the actin cytoskeleton; the chaperone Hsp90; and the redox regulatory proteins peroxiredoxin (Prx) I and Prx II (reviewed in Boyault et al., Oncogene 2007, 26(37):5468-5476; Matthias et al., Cell Cycle 2008, 7(1):7-10; Li et al., J Biol. Chem. 2008, 283(19):12686-12690; Parmigiani et al., Proc. Natl. Acad. Sci. USA 2009, 105(28):9633-9638). Thus, HDAC6 mediates a wide range of cellular functions including microtubule-dependent trafficking and signaling, membrane remodeling and chemotactic motility, involvement in control of cellular adhesion, ubiquitin level sensing, regulation of chaperone levels and activity, and responses to oxidative stress. All of these functions may be important in tumorigenesis, tumor growth and survival as well as metastasis (Simms-Waldrip et al., Mol. Genet. Metabolism 2008, 94(3):283-286; Rodriguez-Gonzalez et al., Cancer Res. 2008, 68(8):2557-2560; Kapoor, Int. J. Cancer 2009, 124:509; Lee et al., Cancer Res. 2008, 68(18):7561-7569). Recent studies have shown HDAC6 to be important in autophagy, an alternative pathway for protein degradation that compensates for deficiencies in the activity of the ubiquitin-proteasome system or the expression of proteins prone to form aggregates and can be activated following treatment with a proteasome inhibitor (Kawaguchi et al., Cell 2003, 115(6):727-738; Iwata et al., J. Biol. Chem. 2005, 280(48): 40282-40292; Ding et al., Am. J. Pathol. 2007, 171:513-524, Pandey et al., Nature 2007, 447(7146):860-864). Although the molecular mechanistic details are not completely understood, HDAC6 binds ubiquitinated or ubiquitin-like conjugated misfolded proteins which would otherwise induce proteotoxic stress and then serves as an adaptor protein to traffic the ubiquitinated cargo to the microtubule organizing center using the microtubule network via its known association with dynein motor protein. The resulting perinuclear aggregates, known as aggresomes, are then degraded by fusion with lysosomes in an HDAC6- and cortactin-dependent process which induces remodeling of the actin cytoskeleton proximal to aggresomes (Lee et al., EMBO J. 2010, 29:969-980). In addition, HDAC6 regulates a variety of biological processes dependent on its association with the microtubular network including cellular adhesion (Tran et al., J. Cell Sci. 2007, 120(8):1469-1479) and migration (Zhang et al., Mol. Cell 2007, 27(2):197-213; reviewed in Valenzuela-Fernandez et al., Trends Cell. Biol. 2008, 18(6):291-297), epithelial to mesenchymal transition (Shan et al., J. Biol. Chem. 2008, 283(30):21065-21073), resistance to anoikis (Lee et al., Cancer Res. 2008, 68(18):7561-7569), epithelial growth factor-mediated Wnt signaling via β-catenin deacetylation (Li et al., J. Biol. Chem. 2008, 283(19):12686-12690) and epithelial growth factor receptor stabilization by endocytic trafficking (Lissanu Deribe et al., Sci. Signal. 2009, 2(102): ra84; Gao et al., J. Biol. Chem. 2010, 285:11219-11226); all events that promote oncogenesis and metastasis (Lee et al., Cancer Res. 2008, 68(18):7561-7569). HDAC6 activity is known to be upregulated by Aurora A kinase in cilia formation (Pugacheva et al., Cell 2007, 129(7):1351-1363) and indirectly by farnesyl transferase with which HDAC6 forms a complex with microtubules (Zhou et al., J. Biol. Chem. 2009, 284(15): 9648-9655). Also, HDAC6 is negatively regulated by tau protein (Perez et al., J. Neurochem. 2009, 109(6):1756-1766).

Diseases in which selective HDAC6 inhibition could have a potential benefit include cancer (reviewed in Simms-Waldrip et al., Mol. Genet. Metabolism 2008, 94(3):283-286 and Rodriguez-Gonzalez et al., Cancer Res. 2008, 68(8):2557-2560), specifically: multiple myeloma (Hideshima et al., Proc. Natl. Acad. Sci. USA 2005, 102(24):8567-8572); lung cancer (Kamemura et al., Biochem. Biophys. Res. Commun. 2008, 374(1):84-89); ovarian cancer (Bazzaro et al., Clin. Cancer Res. 2008, 14(22):7340-7347); breast cancer (Lee et al., Cancer Res. 2008, 68(18):7561-7569); prostate cancer (Mellado et al., Clin. Trans. Onco. 2009, 11(1):5-10); pancreatic cancer (Nawrocki et al., Cancer Res. 2006, 66(7):3773-3781); renal cancer (Cha et al., Clin. Cancer Res. 2009, 15(3):840-850); and leukemias such as acute myeloid leukemia (AML) (Fiskus et al., Blood 2008, 112(7):2896-2905) and acute lymphoblastic leukemia (ALL) (Rodriguez-Gonzalez et al., Blood 2008, 112(11): Abstract 1923).

Inhibition of HDAC6 may also have a role in cardiovascular disease, i.e. cardiovascular stress, including pressure overload, chronic ischemia, and infarction-reperfusion injury (Tannous et al., Circulation 2008, 117(24):3070-3078); bacterial infection, including those caused by uropathogenic Escherichia coli (Dhakal and Mulve, J. Biol. Chem. 2008, 284(1):446-454); neurological diseases caused by accumulation of intracellular protein aggregates such as Huntington's disease (reviewed in Kazantsev et al., Nat. Rev. Drug Disc. 2008, 7(10):854-868; see also Dompierre et al., J. Neurosci. 2007, 27(13):3571-3583; Kozikowski et al., J. Med. Chem. 2007, 50:3054-3061) or central nervous system trauma caused by tissue injury, oxidative-stress induced neuronal or axomal degeneration (Rivieccio et al., Proc. Natl. Acad. Sci. USA 2009, 106(46):19599-195604); and inflammation, including reduction of pro-inflammatory cytokine IL-1β (Carta et al., Blood 2006, 108(5):1618-1626), increased expression of the FOXP3 transcription factor, which induces immunosuppressive function of regulatory T-cells resulting in benefits in chronic diseases such as rheumatoid arthritis, psoriasis, multiple sclerosis, lupus and organ transplant rejection (reviewed in Wang et al., Nat. Rev. Drug Disc. 2009 8(12):969-981).

Given the complex function of HDAC6, selective inhibitors could have potential utility when used alone or in combination with other chemotherapeutics such as microtubule destabilizing agents (Zhou et al., J. Biol. Chem. 2009, 284(15): 9648-9655); Hsp90 inhibitors (Rao et al., Blood 2008, 112(5)1886-1893); inhibitors of Hsp90 client proteins, including receptor tyrosine kinases such as Her-2 or VEGFR (Bhalla et al., J. Clin. Oncol. 2006, 24(18S): Abstract 1923; Park et al., Biochem. Biophys. Res. Commun. 2008, 368(2):318-322), and signaling kinases such as Bcr-Abl, Akt, mutant FLT-3, c-Raf, and MEK (Bhalla et al., J. Clin. Oncol. 2006, 24(18S): Abstract 1923; Kamemura et al., Biochem. Biophys. Res. Commun. 2008, 374(1):84-89); inhibitors of cell cycle kinases Aurora A and Aurora B (Pugacheva et al., Cell 2007, 129(7):1351-1363; Park et al., J. Mol. Med. 2008, 86(1): 117-128; Cha et al., Clin. Cancer Res. 2009, 15(3):840-850); EGFR inhibitors (Lissanu Deribe et al., Sci. Signal. 2009, 2(102): ra84; Gao et al., J. Biol. Chem. E-pub Feb. 4, 2010) and proteasome inhibitors (Hideshima et al., Proc. Natl. Acad. Sci. USA 2005, 102(24):8567-8572) or other inhibitors of the ubiquitin proteasome system such as ubiquitin and ubiqutin-like activating (E1), conjugation (E2), ligase enzymes (E3, E4) and deubiquitinase enzymes (DUBs) as well as modulators of autophagy and protein homeostasis pathways. In addition, HDAC6 inhibitors could be combined with radiation therapy (Kim et al., Radiother. Oncol. 2009, 92(1): 125-132.

Clearly, it would be beneficial to provide novel HDAC6 inhibitors that possess good therapeutic properties, especially for the treatment of proliferative diseases or disorders.

### DETAILED DESCRIPTION OF THE INVENTION

### 1. General Description of Compounds of the Invention

The present invention provides compounds that are effective inhibitors of HDAC6. These compounds are useful for inhibiting HDAC6 activity in vitro and in vivo, and are especially useful for the treatment of various cell proliferative diseases or disorders. The compounds of the invention are represented by formula (I):

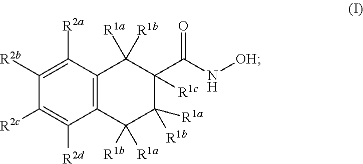

or a pharmaceutically acceptable salt thereof;

wherein:

each occurrence of R^{1a} is independently hydrogen, fluoro, —O—C₁₋₄ alkyl, C₁₋₄ alkyl, or C₁₋₄ fluoroalkyl;

each occurrence of R^{1b} is independently hydrogen, fluoro, C₁₋₄ alkyl, or C₁₋₄ fluoroalkyl;

or one occurrence of R^{1a} and one occurrence of R^{1b} on the same carbon atom can be taken together to form ═O or a 3-6 membered cycloaliphatic;

R^{1c} is hydrogen, fluoro, —O—C₁₋₄ alkyl, hydroxy, C₁₋₄ alkyl, or C₁₋₄ fluoroalkyl;

R^{2a} is G or R¹;

R^{2b} is G or R¹;

R^{2c} is G or R¹;

R^{2d} is G or R¹;

provided that one and only one of R^{2a}, R^{2b}, R^{2c}, and R^{2d} is G;

each occurrence of R¹ is independently hydrogen, chloro, fluoro, —O—C₁₋₄ alkyl, cyano, hydroxy, —C(O)NH₂, —N(C₁₋₄ alkyl)₂, —NH(C₁₋₄ alkyl), —NH₂, C₁₋₄ alkyl, or C₁₋₄ fluoroalkyl;

G is hydrogen, —R³, —V₁—R³, —V₁-L₁-R³, -L₁-V₁—R³, or -L₁-R³;

L₁ is an unsubstituted or substituted C₁₋₃ alkylene chain;

V₁ is —C(O)—, —C(S)—, —C(O)—N(R^{4a})—, —C(O)—O—, —N(R^{4a})—, —N(R^{4a})—C(O)—, —N(R^{4a})—SO₂—, —O—, —N(R^{4a})—C(O)—N(R^{4a})—, —N(R^{4a})—C(O)—O—, —O—C(O)—N(R^{4a})—, or —N(R^{4a})—SO₂—N(R^{4a})—;

R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; and

each occurrence of R^{4a} is independently hydrogen, or unsubstituted or substituted C₁₋₄ aliphatic;

provided that the compound is other than 1,2,3,4-tetrahydro-N-hydroxy-1-oxo-2-naphthalenecarboxamide.

### 2. Compounds and Definitions

Compounds of this invention include those described generally for formula (I) above, and are further illustrated by the classes, subclasses, and species disclosed herein. As used herein, the following definitions shall apply unless otherwise indicated.

As described herein, compounds of the invention may be optionally substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which hydrogen atom can be replaced with the radical of a suitable substituent. Unless otherwise indicated, an “optionally substituted” group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a patient.

The phrase “one or more substituents”, as used herein, refers to a number of substituents that equals from one to the maximum number of substituents possible based on the number of available bonding sites, provided that the above conditions of stability and chemical feasibility are met.

As used herein, the term “independently selected” means that the same or different values may be selected for multiple instances of a given variable in a single compound.

As used herein, the term “aromatic” includes aryl and heteroaryl groups as described generally below and herein.

The term “aliphatic” or “aliphatic group”, as used herein, means an optionally substituted straight-chain or branched C₁₋₁₂ hydrocarbon. For example, suitable aliphatic groups include optionally substituted linear, or branched alkyl, alkenyl, alkynyl groups and hybrids thereof. Unless otherwise specified, in various embodiments, aliphatic groups have 1-12, 1-10, 1-8, 1-6, 1-4, 1-3, or 1-2 carbon atoms.

The term “alkyl”, used alone or as part of a larger moiety, refers to an optionally substituted straight or branched chain hydrocarbon group having 1-12, 1-10, 1-8, 1-6, 1-4, 1-3, or 1-2 carbon atoms.

The term “alkenyl”, used alone or as part of a larger moiety, refers to an optionally substituted straight or branched chain hydrocarbon group having at least one double bond and having 2-12, 2-10, 2-8, 2-6, 2-4, or 2-3 carbon atoms.

The term “alkynyl”, used alone or as part of a larger moiety, refers to an optionally substituted straight or branched chain hydrocarbon group having at least one triple bond and having 2-12, 2-10, 2-8, 2-6, 2-4, or 2-3 carbon atoms.

The terms “cycloaliphatic”, “carbocycle”, “carbocyclyl”, “carbocyclo”, or “carbocyclic”, used alone or as part of a larger moiety, refer to an optionally substituted saturated or partially unsaturated cyclic aliphatic ring system having from 3 to about 14 ring carbon atoms. In some embodiments, the cycloaliphatic group is an optionally substituted monocyclic hydrocarbon having 3-10, 3-8 or 3-6 ring carbon atoms. Cycloaliphatic groups include, without limitation, optionally substituted cyclopropyl, cyclobutyl, cyclopentyl, cyclopentenyl, cyclohexyl, cyclohexenyl, cycloheptyl, cycloheptenyl, cyclooctyl, cyclooctenyl, or cyclooctadienyl. The terms “cycloaliphatic”, “carbocycle”, “carbocyclyl”, “carbocyclo”, or “carbocyclic” also include optionally substituted bridged or fused bicyclic rings having 6-12, 6-10, or 6-8 ring carbon atoms, wherein any individual ring in the bicyclic system has 3-8 ring carbon atoms.

The term “cycloalkyl” refers to an optionally substituted saturated ring system of about 3 to about 10 ring carbon atoms. Exemplary monocyclic cycloalkyl rings include cyclopropyl, cyclobutyl, cyclopentyl, cyclohexyl, and cycloheptyl.

The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

The terms “haloaliphatic”, “haloalkyl”, “haloalkenyl” and “haloalkoxy” refer to an aliphatic, alkyl, alkenyl or alkoxy group, as the case may be, which is substituted with one or more halogen atoms. As used herein, the term “halogen” or “halo” means F, Cl, Br, or I. The term “fluoroaliphatic” refers to a haloaliphatic wherein the halogen is fluoro, including perfluorinated aliphatic groups. Examples of fluoroaliphatic groups include, without limitation, fluoromethyl, difluoromethyl, trifluoromethyl, 2-fluoroethyl, 2,2,2-trifluoroethyl, 1,1,2-trifluoroethyl, 1,2,2-trifluoroethyl, and pentafluoroethyl.

The term “heteroatom” refers to one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

The terms “aryl” and “ar-”, used alone or as part of a larger moiety, e.g., “aralkyl”, “aralkoxy”, or “aryloxyalkyl”, refer to an optionally substituted C₆₋₁₄aromatic hydrocarbon moiety comprising one to three aromatic rings. Preferably, the aryl group is a C₆₋₁₀aryl group. Aryl groups include, without limitation, optionally substituted phenyl, naphthyl, or anthracenyl. The terms “aryl” and “ar-”, as used herein, also include groups in which an aryl ring is fused to one or more cycloaliphatic rings to form an optionally substituted cyclic structure such as a tetrahydronaphthyl, indenyl, or indanyl ring. The term “aryl” may be used interchangeably with the terms “aryl group”, “aryl ring”, and “aromatic ring”.

An “aralkyl” or “arylalkyl” group comprises an aryl group covalently attached to an alkyl group, either of which independently is optionally substituted. Preferably, the aralkyl group is C₆₋₁₀arylC₁₋₆alkyl, including, without limitation, benzyl, phenethyl, and naphthylmethyl.

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, 9, or 10 ring atoms; having 6, 10, or 14 π electrons shared in a cyclic array; and having, in addition to carbon atoms, from one to five heteroatoms. In some embodiments, the heteroaryl group has 5-10 ring atoms, having, in addition to carbon atoms, from one to five heteroatoms. A heteroaryl group may be mono-, bi-, tri-, or polycyclic, preferably mono-, bi-, or tricyclic, more preferably mono- or bicyclic. The term “heteroatom” refers to nitrogen, oxygen, or sulfur, and includes any oxidized form of nitrogen or sulfur, and any quaternized form of a basic nitrogen. For example, a nitrogen atom of a heteroaryl may be a basic nitrogen atom and may also be optionally oxidized to the corresponding N-oxide. When a heteroaryl is substituted by a hydroxy group, it also includes its corresponding tautomer. The terms “heteroaryl” and “heteroar-”, as used herein, also include groups in which a heteroaromatic ring is fused to one or more aryl, cycloaliphatic, or heterocycloaliphatic rings. Nonlimiting examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 4-10 membered ring, preferably a 3- to 8-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR+ (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiomorpholinyl. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. Additionally, a heterocyclic ring also includes groups in which the heterocyclic ring is fused to one or more aryl rings.

As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

The term “alkylene” refers to a bivalent alkyl group. An “alkylene chain” is a polymethylene group, i.e., —(CH₂)_{n′}—, wherein n′ is a positive integer, preferably from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3-7-membered ring. The substituents can be on the same or different atoms.

An alkylene chain also can be optionally interrupted by a functional group. An alkylene chain is “interrupted” by a functional group when an internal methylene unit is interrupted by the functional group. Examples of suitable “interrupting functional groups” are described in the specification and claims herein.

For purposes of clarity, all bivalent groups described herein, including, e.g., the alkylene chain linkers described above, are intended to be read from left to right, with a corresponding left-to-right reading of the formula or structure in which the variable appears.

An aryl (including aralkyl, aralkoxy, aryloxyalkyl and the like) or heteroaryl (including heteroaralkyl and heteroarylalkoxy and the like) group may contain one or more substituents and thus may be “optionally substituted”. In addition to the substituents defined above and herein, suitable substituents on the unsaturated carbon atom of an aryl or heteroaryl group also include and are generally selected from -halo, —NO₂, —CN, —R⁺, —C(R⁺)═C(R⁺)₂, —C≡C—R⁺, —OR⁺, —SR^{o}, —S(O)R^{o}, —SO₂R^{o}, —SO₃R⁺, —SO₂N(R⁺)₂, —N(R⁺)₂, —NR⁺C(O)R⁺, —NR⁺C(S)R⁺, —NR⁺C(O)N(R⁺)₂, —NR⁺C(S)N(R⁺)₂, —N(R⁺)C(═NR⁺)—N(R⁺)₂, —N(R⁺)C(═NR⁺)—R^{o}, —NR⁺CO₂R⁺, —NR⁺SO₂R^{o}, —NR⁺SO₂N(R⁺)², —O—C(O)R⁺, —O—CO₂R⁺, —OC(O)N(R⁺)₂, —C(O)R⁺, —C(S)R^{o}, —CO₂R⁺, —C(O)—C(O)R⁺, —C(O)N(R⁺)₂, —C(S)N(R⁺)₂, —C(O)N(R⁺)—OR⁺, —C(O)N(R⁺)C(═NR⁺)—N(R⁺)₂, —N(R⁺)C(═NR⁺)—N(R⁺)—C(O)R⁺, —C(═NR⁺)—N(R⁺)₂, —C(═NR⁺)—OR⁺, —N(R⁺)—N(R⁺)₂, —C(═NR⁺)—N(R⁺)—OR⁺, —C(R^{o}═N—OR⁺, —P(O)(R⁺)₂, —P(O)(OR⁺)₂, —O—P(O)—OR⁺, and —P(O)(NR⁺)—N(R⁺)₂, wherein R⁺, independently, is hydrogen or an optionally substituted aliphatic, aryl, heteroaryl, cycloaliphatic, or heterocyclyl group, or two independent occurrences of R⁺ are taken together with their intervening atom(s) to form an optionally substituted 5-7-membered aryl, heteroaryl, cycloaliphatic, or heterocyclyl ring. Each R^{o} is an optionally substituted aliphatic, aryl, heteroaryl, cycloaliphatic, or heterocyclyl group.

An aliphatic or heteroaliphatic group, or a non-aromatic carbocyclic or heterocyclic ring may contain one or more substituents and thus may be “optionally substituted”. Unless otherwise defined above and herein, suitable substituents on the saturated carbon of an aliphatic or heteroaliphatic group, or of a non-aromatic carbocyclic or heterocyclic ring are selected from those listed above for the unsaturated carbon of an aryl or heteroaryl group and additionally include the following: ═O, ═S, ═C(R*)₂, ═N—N(R*)₂, ═N—OR*, ═N—NHC(O)R*, ═N—NHCO₂R^{o}═N—NHSO₂R^{o} or ═N—R* where R^{o} is defined above, and each R* is independently selected from hydrogen or an optionally substituted C₁₋₆ aliphatic group.

In addition to the substituents defined above and herein, optional substituents on the nitrogen of a non-aromatic heterocyclic ring also include and are generally selected from —R⁺, —N(R⁺)₂, —C(O)R⁺, —C(O)OR⁺, —C(O)C(O)R⁺, —C(O)CH₂C(O)R⁺, —S(O)₂R⁺, —S(O)₂N(R⁺)₂, —C(S)N(R⁺)₂, —C(═NH)—N(R⁺)₂, or —N(R⁺)S(O)₂R⁺; wherein each R⁺ is defined above. A ring nitrogen atom of a heteroaryl or non-aromatic heterocyclic ring also may be oxidized to form the corresponding N-hydroxy or N-oxide compound. A nonlimiting example of such a heteroaryl having an oxidized ring nitrogen atom is N-oxidopyridyl.

As detailed above, in some embodiments, two independent occurrences of R⁺ (or any other variable similarly defined in the specification and claims herein), are taken together with their intervening atom(s) to form a monocyclic or bicyclic ring selected from 3-13-membered cycloaliphatic, 3-12-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur.

Exemplary rings that are formed when two independent occurrences of R⁺ (or any other variable similarly defined in the specification and claims herein), are taken together with their intervening atom(s) include, but are not limited to the following: a) two independent occurrences of R⁺ (or any other variable similarly defined in the specification or claims herein) that are bound to the same atom and are taken together with that atom to form a ring, for example, N(R⁺)₂, where both occurrences of R⁺ are taken together with the nitrogen atom to form a piperidin-1-yl, piperazin-1-yl, or morpholin-4-yl group; and b) two independent occurrences of R⁺ (or any other variable similarly defined in the specification or claims herein) that are bound to different atoms and are taken together with both of those atoms to form a ring, for example where a phenyl group is substituted with two occurrences of OR⁺

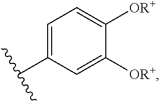

these two occurrences of R⁺ are taken together with the oxygen atoms to which they are bound to form a fused 6-membered oxygen containing ring:

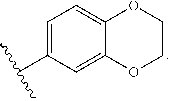

It will be appreciated that a variety of other rings (e.g., Spiro and bridged rings) can be formed when two independent occurrences of R+ (or any other variable similarly defined in the specification and claims herein) are taken together with their intervening atom(s) and that the examples detailed above are not intended to be limiting.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 13C- or 14C-enriched carbon are within the scope of this invention. Such compounds are useful, for example, as analytical tools or probes in biological assays.

The terms “stereoisomer”, “enantiomer”, “diastereomer”, “epimer”, and “chiral center”, are used herein in accordance with the meaning each is given in ordinary usage by those of ordinary skill in the art. Thus, stereoisomers are compounds that have the same atomic connectivity, but differ in the spatial arrangement of the atoms. Enantiomers are stereoisomers that have a mirror image relationship, that is, the stereochemical configuration at all corresponding chiral centers is opposite. Diastereomers are stereoisomers having more than one chiral center, which differ from one another in that the stereochemical configuration of at least one, but not all, of the corresponding chiral centers is opposite. Epimers are diastereomers that differ in stereochemical configuration at only one chiral center.

It is to be understood that, when a disclosed compound has at least one chiral center, the present invention encompasses one enantiomer of the compound, substantially free from the corresponding optical isomer, a racemic mixture of both optical isomers of the compound, and mixtures enriched in one enantiomer relative to its corresponding optical isomer. When a mixture is enriched in one enantiomer relative to its optical isomer, the mixture contains, for example, an enantiomeric excess of at least 50%, 75%, 90%, 95%, 99%, or 99.5%.

The enantiomers of the present invention may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. Where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a disclosed compound has at least two chiral centers, the present invention encompasses a diastereomer substantially free of other diastereomers, an enantiomeric pair of diastereomers substantially free of other stereoisomers, mixtures of diastereomers, mixtures of enantiomeric pairs of diastereomers, mixtures of diastereomers in which one diastereomer is enriched relative to the other diastereomer(s), and mixtures of enantiomeric pairs of diastereomers in which one enantiomeric pair of diastereomers is enriched relative to the other stereoisomers. When a mixture is enriched in one diastereomer or enantiomeric pair of diastereomers pairs relative to the other stereoisomers, the mixture is enriched with the depicted or referenced diastereomer or enantiomeric pair of diastereomers relative to other stereoisomers for the compound, for example, by a molar excess of at least 50%, 75%, 90%, 95%, 99%, or 99.5%.

As used herein, the term “diastereomeric ratio” refers to the ratio between diastereomers which differ in the stereochemical configuration at one chiral center, relative to a second chiral center in the same molecule. By way of example, a chemical structure with two chiral centers provides four possible stereoisomers: R*R, R*S, S*R, and S*S, wherein the asterisk denotes the corresponding chiral center in each stereoisomer. The diastereomeric ratio for such a mixture of stereoisomers is the ratio of one diastereomer and its enantiomer to the other diastereomer and its enantiomer=(R*R+S*S):(R*S+S*R).

One of ordinary skill in the art will recognize that additional stereoisomers are possible when the molecule has more than two chiral centers. For purposes of the present invention, the term “diastereomeric ratio” has identical meaning in reference to compounds with multiple chiral centers as it does in reference to compounds having two chiral centers. Thus, the term “diastereomeric ratio” refers to the ratio of all compounds having R*R or S*S configuration at the specified chiral centers to all compounds having R*S or S*R configuration at the specified chiral centers. For convenience, this ratio is referred to herein as the diastereomeric ratio at the asterisked carbon, relative to the second specified chiral center.

The diastereomeric ratio can be measured by any analytical method suitable for distinguishing between diastereomeric compounds having different relative stereochemical configurations at the specified chiral centers. Such methods include, without limitation, nuclear magnetic resonance (NMR), gas chromatography (GC), and high performance liquid chromatography (HPLC) methods.

The diastereoisomeric pairs may be separated by methods known to those skilled in the art, for example chromatography or crystallization and the individual enantiomers within each pair may be separated as described above. Specific procedures for chromatographically separating diastereomeric pairs of precursors used in the preparation of compounds disclosed herein are provided the examples herein.

### 3. Description of Exemplary Compounds

In some embodiments, the compound of formula (I) is represented by:

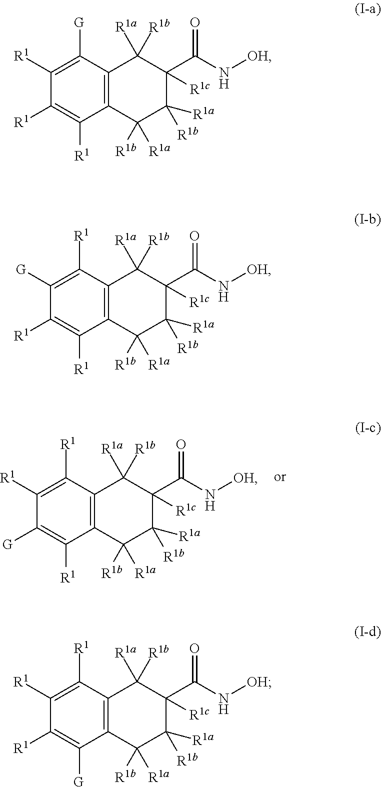

wherein R^{1a}, R^{1b}, R^{1c}, R¹, and G have the values described herein. In certain embodiments, the compound of formula (I) is represented by formula (I-a), wherein R^{1a}, R^{1b}, R^{1c}, R¹, and G have the values described herein. In certain embodiments, the compound of formula (I) is represented by formula (I-b), wherein R^{1a}, R^{1b}, R^{1c}, R¹, and G have the values described herein. In certain embodiments, the compound of formula (I) is represented by formula (I-c), wherein R^{1a}, R^{1b}, R^{1c}, R¹, and G have the values described herein.

In some embodiments, the compound of formula (I) is represented by formula (II):

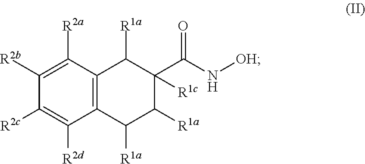

wherein R^{1a}, R^{1c}, R^{2a}, R^{2b}, R^{2c}, and R^{2d} have the values described herein.

In some embodiments, the compound of formula (I) is represented by formula (II-a)-(II-d):

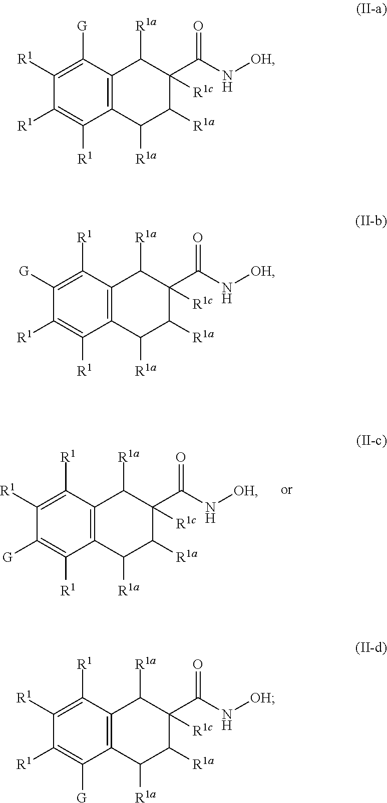

wherein R^{1a}, R^{1c}, R¹, and G have the values described herein. In certain embodiments, the compound of formula (I) is represented by formula (II-a), wherein R^{1a}, R^{1c}, R¹, and G have the values described herein. In certain embodiments, the compound of formula (I) is represented by formula (II-b), wherein R^{1a}, R^{1c}, R¹, and G have the values described herein. In certain embodiments, the compound of formula (I) is represented by formula (II-c), wherein R^{1a}, R^{1c}, R¹, and G have the values described herein.

In some embodiments, the compound of formula (I) is represented by formula (III):

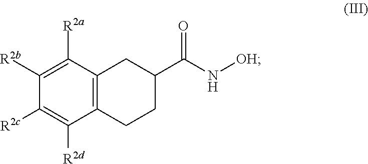

wherein R^{2a}, R^{2b}, R^{2c}, and R^{2d} have the values described herein.

In some embodiments, the compound of formula (I) is represented by formula (III-a)-(III-d):

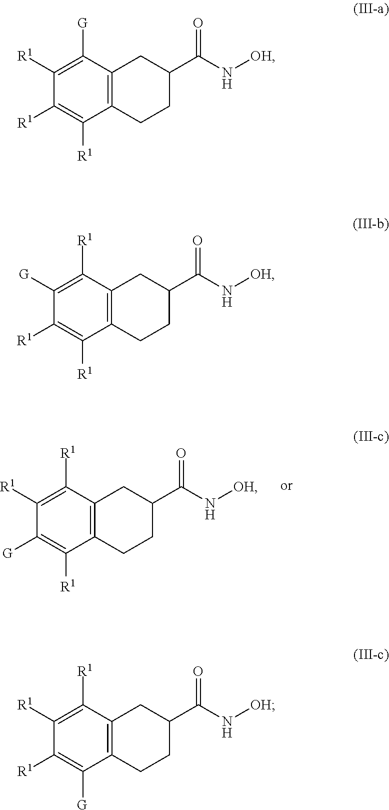

wherein R¹ and G have the values described herein. In certain embodiments, the compound of formula (I) is represented by formula (III-a), wherein R¹ and G have the values described herein. In certain embodiments, the compound of formula (I) is represented by formula (III-b), wherein R¹ and G have the values described herein. In certain embodiments, the compound of formula (I) is represented by formula (III-c), wherein R¹ and G have the values described herein.

In some embodiments, the compound of formula (I) is represented by formula (IV-a)-(IV-d):

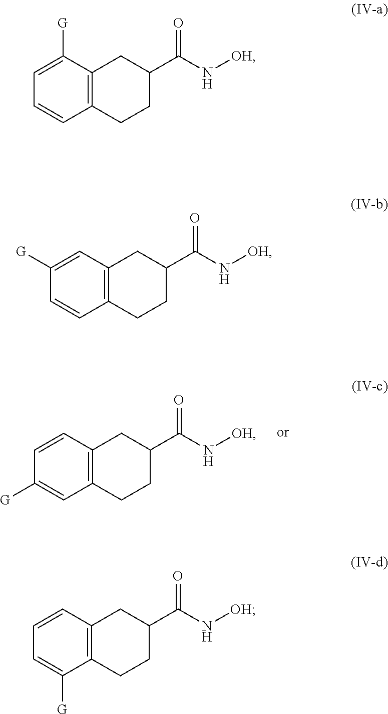

wherein G has the values described herein. In certain embodiments, the compound of formula (I) is represented by formula (IV-a), wherein G has the values described herein. In certain embodiments, the compound of formula (I) is represented by formula (IV-b), wherein G has the values described herein. In certain embodiments, the compound of formula (I) is represented by formula (IV-c), wherein G has the values described herein. In certain embodiments, the compound of formula (I) is represented by formula (IV-d), wherein G has the values described herein.

The values described below for each variable are with respect to any of formulas (I), (II), (III), (IV), or their sub-formulas as described above.

Each occurrence of the variable R^{1a} is independently hydrogen, fluoro, —O—C₁₋₄ alkyl, C₁₋₄ alkyl, or C₁₋₄ fluoroalkyl. In some embodiments, each occurrence of R^{1b} is independently hydrogen, fluoro, methoxy, methyl, ethyl, or trifluoromethyl. In certain embodiments, each occurrence of R^{1a} is independently hydrogen, fluoro, trifluoromethyl, or methyl. In certain embodiments, each occurrence of R^{1a} is independently hydrogen or methyl. In certain embodiments, each occurrence of R^{1a} is hydrogen.

Each occurrence of the variable R^{1b} is independently hydrogen, fluoro, C₁₋₄ alkyl, or C₁₋₄ fluoroalkyl. In some embodiments, each occurrence of R^{1b} is independently hydrogen, fluoro, methyl, ethyl, or trifluoromethyl. In certain embodiments, each occurrence of R^{1b} is independently hydrogen, fluoro, or methyl. In certain embodiments, each occurrence of R^{1b} is independently hydrogen or methyl. In certain embodiments, each occurrence of R^{1b} is hydrogen.

In some embodiments, one occurrence of R^{1a} and one occurrence of R^{1b} on the same carbon atom can be taken together to form ═O or a 3-6 membered cycloaliphatic. In some embodiments, one occurrence of R^{1a} and one occurrence of R^{1b} on the same carbon atom can be taken together to form cyclopropyl, cyclobutyl, cyclopentyl or cyclohexyl. In certain embodiments, one occurrence of R^{1a} and one occurrence of R^{1b} on the same carbon atom can be taken together to form ═O. In certain embodiments, one occurrence of R^{1a} and one occurrence of R^{1b} on the same carbon atom can be taken together to form cyclopropyl.

The variable R^{1c} is hydrogen, fluoro, alkyl, hydroxy, C₁₋₄ alkyl, or C₁₋₄ fluoroalkyl. In some embodiments, R^{1c} is hydrogen, fluoro, methoxy, hydroxy, methyl, ethyl, or trifluoromethyl. In certain embodiments, R^{1c} is hydrogen, hydroxy, fluoro, trifluoromethyl, or methyl. In certain embodiments, R^{1c} is hydrogen, hydroxy, or methyl. In certain embodiments, R^{1c} is hydrogen.

Each occurrence of the variable R¹ is independently hydrogen, chloro, fluoro, —O—C₁₋₄ alkyl, cyano, hydroxy, —C(O)NH₂, —N(C₁₋₄ alkyl)₂, —NH(C₁₋₄ alkyl), —NH₂, C₁₋₄ alkyl, or C₁₋₄ fluoroalkyl. In some embodiments, each occurrence of R¹ is independently hydrogen, chloro, fluoro, cyano, hydroxy, methoxy, ethoxy, trifluoromethyl, —C(O)NH₂, methyl, or ethyl. In some embodiments, each occurrence of R¹ is hydrogen, cyano, or —C(O)NH₂. In certain embodiments, each occurrence of R¹ is independently hydrogen, chloro, fluoro, cyano, hydroxy, methoxy, ethoxy, trifluoromethyl, methyl, or ethyl. In certain embodiments, each occurrence of R¹ is independently hydrogen, fluoro, or methyl. In certain embodiments, each occurrence of R¹ is hydrogen.

One and only one of the variables R^{2a}, R^{2b}, R^{2c} and R^{2d} is G and the others are R¹, wherein R¹ and G have the values described herein. In certain embodiments, R^{2a} is G and R^{2b}, R^{2c}, and R^{2d} are R¹, wherein R¹ and G have the values described herein. In certain embodiments, R^{2b} is G and R^{2a}, R^{2c}, and R^{2d} are R¹, wherein R¹ and G have the values described herein. In certain embodiments, R^{2c} is G and R^{2a}, R^{2b}, and R^{2d} are R¹, wherein R¹ and G have the values described herein.

The variable G is hydrogen, —R³, —V₁—R³, —V₁-L₁-R³, -L₁-V₁—R³, or -L₁-R³, wherein L₁, V₁, and R³ have the values described herein. In some embodiments, G is —R³, —V₁—R³, —V₁-L₁-R³, -L₁-V₁—R³, or -L₁-R³, wherein L₁, V₁, and R³ have the values described herein. In some embodiments, G is —V₁—R³, -L₁-R³, or —R³, wherein L₁, V₁, and R³ have the values described herein. In certain embodiments, G is —V₁—R³, wherein V₁ and R³ have the values described herein. In certain embodiments, G is -L₁-R³, wherein L₁ and R³ have the values described herein. In certain embodiments, G is —R³, wherein R³ has the values described herein.

The variable L₁ is an unsubstituted or substituted C₁₋₃ alkylene chain. In some embodiments, L₁ is —CH₂—, —CH₂CH₂—, —CH₂CH₂CH₂—, —CR^{A}═CR^{A}, or —C≡C. In some embodiments, L₁ is —CH₂—, —CH₂CH₂—, or —CH₂CH₂CH₂—. In certain embodiments, L₁ is —CH₂—. In certain embodiments, L₁ is —CH₂CH₂—.

Each occurrence of the variable R^{A} is independently hydrogen, fluoro, or unsubstituted or substituted C₁₋₄ aliphatic. In some embodiments, each occurrence of R^{A} is independently hydrogen, fluoro or methyl. In certain embodiments, each occurrence of R^{A} is hydrogen.

The variable V₁ is —C(O)—, —C(S)—, —C(O)—N(R^{4a})—, —C(O)—O—, —N(R^{4a})—, —N(R^{4a})—C(O)—, —N(R^{4a})—SO₂—, —O—, —N(R^{4a})—C(O)—N(R^{4a})—, —N(R^{4a})—C(O)—O—, —O—C(O)—N(R^{4a})—, or —N(R^{4a})—SO₂—N(R^{4a})—; wherein R^{4a} has the values described herein. In some embodiments, V₁ is —N(R^{4a})—, —N(R^{4a})—C(O)—, C(O)—N(R^{4a})—, —N(R^{4a})—SO₂—, —O—, —N(R^{4a})—C(O)—O—, or —N(R^{4a})—C(O)—N(R^{4a})—, wherein R^{4a} has the values described herein. In certain embodiments, V₁ is —N(R^{4a})—, —N(R^{4a})—C(O)—, —C(O)—N(R^{4a})—, or —O—, wherein R^{4a} has the values described herein. In certain embodiments, V₁ is —NH—, —NH—C(O)—, —C(O)—NH—, —NH—SO₂—, —O—, —NH—C(O)—O—, or —NH—C(O)—NH—. In certain embodiments, V₁ is —NH—, —NH—C(O)—, —C(O)—NH—, or —O—.

Each occurrence of the variable R^{4a} is independently hydrogen, or unsubstituted or substituted C₁₋₄ aliphatic. In some embodiments, each occurrence of R^{4a} is hydrogen.

The variable R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

In some embodiments, R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; wherein:

each substitutable carbon chain atom in R³ is unsubstituted or substituted with 1-2 occurrences of —R^{5dd};

each substitutable saturated ring carbon atom in R³ is unsubstituted or substituted with ═O, ═C(R⁵)₂, or —R^{5aa};

each substitutable unsaturated ring carbon atom in R³ is unsubstituted or is substituted with —R^{5a};

each substitutable ring nitrogen atom in R³ is unsubstituted or substituted with —R^{9b};

wherein R^{5dd}, R⁵, R^{5a}, R^{5aa}, and R^{9b} have the values described herein.

In some embodiments, R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; wherein:
each substitutable carbon chain atom in R³ is unsubstituted or substituted with 1-2 occurrences of —R^{5dd};
each substitutable saturated ring carbon atom in R³ is unsubstituted or substituted with —R^{5aa};
each substitutable unsaturated ring carbon atom in R³ is unsubstituted or is substituted with —R^{5a};
the total number of R^{5a} and R^{5aa} substituents is p; and
each substitutable ring nitrogen atom in R³ is unsubstituted or substituted with —R^{9b};
wherein R^{5dd}, R^{5a}, R^{5aa}, R^{9b} and p have the values described herein.

Each occurrence of the variable R^{5dd} is independently fluoro, hydroxy, —O(C₁₋₆ alkyl), cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, —NHC(O)N(C₁₋₆ alkyl)₂, or —NHS(O)₂C₁₋₆ alkyl, wherein R⁴ has the values described herein. In some embodiments, each occurrence of R^{5dd} is independently fluoro, hydroxy, methoxy, ethoxy, —NH(C₁₋₆ alkyl), —N(C₁₋₆ alkyl)₂, or —C(O)NHCH₃.

Each occurrence of the variable R^{9b} is independently —C(O)R⁶, —C(O)N(R⁴)₂, —CO₂R⁶, —SO₂R⁶, —SO₂N(R⁴)₂, unsubstituted C₃₋₁₀ cycloaliphatic, C₃₋₁₀ cycloaliphatic substituted with 1-2 independent occurrences of R⁷ or R⁸, unsubstituted C₁₋₆ aliphatic, or C₁₋₆ aliphatic substituted with 1-2 independent occurrences of R⁷ or R⁸, wherein R⁷ and R⁸ have the values described herein. In some embodiments, each occurrence of R^{9b} is independently unsubstituted —C(O)—C₁₋₆ aliphatic, unsubstituted —C(O)—C₃₋₁₀ cycloaliphatic, or unsubstituted C₁₋₆ aliphatic. In some embodiments, each occurrence of R^{9b} is unsubstituted C₁₋₆ aliphatic. In certain embodiments, each occurrence of R^{9b} is independently methyl, ethyl, isopropyl, isobutyl, n-propyl, n-butyl, tert-butyl, —C(O)-methyl, —C(O)-ethyl, —C(O)-cyclopropyl, —C(O)-tert-butyl, —C(O)-isopropyl, or —C(O)-cyclobutyl. In certain embodiments, each occurrence of R^{9b} is independently methyl, ethyl, isopropyl, isobutyl, n-propyl, n-butyl, or tert-butyl.

Each occurrence of the variable R⁴ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two R⁴ on the same nitrogen atom, taken together with the nitrogen atom, form an unsubstituted or substituted 5- to 6-membered heteroaryl or an unsubstituted or substituted 4- to 8-membered heterocyclyl having, in addition to the nitrogen atom, 0-2 ring heteroatoms selected from nitrogen, oxygen, and sulfur.

Each occurrence of the variable R⁵ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

Each occurrence of the variable R⁶ is independently unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

Each occurrence of the variable R⁷ is independently unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

Each occurrence of the variable R⁸ is independently chloro, fluoro, —OH, —O(C₁₋₆ alkyl), —CN, —N(R⁴)₂, —C(O)(C₁₋₆ alkyl), —CO₂H, —CO₂(C₁₋₆ alkyl), —C(O)NH₂, —C(O)NH(C₁₋₆ alkyl), or —C(O)N(C₁₋₆ alkyl)₂, wherein R⁴ has the values described herein.

Each occurrence of the variable R^{5a} is independently halogen, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁶, —NR⁴C(O)N(R⁴)₂, —NR⁴CO₂R⁶, —OC(O)N(R⁴)₂, —C(O)R⁶, —C(O)N(R⁴)₂, —N(R⁴)SO₂R⁶, —N(R⁴)SO₂N(R⁴)₂, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two adjacent R^{5a}, taken together with the intervening ring atoms, form an unsubstituted or substituted fused 5-10 membered aromatic ring or an unsubstituted or substituted 4-10 membered non-aromatic ring having 0-3 heteroatoms independently selected from nitrogen, oxygen, and sulfur, wherein R⁵, R⁶, and R⁴ have the values described herein.

In some embodiments, each occurrence of R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted —O—C₁₋₆ alkyl, —O—C₁₋₆ alkyl substituted with 1-2 independent occurrences of R⁷ or R⁸, C₁₋₆ fluoroalkyl, —O—C₁₋₆ fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆alkyl)₂, 3-10-membered cycloaliphatic substituted with 0-2 occurrences of —R^{7a}, 4-10-membered heterocyclyl having 1-4 heteroatoms independently selected from nitrogen, oxygen, and sulfur substituted with 0-2 occurrences of —R^{7a}, 6-10-membered aryl substituted with 0-2 occurrences of —R^{7a}, or 5-10-membered heteroaryl having 1-5 heteroatoms independently selected from nitrogen, oxygen, and sulfur substituted with 0-2 occurrences of —R^{7a}, wherein R^{7a}, R⁷, and R⁸ have the values described herein has the values described herein.

In certain embodiments, each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)—R¹⁰, —C(O)NH—R¹⁰, —CH₂—N(R⁴)₂, or —NHSO₂CH₃, wherein R¹⁰ has the values described herein.

Each occurrence of the variable R¹⁰ is independently unsubstituted or substituted 4-10-membered heterocyclyl having 1-4 heteroatoms independently selected from nitrogen, oxygen, and sulfur. In some embodiments, each occurrence of R¹⁰ is independently unsubstituted or substituted 4-10-membered heterocyclyl having 1-4 heteroatoms independently selected from nitrogen, oxygen, and sulfur, wherein if substituted R¹⁰ is substituted with 0-2 independent occurrences of —R^{7aa}, wherein R^{7aa} has the values described herein. In some embodiments, each occurrence of R¹⁰ is independently pyrrolidinyl, piperidinyl, pyrrolinyl, piperazinyl, or morpholinyl, wherein each of the foregoing groups is unsubstituted or substituted with 0-1 occurrence of R^{7aa}, wherein R^{7aa} has the values described herein.

Each occurrence of the variable R^{5aa} is independently chloro, fluoro, hydroxy, unsubstituted or substituted C₁₋₆ aliphatic, —O(C₁₋₆ alkyl), —C₁₋₆ fluoroalkyl, —O—C₁₋₆ fluoroalkyl, cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, —NHC(O)N(C₁₋₆ alkyl)₂, or —NHS(O)₂C₁₋₆ alkyl. In some embodiments, each occurrence of R^{5aa} is independently chloro, fluoro, hydroxy, methyl, ethyl, methoxy, ethoxy, trifluoromethyl, trifluoromethoxy, —C(O)NH₂, —N(C₁₋₆ alkyl)₂, —NHC₁₋₆ alkyl, or —CO₂H.

Each occurrence of the variable R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆ alkyl)₂, —C(O)NHC₁₋₆ alkyl, —C(O)N(C₁₋₆ alkyl)₂, —NHC(O)NHC₁₋₆ alkyl, —NHC(O)N(C₁₋₆ alkyl)₂, or —NHS(O)₂C₁₋₆ alkyl.

Each occurrence of the variable R^{7aa} is independently chloro, fluoro, hydroxy, unsubstituted or substituted C₁₋₆ aliphatic, —O(C₁₋₆ alkyl), —C₁₋₆ fluoroalkyl, fluoroalkyl, cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, —NHC(O)N(C₁₋₆ alkyl)₂, or —NHS(O)₂C₁₋₆ alkyl. In some embodiments, each occurrence of R^{7aa} is independently fluoro, hydroxy, methyl, ethyl, methoxy, trifluoromethyl, —C(O)NH₂, or —CO₂H.

The variable p is 1-4. In some embodiments, p is 1-3. In certain embodiments, p is 1-2. In certain embodiments, p is 1.

In some embodiments, R³ is unsubstituted or substituted C₁₋₆ aliphatic. In some embodiments, each substitutable carbon chain atom in R³ is unsubstituted or substituted with 1-2 occurrences of —R^{5dd}, wherein R^{5dd} has the values described herein. In certain embodiments, R³ is methyl, ethyl, n-propyl, isopropyl, tert-butyl, n-butyl, iso-butyl, pentyl, hexyl, butenyl, propenyl, pentenyl, or hexenyl, wherein each of the forementioned groups is unsubstituted or substituted. In certain embodiments, R³ is methyl, ethyl, n-propyl, isopropyl, tert-butyl, n-butyl, iso-butyl, pentyl, hexyl, butenyl, propenyl, pentenyl, or hexenyl, wherein each substitutable carbon chain atom in R³ is unsubstituted or substituted with 1-2 occurrences of —R^{5dd}, wherein R^{5dd} has the values described herein.

In some embodiments, R³ is unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur.

In certain embodiments, R³ is unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur, wherein:

each substitutable saturated ring carbon atom in R³ is unsubstituted or substituted with ═O, ═C(R⁵)₂, or —R^{5aa};

each substitutable unsaturated ring carbon atom in R³ is unsubstituted or is substituted with —R^{5a}; and

each substitutable ring nitrogen atom in R³ is unsubstituted or substituted with —R^{9b};

wherein R⁵, R^{5a}, R^{5aa}, and R^{9b} have the values described herein.

In certain embodiments, R³ is unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur, wherein:
each substitutable saturated ring carbon atom in R³ is unsubstituted or substituted with —R^{5aa};
each substitutable unsaturated ring carbon atom in R³ is unsubstituted or is substituted with —R^{5a};
the total number of R^{5a} and R^{5aa} substituents is p; and
each substitutable ring nitrogen atom in R³ is unsubstituted or substituted with —R^{9b};
wherein R^{5a}, R^{5aa}, R^{9b} and p have the values described herein.

In certain embodiments, R³ is furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, naphthyl, pyranyl, pyridyl, pyridazinyl, pyrimidinyl, pyrazinyl, triazinyl, indolizinyl, imidazopyridyl, indolyl, isoindolyl, indazolyl, benzimidazolyl, benzthiazolyl, benzothienyl, benzofuranyl, benzoxazolyl, benzodioxolyl, benzthiadiazolyl, 2,3-dihydrobenzofuranyl, 4H-furo[3,2-b]pyrrolyl, pyrazolopyrimidinyl, purinyl, quinolyl, isoquinolyl, tetrahydroquinolinyl, tetrahydronaphthyridinyl, tetrahydroisoquinolinyl, cinnolinyl, phthalazinyl, quinazolinyl, quinoxalinyl, naphthyridinyl, pteridinyl, tetrahydrofuranyl, tetrahydropyranyl, tetrahydrothienyl, indanyl, tetrahydroindazolyl, pyrrolidinyl, pyrrolidonyl, piperidinyl, pyrrolinyl, decahydroquinolinyl, oxazolidinyl, piperazinyl, dioxanyl, diazepinyl, oxazepinyl, thiazepinyl, morpholinyl, thiomorpholinyl, quinuclidinyl, phenanthridinyl, tetrahydronaphthyl, indolinyl, benzodioxanyl, chromanyl, cyclopropyl, cyclobutyl, cyclopentyl, cyclohexyl, cycloheptyl, cyclooctyl, cyclopentenyl, cyclohexenyl, cycloheptenyl, cyclooctenyl, bicycloheptanyl, bicyclooctanyl, or adamantyl; wherein:

each substitutable saturated ring carbon atom in R³ is unsubstituted or substituted with ═O, ═C(R⁵)₂, or —R^{5aa};

each substitutable unsaturated ring carbon atom in R³ is unsubstituted or is substituted with —R^{5a}; and

each substitutable ring nitrogen atom in R³ is unsubstituted or substituted with —R^{9b};

wherein R⁵, R^{5a}, R^{5aa}, and R^{9b} have the values described herein.

In certain embodiments, R³ is furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, naphthyl, pyranyl, pyridyl, pyridazinyl, pyrimidinyl, pyrazinyl, triazinyl, indolizinyl, imidazopyridyl, indolyl, isoindolyl, indazolyl, benzimidazolyl, benzthiazolyl, benzothienyl, benzofuranyl, benzoxazolyl, benzodioxolyl, benzthiadiazolyl, 2,3-dihydrobenzofuranyl, 4H-furo[3,2-b]pyrrolyl, pyrazolopyrimidinyl, purinyl, quinolyl, isoquinolyl, tetrahydroquinolinyl, tetrahydronaphthyridinyl, tetrahydroisoquinolinyl, cinnolinyl, phthalazinyl, quinazolinyl, quinoxalinyl, naphthyridinyl, pteridinyl, tetrahydrofuranyl, tetrahydropyranyl, tetrahydrothienyl, indanyl, tetrahydroindazolyl, pyrrolidinyl, pyrrolidonyl, piperidinyl, pyrrolinyl, decahydroquinolinyl, oxazolidinyl, piperazinyl, dioxanyl, diazepinyl, oxazepinyl, thiazepinyl, morpholinyl, thiomorpholinyl, quinuclidinyl, phenanthridinyl, tetrahydronaphthyl, indolinyl, benzodioxanyl, chromanyl, cyclopropyl, cyclobutyl, cyclopentyl, cyclohexyl, cycloheptyl, cyclooctyl, cyclopentenyl, cyclohexenyl, cycloheptenyl, cyclooctenyl, bicycloheptanyl, bicyclooctanyl, or adamantyl; wherein:
each substitutable saturated ring carbon atom in R³ is unsubstituted or substituted with —R^{5aa};
each substitutable unsaturated ring carbon atom in R³ is unsubstituted or is substituted with —R^{5a};
the total number of R^{5a} and R^{5aa} substituents is p; and
each substitutable ring nitrogen atom in R³ is unsubstituted or substituted with —R^{9b};
wherein R^{5a}, R^{5aa}, R^{9b} and p have the values described herein.

In certain embodiments, R³ is furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, pyranyl, pyridyl, pyridazinyl, pyrimidinyl, pyrazinyl, or triazinyl; wherein:

each substitutable unsaturated ring carbon atom in R³ is unsubstituted or substituted with —R^{5a};

each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(R⁴)₂, or —NHSO₂CH₃;

the total number of R^{5a} substituents is p;

each substitutable ring nitrogen atom in R³ is unsubstituted or substituted with —R^{9b}; and

each occurrence of R^{9b} is independently methyl, ethyl, isopropyl, isobutyl, n-propyl, n-butyl, or tert-butyl;

wherein p and R¹⁰ have the values described herein.

In certain embodiments, R³ is indolizinyl, imidazopyridyl, indolyl, indazolyl, benzimidazolyl, benzthiazolyl, benzothienyl, benzofuranyl, benzoxazolyl, benzthiadiazolyl, pyrazolopyrimidinyl, purinyl, quinolyl, isoquinolyl, cinnolinyl, phthalazinyl, quinazolinyl, quinoxalinyl, naphthyridinyl, naphthyl, or pteridinyl; wherein:

each substitutable unsaturated ring carbon atom in R³ is unsubstituted or substituted with —R^{5a};

each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(CH₃)₂, or —NHSO₂CH₃;

the total number of R^{5a} substituents is p;

each substitutable ring nitrogen atom in R³ is unsubstituted or substituted with —R^{9b}; and

each R^{9b} is independently methyl, ethyl, isopropyl, isobutyl, n-propyl, n-butyl, or tert-butyl;

wherein p and R¹⁰ have the values described herein.

In certain embodiments, R³ is tetrahydrofuranyl, tetrahydropyranyl, tetrahydrothienyl, pyrrolidinyl, pyrrolidonyl, piperidinyl, pyrrolinyl, oxazolidinyl, piperazinyl, dioxanyl, diazepinyl, oxazepinyl, thiazepinyl, morpholinyl, thiomorpholinyl, cyclopropyl, cyclobutyl, cyclopentyl, cyclohexyl, cycloheptyl, cyclooctyl, cyclopentenyl, cyclohexenyl, cycloheptenyl, or cyclooctenyl; wherein:
each substitutable saturated ring carbon atom in R³ is unsubstituted or substituted with —R^{5aa};
each substitutable unsaturated ring carbon atom in R³ is unsubstituted or is substituted with —R^{5a};
the total number of R^{5a} and R^{5aa} substituents is p;
each substitutable ring nitrogen atom in R³ is unsubstituted or substituted with —R^{9b};
each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(R⁴)₂, or —NHSO₂CH₃;
each occurrence of R^{5aa} is independently chloro, fluoro, hydroxy, methyl, ethyl, methoxy, ethoxy, trifluoromethyl, trifluoromethoxy, —C(O)NH₂, —N(C₁₋₆ alkyl)₂, —NHC₁₋₆ alkyl, or —CO₂H; and
each R^{9b} is independently methyl, ethyl, isopropyl, isobutyl, n-propyl, n-butyl, tert-butyl, —C(O)-methyl, —C(O)-ethyl, —C(O)-cyclopropyl, —C(O)-tert-butyl, —C(O)-isopropyl, or —C(O)-cyclobutyl;
wherein p and R¹⁰ have the values described herein.

In certain embodiments, R³ is tetrahydroindazolyl, bicycloheptanyl, bicyclooctanyl, adamantyl, isoindolyl, benzodioxolyl, 2,3-dihydrobenzofuranyl, 4H-furo[3,2-b]pyrrolyl, quinuclidinyl, tetrahydroquinolinyl, tetrahydronaphthyridinyl, tetrahydroisoquinolinyl, decahydroquinolinyl, tetrahydronaphthyl, indolinyl, benzodioxanyl, chromanyl, tetrahydroindazolyl, or indanyl; wherein:
each substitutable saturated ring carbon atom in R³ is unsubstituted or substituted with —R^{5aa};
each substitutable unsaturated ring carbon atom in R³ is unsubstituted or is substituted with —R^{5a};
the total number of R^{5a} and R^{5aa} substituents is p;
each substitutable ring nitrogen atom in R³ is unsubstituted or substituted with —R^{9b};
each occurrence of R^{5a} is independently chloro, fluoro, hydroxy, methoxy, ethoxy, cyano, trifluoromethyl, methyl, ethyl, isopropyl, —NHC(O)-tert-butyl, —NHC(O)-cyclopropyl, —NHC(O)R¹⁰, —C(O)NHR¹⁰, —CH₂—N(R⁴)₂, or —NHSO₂CH₃;
each occurrence of R^{5aa} is independently chloro, fluoro, hydroxy, methyl, ethyl, methoxy, ethoxy, trifluoromethyl, trifluoromethoxy, —C(O)NH₂, —N(C₁₋₆ alkyl)₂, —NHC₁₋₆ alkyl, or —CO₂H; and
each R^{9b} is independently methyl, ethyl, isopropyl, isobutyl, n-propyl, n-butyl, tert-butyl, —C(O)-methyl, —C(O)-ethyl, —C(O)-cyclopropyl, —C(O)-tert-butyl, —C(O)-isopropyl, or —C(O)-cyclobutyl;
wherein p and R¹⁰ have the values described herein.

In some embodiments, G is:

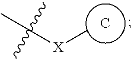

wherein X and Ring C have the values described herein.

The variable X is a bond, —NH—C(O)—, —C(O)—NH—, or —V₂-L₂-R^{3aa}—V₃—, wherein L₂, R^{3aa}, V₂, and V₃ have the values described herein. In some embodiments, X is a bond. In some embodiments, X is —NH—C(O)—. In some embodiments, X is —C(O)—NH—. In some embodiments, X is —V₂-L₂-R^{3aa}—V₃—, wherein L₂, R^{3aa}, V₂, and V₃ have the values described herein. In some embodiments, X is a bond, —NH—C(O)—, —C(O)—NH—,

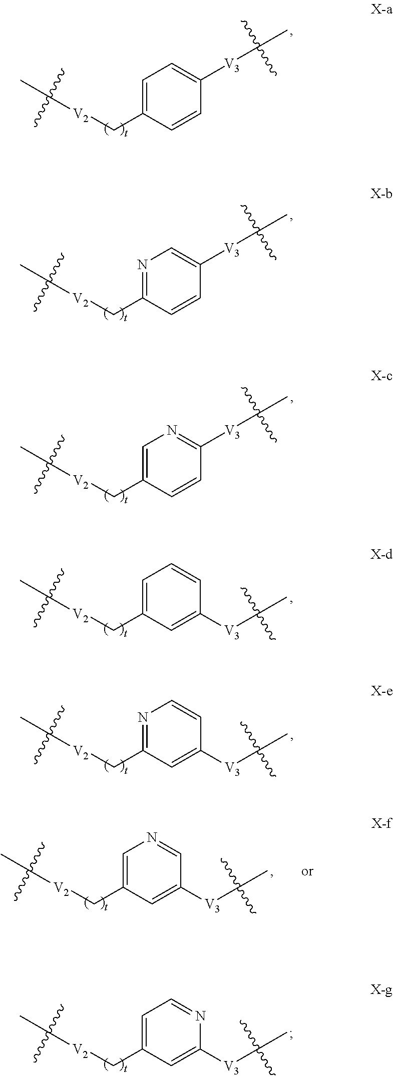

wherein V₂, V₃, and t have the values described herein.

In certain embodiments, X is a bond, —NH—C(O)—, —C(O)—NH—,

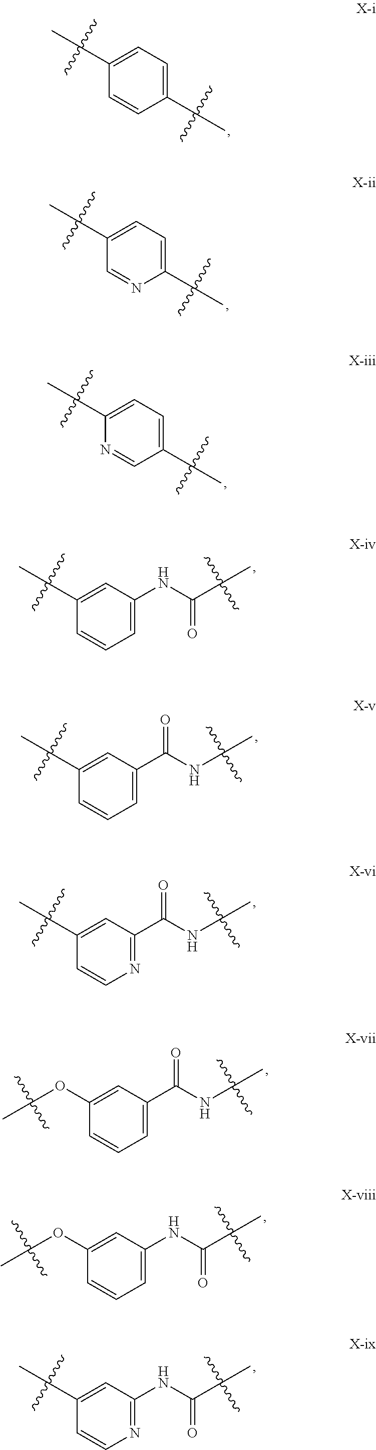

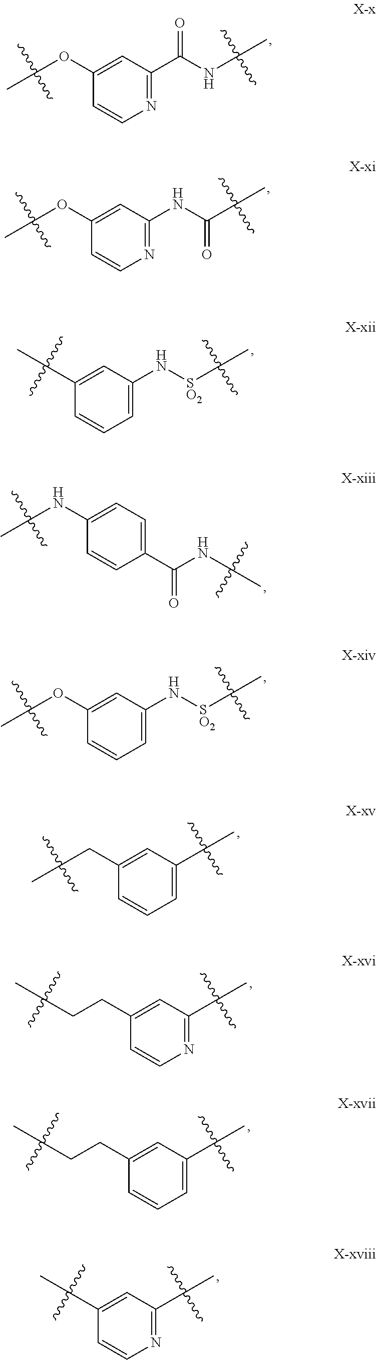

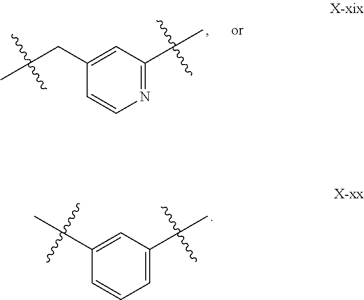

In certain embodiments, X is —NH—C(O)—, —C(O)—NH—, X-iv, X-vi, X-vii, X-viii, X-ix, or X-x.

The variable V₂ is a bond, —NH—C(O)—, —C(O)—NH—, —NH—, or —O—. In some embodiments, V₂ is a bond, —NH—C(O)— or —O—. In certain embodiments, V₂ is a bond. In certain embodiments, V₂ is —O—. In certain embodiments, V₂ is —NH—C(O)—.

The variable L₂ is a bond or unsubstituted or substituted C₁₋₃ alkylene chain. In some embodiments, L₂ is a bond, —CH₂—, —CH₂CH₂—, or —CH₂CH₂CH₂—. In certain embodiments, L₂ is a bond. In certain embodiments, L₂ is In certain embodiments, L₂ is —CH₂CH₂—.

Ring C is a 4-7 membered heterocyclic ring containing one nitrogen atom, wherein the nitrogen atom is not the atom bound to X, and wherein the nitrogen atom in Ring C is substituted with R^{9bb} and Ring C is unsubstituted or substituted by 1-4 occurrences of R^{5b}; wherein R^{9bb}, X, and R^{5b} have the values described herein. In some embodiments, Ring C is a 4-7 membered heterocyclic ring containing one nitrogen atom, wherein the nitrogen atom is not the atom bound to X, and wherein the nitrogen atom in Ring C is substituted with R^{9bb} and Ring C is unsubstituted or substituted by 1-2 occurrences of R^{5b}; wherein R^{9bb}, X, and R^{5b} have the values described herein.

In certain embodiments, Ring C is:

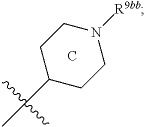

wherein Ring C is unsubstituted or substituted with 1 occurrence of R^{5b}, wherein R^{9bb} and R^{5b} have the values described herein. In certain embodiments, Ring C is:

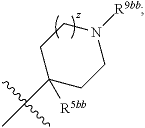

wherein R^{9bb}, z and R^{5bb} have the values described herein.

The variable V₃ is a bond, —NH—C(O)—, —C(O)—NH—, —NH—S(O)₂—, or —NH—C(O)—NH—. In some embodiments, V₃ is a bond, —C(O)—NH—, or —NH—C(O)—. In certain embodiments, V₃ is a bond. In certain embodiments, V₃ is —NH—C(O)—. In certain embodiments, V₃ is —C(O)—NH—.

The variable t is 0-2. In some embodiments, t is 0-1. In certain embodiments, t is 0. In certain embodiments, t is 1. In certain embodiments, t is 2.

The variable R^{3aa} is a 6-membered aromatic ring containing 0-2 nitrogen atoms which is unsubstituted or substituted with 1-2 independent occurrences of R^{4c}, wherein R^{4c} has the values described herein. In some embodiments, R^{3aa} is phenyl or pyridyl, each of which is unsubstituted or substituted with 1-2 independent occurrences of R^{4c}, wherein R^{4c} has the values described herein. In some embodiments, R^{3aa} is:

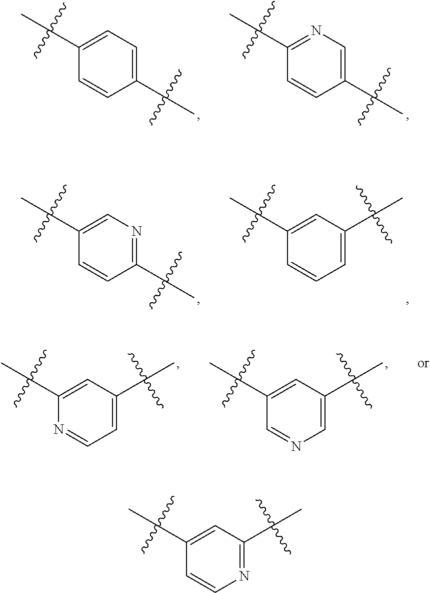

wherein each ring is unsubstituted or substituted with 1-2 independent occurrences of R^{4c}.

The variable R^{4c} is chloro, fluoro, cyano, hydroxy, methoxy, ethoxy, trifluoromethoxy, trifluoromethyl, methyl, or ethyl. In some embodiments, R^{4c} is chloro, fluoro, methyl or ethyl.

The variable z is 0-1. In some embodiments, z is 0. In some embodiments, z is 1.

Each occurrence of the variable R^{5b} is independently chloro, fluoro, hydroxy, methyl, ethyl, methoxy, ethoxy, trifluoromethyl, trifluoromethoxy, —C(O)NH₂, or —CO₂H. In some embodiments, each occurrence of the variable R^{5b} is independently chloro, fluoro, hydroxy, methyl, or ethyl. In certain embodiments, each occurrence of the variable R^{5b} is methyl.

The variable R^{5bb} is hydrogen or methyl. In some embodiments, R^{5bb} is hydrogen. In some embodiments, R^{5bb} is methyl.

The variable R^{9bb} is hydrogen, unsubstituted C(O)—O—C₁₋₆ aliphatic, unsubstituted C(O)—C₁₋₆ aliphatic, unsubstituted C(O)—C₃₋₁₀ cycloaliphatic, or unsubstituted C₁₋₆ aliphatic. In some embodiments, R^{9bb} is hydrogen, methyl, ethyl, isopropyl, or tert-butoxycarbonyl. In some embodiments, R^{9bb} is methyl, ethyl, or isopropyl. In certain embodiments, R^{9bb} is hydrogen.

In certain embodiments for the compounds of formulas (I), (II), (III) and (IV):

G is —V₁—R³, -L₁-R³, or —R³;

L₁ is —CH₂— or —CH₂CH₂—; and

V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—;

wherein R³ and R^{4a} have the values contained herein.

In certain embodiments, for the compound of formula (I):

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

each occurrence of R¹ is independently hydrogen, chloro, fluoro, cyano, hydroxy, methoxy, ethoxy, trifluoromethoxy, trifluoromethyl, methyl, or ethyl.

In certain embodiments, for the compounds of formula (II):

each occurrence of R^{1a} is independently hydrogen, fluoro, trifluoromethyl, or methyl;

R^{1c} is hydrogen, hydroxy, fluoro, trifluoromethyl, or methyl;

each occurrence of R¹ is independently hydrogen, chloro, fluoro, cyano, hydroxy, methoxy, ethoxy, trifluoromethyl, methyl, or ethyl.

In certain embodiments, the compound of formula (I) is represented by:

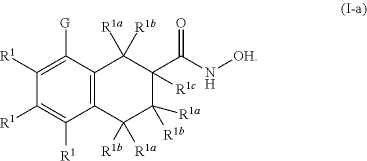

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

each occurrence of R¹ is independently hydrogen, chloro, fluoro, cyano, hydroxy, methoxy, ethoxy, trifluoromethyl, methyl, or ethyl;

G is —V₁—R³, -L₁-R³, or —R³;

L₁ is —CH₂— or —CH₂CH₂—; and

V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—;

wherein R³ and R^{4a} have the values contained herein.

In certain embodiments, the compound of formula (I) is represented by:

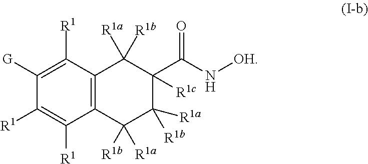

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

each occurrence of R¹ is independently hydrogen, chloro, fluoro, cyano, hydroxy, methoxy, ethoxy, trifluoromethyl, methyl, or ethyl;

G is —V₁—R³, -L₁-R³, or —R³;

L₁ is —CH₂— or —CH₂CH₂—; and

V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—;

wherein R³ and R^{4a} have the values contained herein.

In certain embodiments, the compound of formula (I) is represented by:

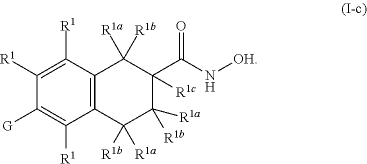

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

each occurrence of R¹ is independently hydrogen, chloro, fluoro, cyano, hydroxy, methoxy, ethoxy, trifluoromethyl, methyl, or ethyl;

G is —V₁—R³, -L₁-R³, or —R³;

L₁ is —CH₂— or —CH₂CH₂—; and

V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—;

wherein R³ and R^{4a} have the values contained herein.

In certain embodiments, the compound of formula (I) is represented by:

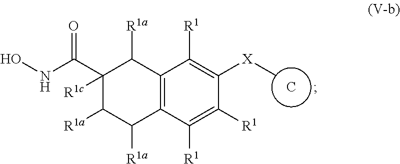

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl; and

X and Ring C have the values described herein.

In certain such embodiments:

R¹ is H;

R^{1c} is H; and

R^{1a} is H.

In certain embodiments, the compound of formula (I) is represented by:

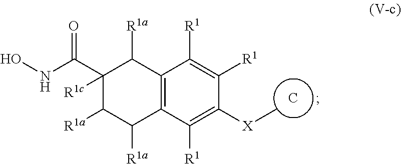

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl; and

X and Ring C have the values described herein.

In certain such embodiments:

R¹ is H;

R^{1c} is H; and

R^{1a} is H.

In certain embodiments, the compound of formula (I) is represented by:

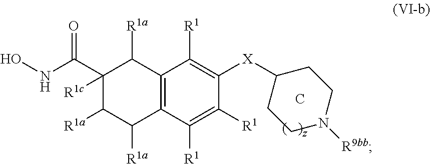

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

R^{9bb} is hydrogen, methyl, ethyl, isopropyl, or tert-butoxycarbonyl;

Ring C is unsubstituted or substituted with one occurrence of R^{5b};

X is a bond, —NH—C(O)—, —C(O)—NH—, X-a, X-b, X-c, X-d, X-e, X-f, or X-g; and

z, R^{5b}, t, V₂, and V₃ have the values described herein.

In certain such embodiments:

R^{5b} is methyl;

R¹ is H;

R^{1c} is H; and

R^{1a} is H.

In certain embodiments, the compound of formula (I) is represented by:

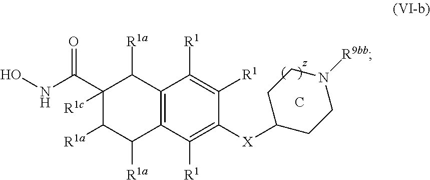

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

R^{9bb} is hydrogen, methyl, ethyl, isopropyl, or tert-butoxycarbonyl;

Ring C is unsubstituted or substituted with one occurrence of R^{5b};

X is a bond, —NH—C(O)—, —C(O)—NH—, X-a, X-b, X-c, X-d, X-e, X-f, or X-g; and

z, R^{5b}, t, V₂, and V₃ have the values described herein.

In certain such embodiments:

R^{5b} is methyl;

R¹ is H;

R^{1c} is H; and

R^{1a} is H.

In certain embodiments, the compound of formula (I) is represented by:

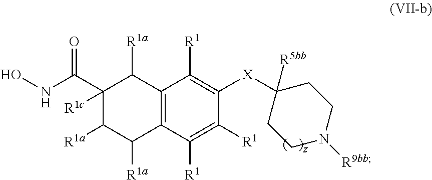

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

R^{9bb} is hydrogen, methyl, ethyl, isopropyl, or tert-butoxycarbonyl;

R^{5bb} is hydrogen or methyl;

X is —NH—C(O)—, —C(O)—NH—, X-iv, X-vi, X-vii, X-viii, X-ix, or X-x; and

z has the values described herein.

In certain such embodiments:

R^{5bb} is methyl;

z is 1;

R¹ is H;

R^{1c} is H; and

R^{1a} is H.

In certain embodiments, the compound of formula (I) is represented by:

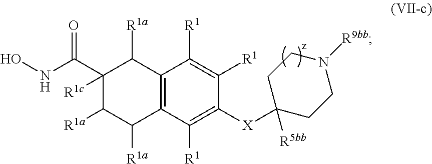

wherein:

each occurrence of R^{1a} is independently hydrogen, fluoro, trifluoromethyl, or methyl;

each occurrence of R^{1b} is hydrogen;

R^{1c} is hydrogen, hydroxy, fluoro, trifluoromethyl, or methyl;

R^{9bb} is hydrogen, methyl, ethyl, isopropyl, or tert-butoxycarbonyl;

R^{5bb} is hydrogen or methyl;

X is —NH—C(O)—, —C(O)—NH—, X-iv, X-vi, X-vii, X-viii, X-ix, or X-x; and

z has the values described herein.

In certain such embodiments:

R^{5bb} is methyl;

z is 1;

R¹ is H;

R^{1c} is H; and

R^{1a} is H.

In some embodiments, the compound of formula (I) is represented by formula (II-a)-(II-d):

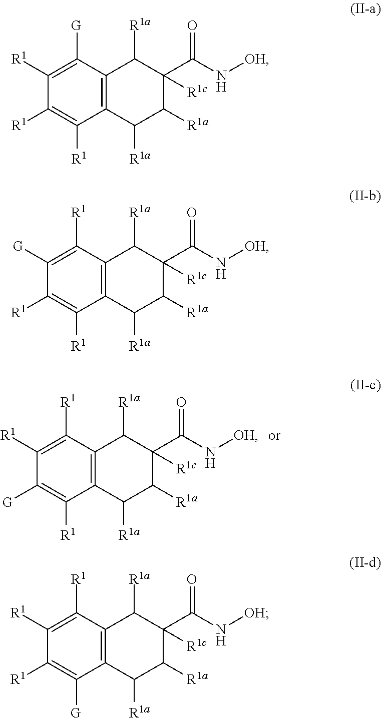

wherein:

G is —V₁—R³, -L₁-R³, or —R³;

L₁ is —CH₂— or —CH₂CH₂—;

V₁ is —NH—, —NH—C(O)—, —C(O)—NH—, or —O—;

each occurrence of R^{1a} is independently hydrogen, fluoro, trifluoromethyl, or methyl;

R^{1c} is hydrogen, hydroxy, fluoro, trifluoromethyl, or methyl; and

each occurrence of R¹ is independently hydrogen, chloro, fluoro, cyano, hydroxy, methoxy, ethoxy, trifluoromethyl, methyl, or ethyl;

wherein R³ has the values described herein.

In certain such embodiments, the compound of formula (I) is represented by formula (II-a). In certain such embodiments, the compound of formula (I) is represented by formula (II-b). In certain such embodiments, the compound of formula (I) is represented by formula (II-c). In certain such embodiments, the compound of formula (I) is represented by formula (II-d).

In some embodiments, the compound of formula (I) is represented by formula (II-a)-(II-d):

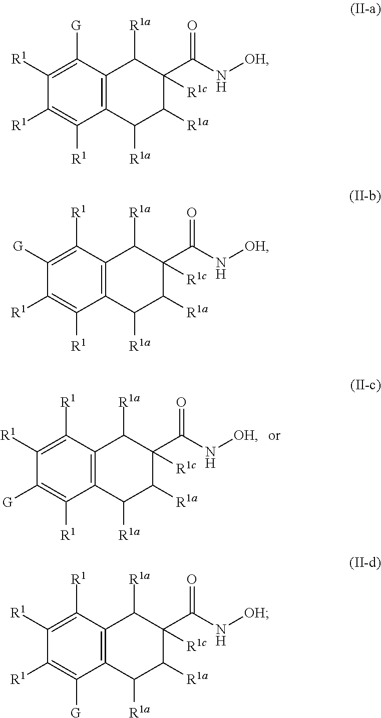

wherein:

G is —V₁—R³, -L₁-R³, or —R³;

L₁ is —CH₂— or —CH₂CH₂—;

V₁ is —NH—, —NH—C(O)—, —C(O)—NH—, or —O—;

each occurrence of R^{1a} is hydrogen;

R^{1c} is hydrogen; and

each occurrence of R¹ is hydrogen;

wherein R³ has the values described herein.

In certain such embodiments, the compound of formula (I) is represented by formula (II-a). In certain such embodiments, the compound of formula (I) is represented by formula (II-b). In certain such embodiments, the compound of formula (I) is represented by formula (II-c). In certain such embodiments, the compound of formula (I) is represented by formula (II-d).

Representative examples of compounds of formula (I) are shown in Table 1:

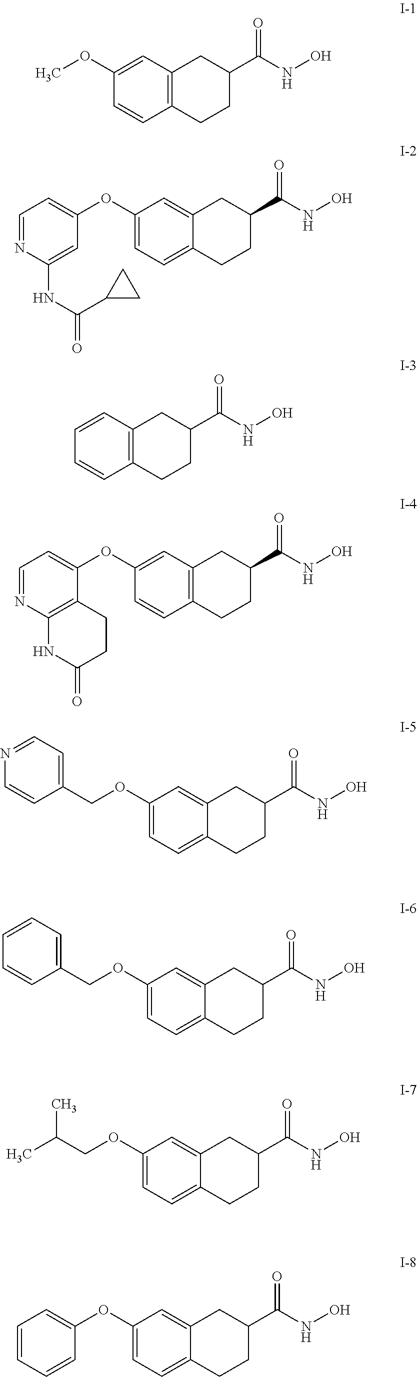

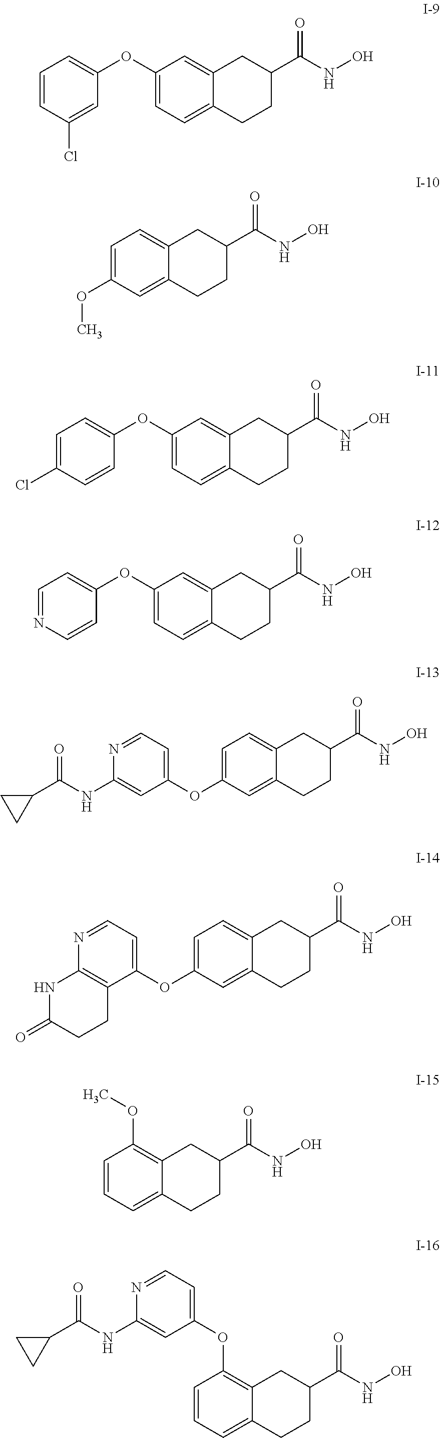

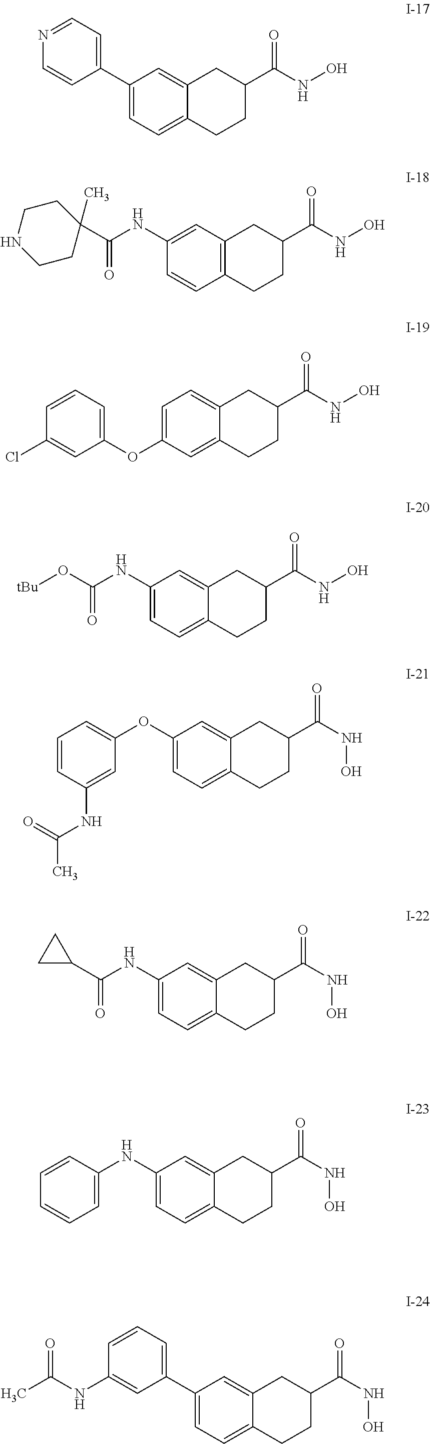

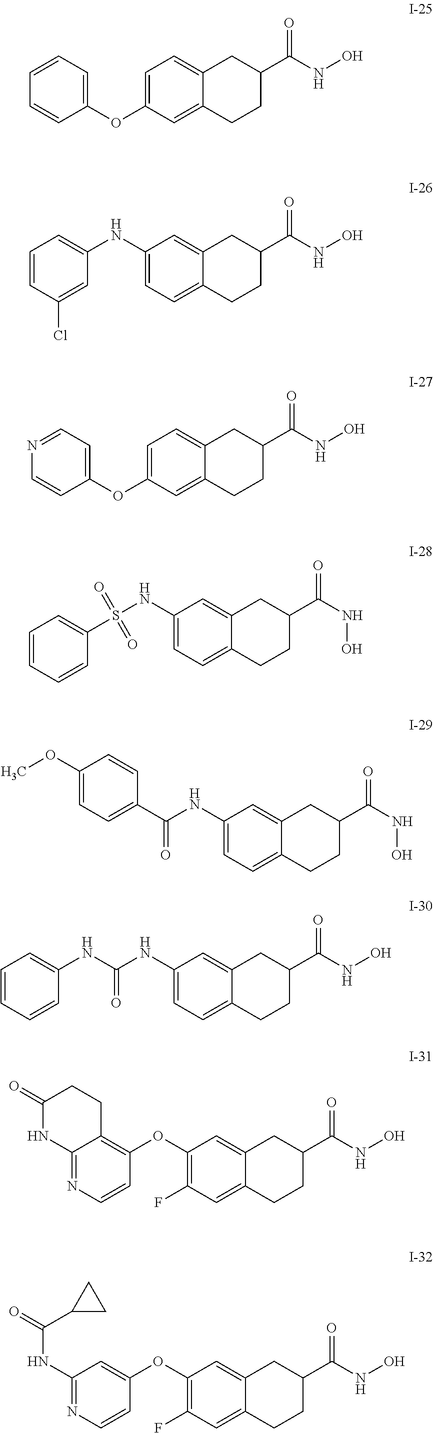

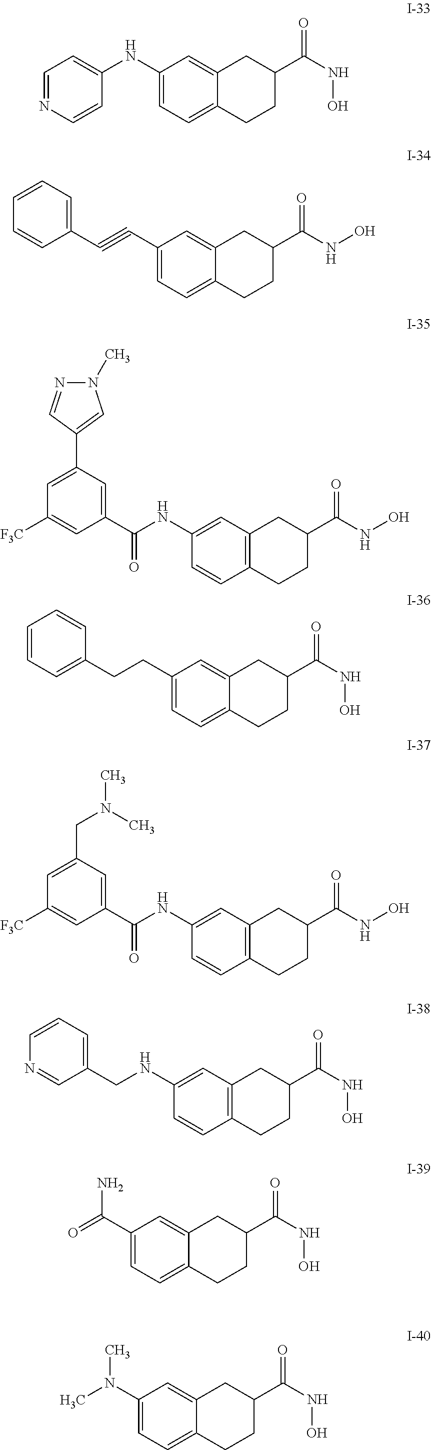

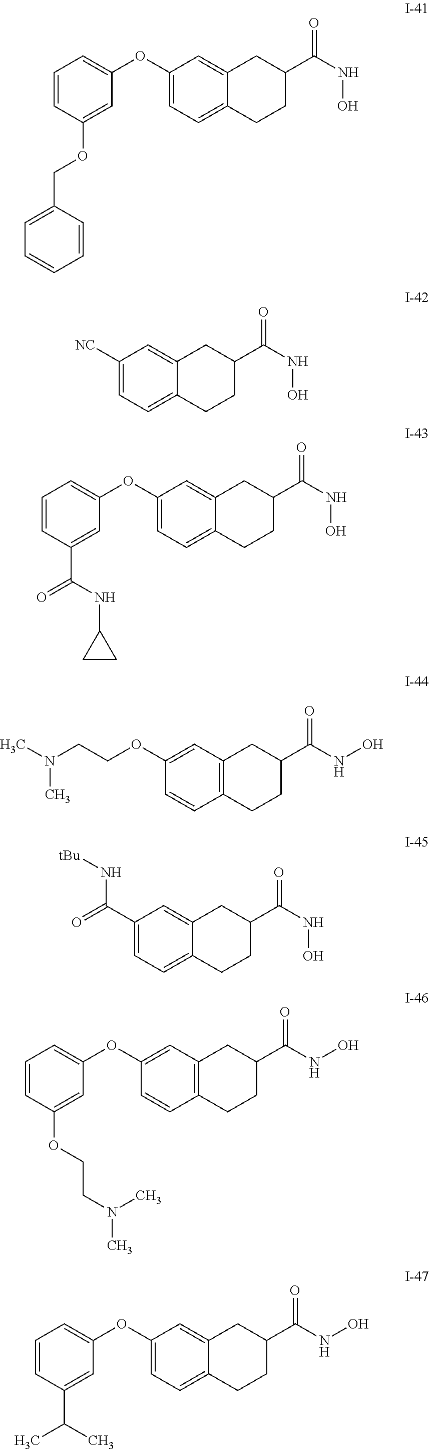

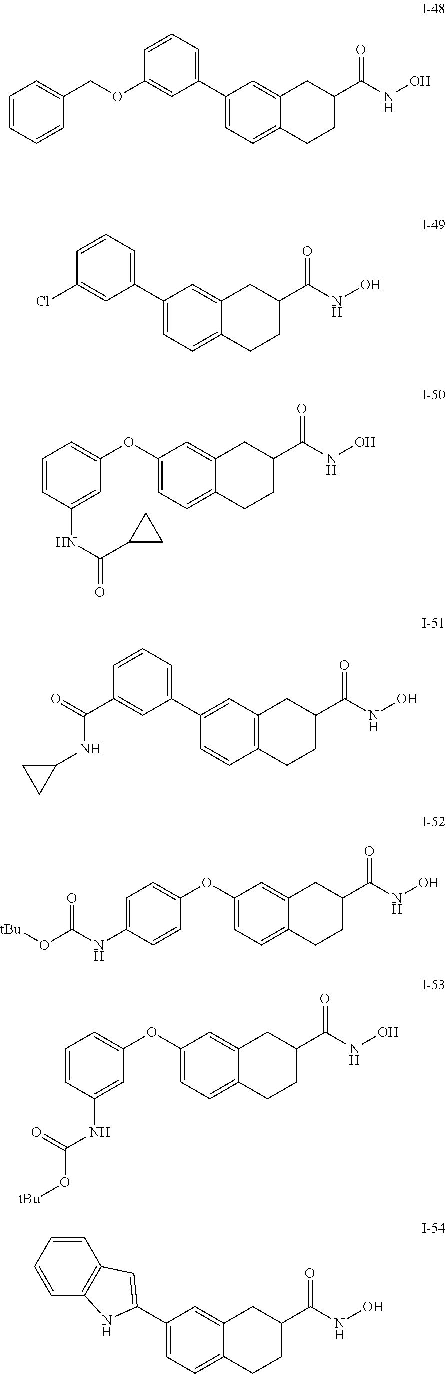

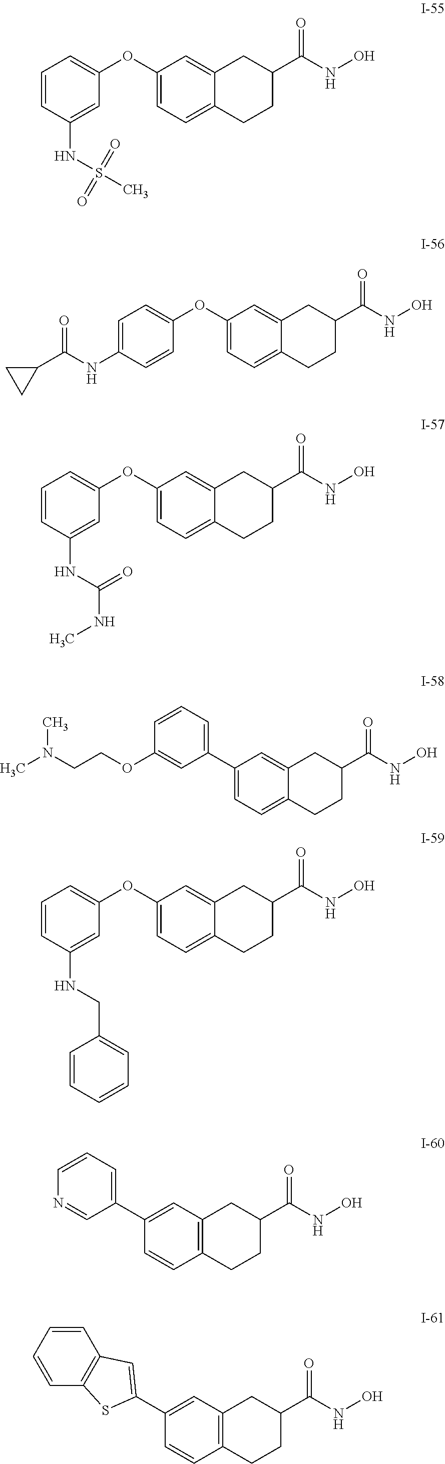

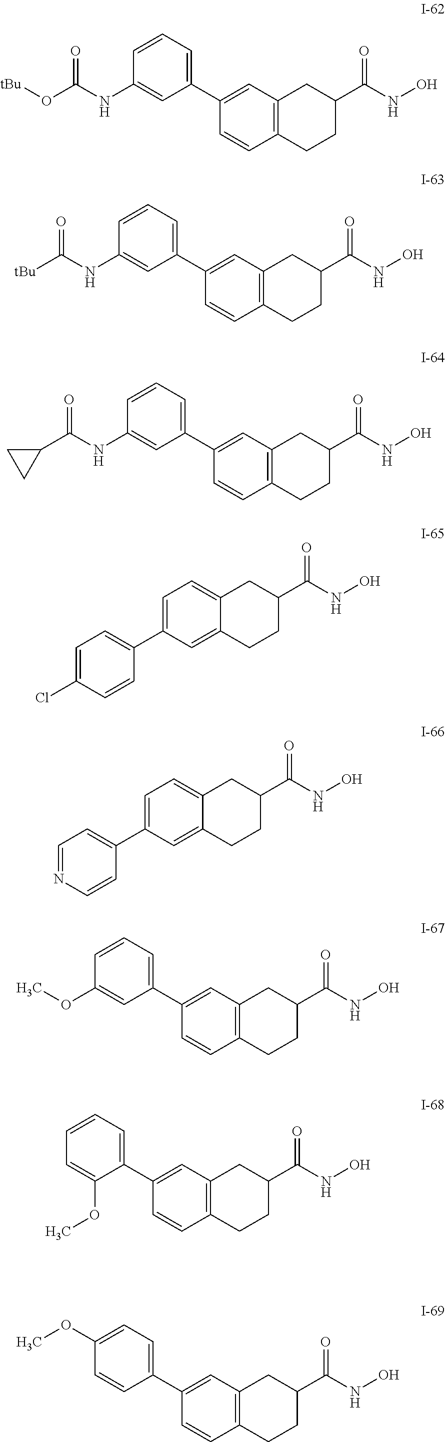

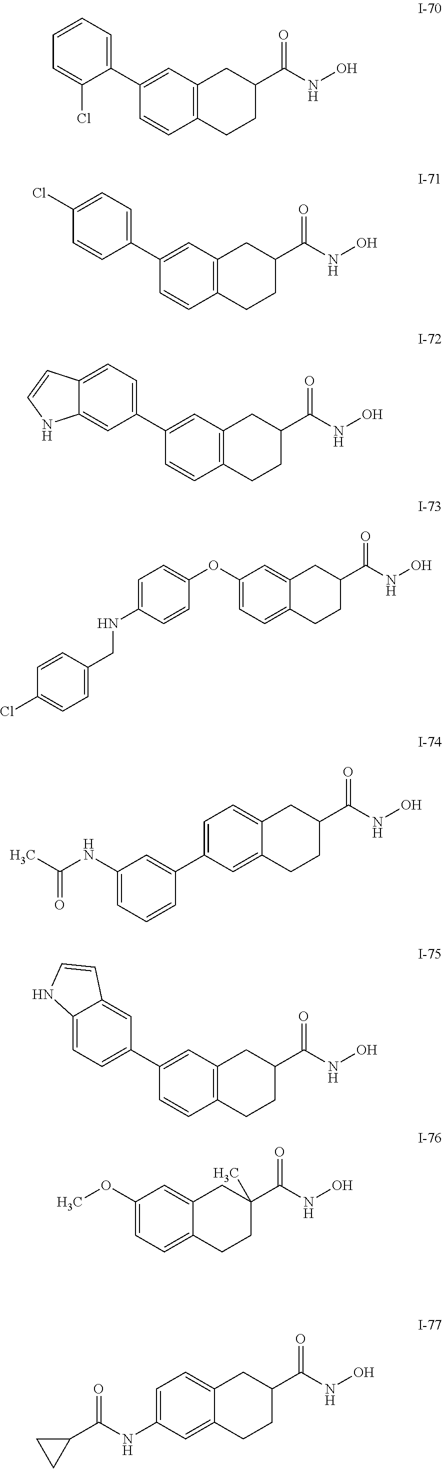

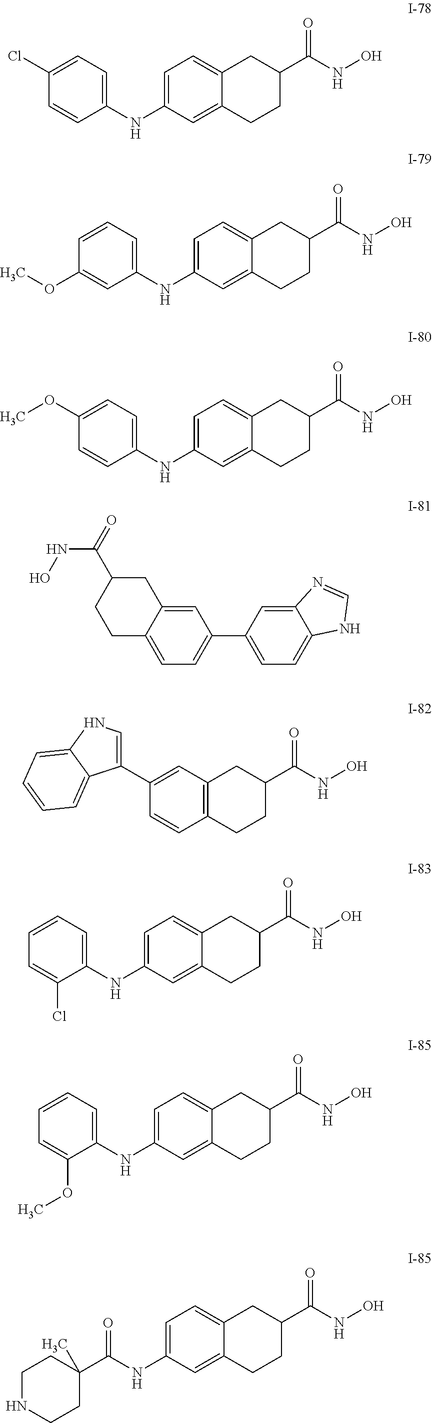

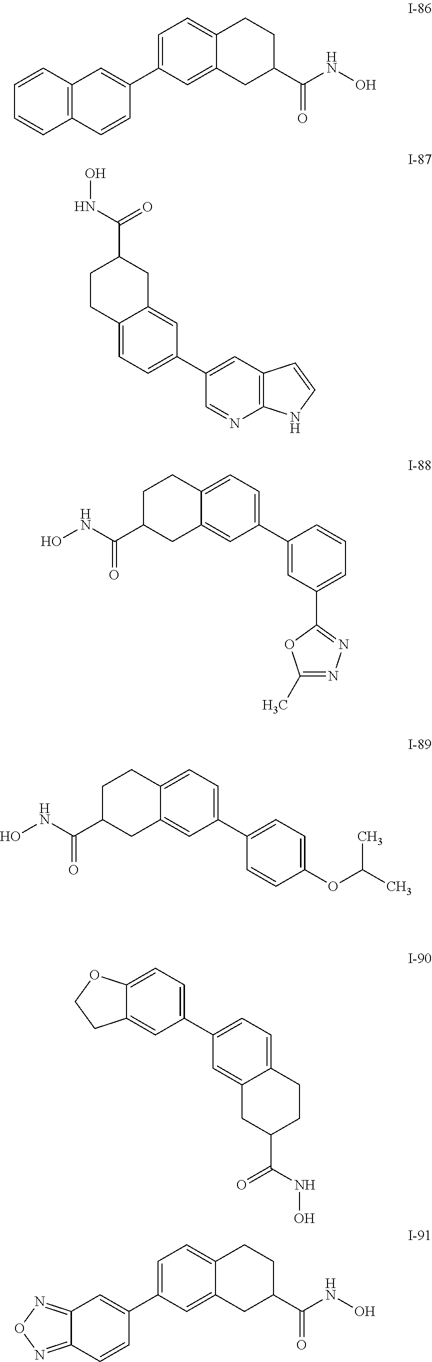

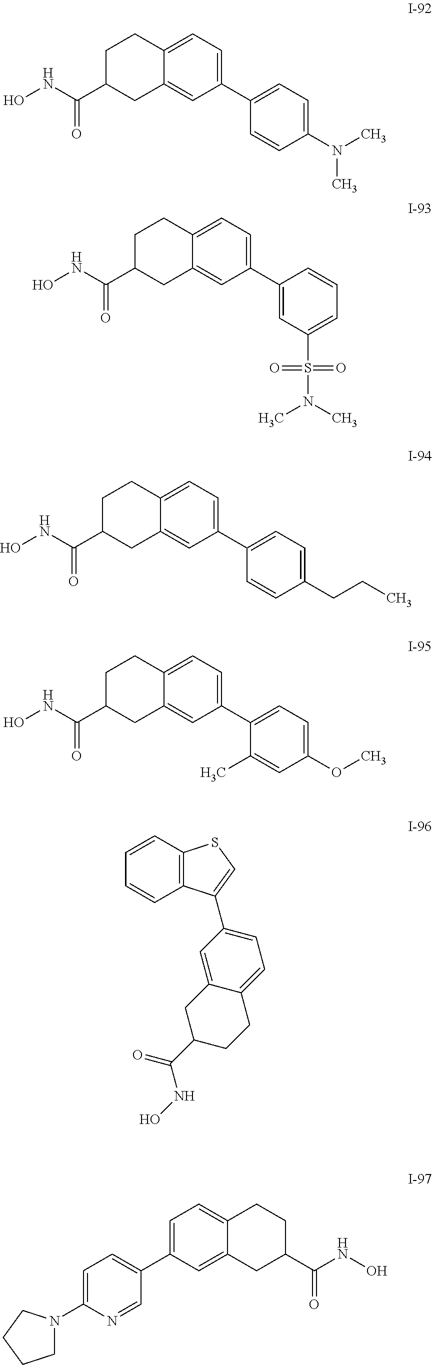

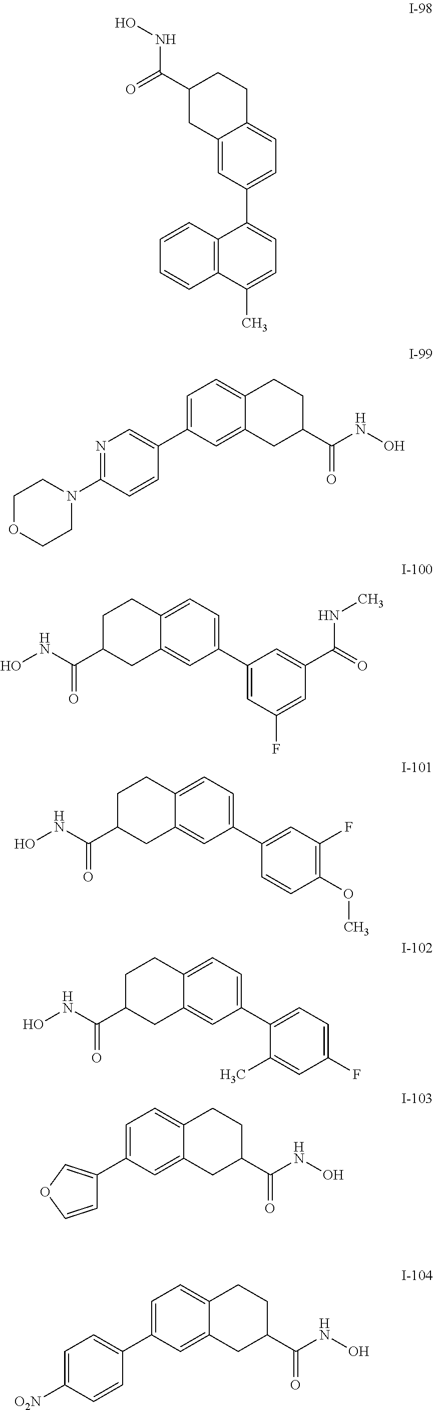

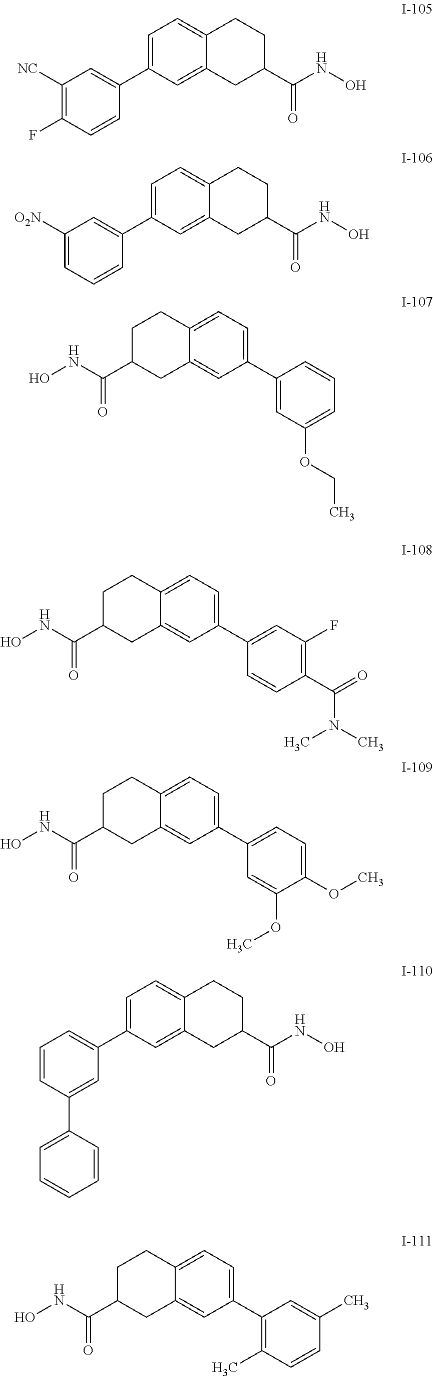

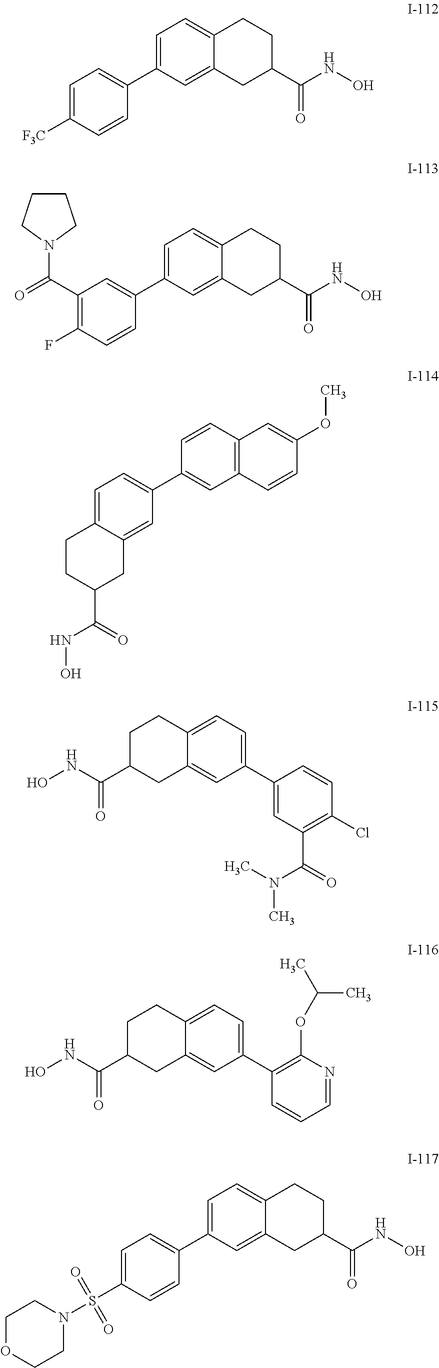

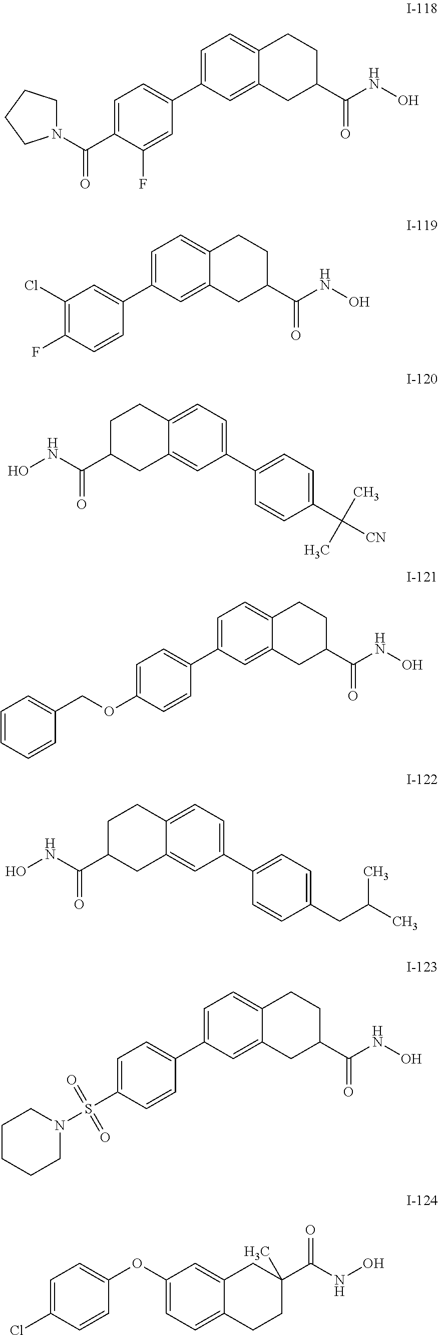

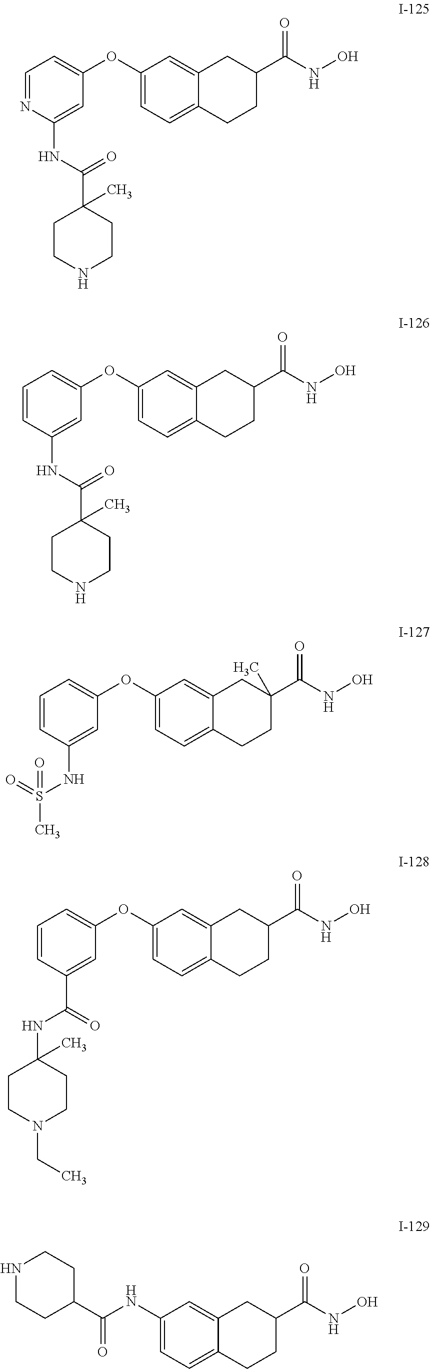

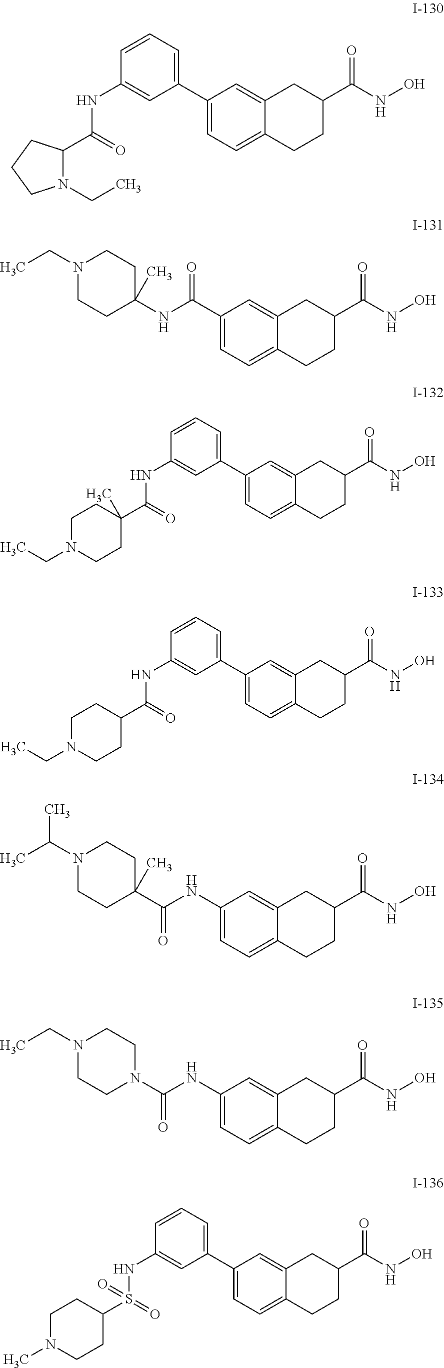

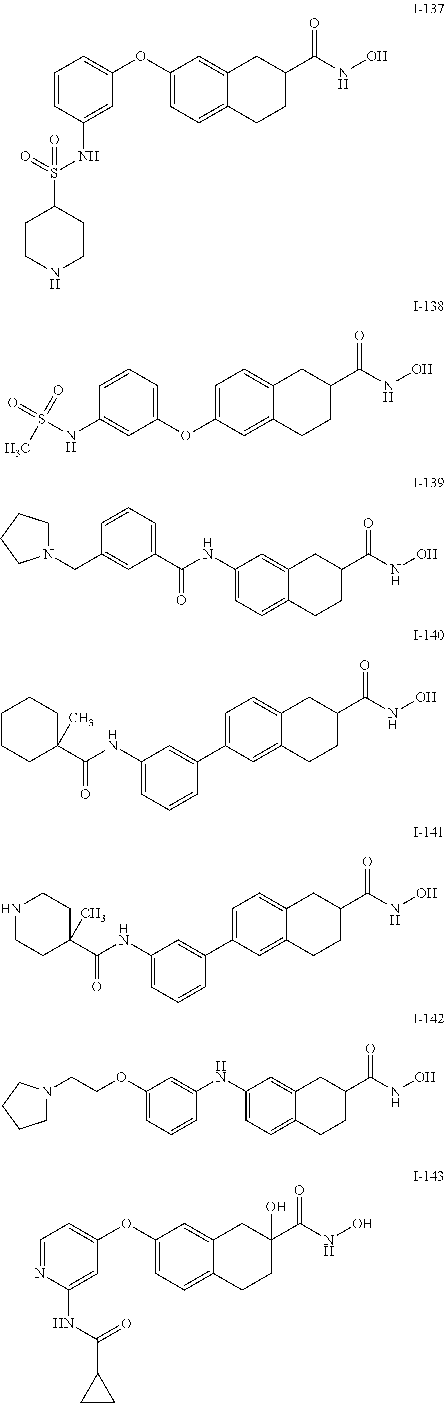

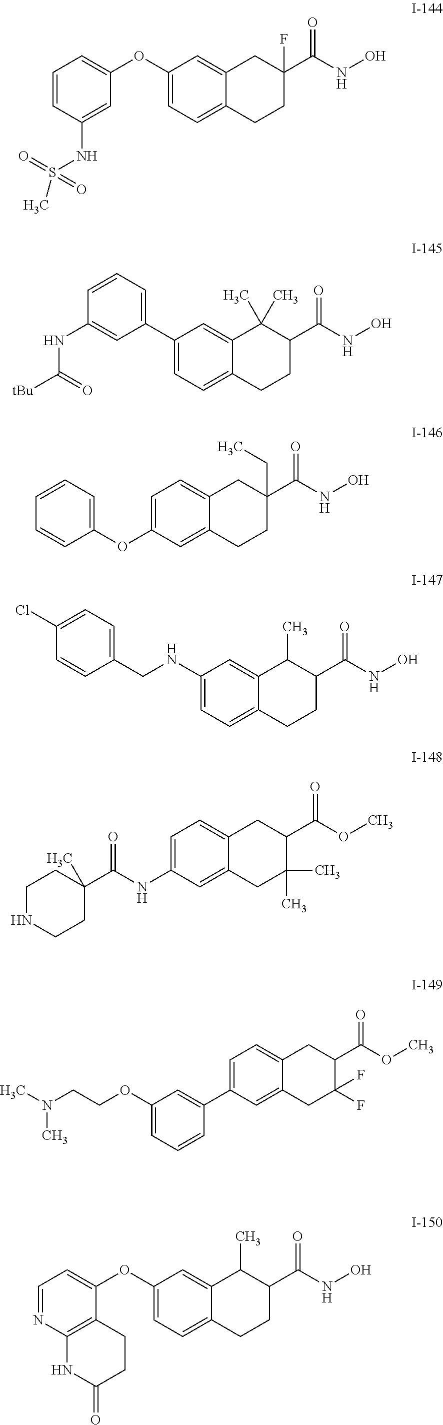

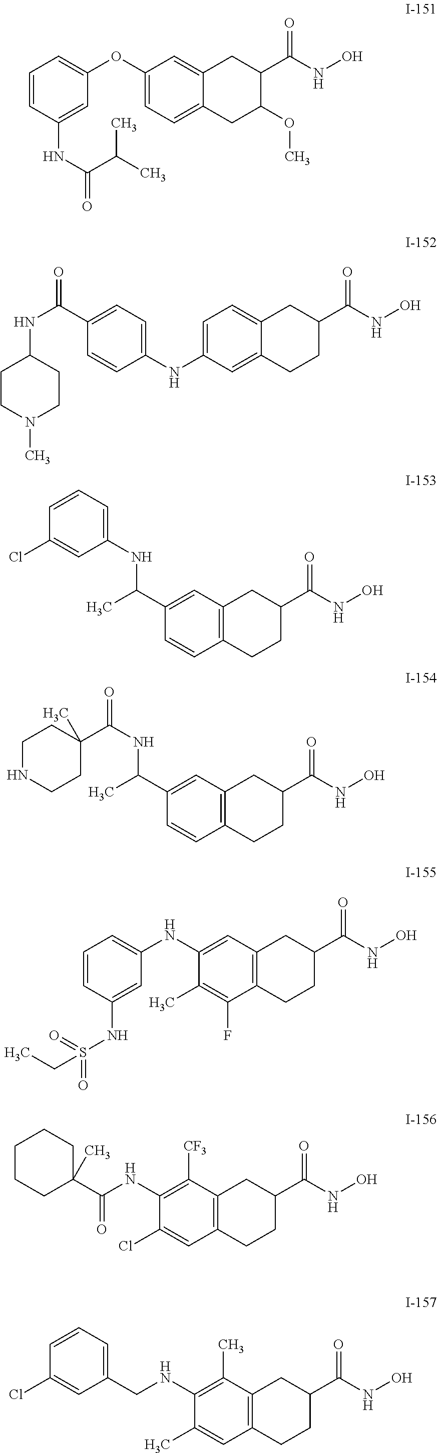

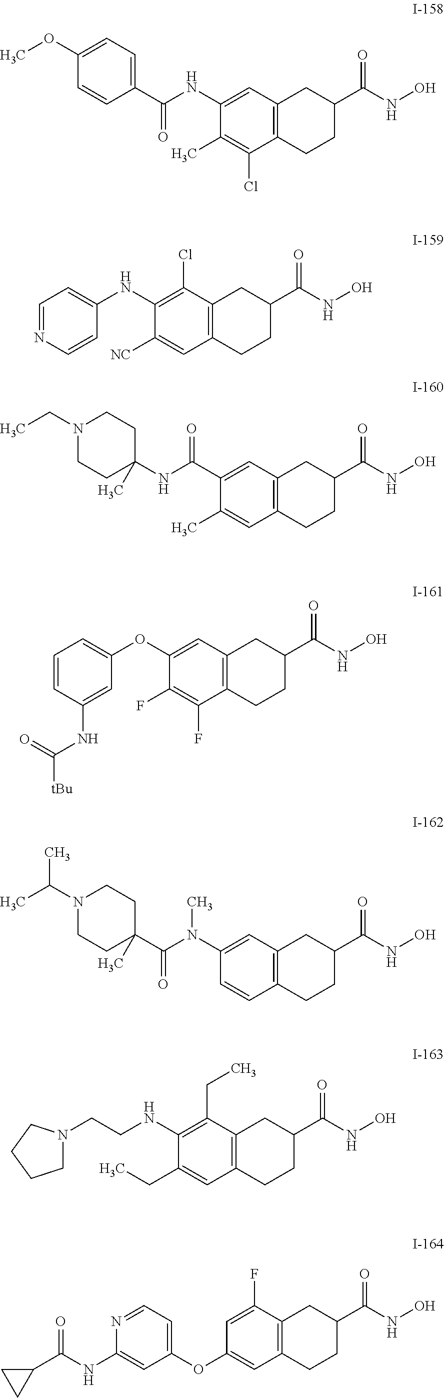

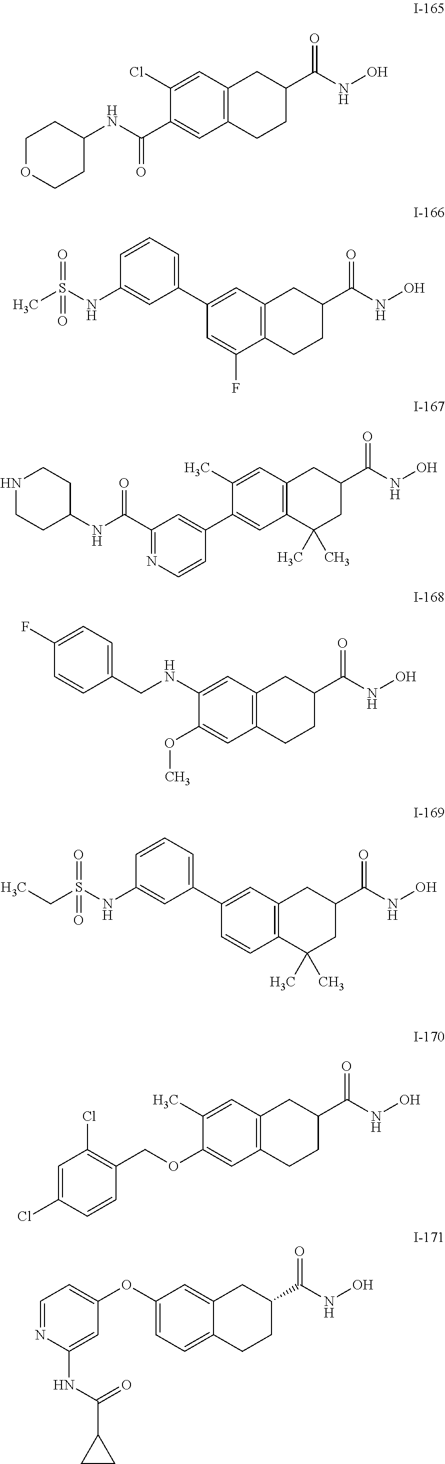

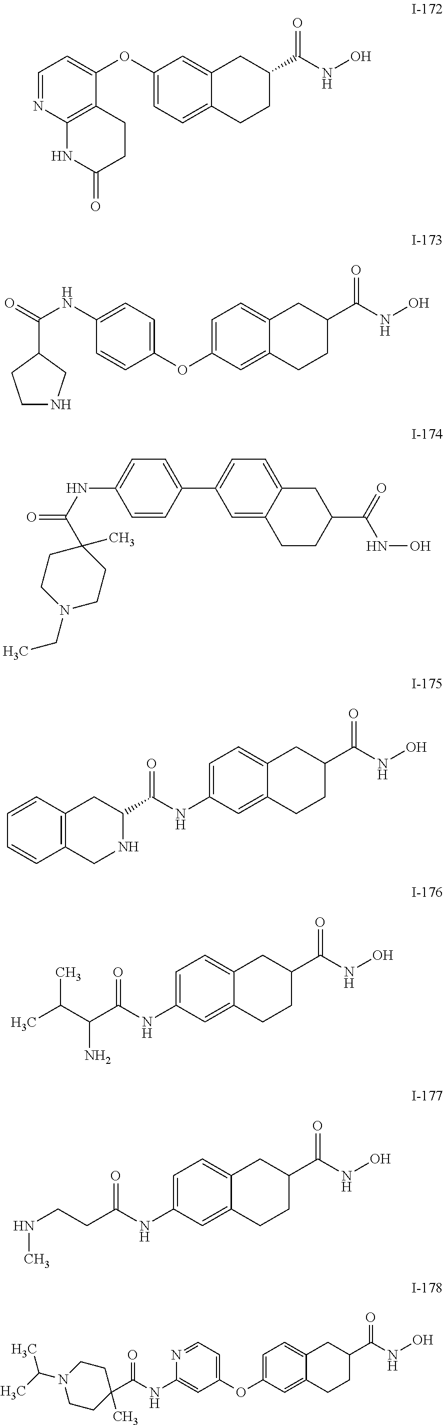

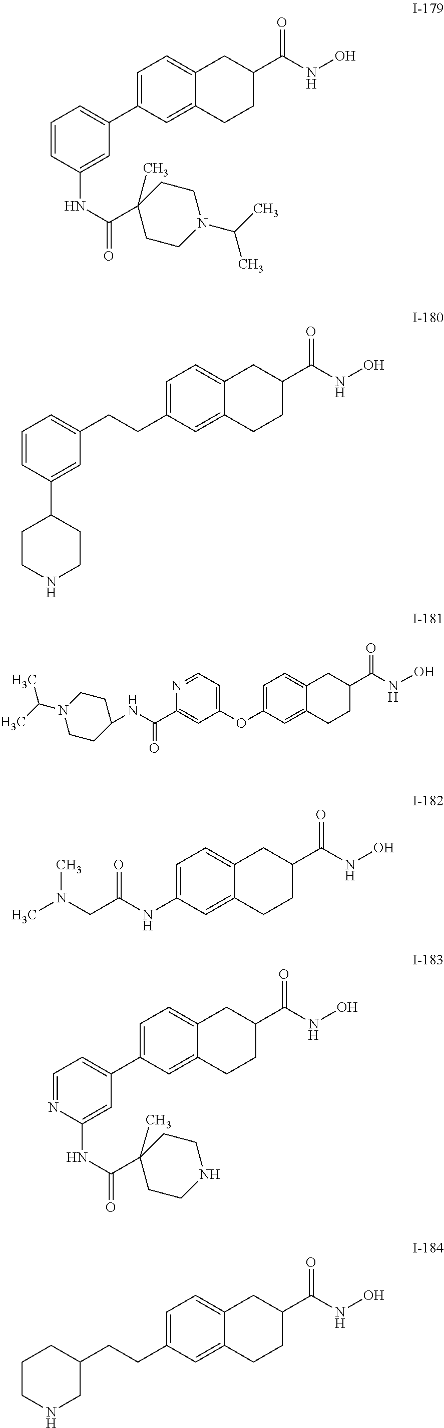

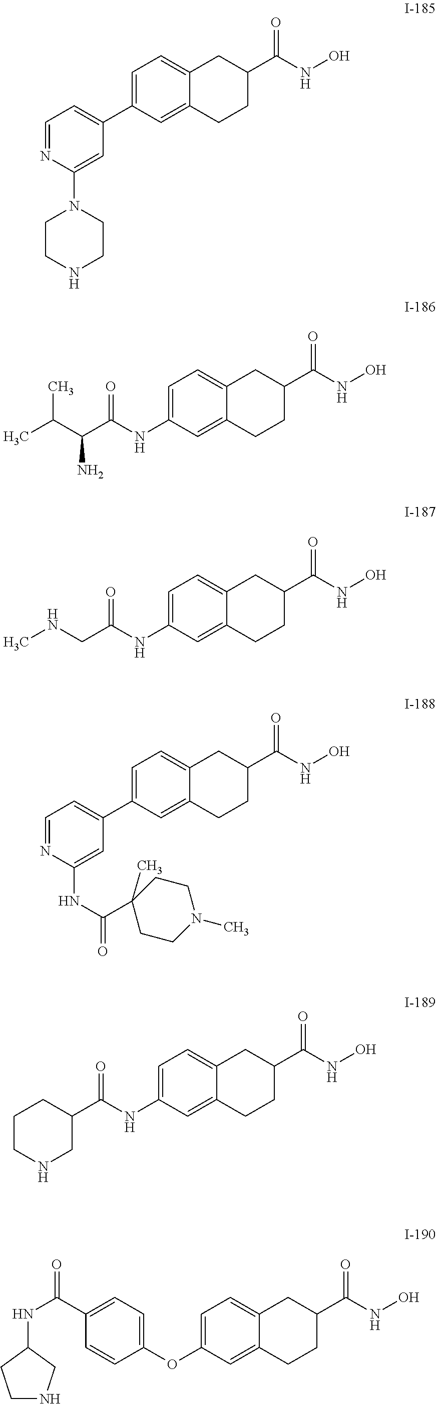

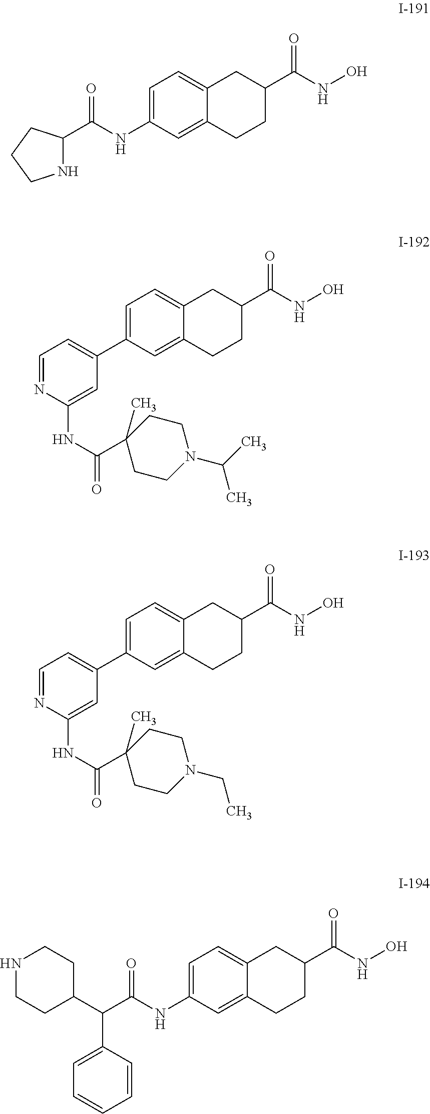

The compounds in Table 1 above may also be identified by the following chemical names:

| | |
|---|---|
| I-1 | N-hydroxy-7-methoxy-1,2,3,4-tetrahydronaphthalene-2- |
| | carboxamide |
| I-2 | (2S)-7-({2-[(cyclopropylcarbonyl)amino]pyridin-4-yl}oxy)-N- |
| | hydroxy-1,2,3,4-tetrahydronaphthalene-2-carboxamide |
| I-3 | N-hydroxy-1,2,3,4-tetrahydronaphthalene-2-carboxamide |
| I-4 | (2S)—N-hydroxy-7-[(7-oxo-5,6,7,8-tetrahydro-1,8-naphthyridin-4- |
| | yl)oxy]-1,2,3,4-tetrahydronaphthalene-2-carboxamide |
| I-5 | N-hydroxy-7-(pyridin-4-ylmethoxy)-1,2,3,4-tetrahydronaphthalene- |
| | 2-carboxamide |
| I-6 | 7-(benzyloxy)-N-hydroxy-1,2,3,4-tetrahydronaphthalene-2- |
| | carboxamide |
| I-7 | N-hydroxy-7-isobutoxy-1,2,3,4-tetrahydronaphthalene-2- |
| | carboxamide |
| I-8 | N-hydroxy-7-phenoxy-1,2,3,4-tetrahydronaphthalene-2- |
| | carboxamide |
| I-9 | 7-(3-chlorophenoxy)-N-hydroxy-1,2,3,4-tetrahydronaphthalene-2- |
| | carboxamide |
| I-10 | N-hydroxy-6-methoxy-1,2,3,4-tetrahydronaphthalene-2- |
| | carboxamide |
| I-11 | 7-(4-chlorophenoxy)-N-hydroxy-1,2,3,4-tetrahydronaphthalene-2- |
| | carboxamide |
| I-12 | N-hydroxy-7-(pyridin-4-yloxy)-1,2,3,4-tetrahydronaphthalene-2- |
| | carboxamide |
| I-13 | 6-({2-[(cyclopropylcarbonyl)amino]pyridin-4-yl}oxy)-N-hydroxy- |
| | 1,2,3,4-tetrahydronaphthalene-2-carboxamide |
| I-14 | N-hydroxy-6-[(7-oxo-5,6,7,8-tetrahydro-1,8-naphthyridin-4- |
| | yl)oxy]-1,2,3,4-tetrahydronaphthalene-2-carboxamide |
| I-15 | N-hydroxy-8-methoxy-1,2,3,4-tetrahydronaphthalene-2- |
| | carboxamide |
| I-16 | 8-({2-[(cyclopropylcarbonyl)amino]pyridin-4-yl}oxy)-N-hydroxy- |
| | 1,2,3,4-tetrahydronaphthalene-2-carboxamide |
| I-17 | N-hydroxy-7-pyridin-4-yl-1,2,3,4-tetrahydronaphthalene-2- |
| | carboxamide |
| I-18 | N-{7-[(hydroxyamino)carbonyl]-5,6,7,8-tetrahydronaphthalen-2- |
| | yl}-4-methylpiperidine-4-carboxamide |
| I-19 | 6-(3-chlorophenoxy)-N-hydroxy-1,2,3,4-tetrahydronaphthalene-2- |
| | carboxamide |
| I-20 | tert-butyl {7-[(hydroxyamino)carbonyl]-5,6,7,8- |
| | tetrahydronaphthalen-2-yl}carbamate |
| I-21 | 7-[3-(acetylamino)phenoxy]-N-hydroxy-1,2,3,4- |
| | tetrahydronaphthalene-2-carboxamide |
| I-22 | 7-[(cyclopropylcarbonyl)amino]-N-hydroxy-1,2,3,4- |
| | tetrahydronaphthalene-2-carboxamide |
| I-23 | 7-anilino-N-hydroxy-1,2,3,4-tetrahydronaphthalene-2-carboxamide |
| I-24 | 7-[3-(acetylamino)phenyl]-N-hydroxy-1,2,3,4- |
| | tetrahydronaphthalene-2-carboxamide |
| I-25 | N-hydroxy-6-phenoxy-1,2,3,4-tetrahydronaphthalene-2- |
| | carboxamide |
| I-26 | 7-[(3-chlorophenyl)amino]-N-hydroxy-1,2,3,4- |
| | tetrahydronaphthalene-2-carboxamide |
| I-27 | N-hydroxy-6-(pyridin-4-yloxy)-1,2,3,4-tetrahydronaphthalene-2- |
| | carboxamide |
| I-28 | N-hydroxy-7-[(phenylsulfonyl)amino]-1,2,3,4- |
| | tetrahydronaphthalene-2-carboxamide |
| I-29 | N-hydroxy-7-[(4-methoxybenzoyl)amino]-1,2,3,4- |
| | tetrahydronaphthalene-2-carboxamide |
| I-30 | 7-[(anilinocarbonyl)amino]-N-hydroxy-1,2,3,4- |
| | tetrahydronaphthalene-2-carboxamide |
| I-31 | 6-fluoro-N-hydroxy-7-[(7-oxo-5,6,7,8-tetrahydro-1,8-naphthyridin- |
| | 4-yl)oxy]-1,2,3,4-tetrahydronaphthalene-2-carboxamide |
| I-32 | 7-({2-[(cyclopropylcarbonyl)amino]pyridin-4-yl}oxy)-6-fluoro-N- |
| | hydroxy-1,2,3,4-tetrahydronaphthalene-2-carboxamide |
| I-33 | N-hydroxy-7-(pyridin-4-ylamino)-1,2,3,4-tetrahydronaphthalene-2- |
| | carboxamide |
| I-34 | N-hydroxy-7-(phenylethynyl)-1,2,3,4-tetrahydronaphthalene-2- |
| | carboxamide |
| I-35 | N-hydroxy-7-{[3-(1-methyl-1H-pyrazol-4-yl)-5- |
| | (trifluoromethyl)benzoyl]amino}-1,2,3,4-tetrahydronaphthalene-2- |
| | carboxamide |
| I-36 | N-hydroxy-7-(2-phenylethyl)-1,2,3,4-tetrahydronaphthalene-2- |
| | carboxamide |
| I-37 | 7-{[3-[(dimethylamino)methyl]-5- |
| | (trifluoromethyl)benzoyl]amino}-N-hydroxy-1,2,3,4- |
| | tetrahydronaphthalene-2-carboxamide |
| I-38 | N-hydroxy-7-[(pyridin-3-ylmethyl)amino]-1,2,3,4- |
| | tetrahydronaphthalene-2-carboxamide |
| I-39 | N²-hydroxy-1,2,3,4-tetrahydronaphthalene-2,7-dicarboxamide |
| I-40 | 7-(dimethylamino)-N-hydroxy-1,2,3,4-tetrahydronaphthalene-2- |
| | carboxamide |
| I-41 | 7-[3-(benzyloxy)phenoxy]-N-hydroxy-1,2,3,4- |
| | tetrahydronaphthalene-2-carboxamide |
| I-42 | 7-cyano-N-hydroxy-1,2,3,4-tetrahydronaphthalene-2-carboxamide |
| I-43 | 7-{3-[(cyclopropylamino)carbonyl]phenoxy}-N-hydroxy-1,2,3,4- |
| | tetrahydronaphthalene-2-carboxamide |
| I-44 | 7-[2-(dimethylamino)ethoxy]-N-hydroxy-1,2,3,4- |
| | tetrahydronaphthalene-2-carboxamide |
| I-45 | N⁷-(tert-butyl)-N²-hydroxy-1,2,3,4-tetrahydronaphthalene-2,7- |
| | dicarboxamide |
| I-46 | 7-{3-[2-(dimethylamino)ethoxy]phenoxy}-N-hydroxy-1,2,3,4- |
| | tetrahydronaphthalene-2-carboxamide |
| I-47 | N-hydroxy-7-(3-isopropylphenoxy)-1,2,3,4-tetrahydronaphthalene- |
| | 2-carboxamide |
| I-48 | 7-[3-(benzyloxy)phenyl]-N-hydroxy-1,2,3,4-tetrahydronaphthalene- |
| | 2-carboxamide |
| I-49 | 7-(3-chlorophenyl)-N-hydroxy-1,2,3,4-tetrahydronaphthalene-2- |
| | carboxamide |
| I-50 | 7-{3-[(cyclopropylcarbonyl)amino]phenoxy}-N-hydroxy-1,2,3,4- |
| | tetrahydronaphthalene-2-carboxamide |
| I-51 | 7-{3-[(cyclopropylamino)carbonyl]phenyl}-N-hydroxy-1,2,3,4- |
| | tetrahydronaphthalene-2-carboxamide |
| I-52 | tert-butyl [4-({7-[(hydroxyamino)carbonyl]-5,6,7,8- |
| | tetrahydronaphthalen-2-yl}oxy)phenyl]carbamate |
| I-53 | tert-butyl [3-({7-[(hydroxyamino)carbonyl]-5,6,7,8- |
| | tetrahydronaphthalen-2-yl}oxy)phenyl]carbamate |
| I-54 | N-hydroxy-7-(1H-indol-2-yl)-1,2,3,4-tetrahydronaphthalene-2- |
| | carboxamide |
| I-55 | N-hydroxy-7-{3-[(methylsulfonyl)amino]phenoxy}-1,2,3,4- |
| | tetrahydronaphthalene-2-carboxamide |
| I-56 | 7-{4-[(cyclopropylcarbonyl)amino]phenoxy}-N-hydroxy-1,2,3,4- |
| | tetrahydronaphthalene-2-carboxamide |
| I-57 | N-hydroxy-7-(3-{[(methylamino)carbonyl]amino}phenoxy)- |
| | 1,2,3,4-tetrahydronaphthalene-2-carboxamide |
| I-58 | 7-{3-[2-(dimethylamino)ethoxy]phenyl}-N-hydroxy-1,2,3,4- |
| | tetrahydronaphthalene-2-carboxamide |
| I-59 | 7-[3-(benzylamino)phenoxy]-N-hydroxy-1,2,3,4- |
| | tetrahydronaphthalene-2-carboxamide |
| I-60 | N-hydroxy-7-pyridin-3-yl-1,2,3,4-tetrahydronaphthalene-2- |
| | carboxamide |
| I-61 | 7-(1-benzothien-2-yl)-N-hydroxy-1,2,3,4-tetrahydronaphthalene-2- |
| | carboxamide |
| I-62 | tert-butyl (3-{7-[(hydroxyamino)carbonyl]-5,6,7,8- |
| | tetrahydronaphthalen-2-yl}phenyl)carbamate |
| I-63 | 7-{3-[(2,2-dimethylpropanoyl)amino]phenyl}-N-hydroxy-1,2,3,4- |
| | tetrahydronaphthalene-2-carboxamide |
| I-64 | 7-{3-[(cyclopropylcarbonyl)amino]phenyl}-N-hydroxy-1,2,3,4- |
| | tetrahydronaphthalene-2-carboxamide |
| I-65 | 6-(4-chlorophenyl)-N-hydroxy-1,2,3,4-tetrahydronaphthalene-2- |
| | carboxamide |
| I-66 | N-hydroxy-6-pyridin-4-yl-1,2,3,4-tetrahydronaphthalene-2- |
| | carboxamide |
| I-67 | N-hydroxy-7-(3-methoxyphenyl)-1,2,3,4-tetrahydronaphthalene-2- |
| | carboxamide |
| I-68 | N-hydroxy-7-(2-methoxyphenyl)-1,2,3,4-tetrahydronaphthalene-2- |
| | carboxamide |
| I-69 | N-hydroxy-7-(4-methoxyphenyl)-1,2,3,4-tetrahydronaphthalene-2- |
| | carboxamide |
| I-70 | 7-(2-chlorophenyl)-N-hydroxy-1,2,3,4-tetrahydronaphthalene-2- |
| | carboxamide |
| I-71 | 7-(4-chlorophenyl)-N-hydroxy-1,2,3,4-tetrahydronaphthalene-2- |
| | carboxamide |
| I-72 | N-hydroxy-7-(1H-indol-6-yl)-1,2,3,4-tetrahydronaphthalene-2- |
| | carboxamide |
| I-73 | 7-{4-[(4-chlorobenzyl)amino]phenoxy}-N-hydroxy-1,2,3,4- |
| | tetrahydronaphthalene-2-carboxamide |
| I-74 | 6-[3-(acetylamino)phenyl]-N-hydroxy-1,2,3,4- |
| | tetrahydronaphthalene-2-carboxamide |
| I-75 | N-hydroxy-7-(1H-indol-5-yl)-1,2,3,4-tetrahydronaphthalene-2- |
| | carboxamide |
| I-76 | N-hydroxy-7-methoxy-2-methyl-1,2,3,4-tetrahydronaphthalene-2- |
| | carboxamide |
| I-77 | 6-[(cyclopropylcarbonyl)amino]-N-hydroxy-1,2,3,4- |
| | tetrahydronaphthalene-2-carboxamide |
| I-78 | 6-[(4-chlorophenyl)amino]-N-hydroxy-1,2,3,4- |
| | tetrahydronaphthalene-2-carboxamide |
| I-79 | N-hydroxy-6-[(3-methoxyphenyl)amino]-1,2,3,4- |
| | tetrahydronaphthalene-2-carboxamide |
| I-80 | N-hydroxy-6-[(4-methoxyphenyl)amino]-1,2,3,4- |
| | tetrahydronaphthalene-2-carboxamide |
| I-81 | 7-(1H-benzimidazol-5-yl)-N-hydroxy-1,2,3,4- |
| | tetrahydronaphthalene-2-carboxamide |
| I-82 | N-hydroxy-7-(1H-indol-3-yl)-1,2,3,4-tetrahydronaphthalene-2- |
| | carboxamide |
| I-83 | 6-[(2-chlorophenyl)amino]-N-hydroxy-1,2,3,4- |
| | tetrahydronaphthalene-2-carboxamide |
| I-84 | N-hydroxy-6-[(2-methoxyphenyl)amino]-1,2,3,4- |
| | tetrahydronaphthalene-2-carboxamide |
| I-85 | N-{6-[(hydroxyamino)carbonyl]-5,6,7,8-tetrahydronaphthalen-2- |
| | yl}-4-methylpiperidine-4-carboxamide |
| I-86 | N-hydroxy-5,6,7,8-tetrahydro-2,2′-binaphthalene-7-carboxamide |
| I-87 | N-hydroxy-7-(1H-pyrrolo[2,3-b]pyridin-5-yl)-1,2,3,4- |
| | tetrahydronaphthalene-2-carboxamide |
| I-88 | N-hydroxy-7-[3-(5-methyl-1,3,4-oxadiazol-2-yl)phenyl]-1,2,3,4- |
| | tetrahydronaphthalene-2-carboxamide |
| I-89 | N-hydroxy-7-(4-isopropoxyphenyl)-1,2,3,4-tetrahydronaphthalene- |
| | 2-carboxamide |
| I-90 | 7-(2,3-dihydro-1-benzofuran-5-yl)-N-hydroxy-1,2,3,4- |
| | tetrahydronaphthalene-2-carboxamide |
| I-91 | 7-(2,1,3-benzoxadiazol-5-yl)-N-hydroxy-1,2,3,4- |
| | tetrahydronaphthalene-2-carboxamide |
| I-92 | 7-[4-(dimethylamino)phenyl]-N-hydroxy-1,2,3,4- |
| | tetrahydronaphthalene-2-carboxamide |
| I-93 | 7-{3-[(dimethylamino)sulfonyl]phenyl}-N-hydroxy-1,2,3,4- |
| | tetrahydronaphthalene-2-carboxamide |
| I-94 | N-hydroxy-7-(4-propylphenyl)-1,2,3,4-tetrahydronaphthalene-2- |
| | carboxamide |
| I-95 | N-hydroxy-7-(4-methoxy-2-methylphenyl)-1,2,3,4- |
| | tetrahydronaphthalene-2-carboxamide |
| I-96 | 7-(1-benzothien-3-yl)-N-hydroxy-1,2,3,4-tetrahydronaphthalene-2- |
| | carboxamide |
| I-97 | N-hydroxy-7-(6-pyrrolidin-1-ylpyridin-3-yl)-1,2,3,4- |
| | tetrahydronaphthalene-2-carboxamide |
| I-98 | N-hydroxy-4-methyl-5′,6′,7′,8′-tetrahydro-1,2′-binaphthalene-7′- |
| | carboxamide |
| I-99 | N-hydroxy-7-(6-morpholin-4-ylpyridin-3-yl)-1,2,3,4- |
| | tetrahydronaphthalene-2-carboxamide |
| I-100 | 7-{3-fluoro-5-[(methylamino)carbonyl]phenyl}-N-hydroxy-1,2,3,4- |
| | tetrahydronaphthalene-2-carboxamide |
| I-101 | 7-(3-fluoro-4-methoxyphenyl)-N-hydroxy-1,2,3,4- |
| | tetrahydronaphthalene-2-carboxamide |
| I-102 | 7-(4-fluoro-2-methylphenyl)-N-hydroxy-1,2,3,4- |
| | tetrahydronaphthalene-2-carboxamide |
| I-103 | 7-(3-furyl)-N-hydroxy-1,2,3,4-tetrahydronaphthalene-2- |
| | carboxamide |
| I-104 | N-hydroxy-7-(4-nitrophenyl)-1,2,3,4-tetrahydronaphthalene-2- |
| | carboxamide |
| I-105 | 7-(3-cyano-4-fluorophenyl)-N-hydroxy-1,2,3,4- |
| | tetrahydronaphthalene-2-carboxamide |
| I-106 | N-hydroxy-7-(3-nitrophenyl)-1,2,3,4-tetrahydronaphthalene-2- |
| | carboxamide |
| I-107 | 7-(3-ethoxyphenyl)-N-hydroxy-1,2,3,4-tetrahydronaphthalene-2- |
| | carboxamide |
| I-108 | 7-{4-[(dimethylamino)carbonyl]-3-fluorophenyl}-N-hydroxy- |
| | 1,2,3,4-tetrahydronaphthalene-2-carboxamide |
| I-109 | 7-(3,4-dimethoxyphenyl)-N-hydroxy-1,2,3,4- |
| | tetrahydronaphthalene-2-carboxamide |
| I-110 | 7-biphenyl-3-yl-N-hydroxy-1,2,3,4-tetrahydronaphthalene-2- |
| | carboxamide |
| I-111 | 7-(2,5-dimethylphenyl)-N-hydroxy-1,2,3,4-tetrahydronaphthalene- |
| | 2-carboxamide |
| I-112 | N-hydroxy-7-[4-(trifluoromethyl)phenyl]-1,2,3,4- |
| | tetrahydronaphthalene-2-carboxamide |
| I-113 | 7-[4-fluoro-3-(pyrrolidin-1-ylcarbonyl)phenyl]-N-hydroxy-1,2,3,4- |
| | tetrahydronaphthalene-2-carboxamide |
| I-114 | N-hydroxy-6′-methoxy-5,6,7,8-tetrahydro-2,2′-binaphthalene-7- |
| | carboxamide |
| I-115 | 7-{4-chloro-3-[(dimethylamino)carbonyl]phenyl}-N-hydroxy- |
| | 1,2,3,4-tetrahydronaphthalene-2-carboxamide |
| I-116 | N-hydroxy-7-(2-isopropoxypyridin-3-yl)-1,2,3,4- |
| | tetrahydronaphthalene-2-carboxamide |
| I-117 | N-hydroxy-7-[4-(morpholin-4-ylsulfonyl)phenyl]-1,2,3,4- |
| | tetrahydronaphthalene-2-carboxamide |
| I-118 | 7-[3-fluoro-4-(pyrrolidin-1-ylcarbonyl)phenyl]-N-hydroxy-1,2,3,4- |
| | tetrahydronaphthalene-2-carboxamide |
| I-119 | 7-(3-chloro-4-fluorophenyl)-N-hydroxy-1,2,3,4- |
| | tetrahydronaphthalene-2-carboxamide |
| I-120 | 7-[4-(1-cyano-1-methylethyl)phenyl]-N-hydroxy-1,2,3,4- |
| | tetrahydronaphthalene-2-carboxamide |
| I-121 | 7-[4-(benzyloxy)phenyl]-N-hydroxy-1,2,3,4-tetrahydronaphthalene- |
| | 2-carboxamide |
| I-122 | N-hydroxy-7-(4-isobutylphenyl)-1,2,3,4-tetrahydronaphthalene-2- |
| | carboxamide |
| I-123 | N-hydroxy-7-[4-(piperidin-1-ylsulfonyl)phenyl]-1,2,3,4- |
| | tetrahydronaphthalene-2-carboxamide |
| I-124 | 7-(4-chlorophenoxy)-N-hydroxy-2-methyl-1,2,3,4- |
| | tetrahydronaphthalene-2-carboxamide |
| I-125 | N-[4-({7-[(hydroxyamino)carbonyl]-5,6,7,8-tetrahydronaphthalen- |
| | 2-yl}oxy)pyridin-2-yl]-4-methylpiperidine-4-carboxamide |
| I-126 | N-[3-({7-[(hydroxyamino)carbonyl]-5,6,7,8-tetrahydronaphthalen- |
| | 2-yl}oxy)phenyl]-4-methylpiperidine-4-carboxamide |
| I-127 | N-hydroxy-2-methyl-7-{3-[(methylsulfonyl)amino]phenoxy}- |
| | 1,2,3,4-tetrahydronaphthalene-2-carboxamide |
| I-128 | 7-(3-{[(1-ethyl-4-methylpiperidin-4-yl)amino]carbonyl}phenoxy)- |
| | N-hydroxy-1,2,3,4-tetrahydronaphthalene-2-carboxamide |
| I-129 | N-{7-[(hydroxyamino)carbonyl]-5,6,7,8-tetrahydronaphthalen-2- |
| | yl}piperidine-4-carboxamide |
| I-130 | 1-ethyl-N-(3-{7-[(hydroxyamino)carbonyl]-5,6,7,8- |
| | tetrahydronaphthalen-2-yl}phenyl)pyrrolidine-2-carboxamide |
| I-131 | N7-(1-ethyl-4-methylpiperidin-4-yl)-N2-hydroxy-1,2,3,4- |
| | tetrahydronaphthalene-2,7-dicarboxamide |
| I-132 | 1-ethyl-N-(3-{7-[(hydroxyamino)carbonyl]-5,6,7,8- |
| | tetrahydronaphthalen-2-yl}phenyl)-4-methylpiperidine-4- |
| | carboxamide |
| I-133 | 1-ethyl-N-(3-{7-[(hydroxyamino)carbonyl]-5,6,7,8- |
| | tetrahydronaphthalen-2-yl}phenyl)piperidine-4-carboxamide |
| I-134 | N-{7-[(hydroxyamino)carbonyl]-5,6,7,8-tetrahydronaphthalen-2- |
| | yl}-1-isopropyl-4-methylpiperidine-4-carboxamide |
| I-135 | 4-ethyl-N-{7-[(hydroxyamino)carbonyl]-5,6,7,8- |
| | tetrahydronaphthalen-2-yl}piperazine-1-carboxamide |
| I-136 | N-hydroxy-7-(3-{[(1-methylpiperidin-4-yl)sulfonyl]amino} |
| | phenyl)-1,2,3,4-tetrahydronaphthalene-2-carboxamide |
| I-137 | N-hydroxy-7-{3-[(piperidin-4-ylsulfonyl)amino]phenoxy}-1,2,3,4- |
| | tetrahydronaphthalene-2-carboxamide |
| I-138 | N-hydroxy-6-{3-[(methylsulfonyl)amino]phenoxy}-1,2,3,4- |
| | tetrahydronaphthalene-2-carboxamide |
| I-139 | N-hydroxy-7-{[3-(pyrrolidin-1-ylmethyl)benzoyl]amino}-1,2,3,4- |
| | tetrahydronaphthalene-2-carboxamide |
| I-140 | N-hydroxy-6-(3-{[(1-methylcyclohexyl)carbonyl]amino}phenyl)- |
| | 1,2,3,4-tetrahydronaphthalene-2-carboxamide |
| I-141 | N-(3-{6-[(hydroxyamino)carbonyl]-5,6,7,8-tetrahydronaphthalen-2- |
| | yl}phenyl)-4-methylpiperidine-4-carboxamide |
| I-142 | N-hydroxy-7-{[3-(2-pyrrolidin-1-ylethoxy)phenyl]amino}-1,2,3,4- |
| | tetrahydronaphthalene-2-carboxamide |
| I-143 | 7-({2-[(cyclopropylcarbonyl)amino]pyridin-4-yl}oxy)-N,2- |
| | dihydroxy-1,2,3,4-tetrahydronaphthalene-2-carboxamide |
| I-144 | 2-fluoro-N-hydroxy-7-{3-[(methylsulfonyl)amino]phenoxy}- |
| | 1,2,3,4-tetrahydronaphthalene-2-carboxamide |
| I-145 | 7-{3-[(2,2-dimethylpropanoyl)amino]phenyl}-N-hydroxy-1,1- |
| | dimethyl-1,2,3,4-tetrahydronaphthalene-2-carboxamide |
| I-146 | 2-ethyl-N-hydroxy-6-phenoxy-1,2,3,4-tetrahydronaphthalene-2- |
| | carboxamide |
| I-147 | 7-[(4-chlorobenzyl)amino]-N-hydroxy-1-methyl-1,2,3,4- |
| | tetrahydronaphthalene-2-carboxamide |
| I-148 | methyl 3,3-dimethyl-6-{[(4-methylpiperidin-4-yl)carbonyl]amino}- |
| | 1,2,3,4-tetrahydronaphthalene-2-carboxylate |
| I-149 | methyl 6-{3-[2-(dimethylamino)ethoxy]phenyl}-3,3-difluoro- |
| | 1,2,3,4-tetrahydronaphthalene-2-carboxylate |
| I-150 | N-hydroxy-1-methyl-7-[(7-oxo-5,6,7,8-tetrahydro-1,8- |
| | naphthyridin-4-yl)oxy]-1,2,3,4-tetrahydronaphthalene- |
| | 2-carboxamide |
| I-151 | N-hydroxy-7-[3-(isobutyrylamino)phenoxy]-3-methoxy-1,2,3,4- |
| | tetrahydronaphthalene-2-carboxamide |
| I-152 | N-hydroxy-6-[(4-{[(1-methylpiperidin-4- |
| | yl)amino]carbonyl}phenyl)amino]-1,2,3,4-tetrahydronaphthalene- |
| | 2-carboxamide |
| I-153 | 7-{1-[(3-chlorophenyl)amino]ethyl}-N-hydroxy-1,2,3,4- |
| | tetrahydronaphthalene-2-carboxamide |
| I-154 | N-(1-{7-[(hydroxyamino)carbonyl]-5,6,7,8-tetrahydronaphthalen-2- |
| | yl}ethyl)-4-methylpiperidine-4-carboxamide |
| I-155 | 7-({3-[(ethylsulfonyl)amino]phenyl}amino)-5-fluoro-N-hydroxy-6- |
| | methyl-1,2,3,4-tetrahydronaphthalene-2-carboxamide |
| I-156 | 6-chloro-N-hydroxy-7-{[(1-methylcyclohexyl)carbonyl]amino}-8- |
| | (trifluoromethyl)-1,2,3,4-tetrahydronaphthalene-2-carboxamide |
| I-157 | 7-[(3-chlorobenzyl)amino]-N-hydroxy-6,8-dimethyl-1,2,3,4- |
| | tetrahydronaphthalene-2-carboxamide |
| I-158 | 5-chloro-N-hydroxy-7-[(4-methoxybenzoyl)amino]-6-methyl- |
| | 1,2,3,4-tetrahydronaphthalene-2-carboxamide |
| I-159 | 8-chloro-6-cyano-N-hydroxy-7-(pyridin-4-ylamino)-1,2,3,4- |
| | tetrahydronaphthalene-2-carboxamide |
| I-160 | N7-(1-ethyl-4-methylpiperidin-4-yl)-N2-hydroxy-6-methyl-1,2,3,4- |
| | tetrahydronaphthalene-2,7-dicarboxamide |
| I-161 | 7-{3-[(2,2-dimethylpropanoyl)amino]phenoxy}-5,6-difluoro-N- |
| | hydroxy-1,2,3,4-tetrahydronaphthalene-2-carboxamide |
| I-162 | N-{7-[(hydroxyamino)carbonyl]-5,6,7,8-tetrahydronaphthalen-2- |
| | yl}-1-isopropyl-N,4-dimethylpiperidine-4-carboxamide |
| I-163 | 6,8-diethyl-N-hydroxy-7-[(2-pyrrolidin-1-ylethyl)amino]-1,2,3,4- |
| | tetrahydronaphthalene-2-carboxamide |
| I-164 | 6-({2-[(cyclopropylcarbonyl)amino]pyridin-4-yl}oxy)-8-fluoro-N- |
| | hydroxy-1,2,3,4-tetrahydronaphthalene-2-carboxamide |
| I-165 | 7-chloro-N2-hydroxy-N6-(tetrahydro-2H-pyran-4-yl)-1,2,3,4- |
| | tetrahydronaphthalene-2,6-dicarboxamide |
| I-166 | 5-fluoro-N-hydroxy-7-{3-[(methylsulfonyl)amino]phenyl}-1,2,3,4- |
| | tetrahydronaphthalene-2-carboxamide |
| I-167 | 4-{6-[(hydroxyamino)carbonyl]-3,8,8-trimethyl-5,6,7,8- |
| | tetrahydronaphthalen-2-yl)-N-piperidin-4-ylpyridine-2-carboxamide |
| I-168 | 7-[(4-fluorobenzyl)amino]-N-hydroxy-6-methoxy-1,2,3,4- |
| | tetrahydronaphthalene-2-carboxamide |
| I-169 | 7-{3-[(ethylsulfonyl)amino]phenyl}-N-hydroxy-4,4-dimethyl- |
| | 1,2,3,4-tetrahydronaphthalene-2-carboxamide |
| I-170 | 6-[(2,4-dichlorobenzyl)oxy]-N-hydroxy-7-methyl-1,2,3,4- |
| | tetrahydronaphthalene-2-carboxamide |
| I-171 | (2R)-7-({2-[(cyclopropylcarbonyl)amino]pyridin-4-yl}oxy)-N- |
| | hydroxy-1,2,3,4-tetrahydronaphthalene-2-carboxamide |
| I-172 | (2R)-N-hydroxy-7-[(7-oxo-5,6,7,8-tetrahydro-1,8-naphthyridin-4- |
| | yl)oxy]-1,2,3,4-tetrahydronaphthalene-2-carboxamide |
| I-173 | N-[4-({6-[(hydroxyamino)carbonyl]-5,6,7,8-tetrahydronaphthalen- |
| | 2-yl}oxy)phenyl]pyrrolidine-3-carboxamide |
| I-174 | 1-ethyl-N-(4-{6-[(hydroxyamino)carbonyl]-5,6,7,8- |
| | tetrahydronaphthalen-2-yl}phenyl)-4-methylpiperidine-4- |
| | carboxamide |
| I-175 | (3R)-N-{6-[(hydroxyamino)carbonyl]-5,6,7,8- |
| | tetrahydronaphthalen-2-yl}-1,2,3,4-tetrahydroisoquinoline- |
| | 3-carboxamide |
| I-176 | 6-[(2-amino-3-methylbutanoyl)amino]-N-hydroxy-1,2,3,4- |
| | tetrahydronaphthalene-2-carboxamide |
| I-177 | N-hydroxy-6-{[3-(methylamino)propanoyl]amino}-1,2,3,4- |
| | tetrahydronaphthalene-2-carboxamide |
| I-178 | N-[4-({6-[(hydroxyamino)carbonyl]-5,6,7,8-tetrahydronaphthalen- |
| | 2-yl}oxy)pyridin-2-yl]-1-isopropyl-4-methylpiperidine-4- |
| | carboxamide |
| I-179 | N-(3-{6-[(hydroxyamino)carbonyl]-5,6,7,8-tetrahydronaphthalen-2- |
| | yl}phenyl)-1-isopropyl-4-methylpiperidine-4-carboxamide |
| I-180 | N-hydroxy-6-[2-(3-piperidin-4-ylphenyl)ethyl]-1,2,3,4- |
| | tetrahydronaphthalene-2-carboxamide |
| I-181 | 4-({6-[(hydroxyamino)carbonyl]-5,6,7,8-tetrahydronaphthalen-2- |
| | yl}oxy)-N-(1-isopropylpiperidin-4-yl)pyridine-2-carboxamide |
| I-182 | 6-{[(dimethylamino)acetyl]amino}-N-hydroxy-1,2,3,4- |
| | tetrahydronaphthalene-2-carboxamide |
| I-183 | N-(4-{6-[(hydroxyamino)carbonyl]-5,6,7,8-tetrahydronaphthalen-2- |
| | yl}pyridin-2-yl)-4-methylpiperidine-4-carboxamide |
| I-184 | N-hydroxy-6-(2-piperidin-3-ylethyl)-1,2,3,4-tetrahydronaphthalene- |
| | 2-carboxamide |
| I-185 | N-hydroxy-6-(2-piperazin-1-ylpyridin-4-yl)-1,2,3,4- |
| | tetrahydronaphthalene-2-carboxamide |
| I-186 | 6-{[(2S)-2-amino-3-methylbutanoyl]amino}-N-hydroxy-1,2,3,4- |
| | tetrahydronaphthalene-2-carboxamide |
| I-187 | N-hydroxy-6-{[(methylamino)acetyl]amino}-1,2,3,4- |
| | tetrahydronaphthalene-2-carboxamide |
| I-188 | N-(4-{6-[(hydroxyamino)carbonyl]-5,6,7,8-tetrahydronaphthalen-2- |
| | yl}pyridin-2-yl)-1,4-dimethylpiperidine-4-carboxamide |
| I-189 | N-{6-[(hydroxyamino)carbonyl]-5,6,7,8-tetrahydronaphthalen- |
| | 2-yl}piperidine-3-carboxamide |
| I-190 | N-hydroxy-6-{4-[(pyrrolidin-3-ylamino)carbonyl]phenoxy}- |
| | 1,2,3,4-tetrahydronaphthalene-2-carboxamide |
| I-191 | N-{6-[(hydroxyamino)carbonyl]-5,6,7,8-tetrahydronaphthalen-2- |
| | yl}pyrrolidine-2-carboxamide |
| I-192 | N-(4-{6-[(hydroxyamino)carbonyl]-5,6,7,8-tetrahydronaphthalen-2- |
| | yl}pyridin-2-yl)-1-isopropyl-4-methylpiperidine-4-carboxamide |
| I-193 | 1-ethyl-N-(4-{6-[(hydroxyamino)carbonyl]-5,6,7,8- |
| | tetrahydronaphthalen-2-yl}pyridin-2-yl)-4-methylpiperidine-4- |
| | carboxamide |
| I-194 | N-hydroxy-6-{[phenyl(piperidin-4-yl)acetyl]amino}-1,2,3,4- |
| | tetrahydronaphthalene-2-carboxamide |

### 4. General Synthetic Methods and Intermediates

The compounds of the present invention can be prepared by methods known to one of ordinary skill in the art and/or by reference to the schemes shown below and the synthetic examples. Exemplary synthetic routes are set forth in Schemes below, and in the Examples.

It will be appreciated that any one of the four available positions on the aromatic ring of the tetralin can be functionalized as shown in Schemes 1-6 and the other three positions are substituted with R¹, wherein R¹ has the values described herein. It will further be appreciated that similar transformations shown in Schemes 1-6 can be carried out on tetralins with substitution on either of the two rings in the R^{1a}, R^{1b}, R¹ and R^{1c} positions, as shown in Formula (I) herein, wherein R^{1a}, R^{1b}, R¹ and R^{1c} have the values described herein. Schemes 7-10 describe general routes to prepare such substituted tetralins.

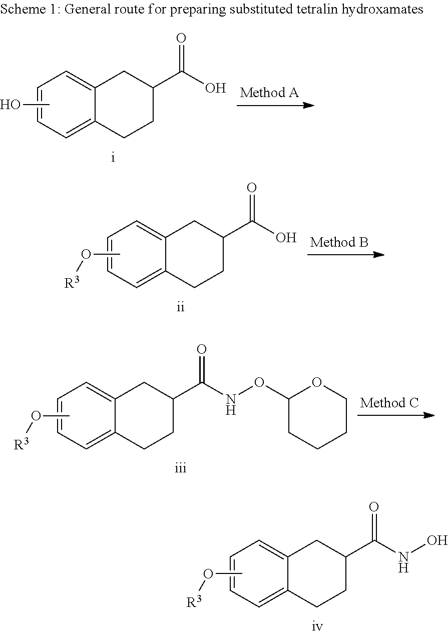

Scheme 1 shows a general route for the preparation of compounds of formula iv from commercially available, or readily derivable, hydroxy tetralin carboxylic acids of the formula i. The phenols i may be arylated through nucleophilic aromatic substitution by treatment with a suitable aryl electrophile in the presence of a carbonate base (R³=pyridyl, aryl, Method A). The resulting carboxylic acid ii may be coupled to THP-protected hydroxylamine by the action of TFFH in N,N-dimethylformamide (Method B; Carpino et al., J. Am. Chem. Soc. 1995, 117(19):5401). The THP acetal group may be hydrolyzed under treatment with mild acid (Method C; Secrist et al., J. Org. Chem. 1979, 44(9):1434) to afford the hydroxamate iv.

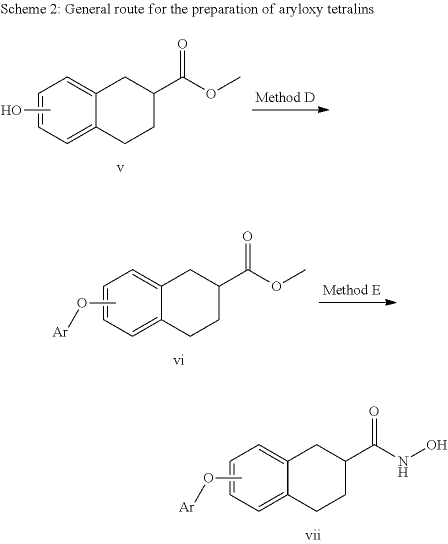

The preparation of O-arylated tetralins is depicted in Scheme 2. Intermediate v, derivable from commercial or readily available starting materials, may be converted to the corresponding aryl ether vi through a copper(II) acetate mediated coupling with a suitable arylboronic acid (Method D; Chan et al., Tetrahedron Lett. 1998, 39(19):2933). Conversion of the methyl ester vi to the corresponding hydroxamate is conducted by reaction with the potassium salt of hydroxylamine (Method E; Huang et al., J. Med. Chem. 2009, 52(21):6757) leading to the formation of compounds of formula vii.

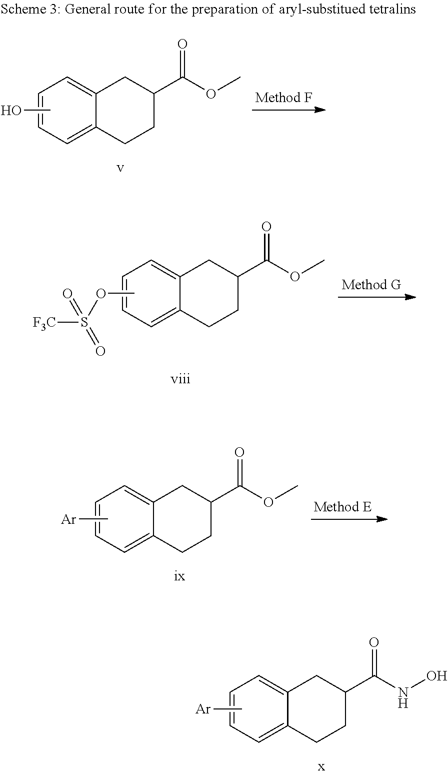

Scheme 3 depicts a general method to prepare arylated tetralin hydroxamates x. Treatment of phenols v with trifluoromethanesulfonic anhydride and triethylamine in a solvent such as methylene chloride affords the corresponding triflate ester viii. Arylation of viii can be achieved through a Suzuki type reaction by treatment with a suitable arylboronic acid under palladium catalysis and microwave heating (Method G; Kotha et al., Tetrahedron 2002, 58(48):9633). Conversion to the corresponding hydroxamate can be completed following Method E.

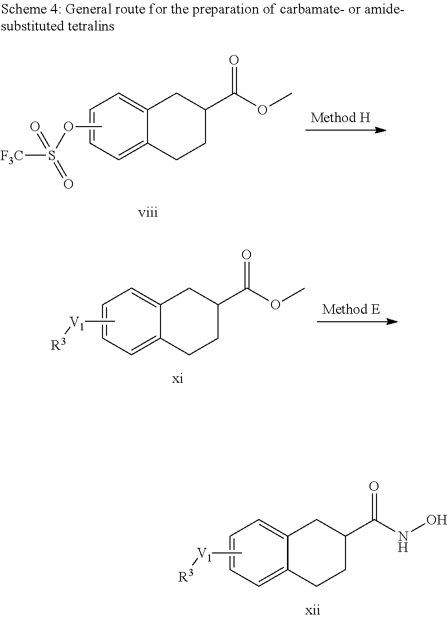

Scheme 4 shows a general method for the preparation of carbamates or amides of formula xii. Intermediate viii may be undergo a Pd/Xantphos-mediated intermolecular amidation with an appropriately functionalized carbamate (V₁═N(R^{4a})—C(O)—O—) or amide (V₁═N(R^{4a})—C(O)—; Method H, Buchwald et al., J. Am. Chem. Soc. 2002, 124(21):6043). Conversion to the corresponding hydroxamate can be completed following Method E.

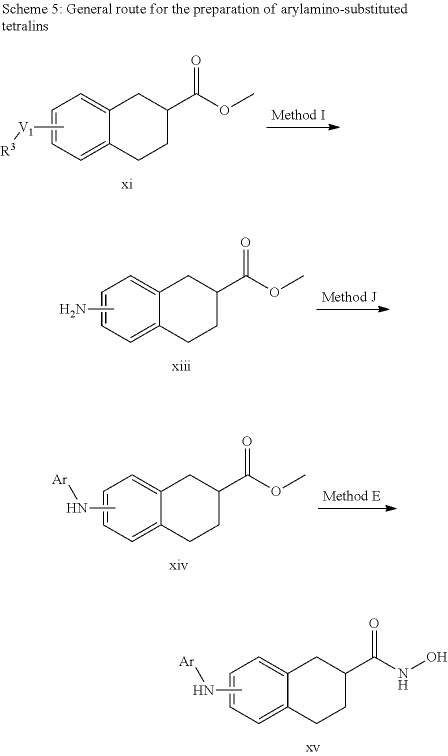

A general method for the preparation of arylamino-substituted tetralin hydroxamates is depicted in Scheme 5. Aniline intermediates xiii may be prepared by appropriate deprotection (Method I) of a suitable carbamate xi (V₁═N(R^{4a})—C(O)—O—), and may in turn be arylated by treatment with an arylboronic acid and copper(II) acetate (Method J, Chan et al., Tetrahedron Lett. 1998, 39(19):2933). The resulting arylamino ester xiv may then be converted to its corresponding hydroxamate xv following Method E.

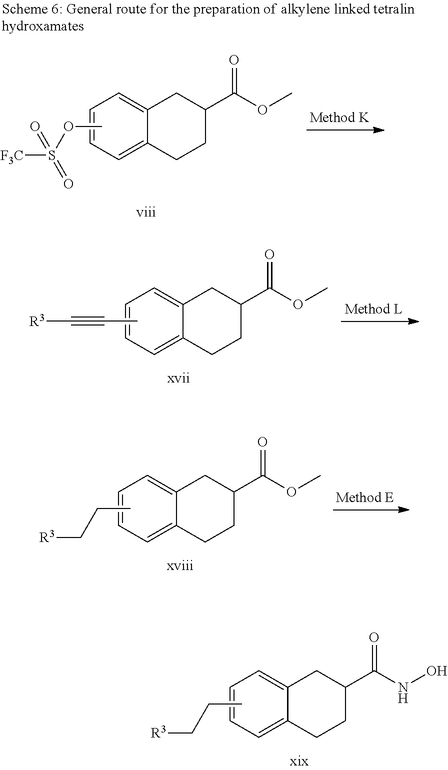

The preparation of alkylene-linked substitutions on tetralin hydroxamates can be carried out as depicted in Scheme 6. Reaction of a suitable terminal alkyne with viii may be carried out under Sonogashira conditions (Method K; Najera et al., Chem. Rev. 2007, 107(3): 874). The resulting disubstituted alkyne may then be reduced through treatment with hydrogen in the presence of palladium on carbon in methanol to afford the alkylene-linked intermediate xviii (Method L). Conversion of the ester to the hydroxamate may be carried out as described previously (Method E).

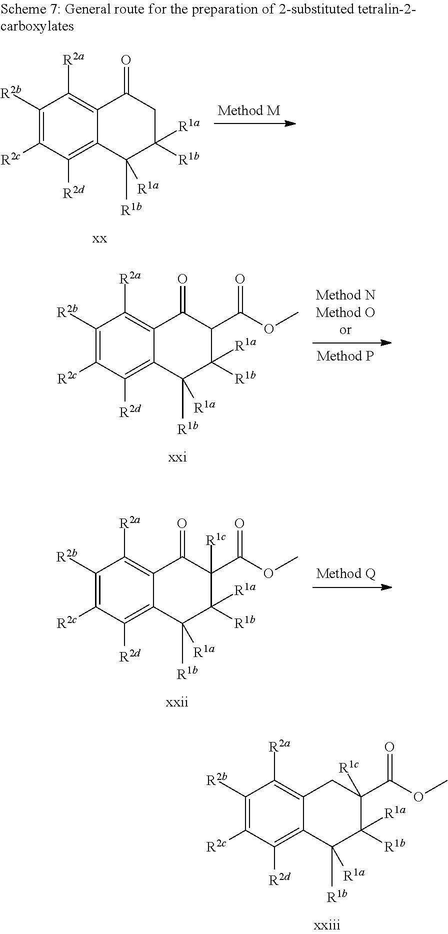

A general route for the preparation of 2-substituted tetralin-2-carboxylates is shown in Scheme 7. A commercially available and suitably substituted tetralin-1-one (xx) may be treated with sodium hydride and dimethyl carbonate in DMF to afford β-ketoester xxi (Method M). The resulting ketoester may then be further functionalized at the 2-position through treatment with sodium hydride and an alkyl iodide (Method N, R^{1c}=alkyl or fluoroalkyl); treatment with Selectfluor (Method O; R^{1c}=fluoro; Banks, et al., J. Chem. Soc. Chem. Commun. 1994: 343), or treatment with IBX (Method P; R^{1c}=hydroxy; Duschek et. al., Chemisry—A European Journal 2009, 15(41): 10713). Ketone reduction may be achieved by reaction with triethylsilane and trifluoroacetic acid in methylene chloride (Method Q; Bonnaud et al., Eur. J. Org. Chem. 2005: 3360). The intermediate xxiii may be further elaborated as described above.

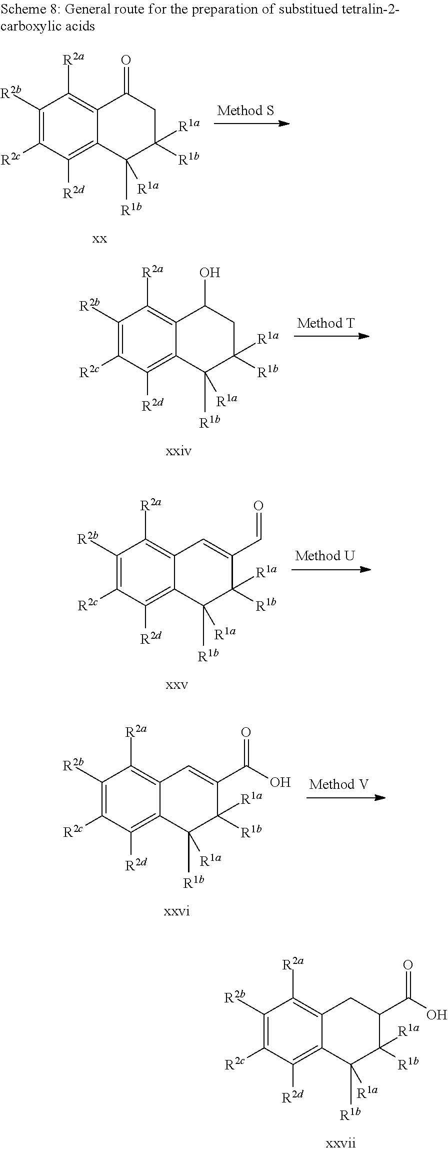

Scheme 8 shows a general method for the preparation of substituted tetralin-2-carboxylic acids (xxvii). A wide range of substitution patterns around the tetralin core of xxvii may be achieved through a four-step sequence starting from commercially available tetralin-1-ones (xx) following the synthesis of Rag et al., Ind. J. Chem. 1981, 20B: 100. Intermediate xx may be reduced to the alcohol xxiv by the action of sodium borohydride in methanol (Method S). A two step, one-pot, elimination/formylation in phosphoryl chloride and DMF may be used to generated alkenal xxv (Method T). Oxidation of the corresponding aldehyde with silver(I) oxide (Method U), followed by palladium-catalyzed olefin hydrogenation (Method V) may then generate tetralin xxvii.

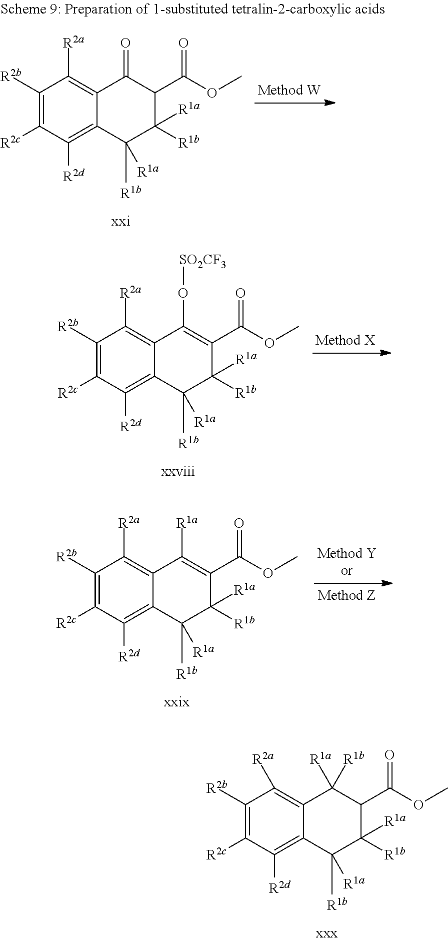

Scheme 9 depicts a general method which may be used to generate 1-alkyl or 1,1-dialkyl-substituted tetralins. Intermediate xxi may be converted to its enol triflate by the action of triflic anhydride (Method W). This intermediate may be alkylated by treatment with an alkyl cuprate (Method X; R₁ₐ=alkyl; Romero et al., Bioorg. Med. Chem. Lett. 1992, 2(12):1691) to generate compounds of formula xxix. This intermediate may be further alkylated by treatment with a second equivalent of alkyl cuprate (Method Y; R^{1b}=alkyl) or may be hydrogenated under palladium catalysis (Method Z, R^{1b}=H) to afford 1-substituted tetralin ester intermediates xxx.

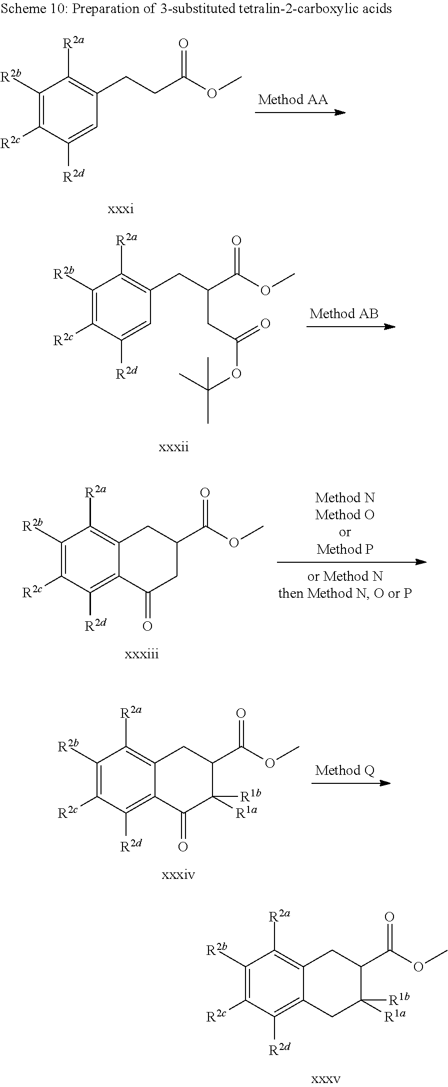

Scheme 10 shows a general method for the preparation of 3-substituted or 3,3-disubstituted tetralins xxxv. A suitably functionalized aryl propionate ester xxxi may be acetylated by treatment with LDA and tert-butyl bromoacetate to generate diester xxxii (Method AA). Friedel-Crafts type acylation in the presence of TFA and TFAA generates tetralone xxxiii (Method AB). The tetralone may be functionalized adjacent to the ketone, via its enolate, following Methods N, O or P described above (Method N, R^{1b}=alkyl; Method O, R^{1b}=fluoro, Method P, R^{1b}=hydroxy). Repeating Methods N or O will allow for multiple substitutions at the 3-position. As described above, the resulting tetralones xxxiv may be deoxygenated following Method Q.

### 5. Uses, Formulation and Administration

As discussed above, the present invention provides compounds and pharmaceutical compositions that are useful as inhibitors of HDAC enzymes, particularly HDAC6, and thus the present compounds are useful for treating proliferative, inflammatory, infectious, neurological or cardiovascular disorders.

The compounds and pharmaceutical compositions of the invention are particularly useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

In some embodiments, therefore, the invention provides the compound of formula (I), or a pharmaceutically acceptable salt thereof, for use in treating cancer. In some embodiments, the invention provides a pharmaceutical composition (as described herein) for the treatment of cancer comprising the compound of formula (I), or a pharmaceutically acceptable salt thereof. In some embodiments, the invention provides the use of the compound of formula (I), or a pharmaceutically acceptable salt thereof, for the preparation of a pharmaceutical composition (as described herein) for the treatment of cancer. In some embodiments, the invention provides the use of an effective amount of the compound of formula (I), or a pharmaceutically acceptable salt thereof, for the treatment of cancer.

Non-limiting examples of solid tumors that can be treated with the disclosed inhibitors include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

Non-limiting examples of hematologic malignancies that can be treated with the disclosed inhibitors include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

In some embodiments, compounds of the invention are suitable for the treatment of breast cancer, lung cancer, ovarian cancer, multiple myeloma, acute myeloid leukemia or acute lymphoblastic leukemia.

In other embodiments, compounds of the invention are suitable for the treatment of inflammatory and cardiovascular disorders including, but not limited to, allergies/anaphylaxis, acute and chronic inflammation, rheumatoid arthritis; autoimmunity disorders, thrombosis, hypertension, cardiac hypertrophy, and heart failure.

Accordingly, in another aspect of the present invention, pharmaceutical compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable prodrugs, salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of HDAC6.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In yet another aspect, a method for treating a proliferative, inflammatory, infectious, neurological or cardiovascular disorder is provided comprising administering an effective amount of a compound, or a pharmaceutical composition to a subject in need thereof. In certain embodiments of the present invention an “effective amount” of the compound or pharmaceutical composition is that amount effective for treating a proliferative, inflammatory, infectious, neurological or cardiovascular disorder, or is that amount effective for treating cancer. In other embodiments, an “effective amount” of a compound is an amount which inhibits binding of HDAC6, and thereby blocks the resulting signaling cascades that lead to the abnormal activity of growth factors, receptor tyrosine kinases, protein serine/threonine kinases, G protein coupled receptors and phospholipid kinases and phosphatases.

The compounds and compositions, according to the method of the present invention, may be administered using any amount and any route of administration effective for treating the disease. The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, a compound of formula (I) or a pharmaceutical composition thereof is administered in conjunction with an anticancer agent. As used herein, the term “anticancer agent” refers to any agent that is administered to a subject with cancer for purposes of treating the cancer. Combination therapy includes administration of the therapeutic agents concurrently or sequentially. Alternatively, the therapeutic agents can be combined into one composition which is administered to the patient.

Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which down-regulates cell replication. In certain embodiments, a compound of the invention is administered in conjunction with a proteasome inhibitor.

Another aspect of the invention relates to inhibiting HDAC6, activity in a biological sample or a patient, which method comprises administering to the patient, or contacting said biological sample with a compound of formula (I), or a composition comprising said compound. The term “biological sample”, as used herein, generally includes in vivo, in vitro, and ex vivo materials, and also includes, without limitation, cell cultures or extracts thereof; biopsied material obtained from a mammal or extracts thereof; and blood, saliva, urine, feces, semen, tears, or other body fluids or extracts thereof.

Still another aspect of this invention is to provide a kit comprising separate containers in a single package, wherein the inventive pharmaceutical compounds, compositions and/or salts thereof are used in combination with pharmaceutically acceptable carriers to treat disorders, symptoms and diseases where HDAC6 plays a role.

### 6. Preparation of Exemplary Compounds

### EXPERIMENTAL PROCEDURES

### Definitions

| | |
|---|---|
| ATP | adenosine triphosphate |
| DCE | dichloroethane |
| DCM | dichloromethane |
| DIPEA | diisopropylethyl amine |
| DMF | N,N-dimethylformamide |
| DMFDMA | N,N-dimethylformamide dimethyl acetal |
| DMSO | dimethylsulfoxide |
| EDC | N-(3-dimethylaminopropyl)-N′-ethylcarbodiimide |
| | hydrochloride |
| EDTA | ethylenediaminetetraacetic acid |
| EtOAc | ethyl acetate |
| EtOH | ethanol |
| FA | formic acid |
| FBS | fetal bovine serum |
| h | hours |
| HATU | N,N,N′,N′-tetramethyl-O-(7-azabenzotriazole-1-yl)uronium |
| | hexafluorophosphate |
| HBTU | O-benzotriazol-1-yl-N,N,N′,N′-tetramethyluronium |
| | hexafluorophosphate |
| HEPES | N-(2-hydroxyethyl)piperazine-N′-(2-ethanesulfonic acid) |
| HOBT | 1-hydroxybenztriazole hydrate |
| HRMS | high resolution mass spectrum |
| IPA | isopropyl alcohol |
| IBX | 2-iodoxybenzoic acid |
| LAH | lithium aluminum hydride |
| LC-MS | liquid chromatography mass spectrum |
| m/z | mass to charge |
| MTBE | methyl tert-butyl ether |
| Me | methyl |
| MEM | minimum essential media |
| MeOH | methanol |
| min | minutes |
| MS | mass spectrum |
| MWI | microwave irradiation |
| NaHMDS | sodium bis(trimethylsilyl)amide |
| NMM | N-methyl morpholine |
| PBS | phosphate buffered saline |
| rt | room temperature |
| TEA | triethylamine |
| TFA | trifluoroacetic acid |
| TFAA | trifluoroacetic anhydride |
| TFFH | 1,1,3,3-tetramethylfluoroformamidinium |
| | hexafluorophosphate |
| THF | tetrahydrofuran |
| TMEDA | N,N,N′,N′-tetramethyl-ethane-1,2-diamine |
| Xantphos | 4,5-Bis(diphenylphosphino)-9,9-dimethylxanthene |
| X-Phos | 2-Dicyclohexylphosphino-2′,4′,6′-triisopropylbiphenyl |

Analytical Methods

NMR: ¹H NMR spectra are run on a 300 or 400 MHz Bruker unless otherwise stated.

LCMS: LC-MS spectra are run using an Agilent 1100 LC fitted with a Waters Symmetry® C18 3.5 μm, 4.6×100 mm column, interfaced to a micromass Waters® Micromass® Zspray™ Mass Detector (ZMD) using the following gradients:
Method Formic Acid (FA): Acetonitrile containing zero to 100 percent 0.1% formic acid in water (2.5 mL/min for a 3 minute run, 1.0 mL/min for a 13 minute run.).
Method Ammonium Acetate (AA): Acetonitrile containing zero to 100 percent 10 mM ammonium acetate in water (2.5 mL/min for a 3 minute run, 1.0 mL/min for a 13 minute run).

HPLC: Preparative HPLC are conducted using 18×150 mm Sunfire C-18 columns eluting with water-MeCN gradients using a Gilson instrument operated by 322 pumps with the UV/visible 155 detector triggered fraction collection set to between 200 nm and 400 nm. Mass gated fraction collection is conducted on an Agilent 1100 LC/MSD instrument.

### Example 1

### 7-(3-chlorophenyl)-N-hydroxy-1,2,3,4-tetrahydronaphthalene-2-carboxamide Compound I-49

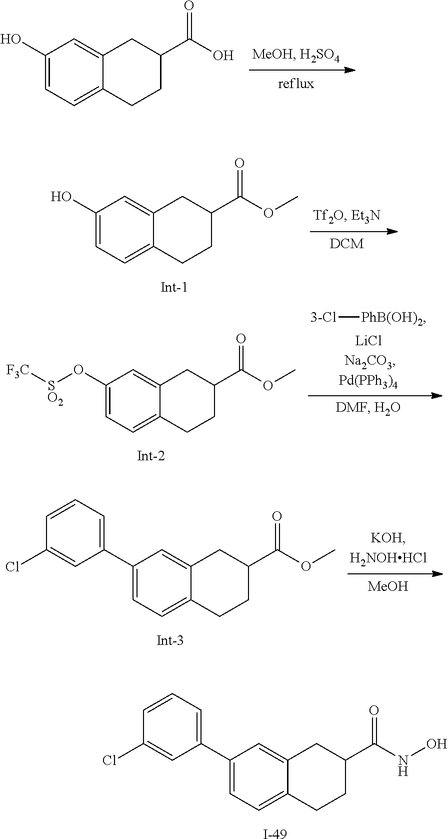

### Step 1: methyl 7-hydroxy-1,2,3,4-tetrahydronaphthalene-2-carboxylate Intermediate 1

To a solution of 7-hydroxy-1,2,3,4-tetrahydronaphthalene-2-carboxylic acid (5.77 g, 30 mmol) in methanol (60 mL) was added sulfuric acid (0.16 mL, 3 mmol). The reaction mixture was heated at reflux overnight and was then allowed to cool to room temperature. The reaction mixture was concentrated under reduced pressure and the crude residue dissolved in ethyl acetate. The resulting solution was washed with saturated aqueous sodium bicarbonate solution and brine, then dried over sodium sulfate, filtered and concentrated under reduced pressure. The crude residue was purified by silica gel chromatography (hexanes to 30/70 hexanes/ethyl acetate gradient) to afford the desired product. (Int-1, 4.78 g, 77%). LC-MS: (FA) ES+ 207; ¹H NMR (400 MHz, CDCl₃) δ ppm 6.95 (d, J=8.2 Hz, 1H), 6.62 (dd, J=8.2, 2.7 Hz, 1H), 6.58 (d, J=2.5 Hz, 1H), 4.65 (s, 1H), 3.73 (s, 3H), 2.99-2.92 (m, 2H), 2.85-2.67 (m, 3H), 2.25-2.14 (m, 1H), 1.83 (dddd, J=13.0, 10.8, 10.7, 6.2 Hz, 1H).

### Step 2: methyl 7-(trifluoromethylsulfonyloxy)-1,2,3,4-tetrahydronaphthalene-2-carboxylate Intermediate 2

Methyl 7-hydroxy-1,2,3,4-tetrahydronaphthalene-2-carboxylate (Int-1, 4.78 g, 23.2 mmol) was dissolved in methylene chloride (100 mL) and triethylamine (9.69 mL, 69.5 mmol) and the resulting solution was cooled in an ice-water bath. To the cooled solution was added trifluoromethanesulfonic anhydride (4.7 mL, 28 mmol) dropwise by syringe. The reaction mixture was then allowed to stir in the ice-water bath for 1 h, then warmed to room temperature and stirred for 2 h. The reaction mixture was poured onto ice and extracted twice with methylene chloride. The extracts were washed with saturated aqueous sodium bicarbonate solution and brine, dried over sodium sulfate, filtered and concentrated under reduced pressure. The crude residue was purified by silica gel chromatography (hexanes to 60/40 hexanes/ethyl acetate gradient) to afford the desired product as a yellow oil. LC-MS: (FA) ES− 337; ¹H NMR (300 MHz, CDCl₃) δ ppm 7.14 (d, J=9.1 Hz, 1H), 7.04-6.99 (m, 2H), 3.73 (s, 3H), 3.08-3.01 (m, 2H), 2.91-2.70 (m, 3H), 2.22 (m, 1H), 1.88 (dddd, J=13.3, 10.7, 10.3, 6.2 Hz, 1H).

### Step 3: methyl 7-(3-chlorophenyl)-1,2,3,4-tetrahydronaphthalene-2-carboxylate Intermediate 3

To a 2-5 mL microwave vial fitted with a stirbar was added methyl 7-{[(trifluoromethyl)sulfonyl]oxy}-1,2,3,4-tetrahydronaphthalene-2-carboxylate (Int-2, 0.218 g, 0.644 mmol) followed by N,N-dimethylformamide (4 mL, 52 mmol), a 2.0 M solution of sodium carbonate in water (0.966 mL, 1.93 mmol), lithium chloride (0.082 g, 1.93 mmol) and 3-chlorophenylboronic acid (0.131 g, 0.838 mmol). The contents of the vial were thoroughly degassed under argon and tetrakis(triphenylphosphine)palladium(0) (0.0372 g, 0.0322 mmol) was added. The reaction mixture was then heated at 100° C. in the microwave for 1 h. After cooling to room temperature, the reaction mixture was poured into ethyl acetate and washed with water. The aqueous phase was extracted twice with additional ethyl acetate. The extracts were combined, washed with brine, dried over sodium sulfate, filtered and concentrated under reduced pressure. The crude residue was purified by silica gel chromatography (hexanes to 60/40 hexanes/ethyl acetate gradient) to afford the desired product (0.123 g, 64%). ¹H NMR (400 MHz, CDCl₃) δ ppm 7.54 (dd, J=1.8, 1.8 Hz, 1H), 7.44 (ddd, J=7.6, 1.5, 1.5 Hz, 1H), 7.37-7.28 (m, 4H), 7.17 (d, J=7.8 Hz, 1H), 3.75 (s, 3H), 3.12-3.05 (m, 2H), 2.97-2.74 (m, 3H), 2.30-2.20 (m, 1H), 1.90 (dddd, J=13.1, 10.6, 10.6, 6.2 Hz, 1H).

### Step 4: 7-(3-chlorophenyl)-N-hydroxy-1,2,3,4-tetrahydronaphthalene-2-carboxamide Compound I-49

A mixture of hydroxylamine hydrochloride (2 g, 29 mmol) in methanol (10 mL) was heated at 90° C. under a dry nitrogen atmosphere until homogeneous. To this heated solution was added a solution of potassium hydroxide (2.85 g, 50.8 mmol) in methanol (6 mL). A precipitate formed on mixing. After heating at 90° C. for 30 minutes, the mixture was cooled to room temperature and the solids were allowed to settle. The resulting solution was carefully removed by syringe to exclude solids and was assumed to contain 1.7 M hydroxylamine.potassium salt. An aliquot of this solution (2.4 mL, 4.09 mmol) was added to a solution of methyl 7-(3-chlorophenyl)-1,2,3,4-tetrahydronaphthalene-2-carboxylate (Int-3, 0.123 g, 0.409 mmol) in methanol (4.5 mL) and N,N-dimethylformamide (1.5 mL). The reaction mixture was stirred at room temperature for 1 day. Excess reagent was quenched by the addition of acetic acid (0.232 mL, 4.09 mmol) with stirring for 10 min. The reaction mixture was concentrated under reduced pressure to dryness and then twice suspended in toluene and concentrated. The residue was further dried in vacuo and purified by preparative reverse phase HPLC. LC-MS: (FA) ES+ 302; ¹H NMR (400 MHz, d₆-DMSO) δ ppm 7.67 (dd, J=1.7, 1.7 Hz, 1H), 7.59 (d, J=7.8 Hz, 1H), 7.46 (dd, J=7.8, 7.8 Hz, 1H), 7.43-7.36 (m, 3H), 7.16 (d, J=7.7 Hz, 1H), 2.97-2.68 (m, 4H), 2.39 (dddd, J=11.2, 11.2, 5.1, 3.1 Hz, 1H), 1.95-1.84 (m, 1H), 1.73 (dddd, J=12.1, 12.1, 12.0, 5.8 Hz, 1H).

### Example 2

The following compounds were prepared in a fashion analogous to that described in Example 1 starting from the intermediates which were prepared as described above and the corresponding boronic acids.

| | LC-MS ES+ |
|---|---|
| Compound | (FA) |
| I-61 | 324 |
| I-60 | 269 |
| I-67 | 298 |
| I-68 | 298 |
| I-69 | 298 |
| I-70 | 302 |
| I-71 | 302 |
| I-72 | 307 |
| I-62 | 383 |
| I-54 | 307 |
| I-82 | 307 |
| I-75 | 307 |
| I-81 | 308 |
| I-24 | 325 |
| I-51 | 351 |
| I-48 | 374 |
| I-17 | 269 |
| I-86 | 318 |
| I-87 | 308 |
| I-88 | 350 |
| I-89 | 326 |
| I-90 | 310 |
| I-91 | 310 |
| I-92 | 311 |
| I-93 | 375 |
| I-94 | 310 |
| I-95 | 312 |
| I-96 | 324 |
| I-97 | 338 |
| I-98 | 332 |
| I-99 | 354 |
| I-100 | 343 |
| I-101 | 316 |
| I-102 | 300 |
| I-103 | 258 |
| I-104 | 313 |
| I-105 | 311 |
| I-106 | 313 |
| I-107 | 312 |
| I-108 | 357 |
| I-109 | 328 |
| I-110 | 344 |
| I-111 | 296 |
| I-112 | 336 |
| I-113 | 383 |
| I-114 | 348 |
| I-115 | 373 |
| I-116 | 327 |
| I-117 | 417 |
| I-118 | 383 |
| I-119 | 320 |
| I-120 | 335 |
| I-121 | 374 |
| I-122 | 324 |
| I-123 | 415 |

### Example 3

### N-hydroxy-6-methoxy-1,2,3,4-tetrahydronaphthalene-2-carboxamide Compound I-10

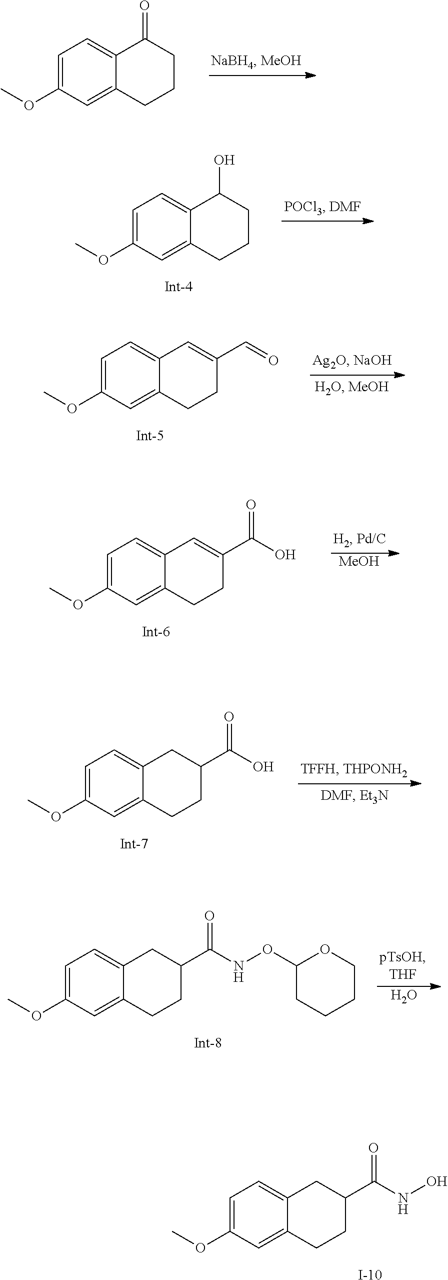

### Step 1: 6-Methoxy-1,2,3,4-tetrahydronaphthalen-1-ol Intermediate 4

Sodium borohydride (5.84 g, 154 mmol) was added in portions over 10 minutes to an ice-cooled solution of 6-methoxy-3,4-dihydronaphthalen-1(2H)-one (13.96 g, 79.22 mmol) in methanol (230 mL). After borohydride addition was complete, the reaction mixture was allowed to stir at room temperature for 1 h. Water (50 mL) was then added and the mixture was concentrated under reduced pressure to remove most of the methanol. The resulting mixture was diluted with water (200 mL) and ethyl acetate (200 mL). The aqueous phase was extracted with additional ethyl acetate (2×200 mL). The extracts were combined, washed with 0.5 M HCl, saturated sodium bicarbonate solution and brine. The extracts were dried over sodium sulfate, filtered and concentrated under reduced pressure. The crude residue was purified by silica gel chromatography (70/30 hexanes/ethyl acetate isocratic) to afford Int-4 as an oil (12.19 g, 86%). ¹H NMR (300 MHz, CDCl₃) δ ppm 7.34 (d, J=8.5 Hz, 1H), 6.77 (dd, J=8.5, 2.7 Hz, 1H), 6.63 (d, J=2.5 Hz, 1H), 4.75 (dd, J=10.1, 4.6 Hz, 1H), 3.79 (s, 3H), 2.87-2.61 (m, 2H), 2.02-1.86 (m, 3H), 1.84-1.71 (m, 1H), 1.58 (d, J=6.3 Hz, 1H).

### Step 2: 6-methoxy-3,4-dihydronaphthalene-2-carbaldehyde Intermediate 5

To a room-temperature solution of 6-methoxy-1,2,3,4-tetrahydronaphthalen-1-ol (Int-4, 12.19 g, 0.0684 mol) in N,N-dimethylformamide (105 mL) was added phosphoryl chloride (12.8 mL, 0.137 mol) dropwise over several minutes. The addition was accompanied by a slight exotherm. Halfway through addition, the reaction mixture turned yellow then orange. After addition of phosphoryl chloride, the reaction mixture was warmed to 50° C. in an oil bath and allowed to stir overnight. The mixture was then cooled to room temperature and concentrated to approximately ⅓ of its volume. The concentrated mixture was poured onto ice water (400 mL) and extracted with 5×200 mL ethyl acetate. Sodium chloride (approx. 20 g) was added to the aqueous phase which was then extracted with 400 mL ethyl acetate. The extracts were combined, washed with water, saturated aqueous sodium bicarbonate and brine, then dried over sodium sulfate, filtered and concentrated under reduced pressure. The crude residue was purified by silica gel chromatography (85/15 hexanes/ethyl acetate isocratic) to afford the product as an oil. ¹H NMR (300 MHz, CDCl₃) δ ppm 9.61 (s, 1H), 7.25-7.20 (m, 2H), 6.80-6.74 (m, 2H), 3.84 (s, 3H), 2.90-2.82 (m, 2H), 2.60-2.50 (m, 2H).

### Step 3: 6-methoxy-3,4-dihydronaphthalene-2-carboxylic acid Intermediate 6

To a solution of 6-methoxy-3,4-dihydronaphthalene-2-carbaldehyde (Int-5, 11.85 g, 0.063 mol) in 1 M aqueous sodium hydroxide (125 mL, 0.125 mol) and methanol (255 mL,) was added silver(I) oxide (29.2 g, 0.126 mol). The black mixture was stirred at 50° C. under a nitrogen atmosphere for 2 h. The reaction mixture was cooled to room temperature and filtered through Celite. The filtrate was concentrated under reduced pressure to remove methanol. The resulting aqueous solution was diluted with water and was washed twice with methylene chloride (50 mL). The aqueous phase was acidified with conc. HCl to a pH of 1-2. A thick pale orange precipitate formed. The thick mixture was extracted three times with methylene chloride. The extracts were combined, washed with brine, dried over sodium sulfate, filtered and concentrated under reduced pressure to afford the product as a pale orange solid which was used as obtained (12.3 g, 96%). LC-MS: (AA) ES− 203; ¹H NMR (300 MHz, d₆-DMSO) ppm 12.25 (s, 1H), 7.43 (s, 1H), 7.25 (d, J=8.2 Hz, 1H), 6.84-6.75 (m, 2H), 3.76 (s, 3H), 2.83-2.72 (m, 2H), 2.46-2.38 (m, 2H).

### Step 4: 6-methoxy-3,4-dihydronaphthalene-2-carboxylic acid Intermediate 7

To an argon-purged, slightly opaque solution of 6-methoxy-3,4-dihydronaphthalene-2-carboxylic acid (Int-6, 7.81 g, 0.0382 mol) in methanol (200 mL) was carefully added palladium hydroxide on carbon (0.8 g, 0.005 mol). The contents of the flask were carefully introduced to a hydrogen atmosphere (50 psi) and vigorously stirred at room temperature for 3 h. The heterogeneous mixture was carefully filtered through Celite and concentrated under reduced pressure and used as obtained in the following step (7.50 g, 95%). LC-MS: (AA) ES− 205; ¹H NMR (300 MHz, d₆-DMSO) δ ppm 12.24 (s, 1H), 6.99 (d, J=8.4 Hz, 1H), 6.66 (dd, J=8.3, 2.7 Hz, 1H), 6.62 (d, J=2.5 Hz, 1H), 3.68 (s, 3H), 2.85 (dd, J=16.1, 5.3 Hz, 1H), 2.79-2.68 (m, 3H), 2.59 (dddd, J=9.5, 9.5, 5.4, 3.1 Hz, 1H), 2.10-1.99 (m, 1H), 1.75-1.60 (m, 1H).

### Step 5: 6-methoxy-N-(tetrahydro-2H-pyran-2-yloxy)-1,2,3,4-tetrahydronaphthalene-2-carboxamide Intermediate 8

6-Methoxy-1,2,3,4-tetrahydro-naphthalene-2-carboxylic acid (Int-7, 0.2 g, 0.97 mmol) was dissolved in N,N-dimethylformamide (7 mL) and triethylamine (0.41 mL, 2.9 mmol) was added. Tetramethylfluoroformamidinium hexafluorophosphate (0.307 g, 1.16 mmol) was added and the solution was stirred at room temperature for 15 min. O-(tetrahydropyran-2-yl)hydroxylamine (0.136 g, 1.16 mmol) was then added and the reaction mixture was stirred at room temperature for 2.5 h. The reaction solution was diluted with water (150 mL), to which was added a few grams of sodium chloride, and was extracted with ethyl acetate (3×75 mL). The extracts were combined, washed with water and brine, dried over sodium sulfate, filtered and concentrated under reduced pressure. The crude residue was purified by silica gel chromatography (50/50 to 20/80 hexanes/ethyl acetate gradient) to afford Int-8 (0.21 g, 71%). LC-MS: (AA) ES+ 306.

### Step 6: N-hydroxy-6-methoxy-1,2,3,4-tetrahydronaphthalene-2-carboxamide I-10

To a solution of 6-methoxy-N-(tetrahydro-2H-pyran-2-yloxy)-1,2,3,4-tetrahydronaphthalene-2-carboxamide (Int-8, 0.21 g, 0.688 mmol) in tetrahydrofuran (20 mL) and water (9 mL) was added p-toluenesulfonic acid monohydrate (0.079 g, 0.42 mmol). The solution was stirred at 50° C. for 6 h. After cooling to room temperature, MP-carbonate beads were added (650 mg) and the mixture gently stirred for 10 minutes before filtering. The filtrate was concentrated under reduced pressure and the crude residue was purified by amine functionalized silica gel chromatography (80/20 to 20/80 ethyl acetate/ethanol gradient) to afford the title compound (0.073 g, 48%). LC-MS: (AA) ES+ 222; ¹H NMR (300 MHz, CD₃OD) δ ppm 6.95 (d, J=8.3 Hz, 1H), 6.65 (dd, J=8.3, 2.7 Hz, 1H), 6.61 (d, J=2.4 Hz, 1H), 4.86 (s, 3H), 2.97-2.68 (m, 4H), 2.42 (dddd, J=11.6, 11.6, 5.0, 3.1 Hz, 1H), 2.02-1.92 (m, 1H), 1.90-1.74 (m, 1H).

### Example 4

### 6-(4-chlorophenyl)-N-hydroxy-1,2,3,4-tetrahydronaphthalene-2-carboxamide Compound I-65

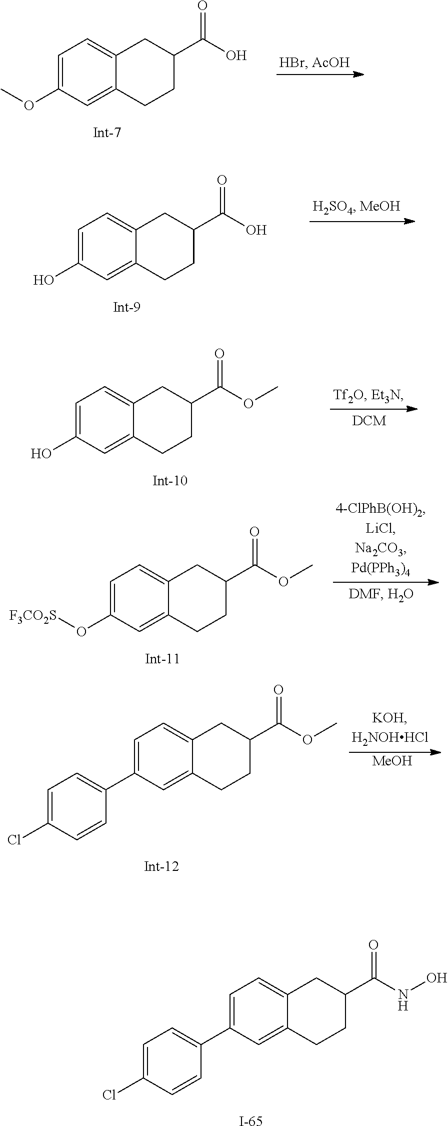

### Step 1: 6-hydroxy-1,2,3,4-tetrahydronaphthalene-2-carboxylic acid Intermediate 9

6-Methoxy-1,2,3,4-tetrahydro-naphthalene-2-carboxylic acid (Int-7, 7.5 g, 36.4 mmol) was suspended in a mixture of acetic acid (60 mL) and aqueous hydrobromic acid (48%, 60 mL). The resulting mixture was heated at reflux in an oil bath for 7 h. The mixture turned homogeneous and dark brown on heating. The reaction mixture was cooled to room temperature and concentrated under reduced pressure to approximately half volume. The concentrated mixture was dissolved in 1M NaOH (350 mL) and water (150 mL). The resulting dark solution was washed with methylene chloride (2×200 mL). The aqueous phase was then acidified (pH 1) with concentrated HCl. The resulting mixture was extracted with methylene chloride (3×250 mL). The extracts were combined, washed with brine, dried over sodium sulfate and used as obtained in the following step (5.33 g, 76%). LC-MS: (FA) ES+ 191; ¹H NMR (300 MHz, d₆-DMSO) δ ppm 12.20 (s, 1H), 9.00 (s, 1H), 6.86 (d, J=8.2 Hz, 1H), 6.49 (dd, J=8.2, 2.5 Hz, 1H), 6.44 (d, J=2.3 Hz, 1H), 2.85-2.63 (m, 4H), 2.56 (dddd, J=8.6, 8.6, 4.6, 2.3 Hz, 1H), 2.02 (dddd, J=6.9, 4.9, 4.9, 3.9 Hz, 1H), 1.64 (dddd, J=12.9, 10.4, 8.2, 8.2 Hz, 1H).

### Step 2: methyl 6-hydroxy-1,2,3,4-tetrahydronaphthalene-2-carboxylate Intermediate 10

To a solution of 6-hydroxy-1,2,3,4-tetrahydronaphthalene-2-carboxylic acid (Int-9, 5.23 g, 27.2 mmol) in methanol (200 mL) was added sulfuric acid (0.15 mL, 2.7 mmol). The resulting solution was heated at reflux overnight. The reaction solution was cooled to room temperature and the methanol was removed under reduced pressure to afford the crude product as a light brown oil. The oil was partitioned between half-saturated sodium bicarbonate solution (150 mL) and ethyl acetate (150 mL). The aqueous phase was extracted with additional ethyl acetate (150 mL). The extracts were combined, washed with saturated aqueous sodium bicarbonate and brine, dried over sodium sulfate, filtered and concentrated under reduced pressure. The crude product was purified by silica gel chromatography (90/10 to 80/20 hexanes/ethyl acetate gradient) to afford the product as a lightly colored oil (3.6 g, 64%). ¹H NMR (300 MHz, d₆-DMSO) δ ppm 9.02 (s, 1H), 6.86 (d, J=8.2 Hz, 1H), 6.50 (dd, J=8.2, 2.5 Hz, 1H), 6.45 (d, J=2.3 Hz, 1H), 3.62 (s, 3H), 2.88-2.63 (m, 5H), 2.09-1.97 (m, 1H), 1.75-1.59 (m, 1H).

### Step 3: methyl 6-(trifluoromethylsulfonyloxy)-1,2,3,4-tetrahydronaphthalene-2-carboxylate Intermediate 11

The title compound was prepared following the procedure detailed in Example 1, step 2, substituting methyl 6-hydroxy-1,2,3,4-tetrahydronaphthalene-2-carboxylate (Int-10) for methyl 7-hydroxy-1,2,3,4-tetrahydronaphthalene-2-carboxylate (Int-1). LC-MS: (FA) ES+ 339; ¹H NMR (300 MHz, d₆-DMSO) δ ppm 7.30 (d, J=8.4 Hz, 1H), 7.23-7.17 (m, 2H), 3.64 (s, 3H), 3.07-2.76 (m, 5H), 2.15-2.03 (m, 1H), 1.82-1.67 (m, 1H).

### Step 4: methyl 6-(4-chlorophenyl)-1,2,3,4-tetrahydronaphthalene-2-carboxylate Intermediate 12

The title compound was prepared following the procedure detailed in Example 1, Step 3 substituting methyl 6-(trifluoromethylsulfonyloxy)-1,2,3,4-tetrahydronaphthalene-2-carboxylate (Int-11) for methyl 7-(trifluoromethylsulfonyloxy)-1,2,3,4-tetrahydronaphthalene-2-carboxylate (Int-2) and 4-chlorophenylboronic acid for 3-chlorophenylboronic acid. ¹H NMR (400 MHz, CDCl₃) δ ppm 7.51-7.46 (m, 2H), 7.41-7.36 (m, 2H), 7.31 (dd, J=7.9, 1.9 Hz, 1H), 7.29-7.27 (m, 1H), 7.18 (d, J=7.9 Hz, 1H), 3.75 (s, 3H), 3.08-3.02 (m, 2H), 2.96-2.88 (m, 2H), 2.83-2.74 (m, 1H), 2.29-2.20 (m, 1H), 1.90 (dddd, J=13.1, 10.6, 10.5, 6.3 Hz, 1H).

### Step 5: 6-(4-chlorophenyl)-N-hydroxy-1,2,3,4-tetrahydronaphthalene-2-carboxamide Compound I-65

The title compound was prepared following the procedure detailed in Example 1, Step 4, substituting methyl 6-(4-chlorophenyl)-1,2,3,4-tetrahydronaphthalene-2-carboxylate for methyl 7-(3-chlorophenyl)-1,2,3,4-tetrahydronaphthalene-2-carboxylate. LC-MS: (FA) ES+ 302; ¹H NMR (400 MHz, d₆-DMSO) δ ppm 10.54 (s, 1H), 8.78 (s, 1H), 7.68-7.62 (m, 2H), 7.50-7.46 (m, 2H), 7.40-7.36 (m, 2H), 7.17 (d, J=7.9 Hz, 1H), 2.94-2.72 (m, 4H), 2.44-2.35 (m, 1H), 1.96-1.87 (m, 1H), 1.73 (dddd, J=12.0, 12.0, 11.9, 5.5 Hz, 1H).

### Example 5

The following compounds were prepared from Intermediate 11 following procedures detailed in Example 1, Steps 3 and 4.

| Compound | LC-MS ES+ (FA) |
|---|---|
| I-66 | 269 |
| I-74 | 325 |

### Example 6

### 7-{3-[2-(dimethylamino)ethoxy]phenyl}-N-hydroxy-1,2,3,4-tetrahydronaphthalene-2-carboxamide Compound I-58

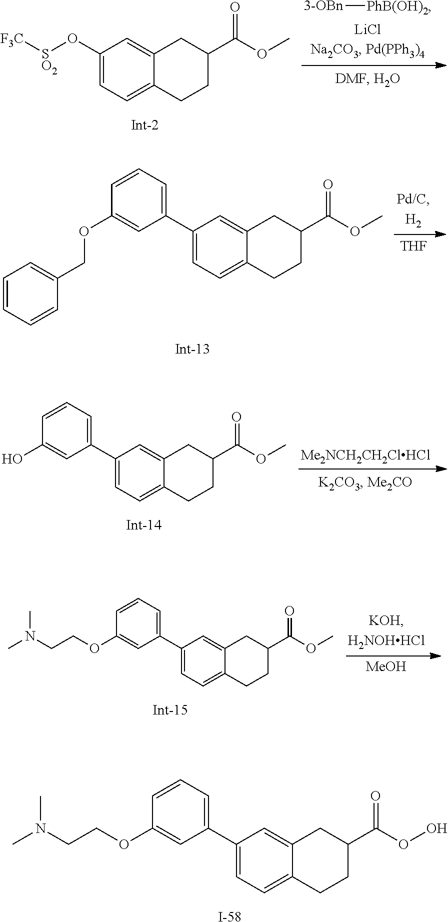

### Step 1: methyl 7-(3-(benzyloxy)phenyl)-1,2,3,4-tetrahydronaphthalene-2-carboxylate Intermediate

The title compound was prepared following the procedure detailed in Example 1, Step 3, substituting 3-benzyloxyphenylboronic acid for 3-chlorophenylboronic acid. Following purification on silica gel, the product was isolated as a mixture with unreacted intermediate 2. The mixture was used as obtained in the following step.

### Step 2: methyl 7-(3-hydroxyphenyl)-1,2,3,4-tetrahydronaphthalene-2-carboxylate Intermediate 14

Methyl 7-[3-(benzyloxy)phenyl]-1,2,3,4-tetrahydronaphthalene-2-carboxylate (Int-13, 0.33 g, 0.886 mmol) was dissolved in tetrahydrofuran (5 mL) in a single neck flask. The contents of the flask were thoroughly purged with argon and then palladium on carbon (10%, 0.033 g) was carefully introduced. The reaction mixture was stirred overnight under an atmosphere of hydrogen. The reaction mixture was then purged with nitrogen, carefully filtered through Celite and the filtrate concentrated under reduced pressure. The crude product was purified by silica gel chromatography (hexanes to 20/80 hexanes/ethyl acetate gradient) to afford the product as an oil (0.19 g, 74%). ¹H NMR (400 MHz, CDCl₃) δ ppm 7.35-7.27 (m, 3H), 7.17-7.12 (m, 2H), 7.04 (dd, J=2.3, 1.8 Hz, 1H), 6.80 (ddd, J=8.0, 2.5, 0.8 Hz, 1H), 4.82 (s, 1H), 3.75 (s, 3H), 3.12-3.04 (m, 2H), 2.97-2.74 (m, 3H), 2.29-2.19 (m, 1H), 1.90 (dddd, J=13.2, 11.0, 10.9, 6.3 Hz, 1H).

### Step 3: methyl 7-(3-(2-(dimethylamino)ethoxy)phenyl)-1,2,3,4-tetrahydronaphthalene-2-carboxylate Intermediate 15

Methyl 7-(3-hydroxyphenyl)-1,2,3,4-tetrahydronaphthalene-2-carboxylate (Int-14, 0.186 g, 0.659 mmol) was dissolved in acetone (5 mL) in a single neck flask under a nitrogen atmosphere. Potassium carbonate (0.273 g, 1.976 mmol) was added followed by β-dimethylaminoethyl chloride hydrochloride (0.114 g, 0.79 mmol). The reaction mixture was then allowed to stir at 60° C. overnight. The contents of the flask were cooled to room temperature and concentrated under reduced pressure. The residue was partitioned between water and ethyl acetate. The aqueous phase was extracted with additional ethyl acetate (2×). The extracts were combined, washed with water and brine then dried over sodium sulfate, filtered and concentrated under reduced pressure. The crude residue was purified by silica gel chromatography (methylene chloride to 90/10 methylene chloride/methanol gradient) to afford Int-15 product (0.16 g, 74%). LC-MS: (FA) ES+ 354; ¹H NMR (400 MHz, CDCl₃) δ ppm 7.37-7.29 (m, 3H), 7.17-7.11 (m, 3H), 6.89 (ddd, J=8.1, 2.5, 0.6 Hz, 1H), 4.15-4.09 (m, 2H), 3.74 (s, 3H), 3.11-3.04 (m, 2H), 2.97-2.86 (m, 2H), 2.83-2.73 (m, 3H), 2.35 (s, 6H), 2.28-2.20 (m, 1H), 1.90 (dddd, J=13.1, 11.0, 10.9, 6.3 Hz, 1H).

### Step 4: 7-{3-[2-(dimethylamino)ethoxy]phenyl}-N-hydroxy-1,2,3,4-tetrahydronaphthalene-2-carboxamide Compound I-58

The title compound was prepared following the procedure detailed in Example 1, Step 4, substituting methyl 7-(3-(2-(dimethylamino)ethoxy)phenyl)-1,2,3,4-tetrahydronaphthalene-2-carboxylate (Int-15) for methyl 7-(3-chlorophenyl)-1,2,3,4-tetrahydronaphthalene-2-carboxylate (Int-3). LC-MS: (FA) ES+ 355; ¹H NMR (400 MHz, d₆-DMSO) δ ppm 10.54 (s, 1H), 8.22 (s, 1H), 7.41-7.29 (m, 3H), 7.20-7.11 (m, 3H), 6.90 (dd, J=8.1, 2.0 Hz, 1H), 4.15-4.08 (m, 2H), 2.96-2.72 (m, 4H), 2.71-2.65 (m, 2H), 2.39 (dddd, J=11.0, 11.0, 5.2, 3.0 Hz, 1H), 2.25 (s, 6H), 1.96-1.85 (m, 1H), 1.73 (dddd, J=11.8, 11.8, 11.8, 5.7 Hz, 1H).

### Example 7

### 7-{3-[(2,2-dimethylpropanoyl)amino]phenyl}-N-hydroxy-1,2,3,4-tetrahydronaphthalene-2-carboxamide Compound I-63

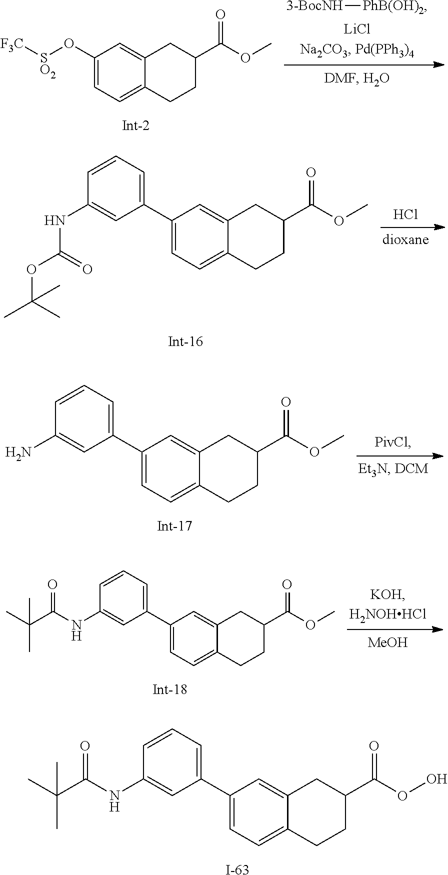

### Step 1: methyl 7-(3-(tert-butoxycarbonylamino)phenyl)-1,2,3,4-tetrahydronaphthalene-2-carboxylate Intermediate 16

The title compound was prepared following the procedure detailed in Example 1, Step 3, substituting 3-(N-Boc-amino)phenylboronic acid for 3-chlorophenylboronic acid. ¹H NMR (400 MHz, CDCl₃) δ ppm 7.57 (s, 1H), 7.37-7.30 (m, 4H), 7.25-7.22 (m, 1H), 7.14 (d, J=7.9 Hz, 1H), 6.53 (s, 1H), 3.74 (s, 3H), 3.13-3.01 (m, 2H), 2.95-2.85 (m, 2H), 2.83-2.73 (m, 1H), 2.28-2.19 (m, 1H), 1.89 (dddd, J=13.1, 10.9, 10.8, 6.2 Hz, 1H).

### Step 2: methyl 7-(3-aminophenyl)-1,2,3,4-tetrahydronaphthalene-2-carboxylate.HCl Intermediate 17

To a solution of methyl 7-(3-(tert-butoxycarbonylamino)phenyl)-1,2,3,4-tetrahydronaphthalene-2-carboxylate (Int-16, 0.233 g, 0.611 mmol) in methylene chloride (10 mL) was added hydrogen chloride in 1,4-dioxane (4.0 M, 1.5 mL, 6.1 mmol). The reaction mixture was then allowed to stir at room temperature overnight. The mixture was concentrated under reduced pressure and the crude residue dissolved in methylene chloride and concentrated again. The crude product was used as obtained without further purification.

### Step 3: methyl 7-(3-pivalamidophenyl)-1,2,3,4-tetrahydronaphthalene-2-carboxylate Intermediate

Methyl 7-(3-aminophenyl)-1,2,3,4-tetrahydronaphthalene-2-carboxylate.HCl (Int-17, 0.09 g, 0.28 mmol) was suspended in methylene chloride (8 mL) to which triethylamine (0.12 mL, 0.85 mmol) was added. Pivaloyl chloride (0.052 mL, 0.43 mmol) was added and the reaction was stirred at room temperature under nitrogen for 2 h. The reaction solution was then concentrated under reduced pressure and the crude residue purified by silica gel chromatography (hexanes to 40/60 hexanes/ethyl acetate gradient. LC-MS: (FA) ES+ 366; ¹H NMR (400 MHz, CDCl₃) δ ppm 7.79 (dd, J=1.8, 1.8 Hz, 1H), 7.47 (ddd, J=7.9, 2.0, 1.2 Hz, 1H), 7.40-7.29 (m, 4H), 7.14 (d, J=7.8 Hz, 1H), 3.74 (s, 3H), 3.15-3.00 (m, 2H), 2.97-2.84 (m, 2H), 2.78 (dddd, J=11.1, 9.6, 6.7, 3.2 Hz, 1H), 2.24 (dddd, J=7.2, 5.9, 4.3, 3.2 Hz, 1H), 1.90 (dddd, J=13.0, 10.8, 10.8, 6.2 Hz, 1H), 1.34 (s, 9H).

### Step 4: 7-{3-[(2,2-dimethylpropanoyl)amino]phenyl}-N-hydroxy-1,2,3,4-tetrahydronaphthalene-2-carboxamide Compound I-63

The title compound was prepared following Example 1, Step 4, substituting methyl 7-(3-pivalamidophenyl)-1,2,3,4-tetrahydronaphthalene-2-carboxylate (Int-18) for methyl 7-(3-chlorophenyl)-1,2,3,4-tetrahydronaphthalene-2-carboxylate (Int-3). LC-MS: (FA) ES+ 367; ¹H NMR (400 MHz, d₆-DMSO) δ ppm 10.54 (s, 1H), 9.25 (s, 1H), 8.80 (s, 1H), 7.91 (dd, J=1.7, 1.7 Hz, 1H), 7.68-7.64 (m, 1H), 7.37-7.31 (m, 3H), 7.28 (ddd, J=7.8, 1.3, 1.3 Hz, 1H), 7.16 (d, J=8.5 Hz, 1H), 2.98-2.69 (m, 4H), 2.45-2.36 (m, 1H), 1.96-1.86 (m, 1H), 1.74 (dddd, J=12.1, 12.1, 11.9, 5.7 Hz, 1H), 1.23 (s, 9H).

### Example 8

### 7-{3-[(cyclopropylcarbonyl)amino]phenyl}-N-hydroxy-1,2,3,4-tetrahydronaphthalene-2-carboxamide Compound I-64

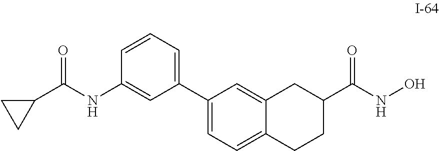

The title compound was prepared following the procedures detailed in Example 7, substituting cyclopropanecarbonyl chloride for pivaloyl chloride. LC-MS: (FA) ES+ 351; ¹H NMR (400 MHz, d₆-DMSO) δ ppm 10.56 (s, 1H), 10.30 (s, 1H), 8.84 (s, 1H), 7.86 (s, 1H), 7.54 (d, J=8.4 Hz, 1H), 7.36-7.30 (m, 3H), 7.26 (d, J=7.8 Hz, 1H), 7.15 (d, J=8.5 Hz, 1H), 2.96-2.65 (m, 4H), 2.46-2.35 (m, 1H), 1.96-1.86 (m, 1H), 1.83-1.67 (m, 2H), 0.84-0.73 (m, 4H).

### Example 9

### (2S)-7-({2-[(cyclopropylcarbonyl)amino]pyridin-4-yl}oxy)-N-hydroxy-1,2,3,4-tetrahydronaphthalene-2-carboxamide Compound I-2

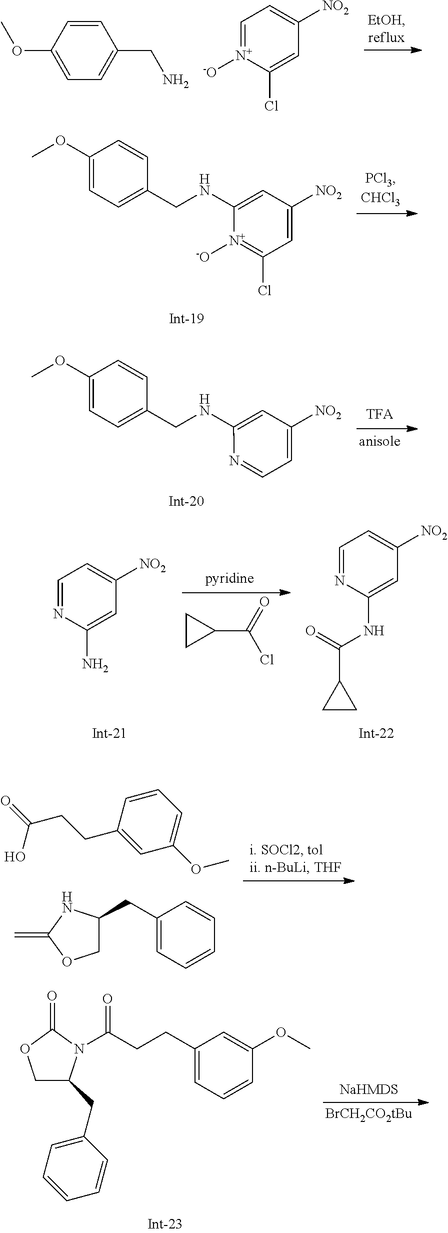

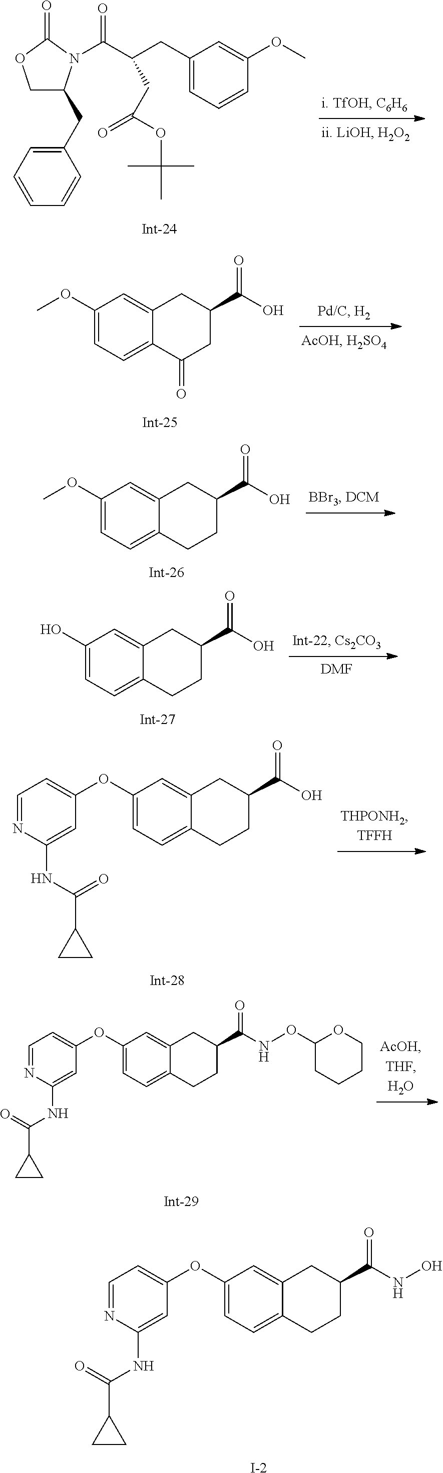

### Step 1: N-(4-methoxybenzyl)-4-nitropyridin-2-amine 1-oxide Intermediate 19

A mixture of 2-chloro-4-nitropyridine-1-oxide (75 g, 0.43 mol) and 1-(4-methoxyphenyl)-methanamine (125 g, 0.91 mol) in ethanol (1 L) was heated at reflux for 5 h. The reaction was allowed to cool to room temperature and cooled in a freezer overnight. The resulting cold mixture was filtered. The isolated solid was further slurried in methanol (100 mL) and filtered to provide N-(4-methoxybenzyl)-4-nitropyridin-2-amine 1-oxide (51.62 g, 40% yield) as an orange solid. LCMS: (FA) ES+ 276; ¹H NMR (400 MHz, d₆-DMSO) δ ppm 8.34 (dd, J=6.5, 0.8 Hz, 1H), 8.27 (dd, J=6.7, 6.7 Hz, 1H), 7.39-7.34 (m, 1H), 7.29 (d, J=8.6 Hz, 1H), 6.89 (d, J=8.6 Hz, 1H), 4.52 (d, J=6.6 Hz, 1H), 3.70 (s, 1H).

### Step 2: N-(4-methoxybenzyl)-4-nitropyridin-2-amine Intermediate 20

To a 2 L 3 neck round bottom flask fitted with a mechanical stirrer, was added N-(4-methoxybenzyl)-4-nitropyridin-2-amine 1-oxide (Int-19, 38.72 g, 0.14 mol) and chloroform (580 mL). The reaction mixture was cooled to 0° C. and phosphorus trichloride (36.8 mL, 0.42 mol) was added dropwise. The reaction mixture was allowed to warm to room temperature and stir overnight. The reaction mixture was filtered and the resulting solid was slurried with hexanes and filtered (repeated several times) to afford N-(4-methoxybenzyl)-4-nitropyridin-2-amine (39.34 g, 102%) as a yellow solid. LCMS: (FA) ES+ 260; ¹H NMR (400 MHz, CD₃OD) δ ppm 8.15 (d, J=6.9 Hz, 1H), 7.70 (d, J=2.1 Hz, 1H), 7.42 (dd, J=6.8, 2.2 Hz, 1H), 7.37-7.32 (m, 2H), 6.97-6.92 (m, 2H), 4.57-4.55 (m, 2H), 3.78 (s, 3H).

### Step 3: 4-nitropyridin-2-amine Intermediate 21

N-(4-Methoxybenzyl)-4-nitropyridin-2-amine (Int-20, 27.8 g, 0.11 mol) and anisole (13 mL, 0.12 mol) were dissolved in trifluoroacetic acid (112 mL) and heated at 80° C. for 2 h. The reaction mixture was allowed to cool to room temperature and concentrated. Trituration of the resulting residue in ethyl acetate and hexanes produced a light yellow solid that was isolated by filtration. The filtrate was allowed to stand overnight producing a second crop of product. The combined solids were dissolved in 1N NaOH (250 mL) and extracted twice with ethyl acetate. The combined organic solutions were dried over magnesium sulfate, filtered and concentrated to give 4-nitropyridin-2-amine as an orange solid (Int-20, 10.2 g, 67%). LCMS: (FA) ES+ 140; ¹H NMR (400 MHz, CD₃OD) δ ppm 8.15 (dd, J=5.7, 0.6 Hz, 1H), 7.23 (dd, J=2.0, 0.6 Hz, 1H), 7.20 (dd, J=5.7, 2.0 Hz, 1H).

### Step 4: N-(4-nitropyridin-2-yl)cyclopropanecarboxamide Intermediate 22

4-Nitropyridin-2-amine (Int-21, 32.4 g, 0.2096 mol) was dissolved in pyridine (200 mL). The reaction was cooled in an ice-water bath and cyclopropanecarbonyl chloride (28.5 mL, 0.314 mol) was added dropwise. Upon completion of addition, the reaction was stirred for 1 h at 0° C. then warmed to room temperature and stirred overnight. The reaction mixture was concentrated under reduced pressure and then diluted with water. Solids were collected by suction filtration. The filter cake was washed with water and hexanes. The isolated solids were then dried under vacuum to afford N-(4-nitropyridin-2-yl)cyclopropanecarboxamide (43.5 g, 100%). LCMS: (FA) ES+ 208; ¹H NMR (300 MHz, CDCl₃) δ ppm 8.90 (s, 1H), 8.50 (s, 1H), 8.50 (br s, 1H), 7.7 (dd, 1H), 1.6 (m, 1H), 1.2 (m, 2H), and 1.0 (m, 2H)

### Step 5: (S)-4-benzyl-3-(3-(3-methoxyphenyl)propanoyl)oxazolidin-2-one Intermediate 23

To a stirred solution of 3-(3-methoxyphenyl)propionic acid (20 g, 111 mmol) in toluene (69.2 mL) was added thionyl chloride (14.4 mL) dropwise at room temperature. The resulting mixture was heated at reflux for 2 h, cooled to room temperature and concentrated under reduced pressure. Meanwhile, to a solution of (S)-4-benzyl-2-oxazolidinone (16.4 g, 92 mmol) in anhydrous THF (232 mL) was added n-butyllithium (63.2 mL, 1.6 M in hexanes) dropwise at −78° C. This reaction mixture was stirred for 1 h at −78° C. after which a solution of the acid chloride in THF (6 mL) was added. This reaction was maintained at −78° C. for 3 h. After quenching with a saturated aqueous solution of ammonium chloride, the mixture was warmed to room temperature and extracted with ethyl acetate three times. The combined extracts were washed with brine, dried over sodium sulfate, filtered and concentrated under reduced pressure. The yellow residue obtained was triturated twice with hexane to give a white powder which was dried under high vacuum to give (S)-4-benzyl-3-(3-phenyl-propionyl)-oxazolidin-2-one (28.2 g, 89%). ¹H NMR (400 MHz, d₆-DMSO) δ ppm 7.33-7.17 (m, 4H), 7.16-7.12 (m, 2H), 6.84-6.81 (m, 3H), 4.64 (ddd, J=10.8, 7.7, 2.9 Hz, 1H), 4.30 (dd, J=8.5, 8.5 Hz, 1H), 4.17 (dd, J=8.8, 2.8 Hz, 1H), 3.72 (s, 3H), 3.17 (ddd, J=16.1, 8.9, 7.1 Hz, 1H), 3.05 (ddd, J=14.8, 8.4, 6.5 Hz, 1H), 2.98 (dd, J=13.5, 3.2 Hz, 1H), 2.94-2.78 (m, 3H).

### Step 6: (R)-tert-butyl 4-((S)-4-benzyl-2-oxooxazolidin-3-yl)-3-(3-methoxybenzyl)-4-oxobutanoate Intermediate 24

To a stirred solution of (S)-4-benzyl-3-(3-phenyl-propionyl)-oxazolidin-2-one (Int-23, 5.2 g, 15 mmol) in anhydrous THF (27.5 mL), cooled to −78° C., was added NaHMDS (19.4 mL, 1 M in THF), dropwise over 15 min. The resulting mixture was stirred for 1 h, then tert-butyl bromoacetate (2.9 mL) was added dropwise and the reaction mixture was stirred for another 4 h at −78° C. The reaction was then quenched by the addition of saturated aqueous ammonium chloride solution and the reaction mixture was allowed to warm to room temperature. The mixture was extracted twice with ethyl acetate. The combined extracts were washed with water and brine, dried over sodium sulfate, filtered and concentrated. The resulting residue was triturated several times with hexanes and the white solid thus obtained was dried to yield (R)-tert-butyl 4-((S)-4-benzyl-2-oxooxazolidin-3-yl)-3-(3-methoxybenzyl)-4-oxobutanoate (5.5 g, 79%). ¹H NMR (400 MHz, d₆-DMSO) δ ppm 7.37-7.12 (m, 6H), 6.83-6.75 (m, 3H), 4.64-4.51 (m, 1H), 4.28-4.18 (m, 2H), 4.14 (dd, J=8.7, 2.4 Hz, 1H), 3.71 (s, 3H), 3.04-2.80 (m, 3H), 2.68 (dd, J=16.8, 10.9 Hz, 1H), 2.55-2.50 (m, 1H), 2.24 (dd, J=16.8, 3.9 Hz, 1H), 1.35 (s, 9H).

### Step 7: (R)-7-methoxy-4-oxo-1,2,3,4-tetrahydronaphthalene-2-carboxylic acid Intermediate 25

To a solution of (R)-tert-butyl 4-((S)-4-benzyl-2-oxooxazolidin-3-yl)-3-(3-methoxybenzyl)-4-oxobutanoate (Int-24, 35 g, 77 mmol) in benzene (42 mL) was added triflic acid (21 mL, 238 mmol) and the reaction mixture was heated at reflux overnight. The reaction mixture was cooled to room temperature and concentrated under reduced pressure. The residue was diluted in THF (140 mL) and water (35 mL). The resulting mixture was cooled to 0° C. and hydrogen peroxide (30%, 35 mL) was added followed by lithium hydroxide (20 g). Stirring was continued for 1 h followed by the addition of an excess of sodium sulfite solution (10% w/v). The reaction mixture was allowed to warm to room temperature and a saturated solution of sodium bicarbonate in water (150 ml) was added. The mixture was then washed with methylene chloride. The aqueous phase was retained and was acidified with conc. HCl (pH 2-3). The off-white precipitate obtained was isolated by suction filtration, washed with cold water and dried to give 7-methoxy-4-oxo-1,2,3,4-tetrahydro-naphthalene-2-carboxylic acid (12 g, 70%). ¹H NMR (400 MHz, d₆-DMSO) δ ppm 12.51 (s, 1H), 7.79 (d, J=8.6 Hz, 1H), 6.92 (d, J=2.3 Hz, 1H), 6.89 (dd, J=8.7, 2.4 Hz, 1H), 3.82 (s, 3H), 124-3.04 (m, 3H), 2.73-2.66 (m, 2H).

### Step 8: (S)-7-methoxy-1,2,3,4-tetrahydronaphthalene-2-carboxylic acid Intermediate 26

(R)-7-Methoxy-4-oxo-1,2,3,4-tetrahydronaphthalene-2-carboxylic acid (Int-25, 18 g, 81 mmol) was combined in a Parr hydrogenation vessel with acetic acid (460 mL) and sulfuric acid (0.28 mL). Palladium on carbon (5.04 g, 10 wt %, wet) was added. The vessel was placed in an autoclave set at 80° C. and was hydrogenated at 20 psi for 2 h. The reaction mixture was filtered through Celite. The filtrate was extracted twice with DCM. The organic layer was dried over sodium sulfate, filtered and concentrated. The solid residue obtained was triturated with diethyl ether and dried to yield (S)-7-methoxy-1,2,3,4-tetrahydronaphthalene-2-carboxylic acid (11 g, 65%). ¹H NMR (400 MHz, d₆-DMSO) δ ppm 12.22 (s, 1H), 6.95 (d, J=9.2 Hz, 1H), 6.69-6.63 (m, 2H), 3.68 (s, 3H), 2.88 (dd, J=16.5, 5.7 Hz, 1H), 2.80 (dd, J=16.5, 9.7 Hz, 1H), 2.71-2.55 (m, 3H), 2.04 (ddd, J=12.9, 8.3, 4.5 Hz, 1H), 1.68 (dddd, J=12.8, 10.1, 9.9, 6.8 Hz, 1H).

### Step 9: (S)-7-hydroxy-1,2,3,4-tetrahydronaphthalene-2-carboxylic acid Intermediate 27

(S)-7-Methoxy-1,2,3,4-tetrahydronaphthalene-2-carboxylic acid (Int-26, 31 g, 0.14 mol) was suspended in DCM (300 mL), cooled in a dry ice/acetone bath under argon, and then a solution of boron tribromide in DCM (1.00 M, 280 mL, 0.28 mol) was added over 4 h. The reaction was stirred an additional 10 min at −78° C. then warmed to 0° C. and stirred for 2 h. The reaction was cooled to −50° C. in a dry ice/methanol bath and ice-cold water (15 mL) was added. The temperature was allowed to warm slowly to −10° C. and the cooling bath was replaced with an ice-water bath. Saturated aqueous sodium bicarbonate solution (100 mL) was added. Additional solid sodium bicarbonate was added to adjust the pH to 9. The phases were separated and the aqueous phase washed twice with DCM. The pH of the aqueous phase was adjusted to 2 with concentrated HCl. The aqueous phase was then extracted with ether three times. The extracts were combined, washed with 1M HCl and brine, dried over sodium sulfate, filtered and concentrated under reduced pressure. The residue was suspended in a small amount of ether and filtered. The solid collected was dried in vacuo to afford the desired product as a white solid. The filtrate was partially concentrated and purified by silica gel chromatography (60% ether in hexanes) to afford additional product. The total combined yield of (S)-7-hydroxy-1,2,3,4-tetrahydronaphthalene-2-carboxylic acid was 20.3 g (74%). ¹H NMR (400 MHz, d₆-DMSO) δ ppm 12.22 (s, 1H), 9.01 (s, 1H), 6.83 (d, J=8.1 Hz, 1H), 6.51-645 (m, 2H), 2.81 (dd, J=16.8, 5.9 Hz, 1H), 2.74 (ddd, J=16.4, 9.8 Hz, 1H), 2.66-2.52 (m, 3H), 2.03 (ddd, J=12.4, 7.9, 4.6 Hz, 1H), 1.65 (dddd, J=12.8, 9.8, 9.7, 6.9 Hz, 1H).

### Step 10: (S)-7-(2-(cyclopropanecarboxamido)pyridin-4-yloxy)-1,2,3,4-tetrahydronaphthalene-2-carboxylic acid Intermediate 28

A mixture of (S)-7-hydroxy-1,2,3,4-tetrahydronaphthalene-2-carboxylic (Int-27, 500 mg, 2.86 mmol), N-(4-nitropyridin-2-yl)cyclopropanecarboxamide (Int-22, 0.539 g, 0.0026 mol), and cesium carbonate (2.54 g, 0.0078 mol) in N,N-dimethylformamide (13 mL) was heated at 80° C. for 48 h. The reaction mixture was cooled to room temperature and the solvent was evaporated under reduced pressure. The resulting crude residue was acidified to approximately pH 3 with 1N HCl. The precipitated solid was isolated by suction filtration, washed with water then hexane and dried under high vacuum (824 mg, 90%). LC-MS: (FA) ES+ 353; ¹H NMR (400 MHz, d₆-DMSO) δ ppm 11.34 (s, 1H), 8.19 (d, J=6.2 Hz, 1H), 7.38 (s, 1H), 7.19 (d, J=8.3 Hz, 1H), 6.97 (d, J=2.2 Hz, 1H), 6.93 (dd, J=8.3, 2.5 Hz, 1H), 6.82-6.77 (m, 1H), 2.95 (dd, J=17.1, 5.3 Hz, 1H), 2.86 (d, J=11.2 Hz, 1H), 2.84-2.77 (m, 2H), 2.66 (dddd, J=9.9, 9.9, 5.5, 3.1 Hz, 1H), 2.16-2.06 (m, 1H), 1.93 (dddd, J=7.5, 7.5, 4.9, 4.9 Hz, 1H), 1.79-1.66 (m, 1H), 0.90-0.77 (m, 4H).

### Step 11: (2S)-7-(2-(cyclopropanecarboxamido)pyridin-4-yloxy)-N-(tetrahydro-2H-pyran-2-yloxy)-1,2,3,4-tetrahydronaphthalene-2-carboxamide Intermediate 29

The title product was prepared following the procedure detailed in Example 3, Step 5 substituting (S)-7-(2-(cyclopropanecarboxamido)pyridin-4-yloxy)-1,2,3,4-tetrahydronaphthalene-2-carboxylic acid (Int-28) for 6-methoxy-1,2,3,4-tetrahydro-naphthalene-2-carboxylic acid (Int-7). LC-MS: (AA) ES+ 452; ¹H NMR (400 MHz, CD₃OD) δ ppm 8.09 (d, J=5.8 Hz, 1H), 7.62 (d, J=2.2 Hz, 1H), 7.15 (d, J=9.0 Hz, 1H), 6.88-6.82 (m, 2H), 6.62 (dd, J=5.8, 2.3 Hz, 1H), 4.91 (s, 1H), 4.01 (ddd, J=10.8, 10.7, 2.3 Hz, 1H), 3.64-3.58 (m, 1H), 3.06-2.77 (m, 4H), 2.53 (dddd, J=11.3, 11.3, 4.8, 2.9 Hz, 1H), 1.95-1.72 (m, 5H), 1.71-1.51 (m, 4H), 0.97-0.82 (m, 4H).

### Step 12: (2S)-7-({2-[(cyclopropylcarbonyl)amino]pyridin-4-yl}oxy)-N-hydroxy-1,2,3,4-tetrahydronaphthalene-2-carboxamide Compound I-2

The title compound was prepared following the procedure detailed in Example 3, Step 6, substituting (2S)-7-(2-(cyclopropanecarboxamido)pyridin-4-yloxy)-N-(tetrahydro-2H-pyran-2-yloxy)-1,2,3,4-tetrahydronaphthalene-2-carboxamide (Int-29) for 6-methoxy-N-(tetrahydro-2H-pyran-2-yloxy)-1,2,3,4-tetrahydronaphthalene-2-carboxamide (Int-8). LC-MS: (AA) ES+ 368; ¹H NMR (400 MHz, CD₃OD) δ ppm 8.10 (d, J=5.8 Hz, 1H), 7.62 (d, J=2.2 Hz, 1H), 7.15 (d, J=9.0 Hz, 1H), 6.88-6.83 (m, 2H), 6.62 (dd, J=5.8, 2.3 Hz, 1H), 3.06-2.77 (m, 4H), 2.48 (dddd, J=11.4, 11.4, 5.0, 3.0 Hz, 1H), 2.07-2.01 (m, 1H), 1.95-1.79 (m, 2H), 0.96-0.81 (m, 4H).

### Example 10

### (2S)—N-hydroxy-7-[(7-oxo-5,6,7,8-tetrahydro-1,8-naphthyridin-4-yl)oxy]-1,2,3,4-tetrahydronaphthalene-2-carboxamide Compound I-4

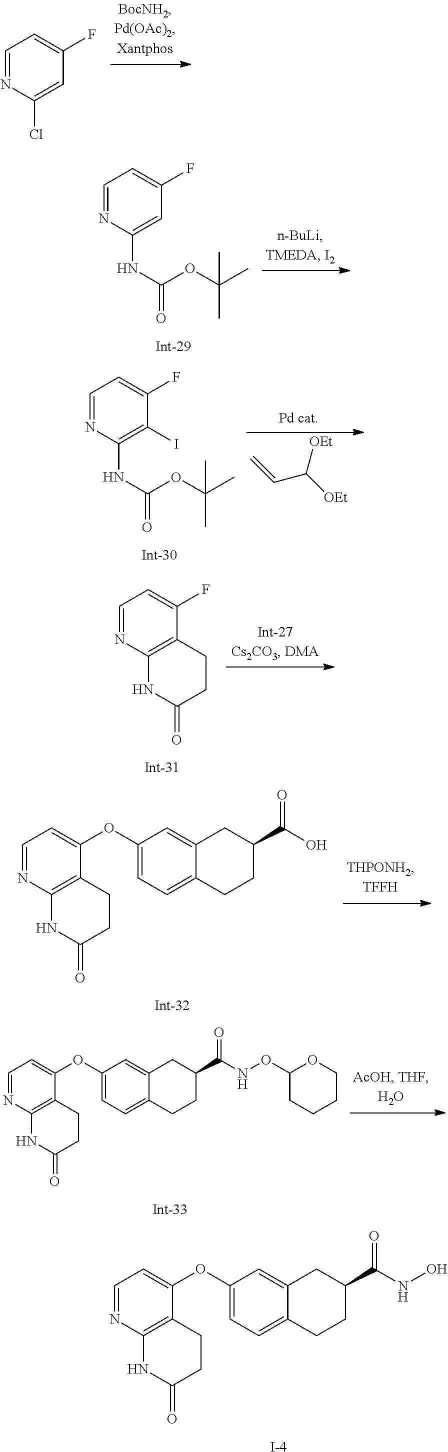

### Step 1: tert-butyl (4-fluoropyridin-2-yl)carbamate Intermediate 29

Palladium(II) acetate (341 mg, 1.52 mmol) and 4,5-bis(diphenylphosphino)-9,9-dimethylxanthene (1.76 g, 3.04 mmol) were added to a 3-necked round bottomed flask, and the flask was purged three times with argon. Degassed 1,4-dioxane (240 mL) was added and the mixture was stirred and degassed again with argon. To this solution was added a solution of 2-chloro-4-fluoropyridine (20 g, 152 mmol) in degassed 1,4-dioxane (120 mL), tert-butyl carbamate (19.6 g, 167 mmol), NaOH (8.88 g, 222 mmol) and degassed water (4 mL, 222 mmol). The resulting mixture was stirred at 100° C. under argon. After 1.5 h, the reaction mixture was cooled to room temperature and filtered through Celite. The pad was washed well with dioxane and the filtrate was concentrated under reduced pressure to dryness. The resulting solid was recrystallized from 2-propanol (approx. 250 mL) to give tert-butyl (4-fluoropyridin-2-yl)carbamate as a pale yellow crystalline solid (25.65 g, 79.5% yield). LC-MS: (FA) ES+ 213; ¹H NMR (400 MHz, d₆-DMSO) δ ppm 10.1 (s, 1H), 8.26 (dd, J=9.5, 5.6 Hz, 1H), 7.60 (dd, J=12.3, 2.3 Hz, 1H), 6.95 (ddd, J=8.3, 5.6, 2.3 Hz, 1H), 1.47 (s, 9H).

### Step 2: tert-butyl (4-fluoro-3-iodopyridin-2-yl)carbamate Intermediate 30

An oven-dried 3-neck round bottom flask equipped with an overhead stirrer, temperature probe, and addition funnel was charged with tert-butyl (4-fluoropyridin-2-yl)carbamate (Int-29, 31.8 g, 150 mmol), TMEDA (56.6 mL, 375 mmol) and THF (200 mL). The solution was cooled to −78° C. and a solution of n-butyllithium (2.50 M in hexane, 150 mL, 375 mmol) was added dropwise such that the reaction mixture temperature remained below −70° C. Upon completion of addition, the reaction mixture was stirred at −78° C. for 1 h, and a solution of h (95.2 g, 375 mmol) in THF (160 mL) was added via addition funnel. The addition was again controlled to keep the reaction mixture temperature below −70° C., and the resulting mixture was stirred at −78° C. for 1 h. A solution of NaHSO₃ (61 g, 580 mmol) in water (200 mL) was added to the reaction mixture as it warmed to room temperature. Ethyl acetate was added and the biphasic mixture was stirred at room temperature for 1 h. Water (500 mL) was added and the phases were separated. The aqueous phase was extracted with EtOAc (3×400 mL); the organic phases were combined, dried over magnesium sulfate, filtered and concentrated to give an off-white solid. This solid was suspended in methylene chloride (50 mL), isolated by suction filtration and washed with a minimum of methylene chloride. The filtrate was concentrated and filtered to give a second crop of product. The solids were combined and dried under vacuum to give tert-butyl (4-fluoro-3-iodopyridin-2-yl)carbamate as a white solid (45.63 g, 86% yield). LC-MS: (FA) ES+ 339. ¹H NMR (400 MHz, d₆-DMSO) δ ppm 9.47 (s, 1H), 8.32 (dd, J=8.9, 5.5 Hz, 1H), 7.18 (dd, J=7.2, 5.5 Hz, 1H), 1.44 (s, 9H).

### Step 3: 5-fluoro-3,4-dihydro-1,8-naphthyridin-2(1H)-one Intermediate 31

A round bottom flask was charged with tert-butyl (4-fluoro-3-iodopyridin-2-yl)carbamate (Int-30, 20 g, 59.2 mmol), 3,3-diethoxy-1-propene (13.5 mL, 88.7 mmol), N,N-dimethylformamide (150 mL), water (50 mL), N,N-diisopropylethylamine (15.4 mL, 88.7 mmol) and the Pd catalyst (“Palladacyle 1” from Corma et. al., Tetrahedron 2005, 61(41):9848; 480 mg, 0.827 mmol) and the reaction mixture was warmed to 140° C. After 5 h, the reaction mixture was cooled in a refrigerator for 2 days. The resulting precipitate was isolated by suction filtration, washed with diethyl ether, and dried to give 3.25 g of pink needles. The filtrate was concentrated to give a reddish semi-solid. This material was redissolved in methylene chloride and the solution was passed through 200 g of silica. Concentration of the resulting solution provided a red/orange residue which was recrystallized from 2-propanol (150 mL) to give 9.4 g of a pink solid. Purification of this pink solid by column chromatography (SiO₂, elution with 0-75% ethyl acetate in methylene chloride) provided 1.41 g of a white powder. Overall, 4.66 g of 5-fluoro-3,4-dihydro-1,8-naphthyridin-2(1H)-one was isolated (47% yield). LC-MS: (FA) ES+ 167; ¹H NMR (400 MHz, d₆-DMSO) δ ppm 10.7 (s, 1H), 8.11 (dd, J=8.5, 5.7 Hz, 1H), 6.91 (dd, J=8.8, 5.7 Hz, 1H), 2.91-2.85 (m, 2H), 2.55-2.50 (m, 2H).

### Step 4: (S)-7-(7-oxo-5,6,7,8-tetrahydro-1,8-naphthyridin-4-yloxy)-1,2,3,4-tetrahydronaphthalene-2-carboxylic acid Intermediate 32

5-Fluoro-3,4-dihydro-1,8-naphthyridin-2(1H)-one (Int-31, 50 mg, 0.301 mmol), (2S)-7-hydroxy-1,2,3,4-tetrahydronaphthalene-2-carboxylic acid (63.6 mg, 0.331 mmol), and cesium carbonate (294 mg, 0.903 mmol) were weighed into a microwave vial (2-5 mL) followed by N,N-dimethylacetamide (1.4 mL). The vial was sealed and the resulting mixture was stirred for 1 h at 150° C. under microwave irradiation. The contents of the vial were cooled to room temperature, water (5 mL) was added and the resulting mixture was stirred for a few minutes. The clear solution was neutralized by addition of 1N HCl and the resulting suspension was filtered through a Celite pad. The solid residue was washed with methanol then the filtrate was concentrated in vacuo. The residue was purified by silica gel column chromatography (2-10% methanol in methylene chloride gradient) to provide 52 mg of product as a colorless solid (41%). The enantiomeric purity of this product was assessed by chiral HPLC (85% ee). Column: IA 4.6×250 mm Elute: 100/0.1 EtOH/TFA; 0.5 mL/min 10 uL injection 50 min. LC-MS: (FA) ES+ 339; ¹H NMR (400 MHz, d₆-DMSO) δ ppm 12.33 (s, 1H), 10.49 (s, 1H), 8.00-7.90 (m, 1H), 7.15 (d, J=8.3 Hz, 1H), 6.90 (d, J=2.4 Hz, 1H), 6.87 (dd, J=8.2, 2.5 Hz, 1H), 6.28 (d, J=5.8 Hz, 1H), 2.98-2.74 (m, 5H), 2.71-2.61 (m, 1H), 2.57-2.51 (m, 3H), 2.14-2.06 (m, 1H), 1.73 (dddd, J=13.0, 10.0, 9.9, 6.7 Hz, 1H).

### Steps 5 and 6: (2S)—N-hydroxy-7-[(7-oxo-5,6,7,8-tetrahydro-1,8-naphthyridin-4-yl)oxy]-1,2,3,4-tetrahydronaphthalene-2-carboxamide Compound I-4

The title compound was prepared following the procedures detailed in Example 3, Steps 5 and 6. LC-MS: (FA) ES+ 354; ¹H NMR (400 MHz, d₆-DMSO/D₂O) δ ppm 7.94 (d, J=5.8 Hz, 1H), 7.15 (d, J=8.0 Hz, 1H), 6.90-6.83 (m, 2H), 6.27 (d, J=5.8 Hz, 1H), 2.95-2.65 (m, 5H), 2.57-2.51 (m, 3H), 2.42-2.30 (m, 1H), 1.94-1.85 (m, 1H), 1.77-1.63 (m, 1H).

### Example 11

### N-hydroxy-8-methoxy-1,2,3,4-tetrahydronaphthalene-2-carboxamide Compound I-15

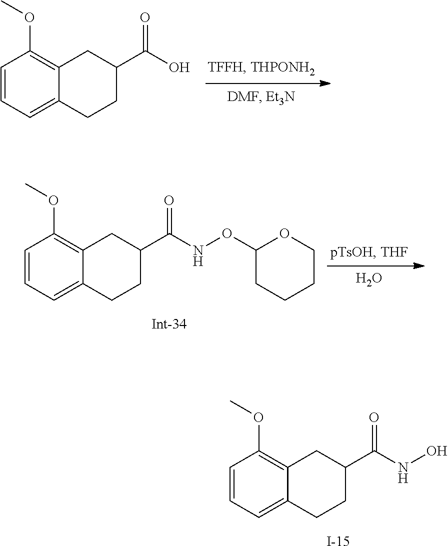

### Steps 1 and 2: N-hydroxy-8-methoxy-1,2,3,4-tetrahydronaphthalene-2-carboxamide I-15

The title compound was prepared from commercially available 8-methoxy-1,2,3,4-tetrahydronaphthalene-2-carboxylic acid following the procedures detailed in Example 3, Steps 5 and 6. LC-MS: (FA) ES+ 222; ¹H NMR (400 MHz, d₆-DMSO) δ ppm 10.49 (s, 1H), 8.75 (s, 1H), 7.05 (dd, J=7.8, 7.8 Hz, 1H), 6.72 (d, J=8.0 Hz, 1H), 6.66 (d, J=7.6 Hz, 1H), 3.74 (s, 3H), 2.83-2.62 (m, 3H), 2.55-2.45 (m, 1H), 2.38-2.22 (m, 1H), 1.89-1.79 (m, 1H), 1.62 (ddd, J=24.5, 12.1, 5.6 Hz, 1H).

### Example 12

The following compounds were prepared in a fashion analogous to that described in Example 11 starting from commercially available tetralins.

| Compound | LC-MS ES+ (FA) |
|---|---|
| I-1 | 222 |
| I-3 | 192 |

### Example 13

The following compounds were prepared from the appropriate tetralins and the pyridines described in Examples 9 and 10, following procedures detailed in Example 10, Steps 4 through 6.

| | LC-MS (FA)/(AA) |
|---|---|
| Compound | ES+ |
| I-13 | (AA) 368 |
| I-14 | (AA) 354 |
| I-16 | (FA) 368 |

### Example 14

### 7-({2-[(cyclopropylcarbonyl)amino]pyridin-4-yl}oxy)-6-fluoro-N-hydroxy-1,2,3,4-tetrahydronaphthalene-2-carboxamide Compound I-32

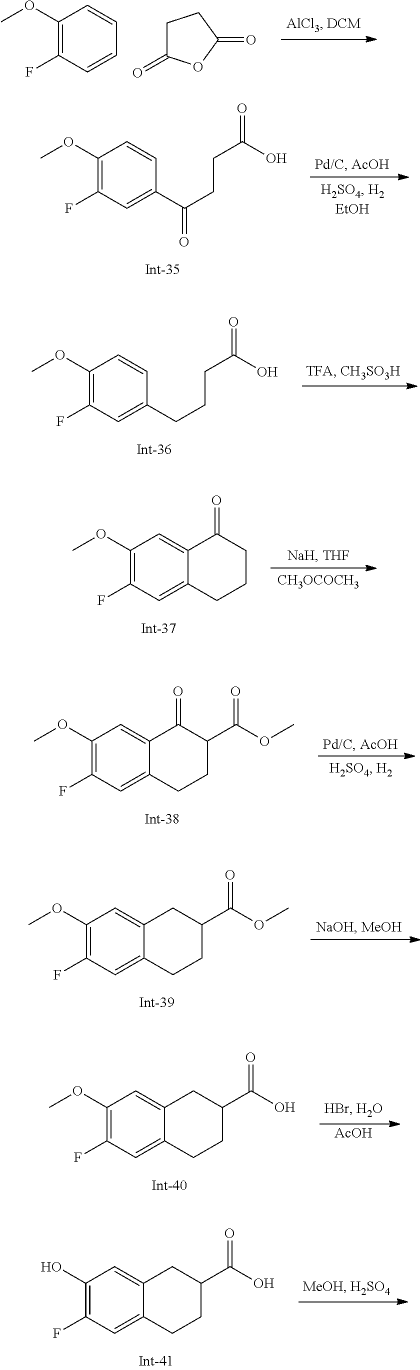

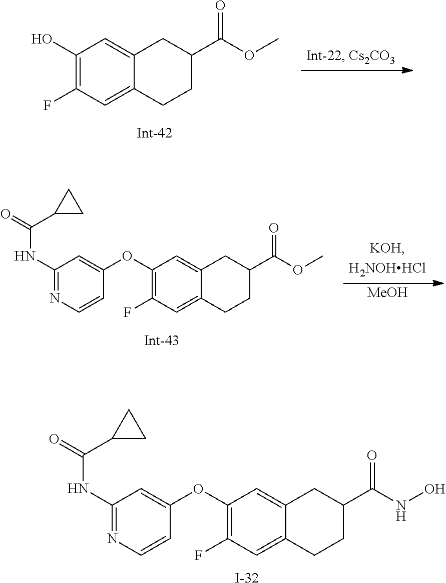

### Step 1: 3-(3-fluoro-4-methoxybenzoyl)propionic acid Intermediate 35

1-Fluoro-2-methoxybenzene (7 mL, 0.062 mol) and succinic anhydride (6.93 g, 0.0692 mol) were dissolved in methylene chloride (100 mL). The resulting solution was cooled in an ice-water bath for 20 min. Aluminum trichloride (18.3 g, 0.137 mol) was then added in portions. Upon completion of addition, the mixture was heated at 65° C. in an oil bath for 2 h and then cooled to room temperature and allowed to stir overnight. The reaction mixture was poured onto ice and 200 mL 1N HCl was added. A white precipitate formed which was isolated by suction filtration. The filter cake was dried under suction and then further dried under vacuum to provide the title compound as a white solid (12.703 g, 90%). LC-MS: (FA) ES− 225; NMR (400 MHz, CD₃OD) δ ppm 7.86 (ddd, J=8.6, 2.1, 1.14 Hz, 1H), 7.72 (dd, J=12.1, 2.1 Hz, 1H), 7.20 (dd, J=8.5, 8.5 Hz, 1H), 3.95 (s, 3H), 3.28-3.23 (m, 2H), 2.71-2.66 (m, 2H).

### Step 2: 4-(3-fluoro-4-methoxyphenyl)butanoic acid Intermediate 36

3-(3-Fluoro-4-methoxybenzoyl)propionic acid (Int-35, 12 g, 0.0531 mol), ethanol (190 mL), acetic acid (27 mL) were combined in a Parr shaker flask. The contents of the flask were flushed with argon and 10% palladium on carbon (0.0654 g) was carefully added followed by sulfuric acid (1.33 mL). The contents of the flask were shaken under 50 psi hydrogen for 5 h. The solids were carefully removed by filtration through Celite and the filtrate was concentrated under reduced pressure. The oily residue was partitioned between equal parts ethyl acetate and water. The extracts were washed with water and brine and dried over sodium sulfate, filtered and concentrated to afford the crude product (10.2 g, 83%) as an oil which was used without further purification. LC-MS: (FA) ES− 211.

### Step 3: 6-fluoro-7-methoxy-3,4-dihydronaphthalen-1(2H)-one Intermediate 37

4-(3-Fluoro-4-methoxyphenyl)butanoic acid (Int-36, 1.114 g, 0.00525 mol) and methanesulfonic acid (2 mL, 0.0308 mol) were combined in trifluoroacetic acid (30 mL) and stirred at 100° C. for 2 h then cooled to room temperature. The mixture was then poured onto ice water and extracted with ethyl acetate. The extracts were washed with saturated aqueous sodium bicarbonate solution and brine, dried over magnesium sulfate, filtered and concentrated under reduced pressure to afford the crude product as a light brown oil. The crude residue was purified by silica gel chromatography (90/10 hexanes/ethyl acetate isocratic) to provide the product as an oil (0.779 g, 76%). LC-MS: (FA) ES+ 195; ¹H NMR (300 MHz, CDCl₃) δ ppm 7.58 (d, J=8.9 Hz, 1H), 6.91 (d, J=11.3 Hz, 1H), 3.87 (s, 3H), 2.85 (dd, J=6.1, 6.1 Hz, 2H), 2.59 (dd, J=7.1, 6.0 Hz, 2H), 2.09 (m, 2H).

### Step 4: Methyl 6-fluoro-7-methoxy-1-oxo-1,2,3,4-tetrahydronaphthalene-2-carboxylate Intermediate 38

To a stirred suspension of sodium hydride (60% in mineral oil, 1.37 g, 0.0343 mol) in tetrahydrofuran (100 mL) under an atmosphere of nitrogen was added dimethyl carbonate (2.81 mL, 0.0334 mol) dropwise followed by a solution of 6-fluoro-7-methoxy-3,4-dihydronaphthalen-1(2H)-one (Int-37, 3.24 g, 0.0167 mol) in tetrahydrofuran (40 mL). The resulting mixture was heated at reflux overnight. The reaction mixture was then cooled to room temperature and 30 mL acetic acid was added dropwise via an addition funnel. The resulting mixture was poured into water and was extracted with ether three times. The extracts were washed twice with 1N NaOH and then with brine, dried over sodium sulfate, filtered and concentrated. The crude residue was purified by silica gel chromatography (methylene chloride to 98/2 methylene chloride/methanol gradient) to afford the product as a white solid (2.14 g, 48%). LC-MS: (FA) ES+ 253.

### Step 5: Methyl 6-fluoro-7-methoxy-1,2,3,4-tetrahydronaphthalene-2-carboxylate Intermediate 39

A carefully combined mixture of methyl 6-fluoro-7-methoxy-1-oxo-1,2,3,4-tetrahydronaphthalene-2-carboxylate (202 mg, 0.0008 mol), sulfuric acid (0.06 mL) and palladium (10% on Carbon, 17 mg) in acetic acid (3.78 mL, 0.0665 mol) was shaken on the Parr hydrogenator at 30 psi. The solids were removed by careful filtration through Celite and the filtrate was concentrated under reduced pressure. The crude residue was partitioned between ethyl acetate and saturated aqueous sodium bicarbonate solution. The extracts were then washed with brine and dried over magnesium sulfate. The dried extracts were filtered and concentrated under reduced pressure. The product was used as obtained. (0.17 g, 89%). LC-MS: (FA) ES+ 239.

### Step 6: 6-fluoro-7-methoxy-1,2,3,4-tetrahydronaphthalene-2-carboxylic acid Intermediate 40

Methyl 6-fluoro-7-methoxy-1,2,3,4-tetrahydronaphthalene-2-carboxylate (Int-39, 170 mg, 0.00071 mol) was dissolved in methanol (3.9 mL). To the resulting solution was added aqueous sodium hydroxide (1.0M, 2.85 mL, 0.00285 mol) and the reaction mixture was stirred overnight at room temperature. The solvents were then concentrated under reduced pressure and the crude residue dissolved in water. The solution was acidified by the addition of 1M HCl and the resulting solids were isolated by suction filtration to afford the product (0.145 g, 91%) which was used as obtained.

### Step 7: 6-fluoro-7-hydroxy-1,2,3,4-tetrahydronaphthalene-2-carboxylic acid Intermediate 41

6-Fluoro-7-methoxy-1,2,3,4-tetrahydronaphthalene-2-carboxylic acid (Int-40, 2.98 g, 0.0106 mol) was added to hydrobromic acid (48% in water, 60 mL) and acetic acid (60 mL) and the resulting solution was heated at reflux for 5 h. The reaction mixture was concentrated under reduced pressure to afford the product as a brown solid which was used without further purification (2.18 g, 91%). LC-MS: (FA) ES− 209.

### Step 8: methyl 6-fluoro-7-hydroxy-1,2,3,4-tetrahydronaphthalene-2-carboxylate Intermediate 42

To a solution of 6-fluoro-7-hydroxy-1,2,3,4-tetrahydronaphthalene-2-carboxylic acid (Int-41, 0.775 g, 3.69 mmol) in methanol (12 mL) was added sulfuric acid (0.0196 mL, 0.369 mmol). The resulting solution was warmed to reflux and stirred overnight. After cooling to room temperature the methanol was removed under reduced pressure affording a dark brown oil. The oil was partitioned between half-saturated sodium bicarbonate solution (50 mL) and ethyl acetate (75 mL). The aqueous phase was extracted with additional ethyl acetate (50 mL). The extracts were combined, washed with saturated aqueous sodium bicarbonate solution and brine, dried over sodium sulfate, filtered and concentrated under reduced pressure. The crude product was purified by silica gel chromatography (90/10 to 70/30 hexanes/ethyl acetate gradient) to afford the titled compound (0.51 g, 62%). ¹H NMR (400 MHz, d₆-DMSO) δ ppm 9.48 (s, 1H), 6.81 (d, J=12.1 Hz, 1H), 6.64 (d, J=9.1 Hz, 1H), 3.62 (s, 3H), 2.85-2.59 (m, 5H), 2.10-1.96 (m, 1H), 1.72-1.59 (m,

### Step 9: methyl 7-({2-[(cyclopropylcarbonyl)amino]pyridin-4-yl}oxy)-6-fluoro-1,2,3,4-tetrahydronaphthalene-2-carboxylate Intermediate 43

To a 2-5 mL microwave vial fitted with a stirbar was added methyl 6-fluoro-7-hydroxy-1,2,3,4-tetrahydronaphthalene-2-carboxylate (Int-42, 254 mg, 1.13 mmol), N-(4-nitropyridin-2-yl)cyclopropanecarboxamide (Int-22, 213.4 mg, 1.03 mmol) and cesium carbonate (1010 mg, 3.09 mmol). N,N-Dimethylformamide (4.5 mL) was added, the vial was capped and the contents of the vial stirred thoroughly. The reaction mixture was heated in the microwave at 150° C. for 45 min. The vial was uncapped and the contents poured into water (20 mL) and extracted with ethyl acetate (50 mL×2). The extracts were combined, washed with water and brine then dried over sodium sulfate, filtered and concentrated under reduced pressure. The crude residue was purified by silica gel chromatography (70/30 to 30/70 hexanes/ethyl acetate gradient) to afford the product (267 mg, 67%). LC-MS: (AA) ES+ 385

### Step 10: 7-({2-[(cyclopropylcarbonyl)amino]pyridin-4-yl}oxy)-6-fluoro-N-hydroxy-1,2,3,4-tetrahydronaphthalene-2-carboxamide Compound I-32

The title compound was prepared following the procedure detailed in Example 1, Step 4. LC-MS: (AA) ES+ 386; ¹H NMR (400 MHz, d₆-DMSO) δ ppm 10.86 (s, 1H), 10.52 (s, 1H), 8.79 (s, 1H), 8.18 (d, J=5.7 Hz, 1H), 7.62 (d, J=2.0 Hz, 1H), 7.15 (d, J=11.5 Hz, 1H), 7.11 (d, J=8.3 Hz, 1H), 6.66 (dd, J=5.7, 2.3 Hz, 1H), 2.90-2.64 (m, 4H), 2.41-2.31 (m, 1H), 2.02-1.85 (m, 2H), 1.70 (ddd, J=17.1, 12.3, 5.7 Hz, 1H), 0.82-0.68 (m, 4H).

### Example 15

### 6-fluoro-N-hydroxy-7-[(7-oxo-5,6,7,8-tetrahydro-1,8-naphthyridin-4-yl)oxy]-1,2,3,4-tetrahydronaphthalene-2-carboxamide Compound I-31

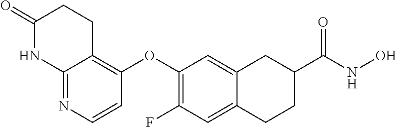

The title compound was prepared following procedures detailed in Example 14, substituting Intermediate 31 for Intermediate 22. LC-MS: (AA) ES+ 372; ¹H NMR (400 MHz, d₆-DMSO) δ ppm 10.51 (s, 1H), 7.95 (d, J=5.8 Hz, 1H), 7.15 (d, J=11.5 Hz, 1H), 7.07 (d, J=8.3 Hz, 1H), 6.23 (d, J=5.8 Hz, 1H), 2.98-2.91 (m, 2H), 2.89-2.65 (m, 4H), 2.57-2.51 (m, 2H), 2.40-2.31 (m, 1H), 1.92-1.84 (m, 1H), 1.69 (ddd, J=24.5, 12.0, 5.7 Hz, 1H).

### Example 16

### N-hydroxy-7-(pyridin-4-yloxy)-1,2,3,4-tetrahydronaphthalene-2-carboxamide Compound I-12

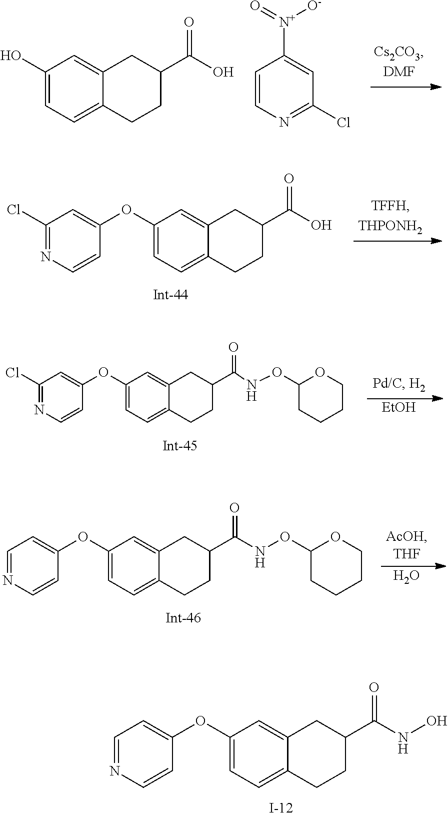

### Step 1: 7-[(2-chloropyridin-4-yl)oxy]-1,2,3,4-tetrahydronaphthalene-2-carboxylic acid Intermediate

2-Chloro-4-nitro-pyridine (289 mg, 0.00182 mol), 7-hydroxy-1,2,3,4-tetrahydronaphthalene-2-carboxylic acid (350 mg, 0.0018 mol), and cesium carbonate (1.78 g, 0.0055 mol) were combined in a single neck flask and suspended in N,N-dimethylformamide (6.5 mL). The reaction mixture was heated at 50° C. under an atmosphere of nitrogen overnight. After cooling to room temperature, the mixture was filtered and the filtrate was acidified to pH 1.5 by the addition of a 1N hydrochloric acid solution. The precipitate which formed on acidification was isolated by suction filtration and used without further purification (540 mg, 97%). LC-MS: (FA) ES+ 304.

### Step 2: 7-[(2-chloropyridin-4-yl)oxy]-N-(tetrahydro-2H-pyran-2-yloxy)-1,2,3,4-tetrahydronaphthalene-2-carboxamide Intermediate 45

The title compound was prepared from 7-[(2-chloropyridin-4-yl)oxy]-1,2,3,4-tetrahydronaphthalene-2-carboxylic acid (Int-44) following the procedure detailed in Example 3, Step 5. LC-MS: (FA) ES+ 403.

### Step 3: 7-(pyridin-4-yloxy)-N-(tetrahydro-2H-pyran-2-yloxy)-1,2,3,4-tetrahydronaphthalene-2-carboxamide Intermediate 46

7-[(2-chloropyridin-4-yl)oxy]-N-(tetrahydro-2H-pyran-2-yloxy)-1,2,3,4-tetrahydronaphthalene-2-carboxamide (Int-45, 50 mg, 0.1 mmol) was dissolved in ethanol (5 mL) in a single neck flask. The contents of the flask were carefully purged with argon and palladium (5% on carbon, 11 mg) was added. The reaction mixture was allowed to stir at room temperature under an atmosphere of hydrogen for 2 h. The flask was purged with argon, and the mixture carefully filtered through Celite. The filtrate was concentrated under reduced pressure and the crude product used without further purification.

### Step 4: N-hydroxy-7-(pyridin-4-yloxy)-1,2,3,4-tetrahydronaphthalene-2-carboxamide compound I-12

The title compound was prepared from Intermediate 46 following the procedure detailed in Example 3, Step 6. LC-MS: (FA) ES+ 285; NMR (400 MHz, d₆-DMSO) δ ppm 10.52 (s, 1H), 8.78 (s, 1H), 8.45-8.39 (m, 2H), 7.17 (d, J=8.2 Hz, 1H), 6.94-6.86 (m, 4H), 2.92-2.66 (m, 4H), 2.37 (dddd, J=11.2, 11.2, 5.2, 3.1 Hz, 1H), 1.95-1.85 (m, 1H), 1.72 (ddd, J=24.7, 11.9, 5.7 Hz, 1H)

### Example 17

### N-hydroxy-6-(pyridin-4-yloxy)-1,2,3,4-tetrahydronaphthalene-2-carboxamide Compound I-27

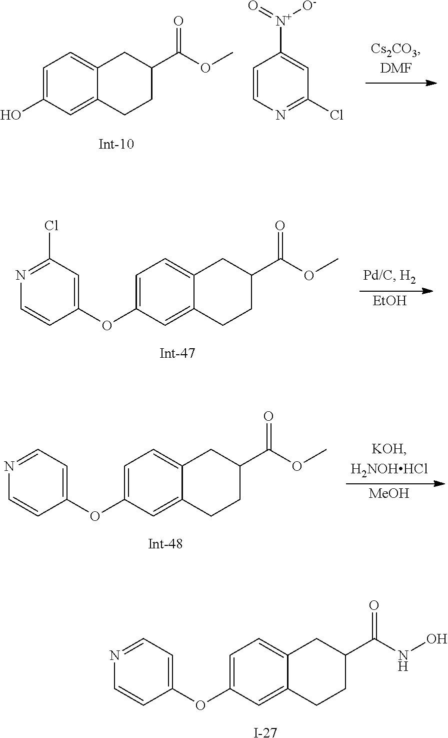

### Step 1: Methyl 6-[(2-chloropyridin-4-yl)oxy]-1,2,3,4-tetrahydronaphthalene-2-carboxylate Intermediate 47

2-Chloro-4-nitro-pyridine (76.9 mg, 0.00049 mol), methyl 6-hydroxy-1,2,3,4-tetrahydronaphthalene-2-carboxylate (100 mg, 0.0005 mol), cesium carbonate (474 mg, 0.00145 mol) and N,N-dimethylformamide (1.74 mL) were combined in a single neck flask. The reaction mixture was stirred at 50° C. under an atmosphere of nitrogen overnight. The reaction mixture was poured into ethyl acetate and washed with water. The aqueous phase was extracted with additional ethyl acetate. The extracts were combined, washed with water and brine and dried over sodium sulfate, then filtered and concentrated under reduced pressure. The crude product was used without further purification in the following step (150 mg, 97%). LC-MS: (FA) ES+ 318.

### Step 2: Methyl 6-(pyridin-4-yloxy)-1,2,3,4-tetrahydronaphthalene-2-carboxylate Intermediate 48

The title compound was prepared from Int-47 following the procedure detailed in Example 16, step 3. LC-MS: (FA) ES+ 284.

### Step 3: N-hydroxy-6-(pyridin-4-yloxy)-1,2,3,4-tetrahydronaphthalene-2-carboxamide Compound I-27

The title compound was prepared from Intermediate 48 following the procedure detailed in Example 1, Step 4. LC-MS: (FA) ES+ 285; ¹H NMR (400 MHz, d₆-DMSO) δ ppm 10.54 (s, 1H), 8.42 (m, 2H), 8.16 (s, 1H), 7.19 (d, J=9.1 Hz, 1H), 6.92-6.86 (m, 4H), 2.90-2.67 (m, 4H), 2.38 (dddd, J=11.1, 11.1, 5.3, 3.0 Hz, 1H), 1.93-1.84 (m, 1H), 1.70 (ddd, J=24.5, 11.7, 5.8 Hz, 1H).

### Example 18

### tert-Butyl [3-({7-[(hydroxyamino)carbonyl]-5,6,7,8-tetrahydronaphthalen-2-yl}oxy)phenyl]carbamate Compound I-53

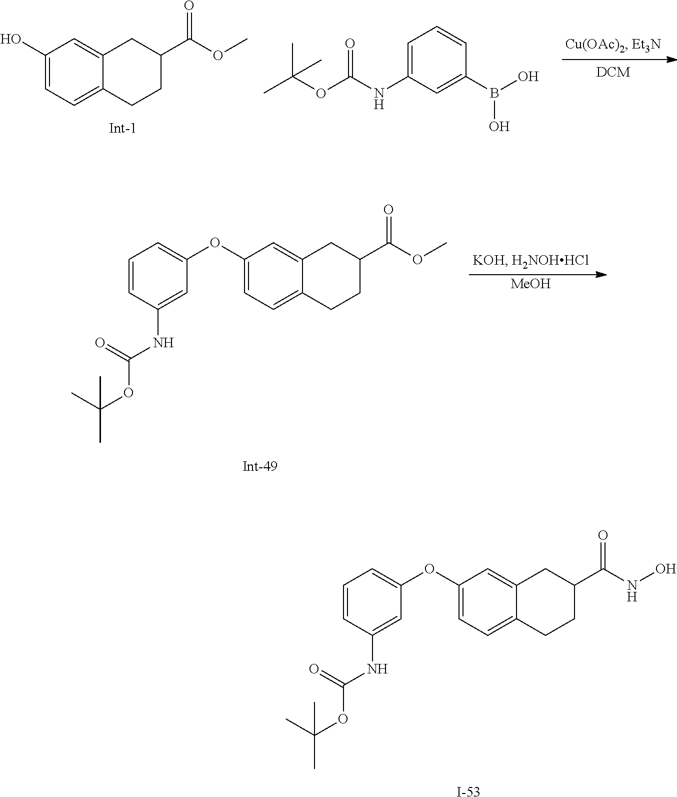

### Step 1: methyl 7-{3-[(tert-butoxycarbonyl)amino]phenoxy}-1,2,3,4-tetrahydronaphthalene-2-carboxylate Intermediate 49

To a mixture of methyl 7-hydroxy-1,2,3,4-tetrahydronaphthalene-2-carboxylate (Int-1, 250 mg, 1.2 mmol) in methylene chloride (10 mL) and triethylamine (1 mL, 9 mmol) were added 3-(N-Boc-amino)phenylboronic acid (664 mg, 2.8 mmol), copper(II) acetate (0.509 g, 2.8 mmol) and 4 Å molecular sieves (approx. 200 mg). The mixture was stirred overnight at room temperature. The reaction mixture was then filtered through Celite and the filtrate concentrated under reduced pressure. The crude residue was purified by silica gel chromatography (0-100% ethyl acetate in hexanes gradient) to provide the product as an impure oil (73 mg, 15%).

### Step 2: tert-Butyl [3-({7-[(hydroxyamino)carbonyl]-5,6,7,8-tetrahydronaphthalen-2-yl}oxy)phenyl]carbamate Compound I-53

The title compound was prepared from Intermediate 49 following the procedure detailed in Example 1, Step 4. LC-MS: (FA) ES+ 399; ¹H NMR (400 MHz, d₆-DMSO) δ ppm 10.51 (s, 1H), 9.40 (s, 1H), 8.78 (s, 1H), 7.25-7.12 (m, 3H), 7.07 (d, J=8.1 Hz, 1H), 6.77-6.70 (m, 2H), 6.55-6.51 (m, 1H), 2.88-2.62 (m, 4H), 2.35 (dddd, J=11.1, 11.1, 4.8, 2.9 Hz, 1H), 1.93-1.82 (m, 1H), 1.69 (ddd, J=24.4, 12.0, 5.6 Hz, 1H), 1.44 (s, 9H).

### Example 19

The following compounds were prepared in a fashion analogous to that described in Example 18 starting from the intermediates which were prepared as described above and the corresponding boronic acids.

| | LC-MS (FA) |
|---|---|
| Compound | ES+ |
| I-8 | 284 |
| I-21 | 341 |
| I-25 | 284 |
| I-19 | 318 |
| I-41 | 390 |
| I-47 | 326 |
| I-52 | 399 |
| I-9 | 318 |
| I-11 | 318 |
| I-43 | 367 |

### Example 20

### 7-{3-[(cyclopropylcarbonyl)amino]phenoxy}-N-hydroxy-1,2,3,4-tetrahydronaphthalene-2-carboxamide Compound I-50

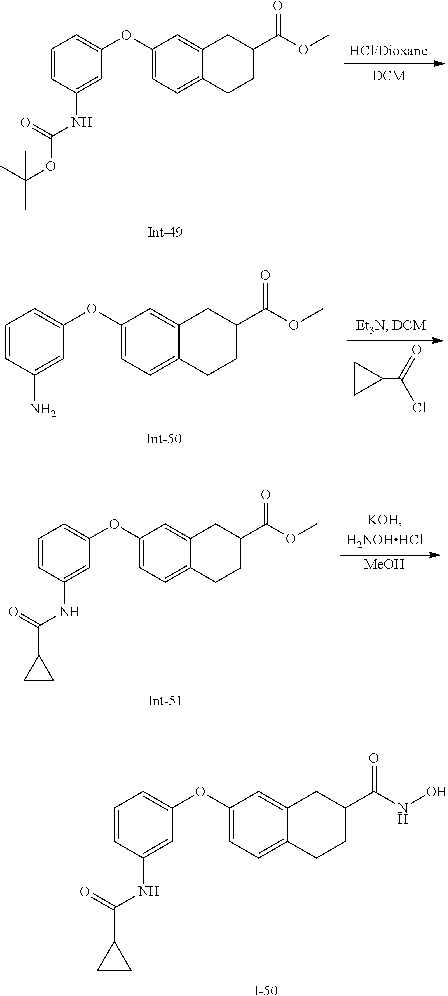

### Step 1: methyl 7-(3-aminophenoxy)-1,2,3,4-tetrahydronaphthalene-2-carboxylate.HCl Intermediate 50

Methyl 7-{3-[(tert-butoxycarbonyl)amino]phenoxy}-1,2,3,4-tetrahydronaphthalene-2-carboxylate (Int-49, 70 mg, 0.2 mmol) was dissolved in methylene chloride (5 mL). A solution of hydrogen chloride in 1,4-dioxane (4M, 0.4 mL) was added and the mixture was stirred at room temperature for 4 h. The reaction mixture was concentrated to dryness under reduced pressure then further dried in vacuo. The crude product was used without further purification in the following step. LC-MS: (FA) ES+ 298.

### Step 2: methyl 7-{3-[(cyclopropylcarbonyl)amino]phenoxy}-1,2,3,4-tetrahydronaphthalene-2-carboxylate Intermediate 51

Methyl 7-(3-aminophenoxy)-1,2,3,4-tetrahydronaphthalene-2-carboxylate.HCl (Int-50, 59 mg, 0.18 mmol) was suspended in methylene chloride (5 mL). Triethylamine (0.06 mL, 0.442 mmol) was added followed by cyclopropanecarbonyl chloride (22.2 mg, 0.212 mmol). The mixture was allowed to stir for 3 h. Upon completion of the reaction, the mixture was concentrated under reduced pressure and the crude product was purified by silica gel chromatography (80/20 to 40/60 hexanes/ethyl acetate gradient) to afford the product contaminated with an impurity (43 mg, 67%). LC-MS: (FA) ES+ 366.

### Step 3: 7-{3-[(cyclopropylcarbonyl)amino]phenoxy}-N-hydroxy-1,2,3,4-tetrahydronaphthalene-2-carboxamide Compound I-50

The title compound was prepared from Intermediate 51 following the procedure detailed in Example 1, Step 4. LC-MS: (FA) ES+ 367; ¹H NMR (400 MHz, d₆-DMSO) δ ppm 10.53 (s, 1H), 10.25 (s, 1H), 8.82 (s, 1H), 7.37-7.19 (m, 3H), 7.08 (d, J=8.1 Hz, 1H), 6.80-6.73 (m, 2H), 6.62 (ddd, J=7.9, 2.4, 1.1 Hz, 1H), 2.88-2.63 (m, 4H), 2.35 (dddd, J=11.4, 11.4, 5.1, 3.2 Hz, 1H), 1.93-1.84 (m, 1H), 1.78-1.62 (m, 2H), 0.79-0.70 (m, 4H).

### Example 21

The compounds below were prepared in an analogous manner to that described for Example 20, substituting appropriate starting materials and acid chlorides.

| | LC-MS (FA) |
|---|---|
| Compound | ES+ |
| I-55 | 377 |
| I-56 | 367 |

### Example 22

### N-hydroxy-7-(3-{[(methylamino)carbonyl]amino}phenoxy)-1,2,3,4-tetrahydronaphthalene-2-carboxamide Compound I-57

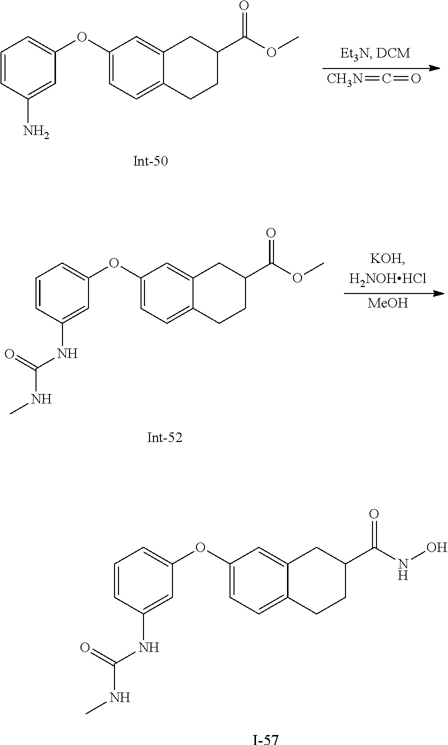

The title compound was prepared following the procedures detailed in Example 20, Steps 2 and 3, substituting methyl isocyanate for cyclopropanecarbonyl chloride. LC-MS: (FA) ES+ 356. ¹H NMR (400 MHz, d₆-DMSO) δ ppm 8.89 (s, 1H), 8.83-8.71 (m, 1H), 7.19-7.11 (m, 2H), 7.09-7.02 (m, 2H), 6.80-6.71 (m, 2H), 6.46 (dd, J=7.7, 2.0 Hz, 1H), 6.28 (dd, J=8.8, 4.3 Hz, 1H), 2.88-2.64 (m, 4H), 2.58 (d, J=4.6 Hz, 3H), 2.36 (m, 1H), 1.93-1.82 (m, 1H), 1.70 (ddd, J=24.2, 12.0, 5.6 Hz, 1H).

### Example 23

### 7-[3-(benzylamino)phenoxy]-N-hydroxy-1,2,3,4-tetrahydronaphthalene-2-carboxamide Compound I-59

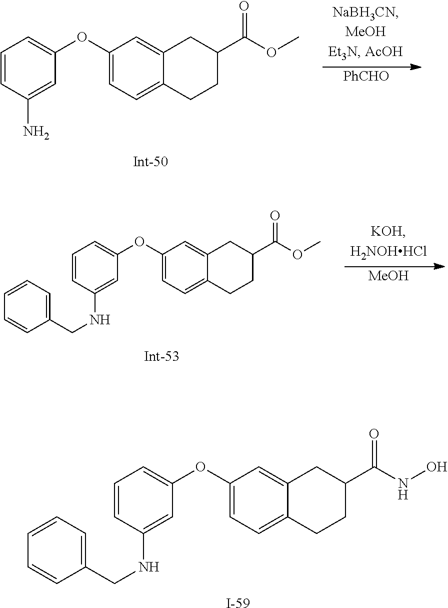

### Step 1: methyl 7-[3-(benzylamino)phenoxy]-1,2,3,4-tetrahydronaphthalene-2-carboxylate Intermediate 53

Methyl 7-(3-aminophenoxy)-1,2,3,4-tetrahydronaphthalene-2-carboxylate.HCl Int-50 (70 mg, 0.2 mmol) was dissolved in methanol (1.7 mL) to which subsequently were added triethylamine (0.029 mL, 0.21 mmol) and benzaldehyde (0.032 mL, 0.314 mmol). The reaction mixture was stirred at room temperature for 1 h before sodium cyanoborohydride (19.8 mg, 0.314 mmol) and acetic acid (0.063 g, 1.05 mmol) were added. Sufficient acetic acid was added to maintain pH<7. After stirring overnight, excess reducing agent was quenched by the addition of water (0.1 mL). The mixture was extracted with methylene chloride. The extracts were washed with brine, dried over sodium sulfate, filtered and concentrated under reduced pressure. The crude product was purified by silica gel chromatography (80/20 to 60/40 hexanes/ethyl acetate gradient) to afford the product as an oil (37 mg, 40%). LC-MS: (FA) ES+ 388. ¹H NMR (400 MHz, CDCl₃) δ ppm 7.40-7.26 (m, 5H), 7.10 (dd, J=8.1, 8.1 Hz, 1H), 7.03 (d, J=8.3 Hz, 1H), 6.82-6.76 (m, 2H), 6.40-6.32 (m, 2H), 6.29 (dd, J=2.2, 2.2 Hz, 1H), 4.29 (s, 2H), 3.74 (s, 3H), 3.00-2.94 (m, 2H), 2.92-2.69 (m, 3H), 2.26-2.17 (m, 1H), 1.87 (dddd, J=13.0, 10.9, 10.9, 6.1 Hz, 1H).

### Step 2: 7-[3-(benzylamino)phenoxy]-N-hydroxy-1,2,3,4-tetrahydronaphthalene-2-carboxamide Compound I-59

The title compound was prepared from Intermediate 53 following the procedure detailed in Example 1, Step 4. LC-MS: (FA) ES+ 389; ¹H NMR (400 MHz, d₆-DMSO) δ ppm 10.52 (s, 1H), 8.78 (s, 1H), 7.45 (dd, J=8.2, 8.2 Hz, 1H), 7.35 (d, J=8.3 Hz, 1H), 7.32-7.20 (m, 4H), 7.10 (d, J=8.0 Hz, 1H), 7.05-7.00 (m, 2H), 6.92 (dd, J=8.1, 1.3 Hz, 1H), 6.83-6.76 (m, 2H), 5.30 (s, 2H), 2.89-2.64 (m, 4H), 2.41-2.31 (m, 1H), 1.95-1.83 (m, 1H), 1.70 (dddd, J=12.1, 12.1, 12.0, 5.5 Hz, 1H).

### Example 24

### 7-{4-[(4-chlorobenzyl)amino]phenoxy}-N-hydroxy-1,2,3,4-tetrahydronaphthalene-2-carboxamide Compound I-73

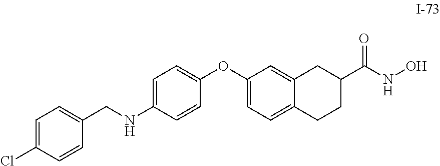

The title compound was prepared following the procedures detailed in Example 23 using the appropriate intermediates and reagents. LC-MS: (FA) ES+ 423; ¹H NMR (300 MHz, d₆-DMSO) δ ppm 10.48 (d, J=0.4 Hz, 1H), 7.42-7.30 (m, 4H), 7.00-6.94 (m, 1H), 6.78-6.71 (m, 2H), 6.61-6.52 (m, 4H), 6.19 (dd, J=5.7, 5.7 Hz, 1H), 4.22 (d, J=5.7 Hz, 2H), 2.86-2.56 (m, 4H), 2.40-2.25 (m, 1H), 1.92-1.81 (m, 1H), 1.66 (dddd, J=11.2, 10.9, 10.9, 5.4 Hz, 1H).

### Example 25

### tert-butyl {7-[(hydroxyamino)carbonyl]-5,6,7,8-tetrahydronaphthalen-2-yl}carbamate Compound I-20

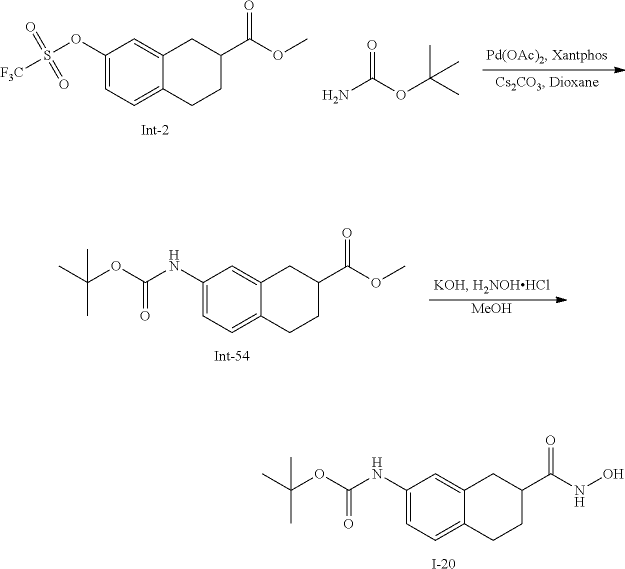

### Step 1: Methyl 7-(tert-butoxycarbonylamino)-1,2,3,4-tetrahydronaphthalene-2-carboxylate Intermediate 54

Palladium(II) acetate (9 mg, 0.04 mmol) and 4,5-bis(diphenylphosphino)-9,9-dimethylxanthene (Xantphos, 50 mg, 0.08 mmol) were combined in a nitrogen-purged flask. Methyl 7-{[(trifluoromethyl)sulfonyl]oxy}-1,2,3,4-tetrahydronaphthalene-2-carboxylate (Int-2, 100 mg, 0.4 mmol), tert-butyl carbamate (0.073 g, 0.62 mmol) and cesium carbonate (0.2 g, 0.8 mmol) were added to the reaction flask, followed by 1,4-dioxane (1 mL). The reaction mixture was stirred under dry nitrogen at 100° C. for 2 h. The reaction mixture was cooled to room temperature and solids were removed by filtration. The filtrate was concentrated under reduced pressure and the crude residue was purified by silica gel chromatography (hexanes to 70/30 hexanes/ethyl acetate) to afford the product as a solid (25 mg, 20%). LC-MS: (FA) ES+ 306.

### Step 2: tert-butyl {7-[(hydroxyamino)carbonyl]-5,6,7,8-tetrahydronaphthalen-2-yl}carbamate Compound I-20

The title compound was prepared from Intermediate 54 following the procedure detailed in Example 1, Step 4. LC-MS: (FA) ES+ 307.

### Example 26

### 7-[(cyclopropylcarbonyl)amino]-N-hydroxy-1,2,3,4-tetrahydronaphthalene-2-carboxamide Compound I-22

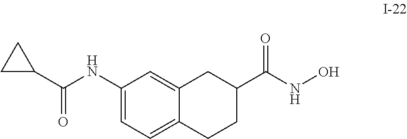

The title compound was prepared following the procedures detailed in Example 25, substituting cyclopropane carboxamide for tert-butyl carbamate. LC-MS: (FA) ES+ 275; ¹H NMR (400 MHz, d₆-DMSO) δ ppm 10.48 (s, 1H), 10.02 (s, 1H), 8.76 (s, 1H), 7.31 (s, 1H), 7.27 (dd, J=8.0, 1.8 Hz, 1H), 6.96 (d, J=8.2 Hz, 1H), 2.88-2.58 (m, 4H), 2.34 (dddd, J=11.4, 8.0, 4.9, 3.1 Hz, 1H), 1.91-1.82 (m, 1H), 1.78-1.60 (m, 2H), 0.81-0.68 (m, 4H).

### Example 27

### 7-[(3-chlorophenyl)amino]-N-hydroxy-1,2,3,4-tetrahydronaphthalene-2-carboxamide Compound I-26

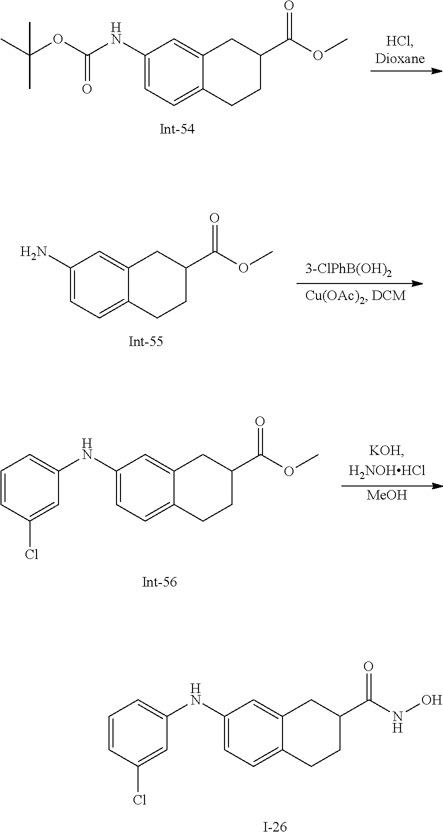

### Step 1: methyl 7-amino-1,2,3,4-tetrahydronaphthalene-2-carboxylate Intermediate 55

Methyl 7-[(tert-butoxycarbonyl)amino]-1,2,3,4-tetrahydronaphthalene-2-carboxylate (Int-54, 740 mg, 2.4 mmol) was dissolved in methylene chloride (8 mL) and a hydrogen chloride solution in 1,4-dioxane (4.0 M, 6.1 mL, 24.4 mmol) was added. After stirring at room temperature for 1 h, the reaction was complete. Solvents were removed under reduced pressure the resulting product was isolated as its HCl salt and used as obtained. LC-MS: (FA) ES+ 206.

### Step 2: methyl 7-(3-chlorophenylamino)-1,2,3,4-tetrahydronaphthalene-2-carboxylate Intermediate

To a mixture of methyl 7-amino-1,2,3,4-tetrahydronaphthalene-2-carboxylate.HCl (Int-55, 100 mg, 0.4 mmol) in methylene chloride (4 mL) and triethylamine (0.501 mL, 3.59 mmol) were added 3-chlorophenylboronic acid (0.13 g, 0.831 mmol), copper(II) acetate (151 mg, 0.831 mmol) and 4 Å molecular sieves (approx. 250 mg). The resulting green mixture was stirred at room temperature overnight. The solids were removed by filtration through Celite and the filtrate was washed with ammonium hydroxide solution then brine. The extracts were dried over sodium sulfate, filtered and concentrated under reduced pressure. The crude residue was purified by silica gel chromatography (hexanes to 70/30 hexanes/ethyl acetate) to afford the product (120 mg, 21%). LC-MS: (FA) ES+ 316.

### Step 3: 7-[(cyclopropylcarbonyl)amino]-N-hydroxy-1,2,3,4-tetrahydronaphthalene-2-carboxamide I-22

The title compound was prepared from Intermediate 56 following the procedure detailed in Example 1, Step 4. LC-MS: (FA) ES+ 317; ¹H NMR (400 MHz, d₆-DMSO) δ ppm 10.51 (s, 1H), 8.77 (s, 1H), 8.20 (s, 1H), 7.17 (dd, J=8.1, 8.1 Hz, 1H), 6.98 (d, J=8.2 Hz, 1H), 6.94-6.89 (m, 2H), 6.86 (dd, J=8.1, 2.2 Hz, 1H), 6.81 (d, J=1.9 Hz, 1H), 6.75-6.71 (m, 1H), 2.89-2.60 (m, 4H), 2.41-2.30 (m, 1H), 1.92-1.82 (m, 1H), 1.68 (ddd, J=24.3, 11.9, 5.7 Hz, 1H).

### Example 28

### 7-anilino-N-hydroxy-1,2,3,4-tetrahydronaphthalene-2-carboxamide Compound I-23

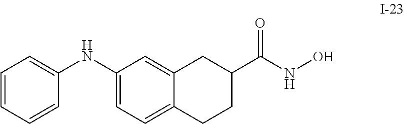

The title compound was prepared following the procedures detailed in Example 27, substituting phenylboronic acid for 3-chlorophenylboronic acid. LC-MS: (FA) ES+ 283; ¹H NMR (300 MHz, d₆-DMSO) δ ppm 10.49 (s, 1H), 8.78 (s, 1H), 7.94 (s, 1H), 7.24-7.12 (m, 2H), 7.02-6.96 (m, 2H), 6.93 (d, J=8.2 Hz, 1H), 6.85-6.70 (m, 3H), 2.88-2.58 (m, 4H), 2.42-2.26 (m, 1H), 1.92-1.80 (m, 1H), 1.67 (ddd, J=24.1, 11.7, 6.0 Hz, 1H).

### Example 29

### N-hydroxy-7-[(phenylsulfonyl)amino]-1,2,3,4-tetrahydronaphthalene-2-carboxamide Compound I-28

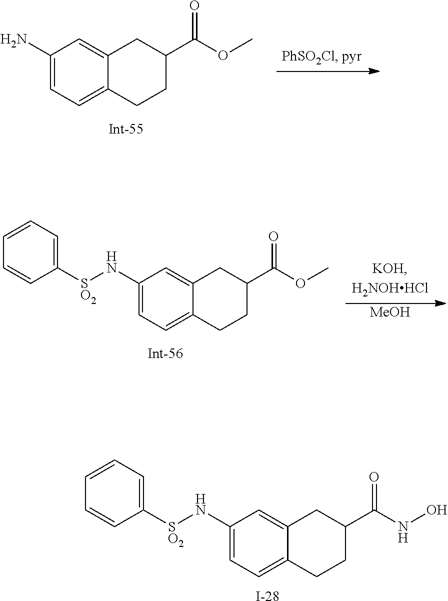

### Step 1: methyl 7-(phenylsulfonamido)-1,2,3,4-tetrahydronaphthalene-2-carboxylate Intermediate

To a mixture of methyl 7-amino-1,2,3,4-tetrahydronaphthalene-2-carboxylate.HCl (Int-55, 100 mg, 0.4 mmol) in methylene chloride (3 mL) and pyridine (0.087 mL, 1.08 mmol) was added benzenesulfonyl chloride (0.055 mL, 0.43 mmol). The reaction mixture was stirred overnight at room temperature. The mixture was concentrated to dryness under reduced pressure. The crude residue was purified by silica gel chromatography (hexanes to 50/50 hexanes/ethyl acetate) to afford the sulfonamide Intermediate 57. LC-MS: (FA) ES+ 346; ¹H NMR (300 MHz, CDCl₃) δ ppm 7.81-7.72 (m, 2H), 7.46-7.39 (m, 2H), 6.92 (d, J=8.2 Hz, 1H), 6.83 (dd, J=8.1, 2.3 Hz, 1H), 6.79 (d, J=1.9 Hz, 1H), 3.70 (s, 3H), 2.91-2.85 (m, 2H), 2.83-2.60 (m, 3H), 2.15 (ddd, J=12.6, 8.5, 4.0 Hz, 1H), 1.79 (dddd, J=13.1, 10.9, 10.3, 6.5 Hz, 1H).

### Step 2: N-hydroxy-7-[(phenylsulfonyl)amino]-1,2,3,4-tetrahydronaphthalene-2-carboxamide Compound I-28

The title compound was prepared from Intermediate 57 following the procedure detailed in Example 1, Step 4. LC-MS: (FA) ES+ 347; ¹H NMR (400 MHz, d₆-DMSO) δ ppm 7.76-7.70 (m, 2H), 7.62-7.50 (m, 3H), 6.89 (d, J=8.2 Hz, 1H), 6.84-6.74 (m, 2H), 2.78-2.54 (m, 4H), 2.34-2.22 (m, 1H), 1.81 (d, J=13.7 Hz, 1H), 1.60 (ddd, J=24.3, 11.8, 5.8 Hz, 1H).

### Example 30

### N-hydroxy-7-{[3-(1-methyl-1H-pyrazol-4-yl)-5-(trifluoromethyl)benzoyl]amino}-1,2,3,4-tetrahydronaphthalene-2-carboxamide Compound I-35

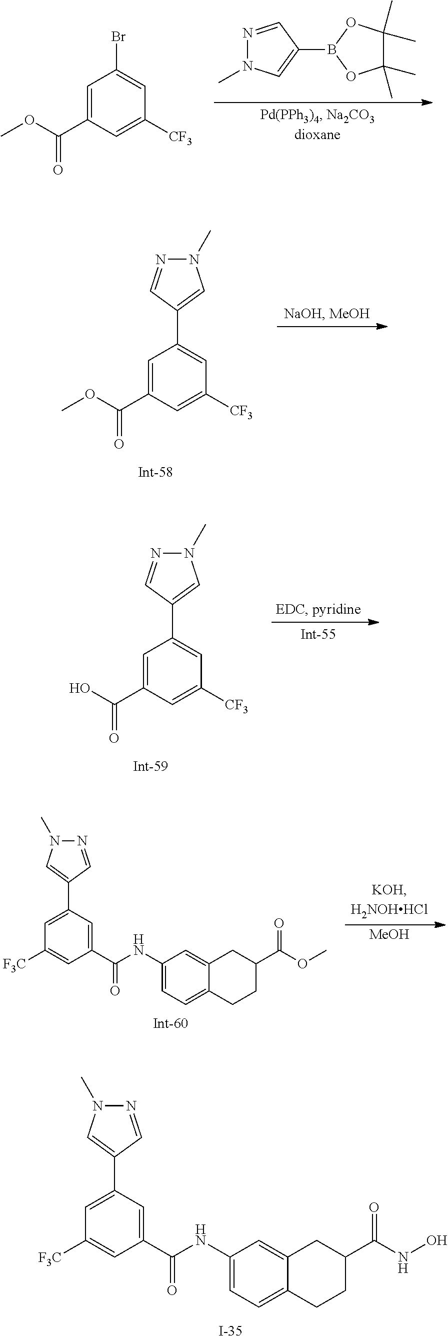

### Step 1: methyl 3-(1-methyl-1H-pyrazol-4-yl)-5-(trifluoromethyl)benzoate Intermediate-58

To a solution of methyl 3-bromo-5-(trifluoromethyl)benzoate (21.8 g, 77 mmol) in 1,4-dioxane (218 mL), and water (131 mL) were added 1-methyl-4-(4,4,5,5-tetramethyl-1,3,2-dioxaborolan-2-yl)-1H-pyrazole (24 g, 116 mmol), sodium carbonate (27.7 g, 261 mmol) and tetrakis(triphenyphosphine)palladium(0) (4.4 g, 3.8 mmol). The reaction mixture was heated at 80° C. for 3 h. The reaction mixture was cooled to room temperature and precipitated solids were removed by filtration. The filtrate was diluted with water and extracted twice with ethyl acetate. The extracts were washed with brine, dried over sodium sulfate, filtered and concentrated. The residue was purified by filtration through silica, eluting with 0% to 40% ethyl acetate in hexanes to provide methyl 3-(1-methyl-1H-pyrazol-4-yl)-5-(trifluoromethyl)benzoate as a pale yellow solid (22.3 g, 100%). LC-MS (FA): ES+ 285; ¹H NMR (400 MHz, CDCl₃) δ ppm 8.29 (s, 1H), 8.14 (s, 1H), 7.87 (s, 1H), 7.86 (s, 1H), 7.76 (s, 1H), 4.01 (s, 3H), 3.98 (s, 3H).

### Step 2: 3-(1-methyl-1H-pyrazol-4-yl)-5-(trifluoromethyl)benzoic acid Intermediate-59

To a solution of methyl 3-(1-methyl-1H-pyrazol-4-yl)-5-(trifluoromethyl)benzoate (Int-58, 22.3 g, 78.5 mmol) in methanol (375 mL), was added 1 N NaOH solution (314 mL, 314 mmol). The reaction was stirred at room temperature for 2 h. The methanol was removed by concentration under reduced pressure and the resulting aqueous mixture was acidified to pH 2 with 1 N HCl. The product was isolated by suction filtration, washed with water and hexane and dried under vacuum to provide 3-(1-methyl-1H-pyrazol-4-yl)-5-(trifluoromethyl)benzoic acid as a white solid (20.3 g, 95.7%). LC-MS: (FA) ES+ 271, ES− 269; ¹H NMR (400 MHz, d₆-DMSO) δ ppm 13.53 (s, 1H), 8.44 (s, 1H), 8.33 (s, 1H), 8.16 (s, 1H), 8.09 (d, J=0.7 Hz, 1H), 7.95 (s, 1H), 3.87 (s, 3H).

### Step 3: methyl 7-{[3-(1-methyl-1H-pyrazol-4-yl)-5-(trifluoromethyl)benzoyl]amino}-1,2,3,4-tetrahydronaphthalene-2-carboxylate Intermediate 60

3-(1-methyl-1H-pyrazol-4-yl)-5-(trifluoromethyl)benzoic acid (Int-59, 0.112 g, 0.414 mmol) was dissolved in pyridine (2 mL). To this solution was added N-(3-dimethylaminopropyl)-N′-ethylcarbodiimide hydrochloride (0.238 g, 1.24 mmol). The reaction mixture was stirred for 15 min at room temperature and then methyl 7-amino-1,2,3,4-tetrahydronaphthalene-2-carboxylate.HCl (Int-55, 100 mg, 0.414 mmol) was added and the resulting reaction mixture was stirred at room temperature overnight. The reaction mixture was concentrated to dryness under reduced pressure and the crude residue was purified by silica gel chromatography (80/20 to 30/70 hexanes/ethyl acetate gradient) to afford pure product (110 mg, 58%). LC-MS: (FA) ES+ 458.

### Step 4: N-hydroxy-7-{[3-(1-methyl-4H-pyrazol-4-yl)-5-(trifluoromethyl)benzoyl]amino}-1,2,3,4-tetrahydronaphthalene-2-carboxamide I-35

The title compound was prepared from Intermediate 60 following the procedure detailed in Example 1, Step 4. LC-MS: (FA) ES+ 459. ¹H NMR (400 MHz, d₆-DMSO) δ ppm 10.36 (s, 1H), 8.42 (s, 1H), 8.40 (s, 1H), 8.11 (s, 2H), 8.04 (s, 1H), 7.52 (dd, J=8.2, 1.7 Hz, 1H), 7.49-7.46 (m, 1H), 7.07 (d, J=8.3 Hz, 1H), 3.89 (s, 3H), 2.93-2.64 (m, 4H), 2.45-2.33 (m, 1H), 1.90 (d, J=11.0 Hz, 1H), 1.78-1.66 (m, 1H).

### Example 31

### 7-{[3-[(dimethylamino)methyl]-5-(trifluoromethyl)benzoyl]amino}-N-hydroxy-1,2,3,4-tetrahydronaphthalene-2-carboxamide Compound I-37

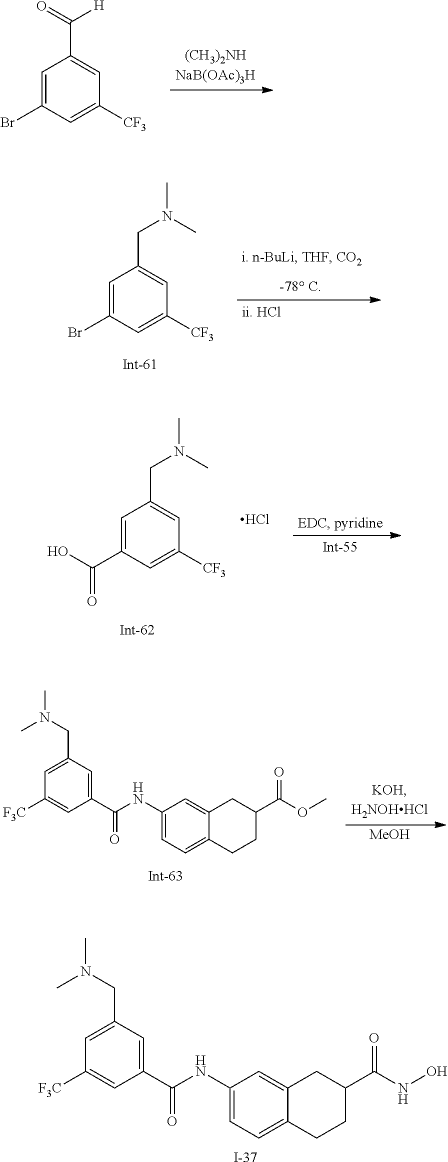

### Step 1: 1-(3-bromo-5-(trifluoromethyl)phenyl)-N,N-dimethylmethanamine Intermediate 61

To a solution of 3-bromo-5-(trifluoromethyl)benzaldehyde (30 g, 118.6 mmol) in methylene chloride (150 mL) was added dimethylamine (2.0 M in THF, 118 mL) and the reaction was stirred at room temperature for 15 min. The reaction was cooled to 0° C. and sodium triacetoxyborohydride (37.7 g, 178 mmol) was added. The resulting mixture was warmed to room temperature and stirred for 3 h. The solvents were removed under reduced pressure and saturated sodium bicarbonate solution was added. The resulting mixture was extracted three times with ethyl acetate. The combined extracts were washed with brine, dried over sodium sulfate, filtered and concentrated under reduced pressure. Silica gel chromatography (90/10 to 60/40 ethyl acetate/hexanes gradient) provided 1-[3-bromo-5-(trifluoromethyl)phenyl]-N,N-dimethylmethanamine as a colorless oil (24.9 g, 74% yield). LC-MS: (FA) ES+ 282; ¹H NMR (300 MHz, CDCl₃) δ ppm 7.68 (s, 1H), 7.65 (s, 1H), 7.52 (s, 1H), 3.44 (s, 2H), 2.25 (s, 6H).

### Step 2: 3-((dimethylamino)methyl)-5-(trifluoromethyl)benzoate.HCl Intermediate 62

To a solution of 1-[3-bromo-5-(trifluoromethyl)phenyl]-N,N-dimethylmethanamine (Int-61, 2 g, 7.1 mmol) in THF (40 mL) at −78° C. was dropwise added a solution of n-butyllithium (2.5 M in hexane, 3.12 mL, 7.81 mmol). The resulting mixture was stirred at −78° C. for 20 min. Excess crushed solid CO₂ was added and the mixture was stirred at −78° C. for another 15 min. The reaction was quenched by the addition of water (0.156 mL) and allowed to warm to room temperature. The solvents were evaporated and the solid was dried overnight under vacuum to give 3-[(dimethylamino)methyl]-5-(trifluoromethyl)benzoic acid.Li salt as a white solid contaminated with valeric acid. The crude acid was dissolved in aqueous hydrochloric acid (1M in water, 5 eq) and water (20 vols). Dissolution was not complete. The solids were removed by suction filtration, washed with methylene chloride and set aside. The resulting aqueous solution was transferred to a separatory funnel and washed with methylene chloride (3×). The washed aqueous phase was transferred to a round bottom flask. The filtered solids were added to the aqueous phase. The mixture was concentrated to dryness under reduced pressure. On heating in the water bath, the mixture turned homogeneous. On concentration, the solution afforded a gummy solid, which was azeotropically dried in toluene until it was a free-flowing solid. The resulting powder was suspended in ether and filtered. The filter cake was briefly dried under suction. The product was transferred to a round bottom flask and dried under high vacuum at 40° C. overnight. LC-MS: (AA) ES+ 248; ¹H NMR (400 MHz, CD₃OD) δ ppm 8.46 (s, 1H), 8.39 (s, 1H), 8.14 (s, 1H), 4.51 (s, 2H), 2.89 (s, 6H).

### Steps 3 and 4

The title compound was prepared from Intermediate 55 and Intermediate 62 following the procedures detailed in Example 30, Steps 3 and 4. LC-MS: (FA) ES+ 436; ¹H NMR (400 MHz, d₆-DMSO) δ ppm 10.53 (s, 1H), 10.34 (s, 1H), 8.78 (s, 1H), 8.20-8.15 (m, 2H), 7.83 (s, 1H), 7.51 (dd, J=8.3, 1.7 Hz, 1H), 7.46 (s, 1H), 7.06 (d, J=8.3 Hz, 1H), 3.59-3.52 (m, 2H), 2.92-2.64 (m, 4H), 2.44-2.33 (m, 1H), 2.18 (s, 6H), 1.94-1.85 (m, 1H), 1.71 (ddd, J=24.3, 12.0, 5.7 Hz, 1H).

### Example 32

### N-hydroxy-7-[(4-methoxybenzoyl)amino]-1,2,3,4-tetrahydronaphthalene-2-carboxamide Compound I-29

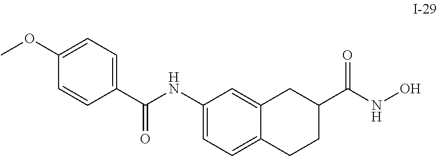

The title compound was prepared from Intermediate 55 and 4-methoxybenzoic acid following procedures detailed in Example 30, Steps 3 and 4. LC-MS: (FA) ES+ 341; ¹H NMR (400 MHz, d₆-DMSO) δ ppm 10.52 (d, J=1.5 Hz, 1H), 9.94 (s, 1H), 8.77 (d, J=1.7 Hz, 1H), 7.96-7.91 (m, 2H), 7.49-7.45 (m, 2H), 7.07-6.99 (m, 3H), 3.84-3.81 (m, 3H), 2.91-2.62 (m, 4H), 2.37 (dddd, J=11.4, 11.4, 4.9, 3.1 Hz, 1H), 1.93-1.84 (m, 1H), 1.70 (dddd, J=11.9, 11.9, 11.9, 5.8 Hz, 1H).

### Example 33

### N-{7-[(hydroxyamino)carbonyl]-5,6,7,8-tetrahydronaphthalen-2-yl}-4-methylpiperidine-4-carboxamide Compound I-18

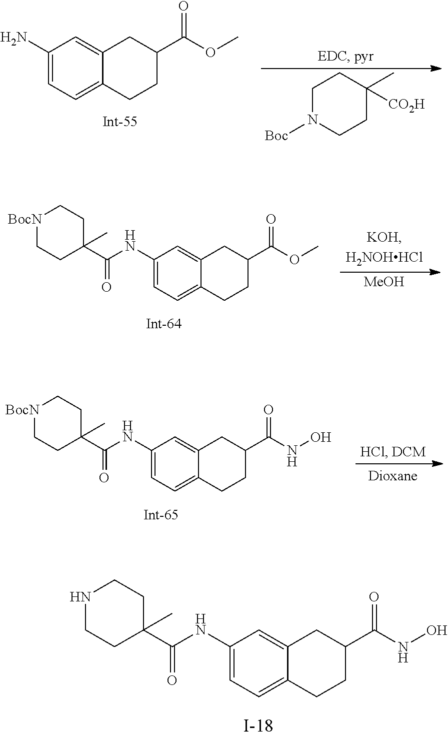

### Step 1: tert-butyl 4-({[7-(methoxycarbonyl)-5,6,7,8-tetrahydronaphthalen-2-yl]amino}carbonyl)-4-methylpiperidine-1-carboxylate Intermediate 64

Methyl 7-amino-1,2,3,4-tetrahydronaphthalene-2-carboxylate.HCl (Int-55, 180 mg, 0.74 mmol), and 4-methyl-4-carboxy-1-N-butoxycarbonyl-piperidine (181 mg, 0.745 mmol) were dissolved in pyridine (6 mL). To the resulting solution and was added N-(3-dimethylaminopropyl)-N′-ethylcarbodiimide hydrochloride (428 mg, 2.23 mmol). The reaction was allowed to stir at room temperature 72 h. The reaction mixture was concentrated under reduced pressure and the crude residue was purified by silica gel chromatography (hexanes to 50/50 hexanes/ethyl acetate gradient) to afford the product as a yellow solid.

### Step 2: tert-butyl 4-[({7-[(hydroxyamino)carbonyl]-5,6,7,8-tetrahydronaphthalen-2-yl}amino)carbonyl]-4-methylpiperidine-1-carboxylate Intermediate 65

The title compound was prepared from Intermediate 64 following the procedure detailed in Example 1, Step 4. The crude product was purified by chromatography on diol-functionalized silica (hexanes to 80/20 hexanes/ethyl acetate) to afford the product as a solid (112 mg, 60%). LC-MS: (FA) ES+ 432; ¹H NMR (400 MHz, CD₃OD) δ ppm 7.25-7.18 (m, 2H), 7.01 (d, J=8.8 Hz, 1H), 3.74-3.63 (m, 2H), 3.27-3.08 (m, 2H), 2.96 (dd, J=16.2, 11.7 Hz, 1H), 2.89-2.70 (m, 3H), 2.44 (dddd, J=11.6, 11.6, 4.9, 3.0 Hz, 1H), 2.18-2.08 (m, 2H), 1.99-1.94 (m, 1H), 1.83 (dddd, J=12.1, 12.1, 12.0, 5.9 Hz, 1H), 1.50-1.39 (m, 11H), 1.29 (s, 3H).

### Step 3: N-{7-[(hydroxyamino)carbonyl]-5,6,7,8-tetrahydronaphthalen-2-yl}-4-methylpiperidine-4-carboxamide Compound I-18

To a solution of tert-butyl 4-[({7-[(hydroxyamino)carbonyl]-5,6,7,8-tetrahydronaphthalen-2-yl}amino)carbonyl]-4-methylpiperidine-1-carboxylate (Int-65, 115 mg, 0.266 mmol) in methylene chloride (3.6 mL) was added a solution of hydrogen chloride in 1,4-dioxane (4.0 M, 3.63 mL, 14.5 mmol). Upon addition of acid, a white precipitate formed. The reaction mixture was stirred at room temperature for 2 h. The product was collected as a hygroscopic white solid by filtration. The solids were dissolved in water and freeze dried to afford the product as a fluffy solid (80 mg, 80%). LC-MS: (FA) ES+ 332; ¹H NMR (400 MHz, CD₃OD δ ppm 7.29-7.22 (m, 2H), 7.04 (d, J=8.2 Hz, 1H), 3.68-3.63 (m, 1H), 3.36-3.32 (m, 1H), 3.17-3.06 (m, 2H), 3.04-2.93 (m, 1H), 2.90-2.72 (m, 3H), 2.52-2.34 (m, 3H), 2.05-1.96 (m, 1H), 1.84 (ddd, J=24.6, 11.8, 5.9 Hz, 1H), 1.78-1.68 (m, 2H), 1.37 (s, 3H).

### Example 34

### 7-[(Anilinocarbonyl)amino]-N-hydroxy-1,2,3,4-tetrahydronaphthalene-2-carboxamide Compound I-30

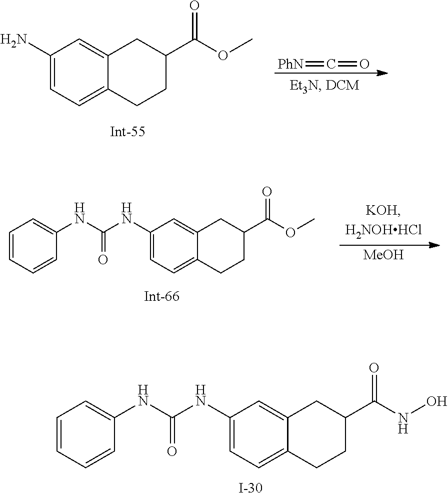

### Step 1: methyl 7-(3-phenylureido)-1,2,3,4-tetrahydronaphthalene-2-carboxylate Intermediate 66

Methyl 7-amino-1,2,3,4-tetrahydronaphthalene-2-carboxylate.HCl (Int-55, 150 mg, 0.55 mmol) was dissolved in N,N-dimethylformamide (4.5 mL) to which triethylamine (0.231 mL, 1.66 mmol) was added. To this solution was added phenyl isocyanate (0.15 mL, 1.38 mmol). The reaction mixture was stirred at room temperature for 3 h. The reaction mixture was partitioned between ethyl acetate and water. The phases were separated and the aqueous phase was extracted twice more with ethyl acetate. The product precipitated in the extraction. Both phases were filtered to afford the product as a solid (200 mg) which was used as obtained in the following step. LC-MS: (FA) ES+ 325.

### Step 2: 7-[(Anilinocarbonyl)amino]-N-hydroxy-1,2,3,4-tetrahydronaphthalene-2-carboxamide Compound I-30

The title compound was prepared from Intermediate 66 following the procedure detailed in Example 1, Step 4. LC-MS: (FA) ES+ 326; NMR (400 MHz, d₆-DMSO) δ ppm 10.50 (s, 1H), 9.66 (s, 1H), 9.55 (s, 1H), 7.54-7.42 (m, 2H), 7.26-7.16 (m, 4H), 6.97-6.86 (m, 2H), 2.88-2.58 (m, 4H), 2.41-2.30 (m, 1H), 1.91-1.82 (m, 1H), 1.68 (dddd, J=12.1, 12.1, 12.0, 5.8 Hz, 1H).

### Example 35

### N-hydroxy-7-[(pyridin-3-ylmethyl)amino]-1,2,3,4-tetrahydronaphthalene-2-carboxamide Compound I-38

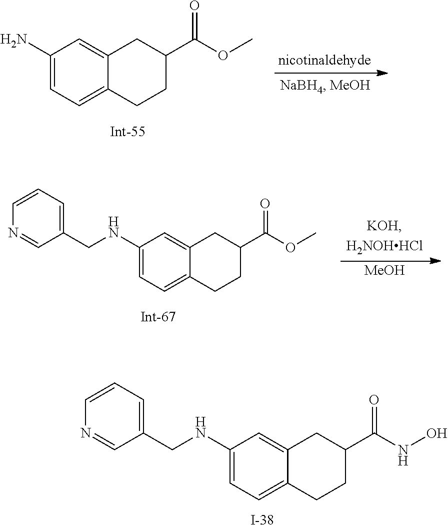

### Step 1: methyl 7-(pyridin-3-ylmethylamino)-1,2,3,4-tetrahydronaphthalene-2-carboxylate Intermediate 67

Methyl 7-amino-1,2,3,4-tetrahydronaphthalene-2-carboxylate.HCl (Int-55, 50 mg, 0.2 mmol) was dissolved in methanol (1.75 mL) in a round-bottom flask. To this solution was added 3-pyridinecarboxaldehyde (0.0202 mL, 0.2175 mmol) and sodium borohydride. The reaction mixture was stirred at room temperature until reaction was complete. The reaction mixture was diluted with methylene chloride (10 mL) and washed with a 1M sodium hydroxide solution. The washings were discarded and the methylene chloride extracts were then extracted with 1M hydrochloric acid (2 mL) diluted in water (5 mL). The aqueous extracts were then basified by the addition of 1N sodium hydroxide and extracted three times with 10% methanol in methylene chloride to afford Intermediate 67 which was used as obtained in the following step. LC-MS: (FA) ES+ 297.

### Step 2: N-hydroxy-7-[(pyridin-3-ylmethyl)amino]-1,2,3,4-tetrahydronaphthalene-2-carboxamide Compound I-38

The title compound was prepared from Intermediate 67 following the procedure detailed in Example 1, Step 4. LC-MS: (FA) ES+ 298; ¹H NMR (400 MHz, d₆-DMSO) δ ppm 8.55 (d, J=1.7 Hz, 1H), 8.43-8.40 (m, 1H), 7.71 (ddd, J=7.8, 1.8, 1.8 Hz, 1H), 7.32 (dd, J=7.2, 4.8 Hz, 1H), 6.73 (d, J=8.2 Hz, 1H), 6.36 (dd, J=8.2, 2.3 Hz, 1H), 6.29 (d, J=2.0 Hz, 1H), 6.05-5.97 (m, 1H), 4.27-4.21 (m, 2H), 2.76-2.52 (m, 4H), 2.34-2.21 (m, 1H), 1.85-1.77 (m, 1H), 1.60 (ddd, J=24.4, 12.0, 6.0 Hz, 1H).

### Example 36

### 7-(dimethylamino)-N-hydroxy-1,2,3,4-tetrahydronaphthalene-2-carboxamide Compound I-40

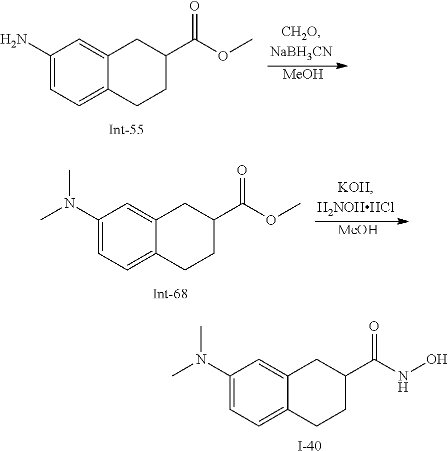

### Step 1: methyl 7-(dimethylamino)-1,2,3,4-tetrahydronaphthalene-2-carboxylate Intermediate 68

To a methanolic (2 mL) solution of methyl 7-amino-1,2,3,4-tetrahydronaphthalene-2-carboxylate.HCl (Int-55, 78 mg, 0.32 mmol) and formaldehyde (37% in water, 0.2 mL, 3 mmol) was added sodium cyanoborohydride (81 mg, 1.3 mmol). The mixture was stirred at room temperature for 2 h and then the reaction mixture was partitioned between water and ethyl acetate. The extracts were washed with brine and dried over sodium sulfate, filtered and concentrated under reduced pressure. The crude residue was purified by silica gel chromatography (hexanes to 80/20 hexanes/ethyl acetate) to afford the product (50 mg, 70%). ¹H NMR (400 MHz, CDCl₃) δ ppm 6.98 (d, J=8.4 Hz, 1H), 6.61 (dd, J=8.4, 2.7 Hz, 1H), 6.50 (d, J=2.5 Hz, 1H), 3.73 (s. 3H), 3.01-2.96 (m, 2H), 2.90 (s, 6H), 2.81-2.69 (m, 3H), 2.27-2.15 (m, 1H), 1.83 (dddd, J=12.9, 11.3, 11.1, 6.4 Hz, 1H).

### Step 2: 7-(dimethylamino)-N-hydroxy-1,2,3,4-tetrahydronaphthalene-2-carboxamide Compound I-40

The title compound was prepared from Intermediate 68 following the procedure detailed in Example 1, Step 4. LC-MS: (FA) ES+ 235; ¹H NMR (400 MHz, d₆-DMSO) δ ppm 6.86 (d, J=8.4 Hz, 1H), 6.52 (dd, J=8.4, 2.3 Hz, 1H), 6.42 (d, J=1.7 Hz, 1H), 2.91-2.53 (m, 11H), 2.36-2.24 (m, 1H), 1.64 (ddd, J=24.3, 11.9, 5.9 Hz, 1H).

### Example 37

### N-hydroxy-7-(pyridin-4-ylamino)-1,2,3,4-tetrahydronaphthalene-2-carboxamide Compound I-33

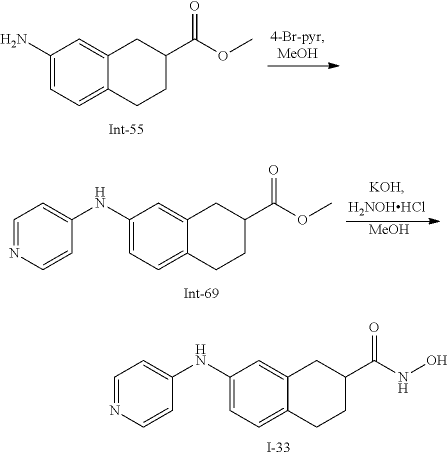

### Step 1: methyl 7-(pyridin-4-ylamino)-1,2,3,4-tetrahydronaphthalene-2-carboxylate Intermediate 69

Methyl 7-amino-1,2,3,4-tetrahydronaphthalene-2-carboxylate.HCl (Int-55, 86 mg, 0.31 mmol), 4-bromopyridine.HCl (66.1 mg, 0.34 mmol) and methanol (2.2 mL) were combined in a microwave vial which was sealed and heated in the microwave at 120° C. for 30 min. The methanol was then removed under reduced pressure and the crude intermediate was used without further purification in the following step. LC-MS: (FA) ES+ 283.

### Step 2: N-hydroxy-7-(pyridin-4-ylamino)-1,2,3,4-tetrahydronaphthalene-2-carboxamide Compound I-33

The title compound was prepared from Intermediate 69 following the procedure detailed in Example 1, Step 4. LC-MS: (FA) ES+ 284. ¹H NMR (400 MHz, d₆-DMSO) δ ppm 8.69 (s, 1H), 8.21 (s, 1H), 8.15-8.11 (m, 2H), 7.04 (d, J=8.0 Hz, 1H), 6.95-6.89 (m, 2H), 6.85-6.81 (m, 2H), 2.90-2.62 (m, 4H), 2.36 (dddd, J=11.2, 11.2, 4.9, 2.9 Hz, 1H), 1.92-1.84 (m, 1H), 1.69 (ddd, J=24.3, 11.8, 5.6 Hz, 1H).

### Example 38

### 6-[(cyclopropylcarbonyl)amino]-N-hydroxy-1,2,3,4-tetrahydronaphthalene-2-carboxamide Compound I-77

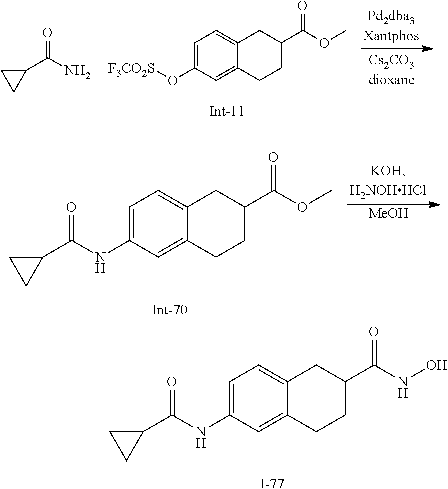

### Step 1: methyl 6-[(cyclopropylcarbonyl)amino]-1,2,3,4-tetrahydronaphthalene-2-carboxylate Intermediate 70

Tris(dibenzylideneacetone)dipalladium(0) (27.1 mg, 0.0296 mmol), 4,5-bis(diphenylphosphino)-9,9-dimethylxanthene (51.3 mg, 0.0887 mmol), cyclopropanecarboxamide (38 mg, 0.44 mmol) and cesium carbonate (0.193 g, 0.591 mmol) were added to a 2-5 mL microwave vial fitted with a stirbar. The vial was capped and purged with argon. A solution of methyl 6-{[(trifluoromethyl)sulfonyl]oxy}-1,2,3,4-tetrahydronaphthalene-2-carboxylate (Int-11, 100 mg, 0.296 mmol) in 1,4-dioxane (2 mL) was prepared under argon. The solution was added to the microwave vial by syringe. The contents of the vial were sonicated under active argon flow for 2 min. The vial was capped, sealed and heated at 100° C. overnight in an oil bath. The mixture was filtered through Celite and the filter pad was generously washed with ethyl acetate. The filtrate was concentrated under reduced pressure and the resulting crude product was purified by silica gel chromatography (90/10 to 60/40 hexanes/ethyl acetate gradient) to afford the product (61 mg, 76%). LC-MS: (AA) ES+ 274; ¹H NMR (300 MHz, CDCl₃) δ ppm 7.43-7.30 (m, 2H), 7.14 (d, J=8.2 Hz, 1H), 7.02 (d, J=8.2 Hz, 1H), 3.72 (s, 3H), 3.04-2.65 (m, 5H), 2.26-2.11 (m, 1H), 1.83 (m, 1H), 1.55-1.41 (m, 1H), 1.11-1.03 (m, 2H), 0.86-0.79 (m, 2H).

### Step 2: 6-[(cyclopropylcarbonyl)amino]-N-hydroxy-1,2,3,4-tetrahydronaphthalene-2-carboxamide Compound I-77

The title compound was prepared from Intermediate 70 following the general procedure detailed in Example 1, Step 4. The crude product was purified by amine-functionalized silica chromatography (ethyl acetate to 60/40 ethyl acetate/ethanol gradient). LC-MS: (AA) ES+ 275; ¹H NMR (300 MHz, d₆-DMSO) δ ppm 7.34 (s, 1H), 7.25 (d, J=8.0 Hz, 1H), 6.97 (d, J=8.4 Hz, 1H), 2.86-2.60 (m, 4H), 2.41-2.24 (m, 1H), 1.93-1.80 (m, 1H), 1.78-1.58 (m, 2H), 0.83-0.67 (m, 4H).

### Example 39

### N-hydroxy-6-[(4-methoxyphenyl)amino]-1,2,3,4-tetrahydronaphthalene-2-carboxamide Compound I-80

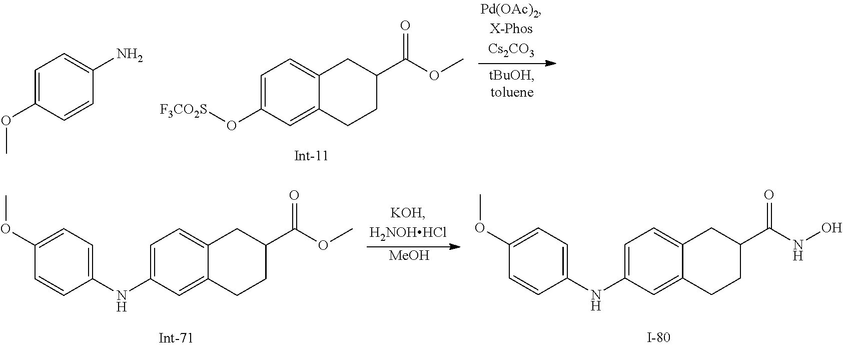

### Step 1: methyl 6-[(4-methoxyphenyl)amino]-1,2,3,4-tetrahydronaphthalene-2-carboxylate Intermediate 71

A microwave vial was fitted with a stirbar. To this vial was added 4-methoxybenzenamine (0.0598 mL, 0.52 mmol), palladium(II) acetate (0.0053 g, 0.0236 mmol), 2-dicyclohexylphosphino-2′,4′,6′-tri-iso-propyl-1,1′-biphenyl (0.0225 g, 0.0473 mmol), and cesium carbonate (0.308 g, 0.946 mmol). The vial was capped and purged with argon. A solution of methyl 6-{[(trifluoromethyl)sulfonyl]oxy}-1,2,3,4-tetrahydronaphthalene-2-carboxylate (Int-11, 0.16 g, 0.473 mmol) in tert-butyl alcohol (0.6 mL) and toluene (3.2 mL) was added. The vial was then purged with argon under sonication for 3 minutes. The reaction mixture was heated in a microwave at 150° C. for 1 h. The crude mixture was adsorbed onto Celite (1 g) and concentrated under reduced pressure. The crude product was purified by silica gel chromatography (hexanes to 70/30 hexanes/ethyl acetate gradient) to afford the product (125 mg, 85%). LC-MS: (AA) ES+ 312; ¹H NMR (300 MHz, CDCl₃) δ ppm 7.09-7.02 (m, 2H), 6.96 (d, J=8.2 Hz, 1H), 6.88-6.82 (m, 2H), 6.72 (dd, J=8.2, 2.0 Hz, 1H), 6.68-6.64 (m, 1H), 3.80 (s, 3H), 3.72 (s, 3H), 3.01-2.65 (m, 5H), 2.26-2.12 (m, 1H), 1.92-1.73 (m, 1H).

### Step 2: N-hydroxy-6-[(4-methoxyphenyl)amino]-1,2,3,4-tetrahydronaphthalene-2-carboxamide Compound I-80

The title compound was prepared from Intermediate 71 following the procedure outlined in Example 1, Step 4. The crude product was purified by chromatography on amine-functionalized silica (ethyl acetate to 60/40 ethyl acetate/ethanol gradient; 83 mg, 66%). LC-MS: (AA) ES+ 313; ¹H NMR (300 MHz, CD₃OD) δ ppm 7.05-6.94 (m, 2H), 6.86 (d, J=8.2 Hz, 1H), 6.83-6.77 (m, 2H), 6.70 (dd, J=8.2, 2.3 Hz, 1H), 6.66 (d, J=2.0 Hz, 1H), 3.74 (s, 3H), 2.93-2.81 (m, 1H), 2.79-2.66 (m, 3H), 2.41 (dddd, J=11.6, 11.6, 5.0, 3.1 Hz, 1H), 1.99-1.89 (m, 1H), 1.90-1.78 (m, 1H).

### Example 40

The following compounds were prepared in a fashion analogous to that described in Example 39 from Intermediate 11 and the appropriate aniline.

| | LC-MS (AA) |
|---|---|
| Compound | ES+ |
| I-78 | 317 |
| I-79 | 313 |
| I-83 | 317 |
| I-84 | 313 |

### Example 41

### 7-cyano-N-hydroxy-1,2,3,4-tetrahydronaphthalene-2-carboxamide Compound I-42

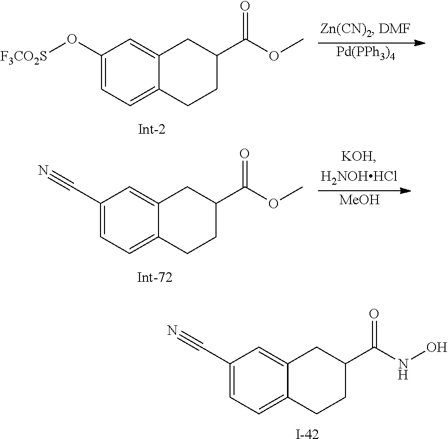

### Step 1: methyl 7-cyano-1,2,3,4-tetrahydronaphthalene-2-carboxylate Intermediate 72

Methyl 7-{[(trifluoromethyl)sulfonyl]oxy}-1,2,3,4-tetrahydronaphthalene-2-carboxylate (Int-2, 1.2 g, 3.55 mmol) was dissolved in N,N-dimethylformamide (17 mL). The solution was purged with argon, charged with zinc cyanide (800 mg, 7 mmol) and tetrakis(triphenylphosphine)palladium(0) (120 mg, 0.1 mmol), and the reaction mixture was heated at 125° C. under stirring overnight. The reaction mixture was allowed to cool to room temperature and was partitioned between ethyl acetate and water. The extracts were washed with water a second time, then washed with brine, dried over sodium sulfate, filtered and concentrated under reduced pressure. The crude product was purified by silica gel chromatography (hexanes to 50/50 hexanes/ethyl acetate gradient) to afford the product as a white solid (657 mg, 86%). LC-MS: (FA) ES+ 216; ¹H NMR (400 MHz, CDCl₃) δ ppm 7.41-7.36 (m, 2H), 7.17 (d, J=7.9 Hz, 1H), 3.74 (s, 3H), 3.05-3.01 (m, 2H), 2.98-2.72 (m, 3H), 2.28-2.18 (m, 1H), 1.90 (dddd, J=13.3, 10.3, 10.3, 6.0 Hz, 1H).

### Step 2: 7-cyano-N-hydroxy-1,2,3,4-tetrahydronaphthalene-2-carboxamide Compound I-42

The title compound was prepared from Intermediate 72 following the procedure detailed in Example 1, Step 4. The reaction time was limited to 10 minutes to minimize undesired reactivity at the nitrile. LC-MS: (FA) ES+ 217; ¹H NMR (400 MHz, d₆-DMSO) δ ppm 10.66 (s, 1H), 8.51 (s, 1H), 7.58 (s, 1H), 7.52 (dd, J=7.9, 1.4 Hz, 1H), 7.27 (d, J=7.9 Hz, 1H), 2.94-2.71 (m, 4H), 2.44-2.34 (m, 1H), 1.93-1.84 (m, 1H), 1.71 (m, 1H).

### Example 42

### N²-hydroxy-1,2,3,4-tetrahydronaphthalene-2,7-dicarboxamide Compound I-39

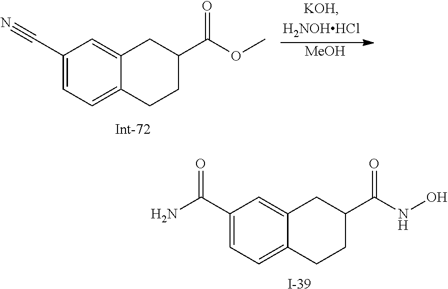

The title compound was prepared from Intermediate 72 following the procedure detailed in Example 1, Step 4. A reaction time of 1 h was required for complete conversion of the nitrile to the primary amide. LC-MS: (FA) ES+ 235; ¹H NMR (400 MHz, d₆-DMSO) δ ppm 7.92 (s, 1H), 7.59-7.51 (m, 2H), 7.24-7.18 (m, 1H), 7.13 (d, J=7.8 Hz, 1H), 2.92-2.67 (m, 4H), 2.55-2.52 (m, 2H), 2.43-2.33 (m, 1H), 1.91-1.81 (m, 1H), 1.69 (dddd, J=12.1, 12.1, 11.9, 5.8 Hz, 1H).

### Example 43

### N⁷-(tert-butyl)-N²-hydroxy-1,2,3,4-tetrahydronaphthalene-2,7-dicarboxamide Compound I-45

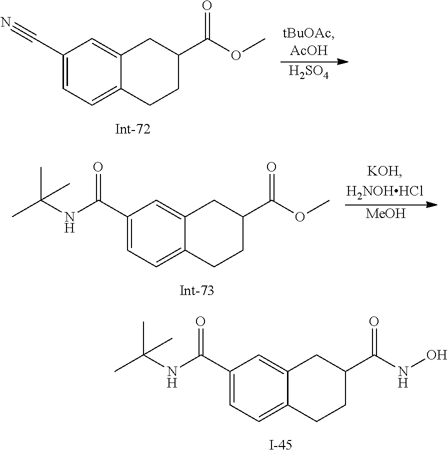

### Step 1: methyl 7-[(tert-butylamino)carbonyl]-1,2,3,4-tetrahydronaphthalene-2-carboxylate Intermediate 73

To a solution of methyl 7-cyano-1,2,3,4-tetrahydronaphthalene-2-carboxylate (Int-72, 50 mg, 0.2 mmol) and tert-butyl acetate (60.53 mg, 0.5211 mmol) in acetic acid (0.06 mL) was added a mixture of sulfuric acid (0.02 mL, 0.5 mmol) and acetic acid (0.048 mL, 0.84 mmol) dropwise. The internal temperature was maintained below 30° C. during the addition with the aid of a cooled water bath. The reaction mixture was stirred at room temperature for 1 h. A solution of sodium acetate trihydrate in water (2.0 M, 0.418 mL) was added and the mixture was extracted with methylene chloride three times. The extracts were combined and washed with brine, dried over sodium sulfate, filtered and concentrated under reduced pressure to afford the product as an oil. The crude product was used as obtained in the following step. LC-MS: (FA) ES+ 290.

### Step 2: N⁷-(tert-butyl)-N²-hydroxy-1,2,3,4-tetrahydronaphthalene-2,7-dicarboxamide Compound I-45

The title compound was prepared from Intermediate 73 following the procedure detailed in Example 1, Step 4. LC-MS: (FA) ES+ 291; ¹H NMR (400 MHz, d₆-DMSO) δ ppm 10.57 (d, J=0.7 Hz, 1H), 8.87 (s, 1H), 7.60 (s, 1H), 7.54-7.48 (m, 2H), 7.10 (d, J=7.8 Hz, 1H), 2.93-2.67 (m, 4H), 2.37 (dddd, J=10.9, 10.9, 4.9, 3.2 Hz, 1H), 1.93-1.84 (m, 1H), 1.70 (dddd, J=11.7, 11.7, 11.7, 5.6 Hz, 1H), 1.35 (s, 9H).

### Example 44

### N-hydroxy-7-(pyridin-4-ylmethoxy)-1,2,3,4-tetrahydronaphthalene-2-carboxamide Compound I-5

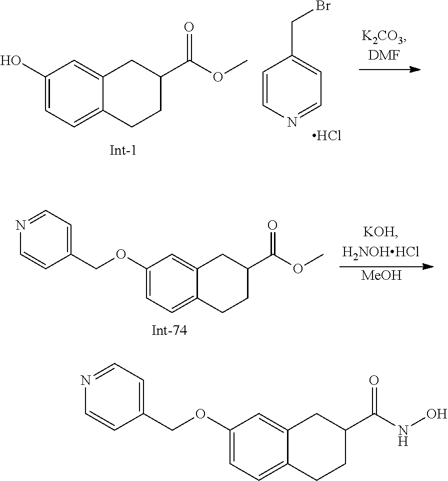

### Step 1: methyl 7-(pyridin-4-ylmethoxy)-1,2,3,4-tetrahydronaphthalene-2-carboxylate Intermediate 74

A mixture of methyl 7-hydroxy-1,2,3,4-tetrahydronaphthalene-2-carboxylate (Int-1, 147 mg, 0.713 mmol), potassium carbonate (0.296 g, 2.14 mmol) and 4-(chloromethyl)pyridine hydrochloride (0.14 g, 0.855 mmol) in N,N-dimethylformamide (6 mL) was stirred overnight at 50° C. The reaction mixture was poured into water and extracted with methylene chloride several times. The extracts were combined and washed with brine, then dried over sodium sulfate, filtered and concentrated under reduced pressure. The crude residue was purified by silica gel chromatography (hexanes to 50/50 hexanes/ethyl acetate gradient) to afford the product as an oil (180 mg, 85%). LC-MS: (FA) ES+ 298; ¹H NMR (400 MHz, CDCl₃) δ ppm 8.62-8.54 (m, 2H), 7.33-7.29 (m, 2H), 6.97 (d, J=8.4 Hz, 1H), 6.71 (dd, J=8.4, 2.7 Hz, 1H), 6.66 (d, J=2.6 Hz, 1H), 5.02-5.01 (m, 2H), 3.69 (s, 3H), 2.96-2.92 (m, 2H), 2.83-2.65 (m, 3H), 2.20-2.12 (m, 1H), 1.80 (dddd, J=13.0, 10.9, 10.8, 6.1 Hz, 1H).

### Step 2: N-hydroxy-7-(pyridin-4-ylmethoxy)-1,2,3,4-tetrahydronaphthalene-2-carboxamide Compound I-5

The title compound was prepared from Intermediate 74 following the procedure detailed in Example 1, Step 4. LC-MS: (FA) ES+ 299; ¹H NMR (300 MHz, d₆-DMSO) δ ppm 10.79-10.33 (m, 1H), 8.78 (s, 1H), 8.63-8.51 (m, 2H), 7.45-7.36 (m, 2H), 7.03-6.92 (m, 1H), 6.80-6.69 (m, 2H), 5.11 (s, 2H), 2.89-2.55 (m, 4H), 2.41-2.25 (m, 1H), 1.91-1.79 (m, 1H), 1.66 (dddd, J=12.0, 12.0, 11.9, 5.8 Hz, 1H).

### Example 45

The following compounds were prepared from Intermediate 1 and the appropriate alkyl bromides following the procedures detailed in Example 44.

| Compound | LC-MS (FA) ES+ |
|---|---|
| I-6 | 264 |
| I-7 | 298 |

### Example 46

### N-hydroxy-7-(phenylethynyl)-1,2,3,4-tetrahydronaphthalene-2-carboxamide Compound I-34

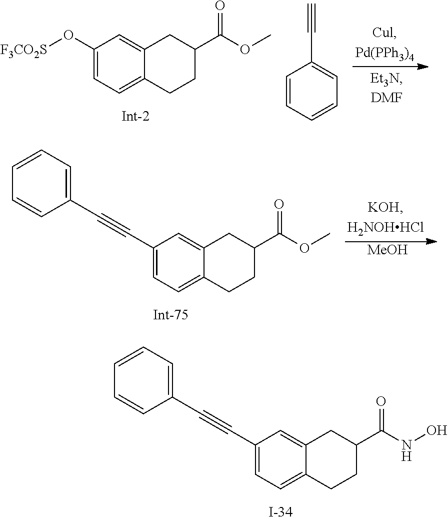

### Step 1: methyl 7-(phenylethynyl)-1,2,3,4-tetrahydronaphthalene-2-carboxylate Intermediate 75

A microwave vial was charged with a solution of methyl 7-{[(trifluoromethyl)sulfonyl]oxy}-1,2,3,4-tetrahydronaphthalene-2-carboxylate (Int-2, 0.2 g, 0.591 mmol) in N,N-dimethylformamide (6.3 mL), copper(I) iodide (11.2 mg, 0.0591 mmol), and tetrakis(triphenylphosphine)palladium(0) (68.3 mg, 0.0591 mmol). The vial was capped and sealed then flushed with argon. Triethylamine (0.165 mL, 1.18 mmol) was added and the contents of the vial were degassed under active argon flow for 1 h. Phenylacetylene (0.215 mL, 1.95 mmol) was added and the reaction mixture was heated at 60° C. overnight. The reaction mixture was allowed to cool to room temperature and then poured into water. The aqueous phase was extracted with ethyl acetate three times. The extracts were combined, washed with brine and dried over sodium sulfate, then filtered and concentrated under reduced pressure. The crude residue was purified by silica gel chromatography (hexanes to 70/30 hexanes/ethyl acetate gradient) to afford the product as a brown oil (150 mg, 87%). LC-MS: (FA) ES+ 291; ¹H NMR (400 MHz, CDCl₃) δ ppm 7.55-7.50 (m, 2H), 7.38-7.31 (m, 3H), 7.31-7.27 (m, 2H), 7.07 (d, J=8.4 Hz, 1H), 3.74 (s, 3H), 3.04-2.99 (m, 2H), 2.96-2.71 (m, 3H), 2.28-2.17 (m, 1H), 1.88 (dddd, J=13.2, 10.8, 10.7, 6.2 Hz, 1H).

### Step 2: N-hydroxy-7-(phenylethynyl)-1,2,3,4-tetrahydronaphthalene-2-carboxamide Compound I-34

The title compound was prepared from Intermediate 75 following the procedure detailed in Example 1, Step 4. ¹H NMR (400 MHz, d₆-DMSO) δ ppm 10.54 (s, 1H), 8.81 (s, 1H), 7.56-7.47 (m, 2H), 7.43-7.38 (m, 3H), 7.31-7.29 (m, 1H), 7.26 (dd, J=7.8, 1.5 Hz, 1H), 7.12 (d, J=7.8 Hz, 1H), 2.90-2.68 (m, 4H), 2.37 (dddd, J=11.1, 11.1, 5.3, 3.1 Hz, 1H), 1.93-1.84 (m, 1H), 1.71 (dddd, J=12.2, 12.2, 12.0, 6.0 Hz, 1H).

### Example 47

### N-hydroxy-7-(2-phenylethyl)-1,2,3,4-tetrahydronaphthalene-2-carboxamide Compound I-36

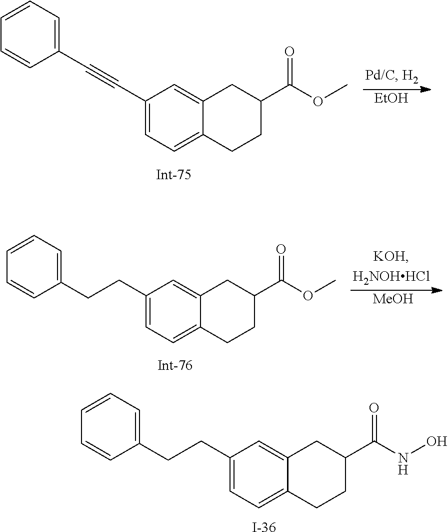

### Step 1: methyl 7-phenethyl-1,2,3,4-tetrahydronaphthalene-2-carboxylate Intermediate 76

To a solution of methyl 7-(phenylethynyl)-1,2,3,4-tetrahydronaphthalene-2-carboxylate (Int-75, 60 mg, 0.2 mmol) in ethanol (1.5 mL) was carefully added palladium (10% on carbon, 22 mg, 0.0207 mmol). The black mixture was stirred under an atmosphere of hydrogen overnight. The mixture was then filtered through Celite and concentrated under reduced pressure. The crude product was used as obtained without purification. LC-MS: (FA) ES+ 295; ¹H NMR (400 MHz, CDCl₃) δ ppm 7.32-7.27 (m, 2H), 7.23-7.17 (m, 3H), 7.04-6.91 (m, 3H), 3.73 (s, 3H), 3.00-2.96 (m, 2H), 2.93-2.79 (m, 6H), 2.74 (dddd, J=11.1, 8.2, 8.1, 3.1 Hz, 1H), 2.25-2.16 (m, 1H), 1.85 (dddd, J=13.2, 10.8, 10.8, 6.4 Hz, 1H).

### Step 2: N-hydroxy-7-(2-phenylethyl)-1,2,3,4-tetrahydronaphthalene-2-carboxamide Compound I-36

The title compound was prepared from Intermediate 76 following the procedure detailed in Example 1, Step 4. LC-MS: (FA) ES+ 296.

### Example 48

### 7-[2-(dimethylamino)ethoxy]-N-hydroxy-1,2,3,4-tetrahydronaphthalene-2-carboxamide Compound I-44

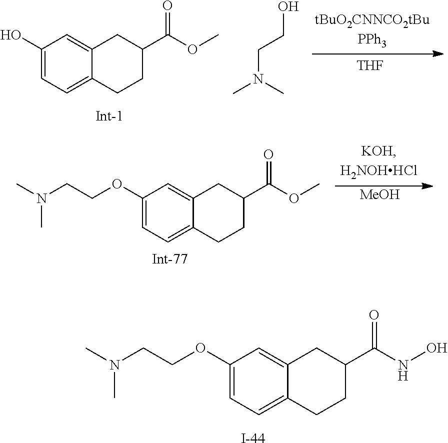

### Step 1: methyl 7-[2-(dimethylamino)ethoxy]-1,2,3,4-tetrahydronaphthalene-2-carboxylate Intermediate 77

To a cooled (0° C.) solution of triphenylphosphine (314.8 mg, 1.2 mmol) in tetrahydrofuran (4 mL) was added a solution of di-tert-butyl azodicarboxylate (276.3 mg, 1.2 mmol) in tetrahydrofuran (4 mL) dropwise. After stirring at 0° C. for 20 min, a solution of N,N-dimethylaminoethanol (107 mg, 1.2 mmol) was added dropwise to the reaction mixture. After an additional 10 min, methyl 7-hydroxy-1,2,3,4-tetrahydronaphthalene-2-carboxylate (Int-1, 225 mg, 1.091 mmol) was added. The reaction was allowed to warm to room temperature and stir overnight. The reaction mixture was diluted with 30 ml DCM, and a dilute hydrochloric acid solution (0.3 mL 1N HCl in 20 mL water) was added. After stirring several minutes, the phases were separated and the aqueous phase was basified by the addition of 1N NaOH to pH 8. The cloudy mixture was extracted with 5% methanol in methylene chloride (3×). The extracts were combined, dried over sodium sulfate, filtered and concentrated under reduced pressure to afford the product as an oil which was used in the following step without further purification (220 mg, 73%). LC-MS: (FA) ES+ 278.

### Step 2: 7-[2-(dimethylamino)ethoxy]-N-hydroxy-1,2,3,4-tetrahydronaphthalene-2-carboxamide Compound I-44

The title compound was prepared from Intermediate 77 following the procedure detailed in Example 1, Step 4. LC-MS: (FA) ES+ 279. ¹H NMR (400 MHz, CD₃OD δ ppm 7.02 (d, J=8.3 Hz, 1H), 6.81-6.72 (m, 2H), 4.33-4.24 (m, 2H), 3.60-3.53 (m, 3H), 2.99-2.95 (m, 6H), 2.87-2.67 (m, 3H), 2.45 (dddd, J=11.2, 11.2, 4.4, 3.1 Hz, 1H), 2.03-1.94 (m, 1H), 1.82 (ddd, J=24.5, 12.0, 5.9 Hz, 1H).

### Example 49

### 7-{3-[2-(dimethylamino)ethoxy]phenoxy}-N-hydroxy-1,2,3,4-tetrahydronaphthalene-2-carboxamide Compound I-46

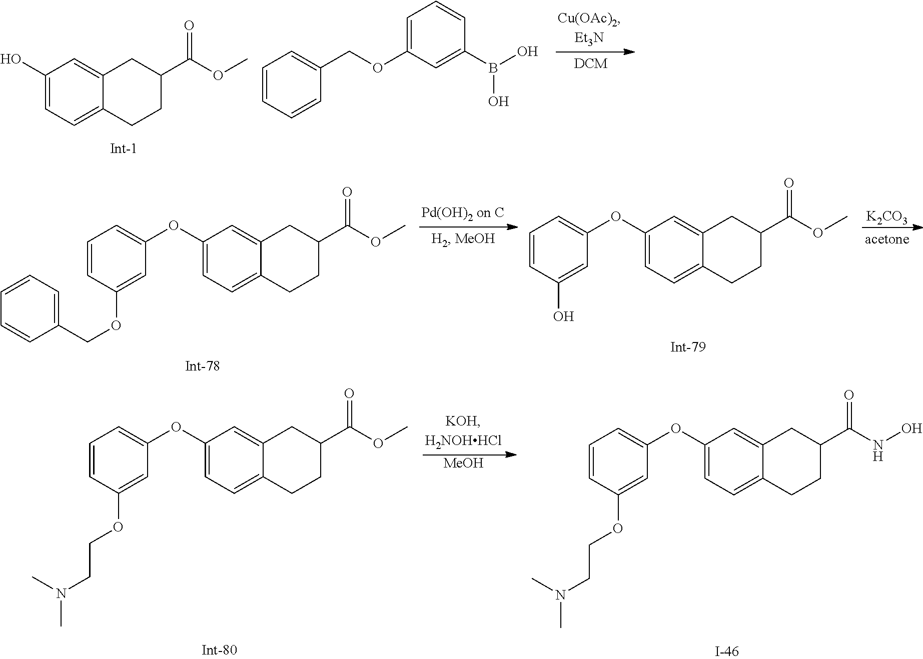

### Step 1: methyl 7-[3-(benzyloxy)phenoxy]-1,2,3,4-tetrahydronaphthalene-2-carboxylate Intermediate 78

The title compound was prepared following the procedure detailed in Example 18, Step 1, substituting 3-benzyloxyphenylboronic acid for 3-(N-Boc-amino)phenylboronic acid. ¹H NMR (400 MHz, CDCl₃) δ ppm 7.47-7.29 (m, 5H), 7.21 (dd, J=8.2, 8.2 Hz, 1H), 7.04 (d, J=8.2 Hz, 1H), 6.79 (dd, J=8.2, 2.6 Hz, 1H), 6.76 (d, J=2.3 Hz, 1H), 6.70 (ddd, J=8.3, 2.4, 0.8 Hz, 1H), 6.61 (dd, J=2.3, 2.3 Hz, 1H), 6.58 (ddd, J=8.0, 2.3, 0.8 Hz, 1H), 5.03-5.01 (m, 2H), 3.73 (s, 3H), 2.99-2.94 (m, 2H), 2.91-2.69 (m, 3H), 2.25-2.16 (m, 1H), 1.86 (dddd, J=13.1, 10.9, 10.9, 6.1 Hz, 1H).

### Step 2: methyl 7-(3-hydroxyphenoxy)-1,2,3,4-tetrahydronaphthalene-2-carboxylate Intermediate 79

To a solution of methyl 7-[3-(benzyloxy)phenoxy]-1,2,3,4-tetrahydronaphthalene-2-carboxylate (Int-78, 140 mg, 0.36 mmol) in methanol (1.5 mL) was carefully added palladium hydroxide (20% on carbon, 2.5 mg). The black mixture was stirred under an atmosphere of hydrogen overnight. The reaction flask was then purged with argon and the mixture carefully filtered through Celite. The filtrate was concentrated under reduced pressure to afford the product, which was used without further purification. LC-MS: (FA) ES+ 299; ¹H NMR (300 MHz, CDCl₃) δ ppm 7.15 (dd, J=8.2, 8.2 Hz, 1H), 7.05 (d, J=8.2 Hz, 1H), 6.83-6.75 (m, 2H), 6.58-6.51 (m, 2H), 6.46 (dd, J=2.3, 2.3 Hz, 1H), 3.73 (s, 3H), 3.02-2.93 (m, 2H), 2.91-2.68 (m, 3H), 2.28-2.15 (m, 1H), 1.86 (m, 1H).

### Step 3: methyl 7-{3-[2-(dimethylamino)ethoxy]phenoxy}-1,2,3,4-tetrahydronaphthalene-2-carboxylate Intermediate 80

To a solution of methyl 7-(3-hydroxyphenoxy)-1,2,3,4-tetrahydronaphthalene-2-carboxylate (Int-79, 74 mg, 0.25 mmol) in acetone (1.8 mL) was added potassium carbonate (103 mg, 0.744 mmol) followed by β-dimethylaminoethyl chloride hydrochloride (43 mg, 0.3 mmol). The resulting mixture was stirred at 60° C. overnight. The solvent was removed under reduced pressure and the residue was suspended in water. The aqueous mixture was extracted three times with ethyl acetate. The extracts were combined, washed with brine, dried over sodium sulfate, filtered and concentrated under reduced pressure. The crude residue was purified by silica gel chromatography (hexanes to ethyl acetate gradient) to afford the product (70 mg, 80%). LC-MS: (FA) ES+ 370.

### Step 4: 7-{3-[2-(dimethylamino)ethoxy]phenoxy}-N-hydroxy-1,2,3,4-tetrahydronaphthalene-2-carboxamide Compound I-46

The title compound was prepared from Intermediate 80 following the procedure detailed in Example 1, Step 4. LC-MS: (FA) ES+ 371; ¹H NMR (400 MHz, CD₃OD) δ ppm 8.50 (s, 1H), 7.22 (dd, J=8.2, 8.2 Hz, 1H), 7.05 (d, J=8.3 Hz, 1H), 6.76-6.68 (m, 3H), 6.60-6.52 (m, 2H), 4.33-4.21 (m, 2H), 3.48-3.42 (m, 2H), 2.99-2.68 (m, 10H), 2.46 (dddd, J=11.4, 11.4, 4.7, 3.0 Hz, 1H), 2.05-1.93 (m, 1H), 1.83 (dddd, J=11.8, 11.8, 11.8, 5.8 Hz, 1H).

### Example 50

### N-hydroxy-7-methoxy-2-methyl-1,2,3,4-tetrahydronaphthalene-2-carboxamide Compound I-76

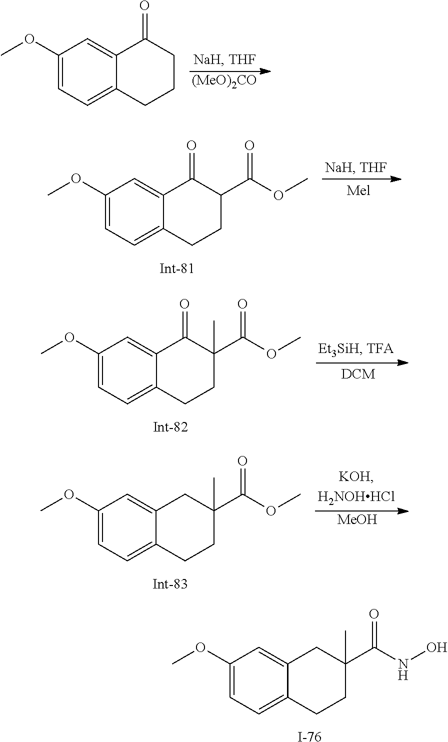

### Step 1: methyl 7-methoxy-1-oxo-1,2,3,4-tetrahydronaphthalene-2-carboxylate Intermediate 81

Solid sodium hydride (60% in mineral oil, 6.8093 g, 170.25 mmol) was weighed into a clean and dry flask. Tetrahydrofuran (200 mL) and dimethyl carbonate (17.9 mL, 213 mmol) were added to the flask and the mixture was warmed to 65° C. in an oil bath. A solution of 7-methoxy-1-tetralone (25 g, 141.9 mmol) in tetrahydrofuran (50 mL) was added dropwise over 0.5 h via canula. The resulting solution was then stirred at 65° C. overnight under nitrogen. The reaction mixture was cooled to room temperature and excess base was quenched by the careful dropwise addition of acetic acid (16.1 mL) followed by water. The product was then extracted into diethyl ether. The aqueous phase was extracted a second time with ether and the combined extracts were washed with water and brine then dried over sodium sulfate, filtered and concentrated under reduced pressure. The crude residue was purified by silica gel chromatography (hexanes to 80/20 hexanes/ethyl acetate gradient) to afford the product (31.5 g, 95%) as a mixture of keto and enol tautomers. LC-MS: (FA) ES+ 235.

### Step 2: methyl 7-methoxy-2-methyl-1-oxo-1,2,3,4-tetrahydronaphthalene-2-carboxylate Intermediate 82

Sodium hydride (60% in mineral oil, 0.525 g, 13.1 mmol) was weighed into a clean dry flask and suspended in tetrahydrofuran (40 mL, 500 mmol) and the resulting mixture was cooled to 0° C. in an ice-water bath. A solution of methyl 7-methoxy-1-oxo-1,2,3,4-tetrahydronaphthalene-2-carboxylate (Int-81, 2.05 g, 8.75 mmol) in THF (15 mL) was added and the mixture was stirred for 0.5 h. Methyl iodide (1.09 mL, 17.5 mmol) was then added and the reaction mixture was stirred overnight with slow warming to room temperature. Excess base was quenched by the careful addition of water. The mixture was then extracted three times with ethyl acetate. The combined extracts were then washed with water and brine, then dried over sodium sulfate, filtered and concentrated under reduced pressure. The crude residue was purified by silica gel chromatography (hexanes to 80/20 hexanes/ethyl acetate) to afford the product (1.57 g, 73%). ¹H NMR (400 MHz, CDCl₃) δ ppm 7.54 (d, J=2.8 Hz, 1H), 7.14 (d, J=8.4 Hz, 1H), 7.06 (dd, J=8.4, 2.8 Hz, 1H), 3.84 (s, 3H), 3.68 (s, 3H), 2.96 (ddd, J=16.8, 9.6, 4.7 Hz, 1H), 2.86 (ddd, J=17.1, 5.3, 5.3 Hz, 1H), 2.59 (ddd, J=13.6, 5.6, 4.8 Hz, 1H), 2.09-1.99 (m, 1H), 1.51 (s, 3H).

### Step 3: methyl 7-methoxy-2-methyl-1,2,3,4-tetrahydronaphthalene-2-carboxylate Intermediate 83

Methyl 7-methoxy-2-methyl-1-oxo-1,2,3,4-tetrahydronaphthalene-2-carboxylate (Int-82, 0.15 g, 0.604 mmol) was dissolved in a mixture of methylene chloride (3 mL) and trifluoroacetic acid (3 mL). The resulting solution was cooled in an ice-water bath and triethylsilane (0.203 mL, 1.27 mmol) was added dropwise via syringe. After addition was complete, the reaction flask was removed from the cooling bath and the mixture allowed to stir at room temperature for 1 h. Concentrated ammonium hydroxide solution was slowly and carefully added until the mixture was basic. The mixture was then extracted with methylene chloride three times. The extracts were combined, washed with water then brine, dried over sodium sulfate, filtered and concentrated under reduced pressure. The crude residue was purified by silica gel chromatography (hexanes to 80/20 hexanes/ethyl acetate gradient) to afford the product (0.125 g, 88%). ¹H NMR (400 MHz, CDCl₃) δ ppm 6.98 (d, J=8.4 Hz, 1H), 6.69 (dd, J=8.4, 2.7 Hz, 1H), 6.62 (d, J=2.6 Hz, 1H), 3.77 (s, 3H), 3.66 (s, 3H), 3.21 (d, J=16.5 Hz, 1H), 2.80-2.72 (m, 2H), 2.63 (d, J=16.5 Hz, 1H), 2.14 (dddd, J=7.6, 6.5, 6.5, 1.1 Hz, 1H), 1.76 (ddd, J=13.9, 6.9, 6.9 Hz, 1H), 1.27 (s, 3H).

### Step 4: N-hydroxy-7-methoxy-2-methyl-1,2,3,4-tetrahydronaphthalene-2-carboxamide Compound I-76

The title compound was prepared from Intermediate 83 following the procedure detailed in Example 1, Step 4. LC-MS: (FA) ES+ 236; ¹H NMR (400 MHz, CD₃OD) δ ppm 6.93 (d, J=8.3 Hz, 1H), 6.64 (dd, J=8.3, 2.7 Hz, 1H), 6.61 (d, J=2.5 Hz, 1H), 3.72 (s, 3H), 3.16-3.07 (m, 1H), 2.84-2.65 (m, 2H), 2.61 (d, J=16.6 Hz, 1H), 2.11 (dddd, J=7.3, 6.4, 6.4, 1.0 Hz, 1H), 1.72 (ddd, J=13.5, 7.2, 6.7 Hz, 1H), 1.22 (s, 3H).

### Example 51

### 7-(4-Chlorophenoxy)-N-hydroxy-2-methyl-1,2,3,4-tetrahydronaphthalene-2-carboxamide I-124

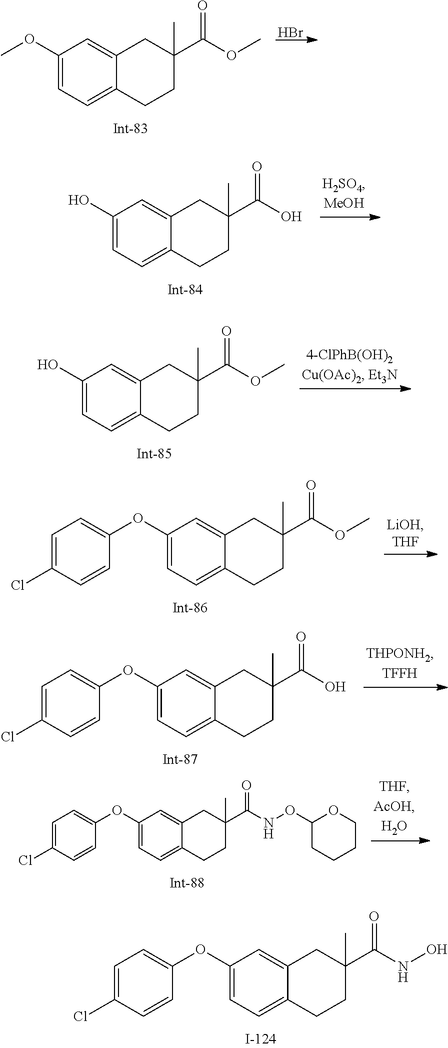

### Step 1: 7-Hydroxy-2-methyl-1,2,3,4-tetrahydronaphthalene-2-carboxylic acid Intermediate 84

Methyl 7-methoxy-2-methyl-1,2,3,4-tetrahydronaphthalene-2-carboxylate (Int-83, 0.85 g, 3.63 mmol) was dissolved in hydrobromic acid (48%. 12 mL, 221 mmol). The reaction mixture was then heated at 100° C. under nitrogen in an oil bath and stirred for 4 hrs. The mixture was cooled to room temperature and diluted with water to afford a precipitate which was isolated by suction filtration. The filter cake was washed with additional water and dried under suction. The solids were then transferred to a vacuum oven and dried at 40° C. for 72 h. The crude product was used as obtained (0.562 g, 75%). LC-MS: (FA) ES− 205.

### Step 2: Methyl 7-hydroxy-2-methyl-1,2,3,4-tetrahydronaphthalene-2-carboxylate Intermediate 85

The titled compound was prepared from Intermediate 84 following the procedure detailed in Example 1, Step 1. LC-MS: (FA) ES+ 221.

### Step 3: Methyl 7-(4-chlorophenoxy)-2-methyl-1,2,3,4-tetrahydronaphthalene-2-carboxylate Intermediate 86

The title compound was prepared from Intermediate 85 and 4-chlorophenylboronic acid following the procedure detailed in Example 18, Step 1. ¹H NMR (400 MHz, CDCl₃) δ ppm 7.29-7.26 (m, 2H), 7.06-7.01 (m, 1H), 6.94-6.89 (m, 2H), 6.75 (dd, J=8.2, 2.6 Hz, 1H), 6.72 (d, J=2.4 Hz, 1H), 3.67 (s, 3H), 3.20 (d, J=16.6 Hz, 1H), 2.82-2.75 (m, 2H), 2.61 (d, J=16.6 Hz, 1H), 2.17 (dddd, J=7.5, 6.3, 6.3, 1.1 Hz, 1H), 1.77 (ddd, J=13.5, 7.1, 6.7 Hz, 1H), 1.28 (s, 3H).

### Step 4: 7-(4-Chlorophenoxy)-2-methyl-1,2,3,4-tetrahydronaphthalene-2-carboxylic acid Intermediate 87

Methyl 7-(4-chlorophenoxy)-2-methyl-1,2,3,4-tetrahydronaphthalene-2-carboxylate (0.207 g, 0.626 mmol) was dissolved in methanol (2 mL) and THF (2 mL). A solution of lithium hydroxide (30 mg, 1.25 mmol) in water (2 mL) was added. The reaction mixture was then stirred at room temperature overnight. The mixture was acidified with dilute aqueous hydrochloric acid and extracted three times with ethyl acetate. The combined extracts were washed with water and brine then dried over sodium sulfate, filtered and concentrated under reduced pressure. The crude product was used as obtained (0.204 g, 100%). LC-MS: (FA) ES− 315.

### Steps 5 and 6: 7-(4-chlorophenoxy)-N-hydroxy-2-methyl-1,2,3,4-tetrahydronaphthalene-2-carboxamide Compound I-124

The title compound was prepared from Intermediate 87 following the two step procedure detailed in Example 3, Steps 5 and 6. LC-MS: (FA) ES+ 332; ¹H NMR (400 MHz, CD₃OD) δ ppm 7.32-7.25 (m, 2H), 7.06 (d, J=9.0 Hz, 1H), 6.94-6.88 (m, 2H), 6.76-6.70 (m, 2H), 3.13 (d, J=16.8 Hz, 1H), 2.93-2.73 (m, 2H), 2.61 (d, J=16.7 Hz, 1H), 2.16 (ddd, J=12.3, 6.1, 6.1 Hz, 1H), 1.75 (ddd, J=14.3, 8.0, 6.8 Hz, 1H), 1.24 (s, 3H).

### Example 52

### N-{6-[(hydroxyamino)carbonyl]-5,6,7,8-tetrahydronaphthalen-2-yl}-4-methylpiperidine-4-carboxamide Compound I-85

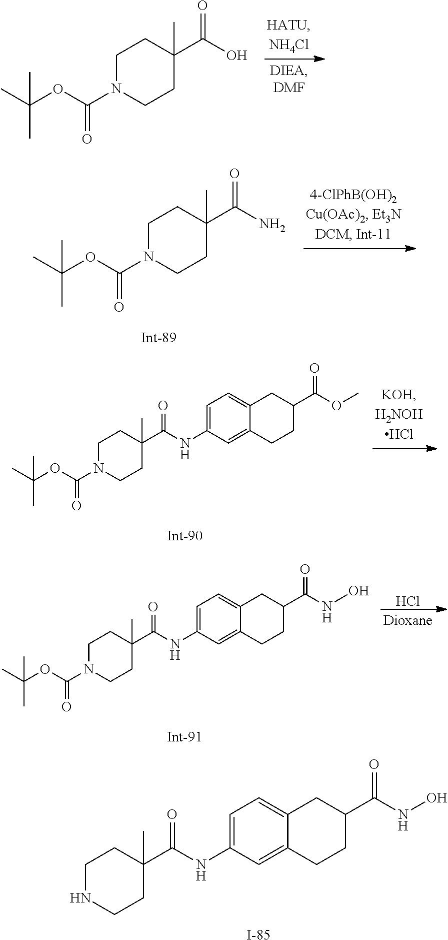

### Step 1: tert-Butyl 4-(aminocarbonyl)-4-methylpiperidine-1-carboxylate Intermediate 89

4-Methyl-4-carboxy-1-N-butoxycarbonyl-piperidine (0.486 g, 2 mmol) was dissolved in N,N-dimethylformamide (20 mL). N,N-diisopropylethylamine (1.4 mL, 8.2 mmol) was added followed by N,N,N′,N′-tetramethyl-O-(7-azabenzotriazol-1-yl)uronium hexafluorophosphate (1.2 g, 3.1 mmol). The reaction solution was stirred at room temperature 20 min and then ammonium chloride (0.22 g, 4.1 mmol) was added. The reaction mixture was stirred overnight at room temperature and then concentrated under reduced pressure. The residue was partitioned between ethyl acetate (100 mL) and water (150 mL). The phases were slow to settle. The phases were separated and the aqueous phase was extracted with additional ethyl acetate. The extracts were combined, washed with 1N HCl, saturated aqueous sodium bicarbonate solution, water and brine then dried over sodium sulfate, filtered and concentrated under reduced pressure. The crude reside was purified by silica gel chromatography (methylene chloride to 90/10 methylene chloride/methanol gradient) to afford the product as a light pink solid (333 mg, 68%).

### Step 2: tert-butyl 4-({[6-(methoxycarbonyl)-5,6,7,8-tetrahydronaphthalen-2-yl]amino}carbonyl)-4-methylpiperidine-1-carboxylate Intermediate 90

Tris(dibenzylideneacetone)dipalladium(0) (27 mg, 0.03 mmol), Xantphos (51 mg, 0.089 mmol), tert-butyl 4-(aminocarbonyl)-4-methylpiperidine-1-carboxylate (Int-89, 110 mg, 0.44 mmol) and cesium carbonate (0.193 g, 0.591 mmol) were added to a 2-5 mL microwave vial fitted with stirbar. The vial was capped and purged with argon. A solution of methyl 6-{[(trifluoromethyl)sulfonyl]oxy}-1,2,3,4-tetrahydronaphthalene-2-carboxylate (Int-11, 0.1 g, 0.296 mmol) in 1,4-dioxane (2 mL) was prepared under argon. The solution was added to the microwave vial by syringe. The contents of the vial were sonicated under active argon flow for 2 min and then heated in an oil bath at 100° C. overnight. After cooling to room temperature, the mixture was filtered through celite. The filter pad was generously washed with ethyl acetate and the filtrate was concentrated under reduced pressure. The crude product was purified by silica gel chromatography (hexanes to 60/40 hexanes/ethyl acetate gradient) to afford the product (90 mg, 71%). LC-MS: (AA) ES+ 431.

### Step 3: tert-butyl 4-[({6-[(hydroxyamino)carbonyl]-5,6,7,8-tetrahydronaphthalen-2-yl}amino)carbonyl]-4-methylpiperidine-1-carboxylate Intermediate 91

The title compound was prepared from Intermediate 90 following the procedure detailed in Example 1, Step 4. LC-MS: (FA) ES− 430.

### Step 4: N-{6-[(hydroxyamino)carbonyl]-5,6,7,8-tetrahydronaphthalen-2-yl}-4-methylpiperidine-4-carboxamide I-85

The title compound was prepared from Intermediate 91 following the procedure detailed in Example 33, Step 3. LC-MS: (FA) ES+ 332.

### Example 53

### HDAC6 Enzyme Assay

To measure the inhibition of HDAC6 activity, purified human HDAC6 (BPS Bioscience; Cat. No. 5006) is incubated with substrate Ac-Arg-Gly-Lys(Ac)-AMC peptide (Bachem Biosciences; Cat. No. I-1925) for 1 hour at 30° C. in the presence of test compounds or vehicle DMSO control. The reaction is stopped with the HDAC inhibitor trichostatin A (Sigma; Cat. No. T8552) and the amount of Arg-Gly-Lys-AMC generated is quantitated by digestion with trypsin (Sigma; Cat. No. T1426) and subsequent measurement of the amount of AMC released using a fluorescent plate reader (Pherastar; BMG Technologies) set at Ex 340 nm and Em 460 nm. Concentration response curves are generated by calculating the fluorescence increase in test compound-treated samples relative to DMSO-treated controls, and enzyme inhibition (IC₅₀) values are determined from those curves.

### Example 54

### Nuclear Extract HDAC Assay

As a screen against Class I HDAC enzymes, HeLa nuclear extract (BIOMOL; Cat. No. KI-140) is incubated with Ac-Arg-Gly-Lys(Ac)-AMC peptide (Bachem Biosciences; Cat. No. 1-1925) in the presence of test compounds or vehicle DMSO control. The HeLa nuclear extract is enriched for Class I enzymes HDAC1, -2 and -3. The reaction is stopped with the HDAC inhibitor Trichostatin A (Sigma; Cat. No. T8552) and the amount of Arg-Gly-Lys-AMC generated is quantitated by digestion with trypsin (Sigma; Cat. No. T1426) and subsequent measurement of the amount of AMC released using a fluorescent plate reader (Pherastar; BMG Technologies) set at Ex 340 nm and Em 460 nm. Concentration response curves are generated by calculating the fluorescence increase in test compound-treated samples relative to DMSO-treated controls, and enzyme inhibition (IC₅₀) values are determined from those curves.

### Example 55

### Western Blot and Immunofluorescence Assays

Cellular potency and selectivity of compounds are determined using a published assay (Haggarty et al., Proc. Natl. Acad. Sci. USA 2003, 100 (8): 4389-4394) using Hela cells (ATCC cat#CCL-2™) which are maintained in MEM medium (Invitrogen) supplemented with 10% FBS; or multiple myeloma cells RPMI-8226 (ATCC cat#CCL-155™) which are maintained in RPMI 1640 medium (Invitrogen) supplemented with 10% FBS. Briefly, cells are treated with inhibitors for 6 or 24 h and either lysed for Western blotting, or fixed for immunofluorescence analyses. HDAC6 potency is determined by measuring K40 hyperacetylation of alpha-tubulin with an acetylation selective monoclonal antibody (Sigma cat#T7451) in IC50 experiments. Selectivity against Class I HDAC activity is determined similarly using an antibody that recognizes hyperacetylation of histone H4 (Upstate cat#06-866) in the Western blotting assay or nuclear acetylation (Abcam cat#ab21623) in the immunofluorescence assay.

### Example 56

### In Vivo Tumor Efficacy Model

Female NCr-Nude mice (age 6-8 weeks, Charles River Labs) are aseptically injected into the subcutaneous space in the right dorsal flank with 1.0-5.0×10⁶ cells (SKOV-3, HCT-116, BxPC3) in 100 μL of a 1:1 ratio of serum-free culture media (Sigma Aldrich) and BD Matrigel™ (BD Biosciences) using a 1 mL 26⅜ gauge needle (Becton Dickinson Ref#309625). Alternatively, some xenograft models require the use of more immunocompromised strains of mice such as CB-17 SCID (Charles River Labs) or NOD-SCID (Jackson Laboratory). Furthermore, some xenograft models require serial passaging of tumor fragments in which small fragments of tumor tissue (approximately 1 mm³) are implanted subcutaneously in the right dorsal flank of anesthetized (3-5% isoflourane/oxygen mixture) NCr-Nude, CB-17 SCID or NOD-SCID mice (age 5-8 weeks, Charles River Labs or Jackson Laboratory) via a 13-ga trocar needle (Popper & Sons 7927). Tumor volume is monitored twice weekly with Vernier calipers. The mean tumor volume is calculated using the formula V=W²×L/2. When the mean tumor volume is approximately 200 mm³, the animals are randomized into treatment groups of ten animals each. Drug treatment typically includes the test compound as a single agent, and may include combinations of the test compound and other anticancer agents. Dosing and schedules are determined for each experiment based on previous results obtained from pharmacokinetic/pharmacodynamic and maximum tolerated dose studies. The control group will receive vehicle without any drug. Typically, test compound (100-200 μL) is administered via intravenous (27-ga needle), oral (20-ga gavage needle) or subcutaneous (27-ga needle) routes at various doses and schedules. Tumor size and body weight are measured twice a week and the study is terminated when the control tumors reach approximately 2000 mm³, and/or if tumor volume exceeds 10% of the animal body weight or if the body weight loss exceeds 20%.

The differences in tumor growth trends over time between pairs of treatment groups are assessed using linear mixed effects regression models. These models account for the fact that each animal is measured at multiple time points. A separate model is fit for each comparison, and the areas under the curve (AUC) for each treatment group are calculated using the predicted values from the model. The percent decrease in AUC (dAUC) relative to the reference group is then calculated. A statistically significant P value suggests that the trends over time for the two treatment groups are different.

The tumor measurements observed on a date pre-specified by the researcher (typically the last day of treatment) are analyzed to assess tumor growth inhibition. For this analysis, a T/C ratio is calculated for each animal by dividing the tumor measurement for the given animal by the mean tumor measurement across all control animals. The T/C ratios across a treatment group are compared to the T/C ratios of the control group using a two-tailed Welch's t-test. To adjust for multiplicity, a False Discovery Rate (FDR) is calculated for each comparison using the approach described by Benjamini and Hochberg, J.R. Stat. Soc. B 1995, 57:289-300.

As detailed above, compounds of the invention inhibit HDAC6. In certain embodiments, compounds of the invention inhibit HDAC6 with the percent inhibition at a concentration of 0.412 μM shown in the table below.

| | Percent Inhibition at |
|---|---|
| Compound | 0.412 μM Compound |
| I-1 | 8 |
| I-2 | 90 |
| I-3 | 33 |
| I-4 | 85 |
| I-5 | 58 |
| I-6 | 53 |
| I-7 | 57 |
| I-8 | 73 |
| I-9 | 74 |
| I-10 | 32 |
| I-11 | 62 |
| I-12 | 61 |
| I-13 | 74 |
| I-14 | 67 |
| I-15 | 25 |
| I-16 | 13 |
| I-17 | 60 |
| I-19 | 23 |
| I-20 | 61 |
| I-21 | 80 |
| I-22 | 72 |
| I-23 | 70 |
| I-24 | 86 |
| I-25 | 27 |
| I-26 | 71 |
| I-27 | 53 |
| I-28 | 65 |
| I-29 | 81 |
| I-30 | 86 |
| I-31 | 79 |
| I-32 | 91 |
| I-33 | 57 |
| I-34 | 57 |
| I-35 | 87 |
| I-36 | 37 |
| I-37 | 55 |
| I-38 | 57 |
| I-39 | 76 |
| I-40 | 46 |
| I-41 | 64 |
| I-42 | 62 |
| I-43 | 19 |
| I-44 | 34 |
| I-45 | 79 |
| I-46 | 78 |
| I-47 | 74 |
| I-48 | 73 |
| I-49 | 82 |
| I-50 | 84 |
| I-51 | 88 |
| I-52 | 74 |
| I-53 | 83 |
| I-54 | 82 |
| I-55 | 93 |
| I-56 | 82 |
| I-57 | 73 |
| I-58 | 87 |
| I-59 | 76 |
| I-60 | 75 |
| I-61 | 73 |
| I-62 | 82 |
| I-63 | 90 |
| I-64 | 90 |
| I-65 | 68 |
| I-66 | 80 |
| I-67 | 81 |
| I-68 | 76 |
| I-69 | 79 |
| I-70 | 78 |
| I-71 | 74 |
| I-72 | 87 |
| I-73 | 64 |
| I-74 | 85 |
| I-75 | 77 |
| I-76 | 0 |
| I-77 | 68 |
| I-78 | 48 |
| I-79 | 43 |
| I-80 | 43 |
| I-81 | 89 |
| I-82 | 83 |
| I-83 | 37 |
| I-84 | 38 |
| I-18 | 54 |
| I-85 | 74 |
| I-86 | 73 |
| I-87 | 85 |
| I-88 | 79 |
| I-89 | 66 |
| I-90 | 85 |
| I-91 | 77 |
| I-92 | 67 |
| I-93 | 79 |
| I-94 | 62 |
| I-95 | 78 |
| I-96 | 79 |
| I-97 | 81 |
| I-98 | 78 |
| I-99 | 82 |
| I-100 | 84 |
| I-101 | 82 |
| I-102 | 76 |
| I-103 | 72 |
| I-104 | 78 |
| I-105 | 61 |
| I-106 | 83 |
| I-107 | 86 |
| I-108 | 75 |
| I-109 | 82 |
| I-110 | 83 |
| I-111 | 74 |
| I-112 | 58 |
| I-113 | 89 |
| I-114 | 76 |
| I-115 | 83 |
| I-116 | 82 |
| I-117 | 74 |
| I-118 | 77 |
| I-119 | 69 |
| I-120 | 55 |
| I-121 | 54 |
| I-122 | 34 |
| I-123 | 61 |
| I-124 | 16 |

As detailed above, compounds of the invention are selective for HDAC6 over other Class I HDAC enzymes. In some embodiments, the ratio of HDAC IC50 (as obtained in the nuclear extract assay described above) to HDAC6 IC50 is less than 5 (HDAC IC50/HDAC6 IC50). In certain embodiments, the ratio of HDAC IC50 to HDAC6 IC50 is between 5 and 10. In certain embodiments, the ratio of HDAC IC50 to HDAC6 IC50 is between 10 and 100.

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of examples.

What is claimed is:

1. A compound of formula (I): 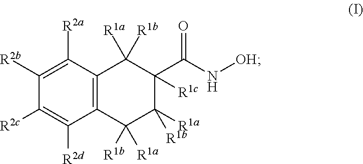
or a pharmaceutically acceptable salt thereof;
wherein:
each occurrence of R^{1a} is independently hydrogen, fluoro, —O—C₁₋₄ alkyl, C₁₋₄ alkyl, or C₁₋₄ fluoroalkyl;
each occurrence of R^{1b} is independently hydrogen, fluoro, C₁₋₄ alkyl, or C₁₋₄ fluoroalkyl;
or one occurrence of R^{1a} and one occurrence of R^{1b} on the same carbon atom can be taken together to form ═O or a 3-6 membered cycloaliphatic;
R^{1c} is hydrogen, fluoro, —O—C₁₋₄ alkyl, hydroxy, C₁₋₄ alkyl, or C₁₋₄ fluoroalkyl;
R^{2a} is G or R¹;
R^{2b} is G or R¹;
R^{2c} is G or R¹;
R^{2d} is G or R¹;
provided that one and only one of R^{2a}, R^{2b}, R^{2c}, and R^{2d} is G;
each occurrence of R¹ is independently hydrogen, chloro, fluoro, —O—C₁₋₄ alkyl, cyano, hydroxy, —C(O)NH₂, —N(C₁₋₄ alkyl)₂, —NH(C₁₋₄ alkyl), —NH₂, C₁₋₄ alkyl, or C₁₋₄ fluoroalkyl;
G is hydrogen, —R³, —V₁—R³, —V₁-L₁-R³, -L₁-V₁—R³, or -L₁-R³;
L₁ is an unsubstituted or substituted C₁₋₃ alkylene chain;
V₁ is —C(O)—, —C(S)—, —C(O)—N(R^{4a})—, —C(O)—O—, —N(R^{4a})—, —N(R^{4a})—C(O)—, —N(R^{4a})—SO₂—, —O—, —N(R^{4a})—C(O)—N(R^{4a})—, —N(R^{4a})—C(O)—O—, —O—C(O)—N(R^{4a})—, or —N(R^{4a})—SO₂—N(R^{4a})—;
R³ is unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur;
each occurrence of R^{4a} is independently hydrogen, or unsubstituted or substituted C₁₋₄ aliphatic; and
provided that the compound is other than 1,2,3,4-tetrahydro-N-hydroxy-1-oxo-2-naphthalenecarboxamide.

2. The compound of claim 1, wherein G is —R³, —V₁—R³, —V₁-L₁-R³, -L₁-V₁—R³, or -L₁-R³.

3. The compound of claim 2, wherein:
R^{2a} is R¹;
R^{2b} is G;
R^{2c} is R¹; and
R^{2d} is R¹.

4. The compound of claim 2, wherein:
R^{2a} is R¹;
R^{2b} is R¹;
R^{2c} is G; and
R^{2d} is R¹.

5. The compound of claim 2, wherein:
G is —V₁—R³, -L₁-R³, or —R³;
L₁ is —CH₂— or —CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

6. The compound of claim 2, wherein:
each occurrence of R^{1a} is independently hydrogen, fluoro, trifluoromethyl, or methyl;
each occurrence of R^{1b} is hydrogen;
R^{1c} is hydrogen, hydroxy, fluoro, trifluoromethyl, or methyl;
each occurrence of R¹ is independently hydrogen, chloro, fluoro, cyano, hydroxy, methoxy, ethoxy, trifluoromethyl, methyl, or ethyl.

7. The compound of claim 2, wherein:
each substitutable carbon chain atom in R³ is unsubstituted or substituted with 1-2 occurrences of —R^{5dd};
each substitutable saturated ring carbon atom in R³ is unsubstituted or substituted with ═O, ═C(R⁵)₂, or —R^{5aa};
each substitutable unsaturated ring carbon atom in R³ is unsubstituted or is substituted with —R^{5a};
each substitutable ring nitrogen atom in R³ is unsubstituted or substituted with —R^{9b};
each R^{5a} is independently halogen, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁶, —NR⁴C(O)N(R⁴)₂, —NR⁴CO₂R⁶, —OC(O)N(R⁴)₂, —C(O)R⁶, —C(O)N(R⁴)₂, —N(R⁴)SO₂R⁶, —N(R⁴)SO₂N(R⁴)₂, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two adjacent R^{5a}, taken together with the intervening ring atoms, form an unsubstituted or substituted fused 5-10 membered aromatic ring or an unsubstituted or substituted 4-10 membered non-aromatic ring having 0-3 heteroatoms independently selected from nitrogen, oxygen, and sulfur;
each occurrence of R^{5aa} is independently chloro, fluoro, hydroxy, unsubstituted or substituted C₁₋₆ aliphatic, —O(C₁₋₆ alkyl), —C₁₋₆ fluoroalkyl, —O—C₁₋₆ fluoroalkyl, cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, —NHC(O)N(C₁₋₆ alkyl)₂, or —NHS(O)₂C₁₋₆ alkyl;
each occurrence of R^{5dd} is independently fluoro, hydroxy, —O(C₁₋₆ alkyl), cyano, —N(R⁴)₂, —C(O)(C₁₋₆ alkyl), —CO₂H, —C(O)NH₂, —C(O)NH(C₁₋₆ alkyl), —C(O)N(C₁₋₆ alkyl)₂, —NHC(O)C₁₋₆ alkyl, —NHC(O)OC₁₋₆ alkyl, —NHC(O)NHC₁₋₆ alkyl, —NHC(O)N(C₁₋₆ alkyl)₂, or —NHS(O)₂C₁₋₆ alkyl;
each R⁴ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; or two R⁴ on the same nitrogen atom, taken together with the nitrogen atom, form an unsubstituted or substituted 5- to 6-membered heteroaryl or an unsubstituted or substituted 4- to 8-membered heterocyclyl having, in addition to the nitrogen atom, 0-2 ring heteroatoms selected from nitrogen, oxygen, and sulfur;
each R⁵ is independently hydrogen, unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur;
each R⁶ is independently unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur;
each R^{9b} is independently —C(O)R⁶, —C(O)N(R⁴)₂, —CO₂R⁶, —SO₂R⁶, —SO₂N(R⁴)₂, unsubstituted C₃₋₁₀ cycloaliphatic, C₃₋₁₀ cycloaliphatic substituted with 1-2 independent occurrences of R⁷ or R⁸, unsubstituted C₁₋₆ aliphatic, or C₁₋₆ aliphatic substituted with 1-2 independent occurrences of R⁷ or R⁸;
each R⁷ is independently unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-5 heteroatoms independently selected from nitrogen, oxygen, and sulfur; and
each R⁸ is independently chloro, fluoro, —OH, —O(C₁₋₆ alkyl), —CN, —N(R⁴)₂, —C(O)(C₁₋₆ alkyl), —CO₂H, —CO₂(C₁₋₆ alkyl), —C(O)NH₂, —C(O)NH(C₁₋₆ alkyl) or —C(O)N(C₁₋₆ alkyl)₂.

8. The compound of claim 7, wherein:
each substitutable saturated ring carbon atom in R³ is unsubstituted or substituted with —R^{5aa};
the total number of R^{5a} and R^{5aa} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted —O—C₁₋₆ alkyl, —O—C₁₋₆ alkyl substituted with 1-2 independent occurrences of R⁷ or R⁸, C₁₋₆ fluoroalkyl, —O—C₁₋₆ fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆ alkyl)₂, 3-10-membered cycloaliphatic substituted with 0-2 occurrences of —R^{7a}, 4-10-membered heterocyclyl having 1-4 heteroatoms independently selected from nitrogen, oxygen, and sulfur substituted with 0-2 occurrences of —R^{7a}, 6-10-membered aryl substituted with 0-2 occurrences of —R^{7a}, or 5-10-membered heteroaryl having 1-5 heteroatoms independently selected from nitrogen, oxygen, and sulfur substituted with 0-2 occurrences of —R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆ alkyl)₂, —C(O)NHC₁₋₆ alkyl, —C(O)N(C₁₋₆ alkyl)₂, —NHC(O)NHC₁₋₆ alkyl, —NHC(O)N(C₁₋₆ alkyl)₂, or —NHS(O)₂C₁₋₆ alkyl.

9. The compound of claim 7, represented by formula (I-a):

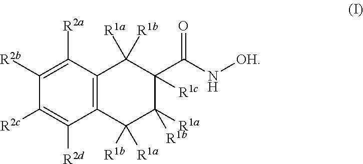

10. The compound of claim 9, wherein:
each occurrence of R^{1a} is independently hydrogen, fluoro, trifluoromethyl, or methyl;
each occurrence of R^{1b} is hydrogen;
R^{1c} is hydrogen, hydroxy, fluoro, trifluoromethyl, or methyl; and
each occurrence of R¹ is independently hydrogen, chloro, fluoro, cyano, hydroxy, methoxy, ethoxy, trifluoromethyl, methyl, or ethyl.

11. The compound of claim 10, wherein:
G is —V₁—R³, -L₁-R³, or —R³;
L₁ is —CH₂— or —CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

12. The compound of claim 10, wherein:
each occurrence of R^{1a} is hydrogen;
R^{1c} is hydrogen; and
each occurrence of R¹ is hydrogen.

13. The compound of claim 10, wherein:
each substitutable saturated ring carbon atom in R³ is unsubstituted or substituted with —R^{5aa};
the total number of R^{5a} and R^{5aa} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted —O—C₁₋₆ alkyl, —O—C₁₋₆ alkyl substituted with 1-2 independent occurrences of R⁷ or R⁸, C₁₋₆ fluoroalkyl, fluoroalkyl, —NHC(O)R⁶, —C(O)NH(R⁴), —NHC(O)O—C₁₋₆ alkyl, —NHC(O)NHC₁₋₆alkyl, —NHS(O)₂C₁₋₆ alkyl, —NHC₁₋₆ alkyl, —N(C₁₋₆ alkyl)₂, 3-10-membered cycloaliphatic substituted with 0-2 occurrences of —R^{7a}, 4-10-membered heterocyclyl having 1-4 heteroatoms independently selected from nitrogen, oxygen, and sulfur substituted with 0-2 occurrences of —R^{7a}, 6-10-membered aryl substituted with 0-2 occurrences of R^{7a}, or 5-10-membered heteroaryl having 1-5 heteroatoms independently selected from nitrogen, oxygen, and sulfur substituted with 0-2 occurrences of —R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆alkyl)₂, —C(O)NHC₁₋₆ alkyl, —C(O)N(C₁₋₆alkyl)₂, —NHC(O)NHC₁₋₆ alkyl, —NHC(O)N(C₁₋₆alkyl)₂, or —NHS(O)₂C₁₋₆ alkyl.

14. The compound of claim 7, represented by formula (I-b):

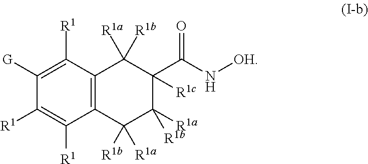

15. The compound of claim 14, wherein:
each occurrence of R^{1a} is independently hydrogen, fluoro, trifluoromethyl, or methyl.
each occurrence of R^{1b} is hydrogen;
R^{1c} is hydrogen, hydroxy, fluoro, trifluoromethyl, or methyl; and
each occurrence of R¹ is independently hydrogen, chloro, fluoro, cyano, hydroxy, methoxy, ethoxy, trifluoromethyl, methyl, or ethyl.

16. The compound of claim 15, wherein:
G is —V₁—R³, -L₁-R³, or —R³;
L₁ is —CH₂— or —CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

17. The compound of claim 15, wherein:
each occurrence of R^{1a} is hydrogen;
R^{1c} is hydrogen; and
each occurrence of R¹ is hydrogen.

18. The compound of claim 15, wherein:
each substitutable saturated ring carbon atom in R³ is unsubstituted or substituted with —R^{5a};
the total number of R^{5a} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted —O—C₁₋₆ alkyl, —O—C₁₋₆ alkyl substituted with 1-2 independent occurrences of R⁷ or R⁸, C₁₋₆ fluoroalkyl, —O—C₁₋₆ fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆alkyl)₂, 3-10-membered cycloaliphatic substituted with 0-2 occurrences of —R^{7a}, 4-10-membered heterocyclyl having 1-4 heteroatoms independently selected from nitrogen, oxygen, and sulfur substituted with 0-2 occurrences of —R^{7a}, 6-10-membered aryl substituted with 0-2 occurrences of —R^{7a}, or 5-10-membered heteroaryl having 1-5 heteroatoms independently selected from nitrogen, oxygen, and sulfur substituted with 0-2 occurrences of —R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆-fluoroalkyl, cyano, hydroxy, —CO₂H, —NHC(O)C₁₋₆ alkyl, —NHC₁₋₆ alkyl, —N(C₁₋₆alkyl)₂, —C(O)NHC₁₋₆ alkyl, —C(O)N(C₁₋₆alkyl)₂, —NHC(O)NHC₁₋₆ alkyl, —NHC(O)N(C₁₋₆alkyl)₂, or —NHS(O)₂C₁₋₆ alkyl.

19. The compound of claim 7, represented by formula (I-c):

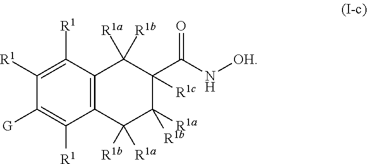

20. The compound of claim 19, wherein:
each occurrence of R^{1a} is independently hydrogen, fluoro, trifluoromethyl, or methyl;
each occurrence of R^{1b} is hydrogen;
R^{1c} is hydrogen, hydroxy, fluoro, trifluoromethyl, or methyl; and
each occurrence of R¹ is independently hydrogen, chloro, fluoro, cyano, hydroxy, methoxy, ethoxy, trifluoromethyl, methyl, or ethyl.

21. The compound of claim 20, wherein:
G is —V₁—R³, -L₁-R³, or —R³;
L₁ is —CH₂— or —CH₂CH₂—; and
V₁ is —N(R^{4a})—, —N(R^{4a})—C(O)—, —C(O)—N(R^{4a})—, —N(R^{4a})—SO₂—, —O—, —N(R^{4a})—C(O)—O—, or —N(R^{4a})—C(O)—N(R^{4a})—.

22. The compound of claim 20, wherein:
each occurrence of R^{1a} is hydrogen;
R^{1c} is hydrogen; and
each occurrence of R¹ is hydrogen.

23. The compound of claim 20, wherein:
each substitutable saturated ring carbon atom in R³ is unsubstituted or substituted with —R^{5a};
the total number of R^{5a} substituents is p;
p is 1-4;
each R^{5a} is independently halogen, cyano, nitro, hydroxy, unsubstituted C₁₋₆ aliphatic, C₁₋₆ aliphatic substituted with 1-2 independent occurrences of R⁷ or R⁸, unsubstituted —O—C₁₋₆ alkyl, —O—C₁₋₆ alkyl substituted with 1-2 independent occurrences of R⁷ or R⁸, C₁₋₆ fluoroalkyl, —O—C₁₋₆ fluoroalkyl, —NHC(O)R⁶, —C(O)NH(R⁴), —NHC(O)O—C₁₋₆ alkyl, —NHC(O)NHC₁₋₆ alkyl, —NHS(O)₂C₁₋₆ alkyl, —NHC₁₋₆ alkyl, —N(C₁₋₆alkyl)₂, 3-10-membered cycloaliphatic substituted with 0-2 occurrences of —R^{7a}, 4-10-membered heterocyclyl having 1-4 heteroatoms independently selected from nitrogen, oxygen, and sulfur substituted with 0-2 occurrences of —R^{7a}, 6-10-membered aryl substituted with 0-2 occurrences of —R^{7a}, or 5-10-membered heteroaryl having 1-5 heteroatoms independently selected from nitrogen, oxygen, and sulfur substituted with 0-2 occurrences of —R^{7a}, and
each occurrence of R^{7a} is independently chloro, fluoro, C₁₋₆ aliphatic, C₁₋₆ fluoroalkyl, —O—C₁₋₆ alkyl, —O—C₁₋₆ fluoroalkyl, cyano, hydroxy, —CO₂H, —NHC(O)C₁₋₆ alkyl, —NHC₁₋₆ alkyl, —N(C₁₋₆ alkyl)₂, —C(O)NHC₁₋₆ alkyl, —C(O)N(C₁₋₆alkyl)₂, —NHC(O)NHC₁₋₆ alkyl, —NHC(O)N(C₁₋₆alkyl)₂, or —NHS(O)₂C₁₋₆ alkyl.

24. A pharmaceutical composition comprising a compound of claim 1 and a pharmaceutically acceptable carrier.

25. A method of treating a proliferative disorder in a patient comprising administering to said patient a therapeutically effective amount of a compound of claim 1.

26. The method of claim 25, wherein the proliferative disorder is breast cancer, lung cancer, ovarian cancer, multiple myeloma, acute myelogenous leukemia, or acute lymphoblastic leukemia.

27. A method for inhibiting HDAC6 activity in a patient comprising administering a pharmaceutical composition comprising an amount of a compound of claim 1 effective to inhibit HDAC6 activity in the patient.
